(19) United States
(12) Patent Application Publication
(10) Pub. No.: US 2023/0058162 A1
(43) Pub. Date: Feb. 23, 2023

(54) MULTIMERIC CORONAVIRUS BINDING MOLECULES AND USES THEREOF

(71) Applicant: IGM Biosciences, Inc., Mountain View, CA (US)

(72) Inventors: Paul R. Hinton, Sunnyvale, CA (US); Bruce Alan Keyt, Hillsborough, CA (US); Dean Ng, San Francisco, CA (US); Kathy Miller, San Francisco, CA (US); Deepal Pandya, Fremont, CA (US); Stephen F. Carroll, Walnut Creek, CA (US)

(21) Appl. No.: 17/386,397

(22) Filed: Jul. 27, 2021

Related U.S. Application Data
(60) Provisional application No. 63/057,244, filed on Jul. 27, 2020.
(60) Provisional application No. 63/133,153, filed on Dec. 31, 2020.
(60) Provisional application No. 63/133,276, filed on Jan. 1, 2021.
(60) Provisional application No. 63/150,491, filed on Feb. 17, 2021.

(51) Int. Cl. C07K 16/10

(52) U.S. Cl. CPC C07K 16/10; C07K 2317/35; A61K 2039/505; C07K 2317/76; C07K 2317/565; C07K 2317/92

(57) ABSTRACT

This disclosure provides multimeric binding molecules that bind to a human coronavirus, e.g., MERS-CoV, SARS-CoV or SARS-CoV-2. This disclosure also provides compositions comprising the multimeric binding molecules, polynucleotides that encode the multimeric binding molecules, and host cells that can produce the binding molecules. Further this disclosure provides methods of using the multimeric binding molecules, including methods for treating and preventing human coronavirus disease, e.g., coronavirus disease 2019 (COVID-19).

### CROSS-REFERENCE TO RELATED APPLICATIONS

This application claims the benefit of U.S. Provisional Patent Application Ser. Nos. 63/057,244, filed Jul. 27, 2020; 63/133,153, filed Dec. 31, 2020; 63/133,276, filed Jan. 1, 2021; and 63/150,491, filed Feb. 17, 2021, which are all each incorporated herein by reference in their entireties.

### SEQUENCE LISTING

The instant application contains a Sequence Listing which has been submitted electronically in ASCII format and is hereby incorporated by reference in its entirety. The ASCII copy, created on Jul. 27, 2021, is named 032US1-Sequence-Listing.txt and is 803,135 bytes in size.

### BACKGROUND

Antibodies and antibody-like molecules that can multimerize, such as IgA and IgM antibodies, have emerged as promising drug candidates, e.g., in the fields of immuno-oncology and infectious diseases, allowing for improved specificity, improved avidity, and the ability to bind to multiple binding targets. See, e.g., U.S. Pat. Nos. 9,951,134, 9,938,347, 10,351,631, 10,400,038, and 10,899,935, U.S. Patent Application Publication Nos. US 2019-0100597, US 2018-0009897, US 2019-0330374, US 2019-0330360, US 2019-0338040, US 2019-0338041, US 2019-0185570, US 2018-0265596, US 2018-0118816, US 2018-0118814, and US 2019-0002566, and PCT Publication Nos. WO 2018/187702, and WO 2019/165340, the contents of which are incorporated herein by reference in their entireties.

Severe acute respiratory syndrome coronavirus 2 (SARS-CoV-2) is a single stranded, positive sense enveloped RNA virus. SARS-CoV-2 causes Coronavirus Disease 2019 (COVID-19), which is currently causing a global pandemic. COVID-19 is highly contagious and commonly causes fever, cough, and shortness of breath, and can lead to pneumonia, blood clots, organ failure, and death. The four main structural proteins of the SARS-CoV-2 include spike (S), envelope (E), membrane (M), and nucleic capsid (N).

The trimeric S protein binds the angiotensin-converting enzyme 2 (ACE2) receptor, alters its conformation to a fusogenic protein, which facilitates fusion of the cellular and viral membranes and thereby enables SARS-CoV-2 to enter cells. The S protein comprises two units: S1 and S2, with the S1 domain comprising the receptor-binding domain (RBD). See, e.g., Lan, J., et al., Nature 581:215-220 (2020). The S protein and specifically the RBD are required for entry into cells, which has made the RBD a favored target of potential therapeutic monoclonal antibodies. Unfortunately, even the IgG antibodies having very potent neutralizing activity against the RBD of SARS-CoV-2 need to be administered by infusion at high doses (up to 8 grams per dose) to effectively treat COVID-19 patients (Weinreich, D M, et al., N Engl J Med doi: 10.1056/NEJMoa2035002 12020 (2021); Chen, et al., N Engl J Med doi: 10.1056/NEJMoa2029849 (2021)).

Despite this interest, antibody-dependent enhancement (ADE), also known as antibody-mediated enhancement (AME), of disease caused by SARS-CoV-2 is a concern and has been demonstrated in infections with other coronaviruses. See, e.g., Houser, K. V., et al., PLoS Pathog. 13: Article e1006565 (2017) (MERS-CoV); Weiss, R. C., and F. W. Scott Comp. Immunol. Microbiol. Infect. Dis 4:175-189 (1981) (feline infectious peritonitis virus); and Kam, Y. W., et al., Vaccine 25:729-740 (2007) (SARS-CoV). For example, Kam et al., showed that antibodies against the SARS-CoV S glycoprotein trimer were able to mediate entry of antibody-bound virus into B cells via FcγRII. If Fcγ receptor dependent entry into cells occurs with antibodies against SARS-CoV-2, conventional IgG antibody therapy may make the infection worse than no treatment at all.

In addition to the difficulties in developing therapeutic monoclonal antibodies in general, current practices for treating COVID-19 are severely limited. Remdesivir, convalescent plasma, and hyperimmune globulin have all received FDA emergency use authorizations. However, remdesivir has only been shown to shorten the hospitalization duration a short period of time. Similarly, convalescent plasma and hyperimmune globulin have shown some early signs of improving COVID-19 symptoms, but results are limited, and the plasma is obtained from patients and not manufactured, making it difficult to obtain and scale up and it inherently has lot to lot variations.

Other human coronaviruses also constitute public health risks. Middle East respiratory syndrome coronavirus (MERS-CoV) is endemic in the Middle East but can be transmitted to other countries by travel activity. For example, the introduction of MERS-CoV into the Republic of Korea by an infected traveler resulted in a hospital outbreak of MERS that entailed 186 cases and 38 deaths (Kleine-Weber et al., J. Virol. 2019, 93(2):e01381-18).

There remains an urgent need for therapeutics to treat and/or prevent human coronavirus infections, e.g., SARS, COVID-19, and diseases caused by MERS-CoV.

### SUMMARY

Provided herein is a multimeric binding molecule that includes two to six bivalent binding units or variants or fragments thereof, where each binding unit includes two IgM or IgA heavy chain constant regions or multimerizing fragments or variants thereof, each associated with a binding domain, where three to twelve of the binding domains are identical and specifically bind to a human coronavirus, and where the binding molecule is more potent than a bivalent reference IgG antibody that includes two of the binding domains that specifically bind to the human coronavirus. In some embodiments, the human coronavirus is SARS-CoV, MERS-CoV, SARS-CoV-2, variants thereof, derivatives thereof, or any combination thereof.

In some embodiments, the multimeric binding molecule can neutralize infectivity of the human coronavirus at a greater potency than the bivalent reference IgG antibody that includes two of the binding domains that specifically bind to the human coronavirus. In some embodiments, the multimeric binding molecule can neutralize infectivity of the human coronavirus at a lower 50% effective concentration (EC₅₀) than the bivalent reference IgG antibody. In some embodiments, the EC₅₀ is at least two-fold, at least five-fold, at least ten-fold, at least fifty-fold, at least 100-fold, at least 500-fold, or at least 1000-fold lower than the EC₅₀ of the bivalent reference IgG antibody.

In some embodiments, the binding molecule can inhibit binding of the human coronavirus to its receptor at a lower 50% inhibitory concentration (IC₅₀) than the bivalent reference IgG antibody. In some embodiments, the human coronavirus bound by the multimeric binding molecule is SARS-CoV or SARS-CoV-2 and the receptor is human angiotensin-converting enzyme 2 (ACE2). In some embodiments, the human coronavirus is MERS-CoV and the receptor is human dipeptidyl-peptidase 4 (DPP4).

In some embodiments, the three to twelve binding domains of the multimeric binding molecule of the disclosure bind a human coronavirus structural protein or fragment thereof. In some embodiments the human coronavirus structural protein is a nucleocapsid (N) protein, a membrane (M) protein, an envelope (E) protein, a spike (S) protein, any fragment thereof, any subunit thereof, or any combination thereof. In some embodiments, the human coronavirus structural protein is the S protein, a fragment thereof, or a subunit thereof. In some embodiments, the three to twelve binding domains of the multimeric binding protein specifically bind to the S protein subunit 1 (S1), the S protein receptor binding domain (RBD), the S protein subunit 2 (S2), the S protein furin cleavage site, or any combination thereof. In certain embodiments the three to twelve binding domains of the multimeric binding protein specifically bind to the SARS-CoV-2 RBD.

In some embodiments, the three to twelve identical binding domains of the multimeric binding molecule are immunoglobulin antigen binding domains that include a heavy chain variable region (VH) and a light chain variable region (VL). In some embodiments, each binding unit includes two heavy chains each including a VH and two light chains each including a VL. In some embodiments, the immunoglobulin antigen-binding domains are human or humanized antigen binding domains. In some embodiments, the three to twelve identical binding domains of the multimeric binding molecule are single-domain variable regions (VHH), and where each binding unit includes two heavy chains each including the VHH.

In some embodiments, the three to twelve identical binding domains of the multimeric binding molecule each specifically bind to SARS-CoV-2, and include a heavy chain variable region (VH) and a light chain variable region (VL), where the VH and VL include six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 include the CDRs of an antibody that includes the VH and VL of SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 104 and SEQ ID NO: 105, SEQ ID NO: 106 and SEQ ID NO: 107, SEQ ID NO: 108 and SEQ ID NO: 109, SEQ ID NO: 110 and SEQ ID NO: 111, SEQ ID NO: 112 and SEQ ID NO: 113, SEQ ID NO: 114 and SEQ ID NO: 115, SEQ ID NO: 116 and SEQ ID NO: 117, SEQ ID NO: 118 and SEQ ID NO: 119, SEQ ID NO: 120 and SEQ ID NO: 121, SEQ ID NO: 122 and SEQ ID NO: 123, SEQ ID NO: 124 and SEQ ID NO: 125, SEQ ID NO: 126 and SEQ ID NO: 127, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 150 and SEQ ID NO: 151, SEQ ID NO: 152 and SEQ ID NO: 153, SEQ ID NO: 154 and SEQ ID NO: 155, SEQ ID NO: 156 and SEQ ID NO: 157, SEQ ID NO: 158 and SEQ ID NO: 159, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 178 and SEQ ID NO: 179, SEQ ID NO: 180 and SEQ ID NO: 181, SEQ ID NO: 182 and SEQ ID NO: 183, SEQ ID NO: 184 and SEQ ID NO: 185, SEQ ID NO: 186 and SEQ ID NO: 187, SEQ ID NO: 188 and SEQ ID NO: 189, SEQ ID NO: 190 and SEQ ID NO: 191, SEQ ID NO: 192 and SEQ ID NO: 193, SEQ ID NO: 194 and SEQ ID NO: 195, SEQ ID NO: 196 and SEQ ID NO: 197, SEQ ID NO: 198 and SEQ ID NO: 199, SEQ ID NO: 200 and SEQ ID NO: 201, SEQ ID NO: 202 and SEQ ID NO: 203, SEQ ID NO: 204 and SEQ ID NO: 205, SEQ ID NO: 206 and SEQ ID NO: 207, SEQ ID NO: 208 and SEQ ID NO: 209, SEQ ID NO: 210 and SEQ ID NO: 211, SEQ ID NO: 212 and SEQ ID NO: 213, SEQ ID NO: 214 and SEQ ID NO: 215, SEQ ID NO: 216 and SEQ ID NO: 217, SEQ ID NO: 218 and SEQ ID NO: 219, SEQ ID NO: 220 and SEQ ID NO: 221, SEQ ID NO: 222 and SEQ ID NO: 223, SEQ ID NO: 224 and SEQ ID NO: 225, SEQ ID NO: 226 and SEQ ID NO: 227, SEQ ID NO: 228 and SEQ ID NO: 229, SEQ ID NO: 230 and SEQ ID NO: 231, SEQ ID NO: 232 and SEQ ID NO: 233, SEQ ID NO: 234 and SEQ ID NO: 235, SEQ ID NO: 236 and SEQ ID NO: 237, SEQ ID NO: 238 and SEQ ID NO: 239, SEQ ID NO: 240 and SEQ ID NO: 241, SEQ ID NO: 242 and SEQ ID NO: 243, SEQ ID NO: 244 and SEQ ID NO: 245, SEQ ID NO: 246 and SEQ ID NO: 247, SEQ ID NO: 248 and SEQ ID NO: 249, SEQ ID NO: 250 and SEQ ID NO: 251, SEQ ID NO: 252 and SEQ ID NO: 253, SEQ ID NO: 254 and SEQ ID NO: 255, SEQ ID NO: 256 and SEQ ID NO: 257, SEQ ID NO: 258 and SEQ ID NO: 259, SEQ ID NO: 260 and SEQ ID NO: 261, SEQ ID NO: 264 and SEQ ID NO: 265, SEQ ID NO: 266 and SEQ ID NO: 267, SEQ ID NO: 268 and SEQ ID NO: 269, SEQ ID NO: 270 and SEQ ID NO: 271, SEQ ID NO: 272 and SEQ ID NO: 273, SEQ ID NO: 274 and SEQ ID NO: 275, SEQ ID NO: 276 and SEQ ID NO: 277, SEQ ID NO: 278 and SEQ ID NO: 279, SEQ ID NO: 280 and SEQ ID NO: 281, SEQ ID NO: 282 and SEQ ID NO: 283, SEQ ID NO: 292 and SEQ ID NO: 293, SEQ ID NO: 294 and SEQ ID NO: 295, SEQ ID NO: 296 and SEQ ID NO: 297, SEQ ID NO: 298 and SEQ ID NO: 299, SEQ ID NO: 300 and SEQ ID NO: 301, SEQ ID NO: 302 and SEQ ID NO: 303, SEQ ID NO: 304 and SEQ ID NO: 305, SEQ ID NO: 306 and SEQ ID NO: 307, SEQ ID NO: 308 and SEQ ID NO: 309, SEQ ID NO: 310 and SEQ ID NO: 311, SEQ ID NO: 312 and SEQ ID NO: 313, SEQ ID NO: 314 and SEQ ID NO: 315, SEQ ID NO: 316 and SEQ ID NO: 317, SEQ ID NO: 318 and SEQ ID NO: 319, SEQ ID NO: 320 and SEQ ID NO: 321, SEQ ID NO: 322 and SEQ ID NO: 323, SEQ ID NO: 324 and SEQ ID NO: 325, SEQ ID NO: 326 and SEQ ID NO: 327, SEQ ID NO: 328 and SEQ ID NO: 329, SEQ ID NO: 330 and SEQ ID NO: 331, SEQ ID NO: 332 and SEQ ID NO: 333, SEQ ID NO: 336 and SEQ ID NO: 337, SEQ ID NO: 338 and SEQ ID NO: 339, SEQ ID NO: 340 and SEQ ID NO: 341, SEQ ID NO: 342 and SEQ ID NO: 343, SEQ ID NO: 344 and SEQ ID NO: 345, SEQ ID NO: 348 and SEQ ID NO: 349, SEQ ID NO: 350 and SEQ ID NO: 351, SEQ ID NO: 352 and SEQ ID NO: 353, SEQ ID NO: 354 and SEQ ID NO: 355, SEQ ID NO: 356 and SEQ ID NO: 357, SEQ ID NO: 358 and SEQ ID NO: 359, SEQ ID NO: 362 and SEQ ID NO: 363, SEQ ID NO: 364 and SEQ ID NO: 365, SEQ ID NO: 366 and SEQ ID NO: 367, SEQ ID NO: 368 and SEQ ID NO: 369, SEQ ID NO: 370 and SEQ ID NO: 371, SEQ ID NO: 372 and SEQ ID NO: 373, SEQ ID NO: 374 and SEQ ID NO: 375, SEQ ID NO: 376 and SEQ ID NO: 377, SEQ ID NO: 378 and SEQ ID NO: 379, SEQ ID NO: 380 and SEQ ID NO: 381, SEQ ID NO: 382 and SEQ ID NO: 383, SEQ ID NO: 384 and SEQ ID NO: 385, SEQ ID NO: 386 and SEQ ID NO: 387, SEQ ID NO: 388 and SEQ ID NO: 389, SEQ ID NO: 390 and SEQ ID NO: 391, SEQ ID NO: 392 and SEQ ID NO: 393, SEQ ID NO: 394 and SEQ ID NO: 395, SEQ ID NO: 396 and SEQ ID NO: 397, SEQ ID NO: 398 and SEQ ID NO: 399, SEQ ID NO: 400 and SEQ ID NO: 401, SEQ ID NO: 402 and SEQ ID NO: 403, SEQ ID NO: 404 and SEQ ID NO: 405, SEQ ID NO: 406 and SEQ ID NO: 407, SEQ ID NO: 408 and SEQ ID NO: 409, SEQ ID NO: 410 and SEQ ID NO: 411, SEQ ID NO: 412 and SEQ ID NO: 413, SEQ ID NO: 414 and SEQ ID NO: 415, SEQ ID NO: 416 and SEQ ID NO: 417, SEQ ID NO: 418 and SEQ ID NO: 419, SEQ ID NO: 420 and SEQ ID NO: 421, SEQ ID NO: 422 and SEQ ID NO: 423, SEQ ID NO: 424 and SEQ ID NO: 425, SEQ ID NO: 426 and SEQ ID NO: 427, SEQ ID NO: 428 and SEQ ID NO: 429, SEQ ID NO: 430 and SEQ ID NO: 431, SEQ ID NO: 432 and SEQ ID NO: 433, SEQ ID NO: 434 and SEQ ID NO: 435, SEQ ID NO: 436 and SEQ ID NO: 437, SEQ ID NO: 438 and SEQ ID NO: 439, SEQ ID NO: 440 and SEQ ID NO: 441, SEQ ID NO: 442 and SEQ ID NO: 443, SEQ ID NO: 444 and SEQ ID NO: 445, SEQ ID NO: 446 and SEQ ID NO: 447, SEQ ID NO: 448 and SEQ ID NO: 449, SEQ ID NO: 450 and SEQ ID NO: 451, SEQ ID NO: 452 and SEQ ID NO: 453, SEQ ID NO: 454 and SEQ ID NO: 455, SEQ ID NO: 456 and SEQ ID NO: 457, SEQ ID NO: 458 and SEQ ID NO: 459, SEQ ID NO: 460 and SEQ ID NO: 461, SEQ ID NO: 462 and SEQ ID NO: 463, SEQ ID NO: 464 and SEQ ID NO: 465, SEQ ID NO: 466 and SEQ ID NO: 467, SEQ ID NO: 468 and SEQ ID NO: 469, SEQ ID NO: 470 and SEQ ID NO: 471, SEQ ID NO: 472 and SEQ ID NO: 473, SEQ ID NO: 474 and SEQ ID NO: 475, SEQ ID NO: 476 and SEQ ID NO: 477, SEQ ID NO: 478 and SEQ ID NO: 479, SEQ ID NO: 480 and SEQ ID NO: 481, SEQ ID NO: 482 and SEQ ID NO: 483, SEQ ID NO: 484 and SEQ ID NO: 485, SEQ ID NO: 486 and SEQ ID NO: 487, SEQ ID NO: 488 and SEQ ID NO: 489, SEQ ID NO: 490 and SEQ ID NO: 491, SEQ ID NO: 492 and SEQ ID NO: 493, SEQ ID NO: 494 and SEQ ID NO: 495, SEQ ID NO: 496 and SEQ ID NO: 497, SEQ ID NO: 498 and SEQ ID NO: 499, SEQ ID NO: 500 and SEQ ID NO: 501, SEQ ID NO: 502 and SEQ ID NO: 503, SEQ ID NO: 504 and SEQ ID NO: 505, SEQ ID NO: 506 and SEQ ID NO: 507, SEQ ID NO: 508 and SEQ ID NO: 509, SEQ ID NO: 628 and SEQ ID NO: 629, SEQ ID NO: 630 and SEQ ID NO: 631, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, SEQ ID NO: 646 and SEQ ID NO: 647, SEQ ID NO: 648 and SEQ ID NO: 649, SEQ ID NO: 650 and SEQ ID NO: 651, or SEQ ID NO: 652 and SEQ ID NO: 653, respectively with zero, one, or two single amino acid substitutions in one or more of the HCDRs or LCDRs. In some embodiments, the VH and VL of the multimeric binding molecule include amino acid sequences at least 80%, at least 85%, at least 90%, at least 95% or 100% identical to the recited VH and VL amino acid sequences, respectively.

In some embodiments, the three to twelve identical binding domains of the multimeric binding molecule each specifically bind to SARS-CoV-2, and the VH and VL of the multimeric binding molecule include six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 include the CDRs of an antibody that includes the VH and VL of SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 106 and SEQ ID NO: 107, SEQ ID NO: 110 and SEQ ID NO: 111, SEQ ID NO: 118 and SEQ ID NO: 119, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 142 and SEQ ID NO: 143, SEQ ID NO: 146 and SEQ ID NO: 147, SEQ ID NO: 150 and SEQ ID NO: 151, SEQ ID NO: 156 and SEQ ID NO: 157, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 180 and SEQ ID NO: 181, SEQ ID NO: 182 and SEQ ID NO: 183, SEQ ID NO: 186 and SEQ ID NO: 187, SEQ ID NO: 188 and SEQ ID NO: 189, SEQ ID NO: 194 and SEQ ID NO: 195, SEQ ID NO: 200 and SEQ ID NO: 201, SEQ ID NO: 206 and SEQ ID NO: 207, SEQ ID NO: 208 and SEQ ID NO: 209, SEQ ID NO: 218 and SEQ ID NO: 219, SEQ ID NO: 220 and SEQ ID NO: 221, SEQ ID NO: 222 and SEQ ID NO: 223, SEQ ID NO: 224 and SEQ ID NO: 225, SEQ ID NO: 228 and SEQ ID NO: 229, SEQ ID NO: 230 and SEQ ID NO: 231, SEQ ID NO: 240 and SEQ ID NO: 241, SEQ ID NO: 244 and SEQ ID NO: 245, SEQ ID NO: 246 and SEQ ID NO: 247, SEQ ID NO: 256 and SEQ ID NO: 257, SEQ ID NO: 258 and SEQ ID NO: 259, SEQ ID NO: 260 and SEQ ID NO: 261, SEQ ID NO: 264 and SEQ ID NO: 265, SEQ ID NO: 266 and SEQ ID NO: 267, SEQ ID NO: 268 and SEQ ID NO: 269, SEQ ID NO: 270 and SEQ ID NO: 271, SEQ ID NO: 272 and SEQ ID NO: 273, SEQ ID NO: 274 and SEQ ID NO: 275, SEQ ID NO: 276 and SEQ ID NO: 277, SEQ ID NO: 278 and SEQ ID NO: 279, SEQ ID NO: 280 and SEQ ID NO: 281, SEQ ID NO: 282 and SEQ ID NO: 283, SEQ ID NO: 292 and SEQ ID NO: 293, SEQ ID NO: 294 and SEQ ID NO: 295, SEQ ID NO: 296 and SEQ ID NO: 297, SEQ ID NO: 298 and SEQ ID NO: 299, SEQ ID NO: 300 and SEQ ID NO: 301, SEQ ID NO: 302 and SEQ ID NO: 303, SEQ ID NO: 304 and SEQ ID NO: 305, SEQ ID NO: 306 and SEQ ID NO: 307, SEQ ID NO: 308 and SEQ ID NO: 309, SEQ ID NO: 310 and SEQ ID NO: 311, SEQ ID NO: 312 and SEQ ID NO: 313, SEQ ID NO: 314 and SEQ ID NO: 315, SEQ ID NO: 316 and SEQ ID NO: 317, SEQ ID NO: 318 and SEQ ID NO: 319, SEQ ID NO: 320 and SEQ ID NO: 321, SEQ ID NO: 322 and SEQ ID NO: 323, SEQ ID NO: 324 and SEQ ID NO: 325, SEQ ID NO: 326 and SEQ ID NO: 327, SEQ ID NO: 328 and SEQ ID NO: 329, SEQ ID NO: 330 and SEQ ID NO: 331, SEQ ID NO: 332 and SEQ ID NO: 333, SEQ ID NO: 336 and SEQ ID NO: 337, SEQ ID NO: 338 and SEQ ID NO: 339, SEQ ID NO: 340 and SEQ ID NO: 341, SEQ ID NO: 342 and SEQ ID NO: 343, SEQ ID NO: 344 and SEQ ID NO: 345, SEQ ID NO: 348 and SEQ ID NO: 349, SEQ ID NO: 350 and SEQ ID NO: 351, SEQ ID NO: 352 and SEQ ID NO: 353, SEQ ID NO: 354 and SEQ ID NO: 355, SEQ ID NO: 356 and SEQ ID NO: 357, SEQ ID NO: 358 and SEQ ID NO: 359, SEQ ID NO: 360 and SEQ ID NO: 361, SEQ ID NO: 362 and SEQ ID NO: 363, SEQ ID NO: 364 and SEQ ID NO: 365, SEQ ID NO: 366 and SEQ ID NO: 367, SEQ ID NO: 368 and SEQ ID NO: 369, SEQ ID NO: 370 and SEQ ID NO: 371, SEQ ID NO: 372 and SEQ ID NO: 373, SEQ ID NO: 374 and SEQ ID NO: 375, SEQ ID NO: 376 and SEQ ID NO: 377, SEQ ID NO: 378 and SEQ ID NO: 379, SEQ ID NO: 380 and SEQ ID NO: 381, SEQ ID NO: 382 and SEQ ID NO: 383, SEQ ID NO: 384 and SEQ ID NO: 385, SEQ ID NO: 386 and SEQ ID NO: 387, SEQ ID NO: 388 and SEQ ID NO: 389, SEQ ID NO: 390 and SEQ ID NO: 391, SEQ ID NO: 392 and SEQ ID NO: 393, SEQ ID NO: 394 and SEQ ID NO: 395, SEQ ID NO: 396 and SEQ ID NO: 397, SEQ ID NO: 398 and SEQ ID NO: 399, SEQ ID NO: 400 and SEQ ID NO: 401, SEQ ID NO: 402 and SEQ ID NO: 403, SEQ ID NO: 404 and SEQ ID NO: 405, SEQ ID NO: 406 and SEQ ID NO: 407, SEQ ID NO: 408 and SEQ ID NO: 409, SEQ ID NO: 410 and SEQ ID NO: 411, SEQ ID NO: 412 and SEQ ID NO: 413, SEQ ID NO: 414 and SEQ ID NO: 415, SEQ ID NO: 416 and SEQ ID NO: 417, SEQ ID NO: 418 and SEQ ID NO: 419, SEQ ID NO: 420 and SEQ ID NO: 421, SEQ ID NO: 422 and SEQ ID NO: 423, SEQ ID NO: 424 and SEQ ID NO: 425, SEQ ID NO: 426 and SEQ ID NO: 427, SEQ ID NO: 428 and SEQ ID NO: 429, SEQ ID NO: 430 and SEQ ID NO: 431, SEQ ID NO: 432 and SEQ ID NO: 433, SEQ ID NO: 434 and SEQ ID NO: 435, SEQ ID NO: 436 and SEQ ID NO: 437, SEQ ID NO: 438 and SEQ ID NO: 439, SEQ ID NO: 440 and SEQ ID NO: 441, SEQ ID NO: 442 and SEQ ID NO: 443, SEQ ID NO: 444 and SEQ ID NO: 445, SEQ ID NO: 446 and SEQ ID NO: 447, SEQ ID NO: 448 and SEQ ID NO: 449, SEQ ID NO: 450 and SEQ ID NO: 451, SEQ ID NO: 452 and SEQ ID NO: 453, SEQ ID NO: 454 and SEQ ID NO: 455, SEQ ID NO: 456 and SEQ ID NO: 457, SEQ ID NO: 458 and SEQ ID NO: 459, SEQ ID NO: 460 and SEQ ID NO: 461, SEQ ID NO: 462 and SEQ ID NO: 463, SEQ ID NO: 464 and SEQ ID NO: 465, SEQ ID NO: 466 and SEQ ID NO: 467, SEQ ID NO: 468 and SEQ ID NO: 469, SEQ ID NO: 470 and SEQ ID NO: 471, SEQ ID NO: 472 and SEQ ID NO: 473, SEQ ID NO: 474 and SEQ ID NO: 475, SEQ ID NO: 476 and SEQ ID NO: 477, SEQ ID NO: 478 and SEQ ID NO: 479, SEQ ID NO: 480 and SEQ ID NO: 481, SEQ ID NO: 482 and SEQ ID NO: 483, SEQ ID NO: 484 and SEQ ID NO: 485, SEQ ID NO: 486 and SEQ ID NO: 487, SEQ ID NO: 488 and SEQ ID NO: 489, SEQ ID NO: 490 and SEQ ID NO: 491, SEQ ID NO: 492 and SEQ ID NO: 493, SEQ ID NO: 494 and SEQ ID NO: 495, SEQ ID NO: 496 and SEQ ID NO: 497, SEQ ID NO: 498 and SEQ ID NO: 499, SEQ ID NO: 500 and SEQ ID NO: 501, SEQ ID NO: 502 and SEQ ID NO: 503, SEQ ID NO: 504 and SEQ ID NO: 505, SEQ ID NO: 506 and SEQ ID NO: 507, SEQ ID NO: 508 and SEQ ID NO: 509, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, SEQ ID NO: 646 and SEQ ID NO: 647, SEQ ID NO: 648 and SEQ ID NO: 649, SEQ ID NO: 650 and SEQ ID NO: 651, or SEQ ID NO: 652 and SEQ ID NO: 653, respectively, with zero, one, or two single amino acid substitutions in one or more HCDRs or LCDRs. In some embodiments, the HCDR1, HCDR2, HCDR3, LCDR1, LCDR2, and LCDR3 of the multimeric binding molecule include the CDRs of an antibody that includes the VH and VL of SEQ ID NO: 88 and SEQ ID NO: 89. In some embodiments, the HCDR1, HCDR2, HCDR3, LCDR1, LCDR2, and LCDR3 of the multimeric binding molecule include the CDRs of an antibody that includes the VH and VL of SEQ ID NO: 260 and SEQ ID NO: 261. In some embodiments, the HCDR1, HCDR2, HCDR3, LCDR1, LCDR2, and LCDR3 of the multimeric binding molecule include the CDRs of an antibody that includes the VH and VL of SEQ ID NO: 264 and SEQ ID NO: 265. In some embodiments, the HCDR1, HCDR2, HCDR3, LCDR1, LCDR2, and LCDR3 of the multimeric binding molecule include the CDRs of an antibody that includes the VH and VL of SEQ ID NO: 266 and SEQ ID NO: 267. In some embodiments, the HCDR1, HCDR2, HCDR3, LCDR1, LCDR2, and LCDR3 of the multimeric binding molecule include the CDRs of an antibody that includes the VH and VL of SEQ ID NO: 274 and SEQ ID NO: 275, SEQ ID NO: 278, and SEQ ID NO: 279, SEQ ID NO: 280 and SEQ ID NO: 281, or SEQ ID NO: 282 and SEQ ID NO: 283. In some embodiments, the HCDR1, HCDR2, HCDR3, LCDR1, LCDR2, and LCDR3 of the multimeric binding molecule include the CDRs of an antibody that includes the VH and VL of SEQ ID NO: 292 and SEQ ID NO: 293. In some embodiments, the VH and VL of the multimeric binding molecule described herein include amino acid sequences at least 80%, at least 85%, at least 90%, at least 95% or 100% identical to the recited VH and VL amino acid sequences, respectively. In some embodiments a bivalent reference IgG antibody that includes two of the binding domains can neutralize SARS-CoV-2.

In some embodiments, the three to twelve identical binding domains of the multimeric binding molecule each specifically bind to SARS-CoV-2, and the VH and VL of the multimeric binding molecule include six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 include the CDRs of an antibody that includes the VH and VL of SEQ ID NO: 118 and SEQ ID NO: 119, SEQ ID NO: 162 and SEQ ID NO: 163, SEQ ID NO: 222 and SEQ ID NO: 223, SEQ ID NO: 260 and SEQ ID NO: 261, SEQ ID NO: 296 and SEQ ID NO: 297, SEQ ID NO: 384 and SEQ ID NO: 385, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, or SEQ ID NO: 646 and SEQ ID NO: 647, respectively. In some embodiments, the VH and VL of the multimeric binding molecule include amino acid sequences at least 80%, at least 85%, at least 90%, at least 95% or 100% identical to the recited VH and VL amino acid sequences, respectively. In some embodiments, a bivalent reference IgG antibody that includes two of the binding domains can neutralize SARS-CoV-2, and specifically binds to SARS-CoV.

In some embodiments, the VH and VL of the multimeric binding molecule include six immunoglobulin complementarity determining regions HCDR1, HCDR2, HCDR3, LCDR1, LCDR2, and LCDR3, and where the HCDR1, HCDR2, HCDR3, LCDR1, LCDR2, and LCDR3 include the CDRs of an antibody that includes the VH and VL of SEQ ID NO: 260 and SEQ ID NO: 261, SEQ ID NO: 296 and SEQ ID NO: 297, SEQ ID NO: 384 and SEQ ID NO: 385, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, or SEQ ID NO: 646 and SEQ ID NO: 647, respectively. In certain embodiments, the VH and VL include the CDRs of an antibody that includes the VH and VL of SEQ ID NO:384 and SEQ ID NO: 385. In certain embodiments, the VH and VL include the CDRs of an antibody that includes the VH and VL of SEQ ID NO: 646 and SEQ ID NO: 647. In some of these embodiments, the VH and VL of the multimeric binding molecule include amino acid sequences at least 80%, at least 85%, at least 90%, at least 95% or 100% identical to the recited VH and VL amino acid sequences, respectively. In some embodiments, a bivalent reference IgG antibody that includes two of the binding domains can neutralize SARS-CoV and SARS-CoV-2.

In some embodiments, the three to twelve identical binding domains of the multimeric binding molecule each specifically bind to SARS-CoV-2 and each include a single domain variable region (VHH), where the VHH includes three immunoglobulin complementarity determining regions HCDR1, HCDR2, and HCDR3, where the HCDR1, HCDR2, and HCDR3 include the CDRs of an antibody that includes the VHH of SEQ ID NO: SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, or SEQ ID NO:83, with zero, one, or two single amino acid substitutions in one or more of the HCDRs. In some embodiments, the VHH of the multimeric binding molecule includes an amino acid sequence at least 80%, at least 85%, at least 90%, at least 95% or 100% identical to the recited VHH amino acid sequence.

In some embodiments, the three to twelve identical binding domains of the multimeric binding molecule as described herein specifically bind to a human coronavirus that includes an extracellular SARS-CoV-2 RBD-binding fragment of angiotensin-converting enzyme 2 (ACE2).

In some embodiments, the three to twelve identical binding domains of the multimeric binding molecule as described herein each specifically bind to SARS-CoV and include a heavy chain variable region (VH) and a light chain variable region (VL), where the VH and VL include six immunoglobulin complementarity determining regions HCDR1, HCDR2, HCDR3, LCDR1, LCDR2, and LCDR3, and where the HCDR1, HCDR2, HCDR3, LCDR1, LCDR2, and LCDR3 include the CDRs of an antibody that includes the VH and VL SEQ ID NO: 84 and SEQ ID NO: 85, SEQ ID NO: 118 and SEQ ID NO: 119, SEQ ID NO: 120 and SEQ ID NO: 121, SEQ ID NO: 138 and SEQ ID NO: 139, SEQ ID NO: 162 and SEQ ID NO: 163, SEQ ID NO: 222 and SEQ ID NO: 223, SEQ ID NO: 236 and SEQ ID NO: 237, SEQ ID NO: 252 and SEQ ID NO: 253, SEQ ID NO: 260 and SEQ ID NO: 261, SEQ ID NO: 262 and SEQ ID NO: 263, SEQ ID NO: 296 and SEQ ID NO: 297, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, or SEQ ID NO: 644 and SEQ ID NO: 645. In some embodiments, the VH and VL of the multimeric binding molecule include amino acid sequences at least 80%, at least 85%, at least 90%, at least 95% or 100% identical to the recited VH and VL amino acid sequences, respectively.

In certain embodiments, the VH and VL of the multimeric binding molecule include six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 include the CDRs of an antibody that includes the VH and VL of SEQ ID NO: 84 and SEQ ID NO: 85, SEQ ID NO: 260 and SEQ ID NO: 261, SEQ ID NO: 262 and SEQ ID NO: 263, SEQ ID NO: 296 and SEQ ID NO: 297, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, or SEQ ID NO: 644 and SEQ ID NO: 645. In some embodiments, the VH and VL of the multimeric binding molecule include amino acid sequences at least 80%, at least 85%, at least 90%, at least 95% or 100% identical to the recited VH and VL amino acid sequences, respectively. In some embodiments the bivalent reference IgG antibody that includes two of the binding domains can neutralize SARS-CoV.

In some embodiments, the three to twelve identical binding domains of the multimeric binding molecule each specifically bind to MERS-CoV and include a heavy chain variable region (VH) and a light chain variable region (VL), where the VH and VL include six immunoglobulin complementarity determining regions HCDR1, HCDR2, HCDR3, LCDR1, LCDR2, and LCDR3, and where the HCDR1, HCDR2, HCDR3, LCDR1, LCDR2, and LCDR3 include the CDRs of an antibody that includes the VH and VL SEQ ID NO:510 and SEQ ID NO:511, SEQ ID NO:512 and SEQ ID NO:513, SEQ ID NO:514 and SEQ ID NO:515, SEQ ID NO:516 and SEQ ID NO:517, SEQ ID NO:518 and SEQ ID NO:519, SEQ ID NO:520 and SEQ ID NO:521, SEQ ID NO:522 and SEQ ID NO:523, SEQ ID NO:524 and SEQ ID NO:525, SEQ ID NO:526 and SEQ ID NO:527, SEQ ID NO:528 and SEQ ID NO:529, SEQ ID NO:530 and SEQ ID NO:531, SEQ ID NO:532 and SEQ ID NO:533, SEQ ID NO:534 and SEQ ID NO:535, SEQ ID NO:536 and SEQ ID NO:537, SEQ ID NO:538 and SEQ ID NO:539, SEQ ID NO:540 and SEQ ID NO:541, SEQ ID NO:542 and SEQ ID NO:543, SEQ ID NO:544 and SEQ ID NO:545, SEQ ID NO:546 and SEQ ID NO:547, SEQ ID NO:548 and SEQ ID NO:549, SEQ ID NO:550 and SEQ ID NO:551, SEQ ID NO:552 and SEQ ID NO:553, SEQ ID NO:554 and SEQ ID NO:555, SEQ ID NO:556 and SEQ ID NO:557, SEQ ID NO:558 and SEQ ID NO:559, SEQ ID NO:560 and SEQ ID NO:561, SEQ ID NO:562 and SEQ ID NO:563, SEQ ID NO:564 and SEQ ID NO:565, SEQ ID NO:566 and SEQ ID NO:567, SEQ ID NO:568 and SEQ ID NO:569, SEQ ID NO:570 and SEQ ID NO:571, SEQ ID NO:572 and SEQ ID NO:573, SEQ ID NO:574 and SEQ ID NO:575, SEQ ID NO:576 and SEQ ID NO:577, SEQ ID NO:578 and SEQ ID NO:579, SEQ ID NO:580 and SEQ ID NO:581, SEQ ID NO:582 and SEQ ID NO:583, SEQ ID NO:584 and SEQ ID NO:585, SEQ ID NO:586 and SEQ ID NO:587, SEQ ID NO:588 and SEQ ID NO:589, SEQ ID NO:590 and SEQ ID NO:591, SEQ ID NO:592 and SEQ ID NO:593, SEQ ID NO:594 and SEQ ID NO:595, SEQ ID NO:596 and SEQ ID NO:597, SEQ ID NO:598 and SEQ ID NO:599, SEQ ID NO:600 and SEQ ID NO:601, SEQ ID NO:602 and SEQ ID NO:603, SEQ ID NO:604 and SEQ ID NO:605, SEQ ID NO:606 and SEQ ID NO:607, SEQ ID NO:608 and SEQ ID NO:609, SEQ ID NO:610 and SEQ ID NO:611, SEQ ID NO:612 and SEQ ID NO:613, SEQ ID NO:614 and SEQ ID NO:615, SEQ ID NO:616 and SEQ ID NO:617, SEQ ID NO:618 and SEQ ID NO:619, SEQ ID NO:620 and SEQ ID NO:621, SEQ ID NO:622 and SEQ ID NO:623, SEQ ID NO:624 and SEQ ID NO:625, SEQ ID NO:626 and SEQ ID NO:627, or SEQ ID NO:630 and SQ ID NO:631, respectively, with zero, one, or two single amino acid substitutions in one or more of the HCDRs or LCDRs. In some embodiments, the VH and VL of the multimeric binding molecule include amino acid sequences at least 80%, at least 85%, at least 90%, at least 95% or 100% identical to the recited VH and VL amino acid sequences, respectively.

In some embodiments, the HCDR1, HCDR2, HCDR3, LCDR1, LCDR2, and LCDR3 of the multimeric binding molecule include the CDRs of an antibody that includes the VH and VL of SEQ ID NO: 510 and SEQ ID NO: 511, SEQ ID NO: 512 and SEQ ID NO: 513, SEQ ID NO: 514 and SEQ ID NO: 515, SEQ ID NO: 516 and SEQ ID NO: 517, SEQ ID NO: 518 and SEQ ID NO: 519, SEQ ID NO: 520 and SEQ ID NO: 521, SEQ ID NO: 522 and SEQ ID NO: 523, SEQ ID NO: 524 and SEQ ID NO: 525, SEQ ID NO: 526 and SEQ ID NO: 527, SEQ ID NO: 528 and SEQ ID NO: 529, SEQ ID NO: 530 and SEQ ID NO: 531, SEQ ID NO: 532 and SEQ ID NO: 533, SEQ ID NO: 534 and SEQ ID NO: 535, SEQ ID NO: 536 and SEQ ID NO: 537, SEQ ID NO: 538 and SEQ ID NO: 539, SEQ ID NO: 540 and SEQ ID NO: 541, SEQ ID NO: 542 and SEQ ID NO: 543, SEQ ID NO: 544 and SEQ ID NO: 545, SEQ ID NO: 546 and SEQ ID NO: 547, SEQ ID NO: 548 and SEQ ID NO: 549, SEQ ID NO: 550 and SEQ ID NO: 551, SEQ ID NO: 552 and SEQ ID NO: 553, SEQ ID NO: 554 and SEQ ID NO: 555, SEQ ID NO: 556 and SEQ ID NO: 557, SEQ ID NO: 558 and SEQ ID NO: 559, SEQ ID NO: 560 and SEQ ID NO: 561, SEQ ID NO: 562 and SEQ ID NO: 563, SEQ ID NO: 564 and SEQ ID NO: 565, SEQ ID NO: 566 and SEQ ID NO: 567, SEQ ID NO: 568 and SEQ ID NO: 569, SEQ ID NO: 574 and SEQ ID NO: 575, SEQ ID NO: 576 and SEQ ID NO: 577, SEQ ID NO: 578 and SEQ ID NO: 579, SEQ ID NO: 580 and SEQ ID NO: 581, SEQ ID NO: 582 and SEQ ID NO: 583, SEQ ID NO: 584 and SEQ ID NO: 585, SEQ ID NO: 586 and SEQ ID NO: 587, SEQ ID NO: 588 and SEQ ID NO: 589, SEQ ID NO: 590 and SEQ ID NO: 591, SEQ ID NO: 592 and SEQ ID NO: 593, SEQ ID NO: 594 and SEQ ID NO: 595, SEQ ID NO: 596 and SEQ ID NO: 597, SEQ ID NO: 598 and SEQ ID NO: 599, SEQ ID NO: 600 and SEQ ID NO: 601, SEQ ID NO: 602 and SEQ ID NO: 603, SEQ ID NO: 604 and SEQ ID NO: 605, SEQ ID NO: 606 and SEQ ID NO: 607, SEQ ID NO: 608 and SEQ ID NO: 609, SEQ ID NO: 610 and SEQ ID NO: 611, SEQ ID NO: 612 and SEQ ID NO: 613, SEQ ID NO: 614 and SEQ ID NO: 615, SEQ ID NO: 616 and SEQ ID NO: 617, SEQ ID NO: 618 and SEQ ID NO: 619, SEQ ID NO: 620 and SEQ ID NO: 621, SEQ ID NO: 622 and SEQ ID NO: 623, SEQ ID NO: 624 and SEQ ID NO: 625, SEQ ID NO: 626 and SEQ ID NO: 627, or SEQ ID NO: 630 and SEQ ID NO: 631, respectively. In some embodiments, the HCDR1, HCDR2, HCDR3, LCDR1, LCDR2, and LCDR3 of the multimeric binding molecule include the CDRs of an antibody that includes the VH and VL of SEQ ID NO: 630 and SEQ ID NO: 631, respectively. In some of these embodiments, the VH and VL of the multimeric binding molecule include amino acid sequences at least 80%, at least 85%, at least 90%, at least 95% or 100% identical to the recited VH and VL amino acid sequences, respectively. In some of these embodiments, the bivalent reference IgG antibody that includes two of the binding domains can neutralize MERS-CoV.

In some embodiments, the three to twelve identical binding domains of the multimeric binding molecule include an extracellular MERS-CoV RBD-binding fragment of dipeptidyl peptidase 4 (DPP4).

In some embodiments the multimeric binding molecule can neutralize escape mutants of a bivalent reference IgG antibody that includes two of the binding domains.

In some embodiments, the multimeric binding molecule includes two or four bivalent IgA or IgA-like binding units and a J chain or functional fragment or variant thereof, where each binding unit includes two IgA heavy chain constant regions or multimerizing fragments or variants thereof, each including an IgA Cα3 domain and an IgA tailpiece domain. In some embodiments, the multimeric binding molecule is a dimeric binding molecule that includes two bivalent IgA or IgA-like binding units. In some embodiments, each IgA heavy chain constant region or multimerizing fragment or variant thereof further includes a Cα1 domain, a Cα2 domain, an IgA hinge region, or any combination thereof. In some embodiments, the IgA heavy chain constant regions or multimerizing fragments or variants thereof are human IgA constant regions. In some embodiments, each binding unit of the multimeric binding molecule includes two IgA heavy chains each including a VH situated amino terminal to the IgA constant region or multimerizing fragment thereof, and two immunoglobulin light chains each including a VL situated amino terminal to an immunoglobulin light chain constant region.

In some embodiments, the multimeric binding molecule includes five or six bivalent IgM or IgM-like binding units, where each binding unit includes two IgM heavy chain constant regions or multimerizing fragments or variants thereof, each including an IgM Cμ4 and IgM tailpiece domain. In some embodiments, each IgM heavy chain constant region or multimerizing fragment or variant thereof of the multimeric binding molecule further includes a Cμ1 domain, a Cμ2 domain, a Cμ3 domain, or any combination thereof. In some of these embodiments, the IgM heavy chain constant regions or multimerizing fragments or variants thereof are human IgM constant regions. In some embodiments, the IgM heavy chain constant regions each include the amino acid sequence SEQ ID NO: 1, SEQ ID NO: 2, or a multimerizing fragment or variant thereof. In some embodiments, each binding unit of the multimeric binding molecule includes two IgM heavy chains, each including a VH situated amino terminal to the IgM constant region or fragment thereof, and two immunoglobulin light chains, each including a VL situated amino terminal to an immunoglobulin light chain constant region.

In some embodiments the IgM constant regions of the multimeric binding molecule each include one or more amino acid substitutions corresponding to a wild-type human IgM constant region at position 310, 311, 313, and/or 315 of SEQ ID NO: 1 or SEQ ID NO: 2, and the multimeric binding molecule exhibits reduced complement-dependent cytotoxicity (CDC) activity to cells in the presence of complement, relative to a reference binding molecule that is identical except for the one or more amino acid substitutions. In some embodiments, the IgM constant regions of the multimeric binding molecule each include one or more substitutions corresponding to a wild-type human IgM constant region at positions 46, 209, 272, or 440 of SEQ ID NO: 1 or SEQ ID NO: 2, where the one or more amino acid substitutions prevent asparagine (N)-linked glycosylation. In some of these embodiments, the multimeric binding molecule is pentameric, and further includes a J-chain or functional fragment or variant thereof. In some embodiments, the multimeric binding molecule can transport across vascular endothelial cells via J-chain binding to the polymeric Ig receptor (PIgR). In some embodiments, the multimeric binding molecule further includes a secretory component, or fragment or variant thereof.

In some embodiments the J-chain or functional fragment or variant thereof the multimeric binding molecule that includes five or six bivalent IgM or IgM-like binding units further includes a heterologous polypeptide, where the heterologous polypeptide is directly or indirectly fused to the J-chain or functional fragment or variant thereof. In some embodiments, the heterologous polypeptide is fused to the J-chain or fragment thereof via a peptide linker. In some embodiments, the peptide linker includes at least 5 amino acids, but no more than 25 amino acids. In some embodiments, the peptide linker consists of the amino acid sequence GGGGS (SEQ ID NO: 9), GGGGSGGGGS (SEQ ID NO: 10), GGGGSGGGGSGGGGS (SEQ ID NO: 11), GGGGSGGGGSGGGGSGGGGS (SEQ ID NO: 12), or GGGGSGGGGSGGGGSGGGGSGGGGS (SEQ ID NO: 13).

In some embodiments, the heterologous polypeptide of the multimeric binding molecule that includes five or six bivalent IgM or IgM-like binding units is fused to the N-terminus of the J-chain or fragment or variant thereof, the C-terminus of the J-chain or fragment or variant thereof, or to both the N-terminus and C-terminus of the J-chain or fragment or variant thereof.

In some embodiments, the heterologous polypeptide of the multimeric binding molecule that includes five or six bivalent IgM or IgM-like binding units can influence the absorption, distribution, metabolism and/or excretion (ADME) of the multimeric binding molecule. In some embodiments, the heterologous polypeptide includes an albumin or an albumin binding domain, human serum albumin, or an antigen binding domain. In some embodiments, the antigen binding domain binds to the human coronavirus. In some embodiments, the antigen binding domain binds to a different epitope of the human coronavirus than the three to twelve identical binding domains of the multimeric binding molecule that specifically bind to the human coronavirus. In some embodiments, the antigen binding domain of the heterologous polypeptide is an antibody or antigen-binding fragment thereof. In some of these embodiments, the antigen-binding fragment includes a Fab fragment, a Fab′ fragment, a F(ab′)2 fragment, a Fd fragment, a Fv fragment, a single-chain Fv (scFv) fragment, a disulfide-linked Fv (sdFv) fragment, or any combination thereof. In some embodiments, the antigen-binding fragment is a scFv fragment. In some embodiments, the heterologous polypeptide includes an extracellular SARS-CoV-2 RBD-binding fragment of angiotensin-converting enzyme 2 (ACE2). In some embodiments, the heterologous polypeptide includes an extracellular MERS-COV RBD-binding fragment of dipeptidyl peptidase 4 (DPP4).

In some embodiments, the J-chain or functional fragment or variant thereof of the multimeric binding molecule that includes five or six bivalent IgM or IgM-like binding units further includes an additional heterologous polypeptide, where the additional heterologous polypeptide is directly or indirectly fused to the J-chain or functional fragment or variant thereof. In some embodiments, the additional heterologous polypeptide can influence the absorption, distribution, metabolism and/or excretion (ADME) of the multimeric binding molecule. In some embodiments, the additional heterologous polypeptide includes an albumin, an albumin binding protein, or human serum albumin.

Some embodiments of the disclosure are directed to a composition that includes a multimeric binding molecule as described herein. In some embodiments, the composition includes two or more nonidentical multimeric binding molecules as described herein, where the two or more multimeric binding molecules bind to different epitopes of a single human coronavirus.

Some embodiments of the disclosure are directed to a polynucleotide that includes a nucleic acid sequence that encodes a polypeptide subunit of the binding molecule described herein.

Some embodiments of the disclosure are directed to a vector that includes the polynucleotide as described herein.

Some embodiments of the disclosure are directed to a host cell that includes a polynucleotide of the disclosure, or a vector of the disclosure, where the host cell can express a multimeric binding molecule as described herein.

Some embodiments of the disclosure relate to methods of producing a multimeric binding molecule as described herein, which includes culturing a host cell as described herein, and recovering the multimeric binding molecule. In some embodiments, the method further includes contacting the multimeric binding molecule with a secretory component, or fragment or variant thereof.

Some embodiments as described herein are directed to a method for treating a human coronavirus disease in a subject in need of treatment that includes administering to the subject an effective amount of a multimeric binding molecule as described herein, where the multimeric binding molecule exhibits greater potency than an equivalent amount of a bivalent reference IgG antibody that includes two of the binding domains that specifically bind to the human coronavirus. In some embodiments the human coronavirus disease is coronavirus disease 2019 (COVID-19). In some embodiments, the human coronavirus disease is Middle East Respiratory Syndrome (MERS). In some methods, the subject is human. In some embodiments, the method includes intravenous, subcutaneous, intramuscular, intranasal, and/or inhalation administration of a multimeric binding molecule as described herein.

Some embodiments of the disclosure are directed to a method for preventing a human coronavirus disease in a subject, which includes administering to the subject an effective amount of a multimeric binding molecule as described herein, where the multimeric binding molecule exhibits greater potency than an equivalent amount of a bivalent reference IgG antibody that includes two of the binding domains that specifically bind to the human coronavirus. In some embodiments the human coronavirus disease is coronavirus disease 2019 (COVID-19). In some embodiments, the human coronavirus disease is Middle East Respiratory Syndrome (MERS). In some methods, the subject is human. In some embodiments, the method includes intravenous, subcutaneous, intramuscular, intranasal, and/or inhalation administration of a multimeric binding molecule as described herein.

### BRIEF DESCRIPTION OF THE DRAWINGS/FIGURES

FIGS. 1A-1B show binding of CR3022 IgM, IgA1, IgA2m2, and IgG to SARS-CoV-1 (FIG. 1A) or SARS-CoV-2 (FIG. 1B) receptor binding domain (RBD) in an ELISA assay.

FIGS. 2A-2F show the percent inhibition of SARS-CoV-1 pseudovirus by CR3022 IgG1 (FIG. 2A), CR3022 IgA1 (FIG. 2B), CR3022 IgA2m2 (FIG. 2C), CR3022 IgM (FIG. 2D), CR3014 IgG1 (FIG. 2E), and CR3014 IgM (FIG. 2F). The half maximal inhibitory concentration (IC₅₀) is denoted by a dashed line.

FIG. 3 shows the concentration of IgG, IgA1, IgA2m2, and IgM in the apical chamber following pIgR-mediated transcytosis.

FIGS. 4A-4G show binding to SARS-CoV-2 receptor binding domain (RBD) (FIGS. 4A-4D) of IgM or IgG Ab1 (FIG. 4A), IgM or IgG Ab2 (FIG. 4B), IgM or IgG Ab3 (FIG. 4C), IgM or IgG Ab4 (FIG. 4D), IgM or IgG Ab10 (FIG. 4E), IgM or IgG Ab11 (FIG. 4F), and IgM or IgG Ab13 (FIG. 4G) in an ELISA assay.

FIGS. 5A-5D show the percent neutralization of SARS-CoV-2 by IgM or IgG Ab10 (FIG. 5A), IgM or IgG Ab11 (FIG. 5B), IgM or IgG Ab12 (FIG. 5C), IgM or IgG Ab13 (FIG. 5D). The half maximal inhibitory concentration (IC₅₀) is denoted by a dashed line.

FIGS. 6A-6L show the percent neutralization of SARS-CoV-2 pseudovirus by IgM or IgG Ab1 (FIG. 6A), IgM or IgG Ab2 (FIG. 6B), IgM or IgG Ab3 (FIG. 6C), IgM or IgG Ab4 (FIG. 6D), IgM or IgG Ab5 (FIG. 6E), IgM or IgG Ab6 (FIG. 6F), IgM or IgG Ab7 (FIG. 6G), IgM or IgG Ab8 (FIG. 6H), IgM or IgG Ab10 (FIG. 6I), IgM or IgG Ab11 (FIG. 6J), IgM or IgG Ab12 (FIG. 6K), or IgM or IgG Ab13 (FIG. 6L).

### DETAILED DESCRIPTION

### Definitions

As used herein, the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments or embodiments of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt many different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine. Asparagine (N)-linked glycans are described in more detail elsewhere in this disclosure.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain embodiments, variants can be non-naturally occurring. Non-naturally occurring variants can be produced using art-known mutagenesis techniques. Variant polypeptides can comprise conservative or non-conservative amino acid substitutions, deletions, or additions. Derivatives are polypeptides that have been altered so as to exhibit additional features not found on the original polypeptide. Examples include fusion proteins. As used herein a “derivative” of a polypeptide can also refer to a subject polypeptide having one or more amino acids chemically derivatized by reaction of a functional side group. Also included as “derivatives” are those polypeptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and omithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides, binding molecules, and antibodies of the present disclosure do not abrogate the binding of the polypeptide, binding molecule, or antibody containing the amino acid sequence, to the antigen to which the antibody binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12:879-884 (1999); and Burks et al., Proc. Natl. Acad. Sci. USA 94: 412-417 (1997)).

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

As used herein, the term “binding molecule” refers in its broadest sense to a molecule that specifically binds to a receptor or target, e.g., an epitope or an antigenic determinant. As described further herein, a binding molecule can comprise one of more “binding domains,” e.g., “antigen-binding domains” described herein. A non-limiting example of a binding molecule is an antibody or antibody-like molecule as described in detail herein that retains antigen-specific binding. In certain embodiments a “binding molecule” comprises an antibody or antibody-like or antibody-derived molecule as described in detail herein.

As used herein, the terms “binding domain” or “antigen-binding domain” (can be used interchangeably) refer to a region of a binding molecule, e.g., an antibody or antibody-like, or antibody-derived molecule, that is necessary and sufficient to specifically bind to a target, e.g., an epitope, a polypeptide, a cell, or an organ. For example, an “Fv,” e.g., a heavy chain variable region and a light chain variable region of an antibody, either as two separate polypeptide subunits or as a single chain, is considered to be a “binding domain.” Other antigen-binding domains include, without limitation, a single domain heavy chain variable region (VHH) of an antibody derived from a camelid species, or six immunoglobulin complementarity determining regions (CDRs) expressed in a fibronectin scaffold. A “binding molecule,” e.g., an “antibody” as described herein can include one, two, three, four, five, six, seven, eight, nine, ten, eleven, twelve, or more “antigen-binding domains.”

The terms “antibody” and “immunoglobulin” can be used interchangeably herein. An antibody (or a fragment, variant, or derivative thereof as disclosed herein, e.g., an IgM-like antibody) includes at least the variable domain of a heavy chain (e.g., from a camelid species) or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four complete heavy chains and four complete light chains and includes a J-chain and/or a secretory component, or an IgM-derived binding molecule, e.g., an IgM antibody or IgM-like antibody, that includes ten or twelve complete heavy chains and ten or twelve complete light chains and optionally includes a J-chain or functional fragment or variant thereof.

The term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgD, or IgE, respectively. The immunoglobulin subclasses (subtypes) e.g., IgG₁, IgG₂, IgG₃, IgG₄, IgA₁, IgA₂, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody, antibody-like molecule, or antibody-derived molecule, antigen-binding fragment thereof, or multimerizing fragment thereof, which corresponds to a standard “H2L2” immunoglobulin structure, i.e., two heavy chains or fragments thereof and two light chains or fragments thereof. In certain embodiments, e.g., where the binding molecule is a bivalent IgG antibody or antigen-binding fragment thereof, the terms “binding molecule” and “binding unit” are equivalent. Such binding molecules are also referred to herein as “monomeric.” In other embodiments, e.g., where the binding molecule is a “multimeric binding molecule,” e.g., a dimeric or tetrameric IgA antibody, a dimeric or tetrameric IgA-like antibody, a dimeric or tetrameric IgA-derived binding molecule, a pentameric or hexameric IgM antibody, a pentameric or hexameric IgM-like antibody, or a pentameric or hexameric IgM-derived binding molecule or any derivative thereof, the binding molecule comprises two or more “binding units.” Two in the case of an IgA dimer, four in the case of an IgA tetramer, or five or six in the case of an IgM pentamer or hexamer, respectively. A binding unit need not include full-length antibody heavy and light chains, but will typically be bivalent, i.e., will include two “antigen-binding domains,” as defined above. As used herein, certain binding molecules provided in this disclosure are “dimeric,” and include two bivalent binding units that include IgA constant regions or multimerizing fragments thereof. Certain binding molecules provided in this disclosure are “pentameric” or “hexameric,” and include five or six bivalent binding units that include IgM constant regions or multimerizing fragments or variants thereof. A binding molecule, e.g., an antibody or antibody-like molecule or antibody-derived binding molecule, comprising two or more, e.g., two, five, or six binding units, is referred to herein as “multimeric.”

The term “J-chain” as used herein refers to the J-chain of IgM or IgA antibodies of any animal species, any functional fragment thereof, derivative thereof, and/or variant thereof, including a mature human J-chain, the amino acid sequence of which is presented as SEQ ID NO: 7. Various J-chain variants and modified J-chain derivatives are disclosed herein. As persons of ordinary skill in the art will recognize, “a functional fragment” or “a functional variant” includes those fragments and variants that can associate with IgM heavy chain constant regions to form a pentameric IgM antibody or can associate with IgA heavy chain constant regions to form a dimeric IgA antibody.

The term “modified J-chain” is used herein to refer to a derivative of a J-chain polypeptide comprising a heterologous moiety, e.g., a heterologous polypeptide, e.g., an extraneous binding domain or functional domain introduced into or attached to the J-chain sequence. The introduction can be achieved by any means, including direct or indirect fusion of the heterologous polypeptide or other moiety or by attachment through a peptide or chemical linker. The term “modified human J-chain” encompasses, without limitation, the human J-chain comprising the amino acid sequence of SEQ ID NO: 7 or functional fragment thereof, or functional variant thereof, modified by the introduction of a heterologous moiety, e.g., a heterologous polypeptide, e.g., an extraneous binding domain. In certain embodiments the heterologous moiety does not interfere with efficient polymerization of IgM into a pentamer or IgA into a multimer, e.g., a dimer or tetramer, and binding of such polymers to a target. Exemplary modified J-chains can be found, e.g., in U.S. Pat. Nos. 9,951,134, 10,400,038, and 10,618,978, and in U.S. Patent Application Publication No. US-2019-0185570, each of which is incorporated herein by reference in its entirety.

As used herein the term “IgM-derived binding molecule” refers collectively to native IgM antibodies, IgM-like antibodies, as well as other IgM-derived binding molecules comprising non-antibody binding and/or functional domains instead of an antibody antigen binding domain or subunit thereof, and any fragments, e.g., multimerizing fragments, variants, or derivatives thereof.

As used herein, the term “IgM-like antibody” refers generally to a variant antibody or antibody-derived binding molecule that still retains the ability to form hexamers or pentamers, e.g., in association with a J-chain. An IgM-like antibody or other IgM-derived binding molecule typically includes at least the Cμ4-tp domains of the IgM constant region but can include heavy chain constant region domains from other antibody isotypes, e.g., IgG, from the same species or from a different species. An IgM-like antibody or other IgM-derived binding molecule can likewise be an antibody fragment in which one or more constant regions are deleted, as long as the IgM-like antibody is capable of forming hexamers and/or pentamers. Thus, an IgM-like antibody or other IgM-derived binding molecule can be, e.g., a hybrid IgM/IgG antibody or can be a “multimerizing fragment” of an IgM antibody.

As used herein the term “IgA-derived binding molecule” refers collectively to native IgA antibodies, IgA-like antibodies, as well as other IgA-derived binding molecules comprising non-antibody binding and/or functional domains instead of an antibody antigen binding domain or subunit thereof, and any fragments, e.g., multimerizing fragments, variants, or derivatives thereof.

As used herein, the term “IgA-like antibody” refers generally to a variant antibody or antibody-derived binding molecule that still retains the ability to form multimers, e.g., dimers, trimers, tetramers, and/or pentamers e.g., dimers and/or tetramers, e.g., in association with a J-chain. An IgA-like antibody or other IgA-derived binding molecule typically includes at least the Cα3-tp domains of the IgA constant region but can include heavy chain constant region domains from other antibody isotypes, e.g., IgG, from the same species or from a different species. An IgA-like antibody or other IgA-derived binding molecule can likewise be an antibody fragment in which one or more constant regions are deleted, as long as the IgA-like antibody can form multimers, e.g., dimers and/or tetramers. Thus, an IgA-like antibody or other IgA-derived binding molecule can be, e.g., a hybrid IgA/IgG antibody or can be a “multimerizing fragment” of an IgA antibody.

The terms “valency,” “bivalent,” “multivalent” and grammatical equivalents, refer to the number of binding domains, e.g., antigen-binding domains in given binding molecule, e.g., antibody, antibody-derived, or antibody-like molecule, or in a given binding unit. As such, the terms “bivalent”, “tetravalent”, and “hexavalent” in reference to a given binding molecule, e.g., an IgM antibody, IgM-like antibody, other IgM-derived binding molecule, or multimerizing fragment thereof, denote the presence of two antigen-binding domains, four antigen-binding domains, and six antigen-binding domains, respectively. A typical IgM antibody, IgM-like antibody, or other IgM-derived binding molecule, where each binding unit is bivalent, can have 10 or 12 valencies. A bivalent or multivalent binding molecule, e.g., antibody or antibody-derived molecule, can be monospecific, i.e., all of the antigen-binding domains are the same, or can be bispecific or multispecific, e.g., where two or more antigen-binding domains are different, e.g., bind to different epitopes on the same antigen, or bind to entirely different antigens.

The term “epitope” includes any molecular determinant capable of specific binding to an antigen-binding domain of an antibody, antibody-like, or antibody-derived molecule. In certain embodiments, an epitope can include chemically active surface groupings of molecules such as amino acids, sugar side chains, phosphoryl groups, or sulfonyl groups, and, in certain embodiments, can have three-dimensional structural characteristics, and or specific charge characteristics. An epitope is a region of a target that is bound by an antigen-binding domain of an antibody.

The term “target” is used in the broadest sense to include substances that can be bound by a binding molecule, e.g., antibody, antibody-like, or antibody-derived molecule. A target can be, e.g., a polypeptide, a nucleic acid, a carbohydrate, a lipid, or other molecule, or a minimal epitope on such molecule. Moreover, a “target” can, for example, be a cell, an organ, or an organism, e.g., an animal, plant, microbe, or virus, that comprises an epitope that can be bound by a binding molecule, e.g., antibody, antibody-like, or antibody-derived molecule.

Both the light and heavy chains of antibodies, antibody-like, or antibody-derived molecules are divided into regions of structural and functional homology. The terms “constant” and “variable” are used functionally. In this regard, it will be appreciated that the variable domains of both the variable light (VL) and variable heavy (VH) chain portions determine antigen recognition and specificity. Conversely, the constant region domains of the light chain (CL) and the heavy chain (e.g., CH1, CH2, CH3, or CH4) confer biological properties such as secretion, transplacental mobility, Fc receptor binding, complement binding, and the like. By convention, the numbering of the constant region domains increases as they become more distal from the antigen-binding site or amino-terminus of the antibody. The N-terminal portion is a variable region and at the C-terminal portion is a constant region; the CH3 (or CH4, e.g., in the case of IgM) and CL domains actually comprise the carboxy-terminus of the heavy and light chain, respectively.

A “full length IgM antibody heavy chain” is a polypeptide that includes, in N-terminal to C-terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tailpiece.

A “full length IgA antibody heavy chain” is a polypeptide that includes, in N-terminal to C-terminal direction, an antibody heavy chain variable domain (VH), an antibody heavy chain constant domain 1 (CA1 or Cα1), an IgA hinge region, an antibody heavy chain constant domain 2 (CA2 or Cα2), and an antibody heavy chain constant domain 3 (CA3 or Cα3) that can include an IgA tailpiece.

As indicated above, variable region(s) allow a binding molecule, e.g., antibody, antibody-like, or antibody-derived molecule, to selectively recognize and specifically bind epitopes on antigens. That is, the VL domain and VH domain, or subset of the complementarity determining regions (CDRs), of a binding molecule, e.g., an antibody, antibody-like, or antibody-derived molecule, combine to form the antigen-binding domain. More specifically, an antigen-binding domain can be defined by three CDRs on each of the VH and VL chains. Certain antibodies form larger structures. For example, IgA can form a molecule that includes two H2L2 binding units and a J-chain covalently connected via disulfide bonds, which can be further associated with a secretory component, and IgM can form a pentameric or hexameric molecule that includes five or six H2L2 binding units and optionally a J-chain covalently connected via disulfide bonds.

The six “complementarity determining regions” or “CDRs” present in an antibody antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Antibody variable domains can also be analyzed, e.g., using the IMGT information system (imgt.cines.fr) (IMGT®/V-Quest) to identify variable region segments, including CDRs. (See, e.g., Brochet et al., Nucl. Acids Res. 36:W503-508, 2008). IMGT uses a different numbering system than Kabat. See, e.g., Lefranc, M.-P. et al., Dev. Comp. Immunol. 27:55-77 (2003). Correspondences are listed, for example, at imgt.org/IMGTScientificChart/Numbering/CDR1-IMGTgaps.html.

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

The Kabat numbering system for the human IgM constant domain can be found in Kabat, et. al. “Tabulation and Analysis of Amino acid and nucleic acid Sequences of Precursors, V-Regions, C-Regions, J-Chain, T-Cell Receptors for Antigen, T-Cell Surface Antigens, 0-2 Microglobulins, Major Histocompatibility Antigens, Thy-1, Complement, C-Reactive Protein, Thymopoietin, Integrins, Post-gamma Globulin, α-2 Macroglobulins, and Other Related Proteins,” U.S. Dept. of Health and Human Services (1991). IgM constant regions can be numbered sequentially (i.e., amino acid #1 starting with the first amino acid of the constant region, or by using the Kabat numbering scheme. A comparison of the numbering of two alleles of the human IgM constant region sequentially (presented herein as SEQ ID NO: 1 (allele IGHM*03) and SEQ ID NO: 2 (allele IGHM*04)) and by the Kabat system is set out below. The underlined amino acid residues are not accounted for in the Kabat system (“X,” double underlined below, can be serine (S) (SEQ ID NO: 1) or glycine (G) (SEQ ID NO: 2)): Sequential (SEQ ID NO: 1 or SEQ ID NO: 2)/KABAT numbering key for IgM heavy chain

```
    1/127 GSASAPTLFP LVSCENSPSD TSSVAVGCLA QDFLPDSITF SWKYKNNSDI
  51/176 SSTRGFPSVL RGGKYAATSQ VLLPSKDVMQ GTDEHVVCKV QHPNGNKEKN
101/226 VPLPVIAELP PKVSVFVPPR DGFFGNPRKS KLICQATGFS PRQIQVSWLR
151/274 EGKQVGSGVT TDQVQAEAKE SGPTTYKVTS TLTIKESDWL XQSMFTCRVD
201/324 HRGLTFQQNA SSMCVPDQDT AIRVFAIPPS FASIFLTKST KLTCLVTDLT
251/374 TYDSVTISWT RQNGEAVKTH TNISESHPNA TFSAVGEASI CEDDWNSGER
301/424 FTCTVTHTDL PSPLKQTISR PKGVALHRPD VYLLPPAREQ LNLRESATIT
351/474 CLVTGFSPAD VFVQWMQRGQ PLSPEKYVTS APMPEPQAPG RYFAHSILTV
401/524 SEEEWNTGET YTCVVAHEAL PNRVTERTVD KSTGKPTLYN VSLVMSDTAG
451/574 TCY
```

Binding molecules, e.g., antibodies, antibody-like, or antibody-derived molecules, antigen-binding fragments, variants, or derivatives thereof, and/or multimerizing fragments thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen-binding domain, and that the binding entails some complementarity between the antigen-binding domain and the epitope. According to this definition, a binding molecule, e.g., antibody, antibody-like, or antibody-derived molecule, is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen with an on rate (k(on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹, 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A binding molecule, e.g., an antibody or fragment, variant, or derivative thereof is said to competitively inhibit binding of a reference antibody or antigen-binding fragment to a given epitope if it preferentially binds to that epitope to the extent that it blocks, to some degree, binding of the reference antibody or antigen-binding fragment to the epitope. Competitive inhibition can be determined by any method known in the art, for example, competition ELISA assays. A binding molecule can be said to competitively inhibit binding of the reference antibody or antigen-binding fragment to a given epitope by at least 90%, at least 80%, at least 70%, at least 60%, or at least 50%.

As used herein, the term “affinity” refers to a measure of the strength of the binding of an individual epitope with one or more antigen-binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of antigen-binding domains and an antigen. See, e.g., Harlow at pages 29-34. Avidity is related to both the affinity of individual antigen-binding domains in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a bivalent monoclonal antibody with a receptor present at a high density on a cell surface would also be of high avidity.

Binding molecules, e.g., antibodies or fragments, variants, or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross-reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or K_{D} no greater than 5×10⁻² M, 10⁻² M, 5×10⁻³M, 10⁻³M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹² M, 10⁻¹² M, 5×10⁻¹³M, 10⁻¹³M, 5×10⁻¹⁴ M, 10⁻¹⁴ M, 5×10⁻¹⁵ M, or 10⁻¹⁵ M.

“Antigen-binding antibody fragments” including single-chain antibodies or other antigen-binding domains can exist alone or in combination with one or more of the following: hinge region, CH1, CH2, CH3, or CH4 domains, J-chain, or secretory component. Also included are antigen-binding fragments that can include any combination of variable region(s) with one or more of a hinge region, CH1, CH2, CH3, or CH4 domains, a J-chain, or a secretory component. Binding molecules, e.g., antibodies, or antigen-binding fragments thereof can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al. According to embodiments of the present disclosure, an IgM antibody, IgM-like antibody, or other IgM-derived binding molecule as provided herein can include an antigen-binding fragment of an antibody, e.g., a scFv fragment, so long as the IgM antibody, IgM-like antibody, or other IgM-derived binding molecule is able to form a multimer, e.g., a hexamer or a pentamer, and an IgA antibody, IgA-like antibody, or other IgA-derived binding molecule as provided herein can include an antigen-binding fragment of an antibody, e.g., a scFv fragment, so long as the IgA antibody, IgA-like antibody, or other IgA-derived binding molecule is able to form a multimer, e.g., a dimer and/or a tetramer. As used herein such a fragment comprises a “multimerizing fragment.”

As used herein, the term “heavy chain subunit” includes amino acid sequences derived from an immunoglobulin heavy chain, a binding molecule, e.g., an antibody, antibody-like, or antibody-derived molecule comprising a heavy chain subunit can include at least one of: a VH domain, a CH1 domain, a hinge (e.g., upper, middle, and/or lower hinge region) domain, a CH2 domain, a CH3 domain, a CH4 domain, or a variant or fragment thereof. For example, a binding molecule, e.g., an antibody, antibody-like, or antibody-derived molecule, or fragment, e.g., multimerizing fragment, variant, or derivative thereof can include without limitation, in addition to a VH domain: a CH1 domain; a CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain embodiments a binding molecule, e.g., an antibody, antibody-like, or antibody-derived molecule, or fragment, e.g., multimerizing fragment, variant, or derivative thereof can include, in addition to a VH domain, a CH3 domain and a CH4 domain; or a CH3 domain, a CH4 domain, and a J-chain. Further, a binding molecule, e.g., an antibody, antibody-like, or antibody-derived molecule, for use in the disclosure can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule. According to embodiments of the present disclosure, an IgM antibody, IgM-like antibody, or other IgM-derived binding molecule as provided herein comprises sufficient portions of an IgM heavy chain constant region to allow the IgM antibody, IgM-like antibody, or other IgM-derived binding molecule to form a multimer, e.g., a hexamer or a pentamer. As used herein such a fragment comprises a “multimerizing fragment.” According to embodiments of the present disclosure, an IgA antibody, IgA-like antibody, or other IgA-derived binding molecule as provided herein comprises sufficient portions of an IgA heavy chain constant region to allow the IgA antibody, IgA-like antibody, or other IgA-derived binding molecule to form a multimer, e.g., a dimer or a tetramer. As used herein such a fragment comprises a “multimerizing fragment.”

As used herein, the term “light chain subunit” includes amino acid sequences derived from an immunoglobulin light chain. The light chain subunit includes at least a VL, and can further include a CL (e.g., Cκ or Cλ) domain.

Binding molecules, e.g., antibodies, antibody-like molecules, antibody-derived molecules, antigen-binding fragments, variants, or derivatives thereof, or multimerizing fragments thereof can be described or specified in terms of the epitope(s) or portion(s) of a target, e.g., a target antigen that they recognize or specifically bind. The portion of a target antigen that specifically interacts with the antigen-binding domain of an antibody is an “epitope,” or an “antigenic determinant.” A target antigen can comprise a single epitope or at least two epitopes, and can include any number of epitopes, depending on the size, conformation, and type of antigen.

As used herein, the term “hinge region” includes the portion of a heavy chain molecule that joins the CH1 domain to the CH2 domain in IgG, IgA, and IgD heavy chains, and provides flexibility to the molecule.

As used herein the term “disulfide bond” includes the covalent bond formed between two sulfur atoms, e.g., in cysteine residues of a polypeptide. The amino acid cysteine comprises a thiol group that can form a disulfide bond or bridge with a second thiol group. Disulfide bonds can be “intra-chain,” i.e., linking to cysteine residues in a single polypeptide or polypeptide subunit, or can be “inter-chain,” i.e., linking two separate polypeptide subunits, e.g., an antibody heavy chain and an antibody light chain, to antibody heavy chains, or an IgM or IgA antibody heavy chain constant region and a J-chain.

As used herein, the term “reference antibody” refers to an antibody with function similar to a multimeric binding molecule provided by this disclosure, e.g., an antibody functionally interacting with the target protein of interest that can include similar or identical antigen-binding domains. Reference antibodies may be monoclonal or polyclonal antibodies. In a particular embodiment, a “reference antibody” is a single-binding unit antibody with identical binding domains to a corresponding multimeric binding molecule as provided herein, e.g., a multimeric antibody with two, four, five, or six binding units.

As used herein, the term “chimeric antibody” refers to an antibody in which the immunoreactive region or site is obtained or derived from a first species and the constant region (which can be intact, partial, or modified) is obtained from a second species. In some embodiments the target binding region or site will be from a non-human source (e.g., mouse or primate) and the constant region is human.

The terms “multispecific antibody” or “bispecific antibody” refer to an antibody, antibody-like, or antibody-derived molecule that has antigen-binding domains for two or more different epitopes within a single antibody molecule. Other binding molecules in addition to the canonical antibody structure can be constructed with two binding specificities. Epitope binding by bispecific or multispecific antibodies can be simultaneous or sequential. Triomas and hybrid hybridomas are two examples of cell lines that can secrete bispecific antibodies. Bispecific antibodies can also be constructed by recombinant means. (Strohlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which a variable domain, constant region, and/or J-chain is altered by at least partial replacement of one or more amino acids. In certain embodiments entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain embodiments not all of the CDRs are replaced with the complete CDRs from the donor variable region and yet the antigen-binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial-and-error testing, to obtain a functional engineered or humanized antibody.

As used herein the term “engineered” includes manipulation of nucleic acid or polypeptide molecules by synthetic means (e.g., by recombinant techniques, in vitro peptide synthesis, by enzymatic or chemical coupling of peptides, nucleic acids, or glycans, or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

The terms “neutralizing” or “neutralize” as used herein refer to the ability of a therapeutic, e.g., a therapeutic antibody, to reduce and/or prevent viral infectivity. The term “infectivity” as used herein refers to the ability of the virus to do one or more of attach to cells, enter cells, release its nucleic acid, replicate its nucleic acid, and synthesize viral proteins, and package its nucleic acid into new virions that can be released from the infected cell. A virus can be neutralized, e.g., by a therapeutic antibody, via the antibody's ability to specifically bind to the virion and inhibit its ability to attach to a host cell receptor, thereby preventing entry into the host cell.

The terms “potent” or “potency” as used herein refer to the amount required to produce an effect, e.g., the amount of a binding molecule required to neutralize infectivity of a human coronavirus. In some embodiments, the potency is measured as the 50% effective concentration (EC₅₀) or 50% inhibitory concentration (IC₅₀) to neutralize or otherwise block infectivity (e.g., block attachment of the virus to the cellular receptor) of the human coronavirus, or therapeutic protection of a subject infected with a human coronavirus, measured, e.g., as a 50% effective dose (ED₅₀), or prophylactic protection of a subject susceptible to human infection, measured, e.g., as a 50% effective dose (ED₅₀). As used herein in the context of virus neutralization, the terms “50% effective concentration (EC₅₀)” and “50% inhibitory concentration (IC₅₀)” can be used interchangeably.

As used herein, a “human coronavirus” or “H-CoV” is a virus of the family Coronaviridae that is capable of infecting humans. Some coronaviruses can be traced to zoonotic sources, e.g., bats. Certain betacoronaviruses can cause severe respiratory syndromes in humans and include, without limitation, SARS-CoV, MERS-CoV, and SARS-CoV-2. See, e.g., Sadia, A., and Basra, M. A. R., Drug Dev. Res. DOI: 10.1002/ddr.21710 (2020). The terms “SARS-CoV” and “SARS-CoV-1” are used interchangeably herein. Included herein are existing strains of human corona viruses, including but not limited to H-CoV strains 229E, NL63, OCH3, and HKV1. Further included are emerging variant strains of known human coronaviruses and escape variants including the 20H, 20I, 21A (Delta) Delta Plus, 21B (Kappa), 21D (Eta), and B.1.1.318 variants. Of particular relevance are emerging variants that are resistant to established therapeutics.

The phrase “structural protein” of a virus as used herein refer to a protein that is a component of a mature assembled viral particle and includes naturally-occurring variants. The four main structural proteins of the SARS-CoV and SARS-CoV-2 viruses include spike (S), envelope (E), membrane (M), and nucleic capsid (N). See, e.g., Sadia and Basra M. A. R., Drug Dev. Res. DOI: 10.1002/ddr.21710 (2020).

The phrases “emerging variant”, “escape mutant” or “escape variant” as used herein refer to strains of a human coronavirus comprising one or more mutations, such as a substitution, addition, or deletion, that reduces or prevents neutralization by a treatment or prophylactic, such as an antibody treatment or prophylaxis. An exemplary escape variant is a variant of an initial strain of SARS-CoV-2 that arises following contact of the initial strain of SARS-CoV-2, or cells infected with the initial strain of SARS-CoV-2, with an antibody capable of neutralizing the initial strain of SARS-CoV-2, where the escape variant is more resistant to neutralization by the antibody or is no longer capable of being neutralized by the antibody. SARS-CoV-2 escape variants can include one or more mutations, such as an amino acid substitutions, additions, or deletions, typically in the spike (S) protein, and typically in the receptor binding domain (RBD) of the S protein. Known SARS-CoV-2 escape variants that may be treated by compositions and methods described herein include, but are not limited to, 20I/501Y.V1 VOC 202012/01 (also referred to as B.1.1.7), 20H/501Y.V2 (also referred to as B.1.351), Delta variant (also referred to as B.1.617.2), Delta Plus, 20H, 20I, 21A (Delta) Delta Plus, 21B (Kappa), 21D (Eta), and B.1.1.318 variants and P.1 variants.

MERS escape variants or escape mutants can include one or more mutations, such as an amino acid substitutions, additions, or deletions, typically in the spike (S) protein, and typically in the receptor binding domain (RBD) of the S protein. Such mutants may be resistant to treatment, including immunotherapies. For example, mutations in the MERS-CoV spike (S) protein can result in reduced binding to DPP4. D510G and I528T mutations reduce S protein binding to DPP4, which can reduce levels of viral entry into cells having low levels of DPP4 (Kleine-Weber et al., J. Virol. 2019).

Terms such as “treating” or “treatment” or “to treat” or “alleviating” or “to alleviate” refer to therapeutic measures that cure, slow down, lessen symptoms of, lessen the severity of symptoms of, and/or halt or slow the progression of an existing diagnosed pathologic condition or disorder. Terms such as “prevent,” “prevention,” “avoid,” “deterrence,” “prophylactic,” and the like refer to prophylactic or preventative measures that can prevent the development of, or can reduce the symptoms of, a targeted pathologic condition or disorder in a subject who has not yet contracted the targeted pathologic condition or disorder. The targeted pathologic condition or disorder can be, for example, COVID-19. Thus, “those in need of treatment” can include those already infected with the human coronavirus, as well as those who wish to prevent infection, or reduce or alleviate symptoms associated with an infection should they become infected.

The terms “protect,” “protection,” “protective,” and other related terms, as used herein, refer to the ability of a therapeutic or prophylactic agent to confer a desirable effect on a subject diagnosed with or susceptible to an infectious disease resulting from a human coronavirus infection, such as COVID-19. Protection can include, for example, alleviation of or a reduction in infection-related symptoms in a subject infected with a human coronavirus, such as SARS-CoV-2, such that, for example, the subject does not need to be hospitalized or put on a ventilator. Protection can also include, for example, preventing healthcare workers, family members, or other contacts of infected patients from becoming infected with the human coronavirus, e.g., SARS-CoV-2, or if they do become infected, reducing the symptoms related to infection. As it applies to a therapeutic or prophylactic animal model, “protection” can include a lower 50% effective dose (ED₅₀) among a group of animal subjects challenged with the therapeutic agent either before or after challenge with the human coronavirus. Data points that can be used to measure ED₅₀ vary, e.g., with the animal model or the amount of human coronavirus used to challenge the animal subjects. Data points can include, e.g., measurement of the virus titer in the lungs of the animals, weight loss, death, or disease symptoms such as fever or difficulty breathing.

The terms “antibody-dependent enhancement” and “ADE” refer to the situation where the binding of an antibody or related binding molecule can increase infectivity of an infectious virus, including coronaviruses. See, e.g., Wen, J., et al., Int. J. Infect. Dis. 100:483-489 (2020).

As used herein the terms “serum half-life” or “plasma half-life” refer to the time it takes (e.g., in minutes, hours, or days) following administration for the serum or plasma concentration of a drug, e.g., a binding molecule such as an antibody, antibody-like, or antibody-derived molecule or fragment, e.g., multimerizing fragment thereof as described herein, to be reduced by 50%. Two half-lives can be described: the alpha half-life, a half-life, or t_{1/2}α, which is the rate of decline in plasma concentrations due to the process of drug redistribution from the central compartment, e.g., the blood in the case of intravenous delivery, to a peripheral compartment (e.g., a tissue or organ), and the beta half-life, β half-life, or t_{1/2}β which is the rate of decline due to the processes of excretion or metabolism.

As used herein the term “area under the plasma drug concentration-time curve” or “AUC” reflects the actual body exposure to drug after administration of a dose of the drug and is expressed in mg*h/L. This area under the curve can be measured, e.g., from time 0 (t₀) to infinity (∞) and is dependent on the rate of elimination of the drug from the body and the dose administered.

As used herein, the term “mean residence time” or “MRT” refers to the average length of time the drug remains in the body.

By “subject” or “individual” or “animal” or “patient” is meant any subject. In certain embodiments the subject is a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, as the term “a subject that would benefit from therapy” refers to a subset of subjects, from amongst all prospective subjects, which would benefit from administration of a given therapeutic agent, e.g., a binding molecule such as an antibody, comprising one or more antigen-binding domains. Such binding molecules, e.g., antibodies, can be used, e.g., for a diagnostic procedure and/or for treatment or prevention of a disease.

### Human Coronavirus, e.g., SARS-CoV-1, SARS-CoV-2, and MERS-CoV Binding Molecules

Provided herein are multimeric binding molecules comprising two to six bivalent binding units or variants or fragments thereof, where each binding unit comprises two IgA or IgM heavy chain constant regions or multimerizing fragments or variants thereof, each associated with a binding domain, where three to twelve of the binding domains are identical and specifically bind to a human coronavirus-, e.g., HCoV-229, HCoV-OC43, HCov-NL63, HCoV-HKU1, SARS-CoV, MERS-CoV, or SARS-CoV-2, any derivative or variant thereof, e.g., any naturally-occurring or non-naturally-occurring mutant (e.g., an escape mutant or emerging variant), or any combination thereof. The provided binding molecules can be used to treat or prevent human severe respiratory diseases caused by human coronaviruses, e.g., Coronavirus Disease 2019 (COVID-19).

In some embodiments, the binding molecules comprise three to twelve identical binding domains of the multimeric binding molecule that specifically bind to a human coronavirus comprise one or more heavy chain variable region (VH) and/or light chain variable region (VL) sequences or fragments thereof derived from antibodies with capacity to neutralize one or more human coronaviruses that are published in the Coronavirus Antibody Database (CoV-AbDAB), opig.stats.ox.ac.uk/webapps/covabdab/.

In some embodiments, the bivalent reference IgG antibody can bind SARS-CoV. In some embodiments, the bivalent reference IgG antibody can bind SARS-CoV-2. In some embodiments the bivalent reference antibody can bind MERS-CoV. In some embodiments, the bivalent reference molecule can bind more than one type of human coronavirus. In some embodiments, the bivalent reference IgG antibody can bind both SARS-CoV-2 and SARS-CoV. In some embodiments the bivalent reference antibody can bind SARS-CoV2, SARS-COV-1, and MERS-CoV. The ability of an antibody to bind a human coronavirus can readily be determined by one of skill in the art, such as by measuring binding in vitro using a technique such as enzyme-linked immunosorbent assay (ELISA). In some embodiments, the binding is determined at a specific concentration or within a range of concentrations of reference IgG antibody, e.g., at 0.1 nM, 1 nM, or 0.1 nM to 10 nM.

In some embodiments, the bivalent reference IgG antibody can neutralize a human coronavirus. In some embodiments, the bivalent reference IgG antibody can neutralize SARS-CoV-2. In some embodiments, the bivalent reference IgG antibody can neutralize SARS-CoV. In some embodiments the bivalent reference antibody can neutralize MERS-CoV. In some embodiments, the bivalent reference molecule can neutralize more than one type of human coronavirus. In some embodiments, the bivalent reference IgG antibody can neutralize SARS-CoV and SARS-CoV-2. In some embodiments, the bivalent reference IgG antibody can neutralize SARS-CoV-2 and cannot neutralize SARS-CoV. In some embodiments, the bivalent reference IgG antibody can neutralize SARS-CoV and cannot neutralize SARS-CoV-2. In some embodiments the bivalent reference antibody can neutralize SARS-CoV2, SARS-CoV, and MERS-CoV. In some embodiments the bivalent reference antibody can neutralize SARS-CoV2 and MERS-CoV.

In some embodiments, the provided binding molecule comprises two to six bivalent binding units or variants or multimerizing fragments thereof has greater avidity than the corresponding IgG reference antibody.

The ability of an antibody to neutralize a human coronavirus can readily be determined by one of skill in the art, such as by measuring infectivity in vitro using a viral or pseudoviral infectivity assay, such as an assay adapted from Richman et al. (PNAS, 2003, 100(7): 4144-4149) or described in Yuan et al., Science 10.1126/science.abb7269; (2020) or Muruato et al. (2020, Nat Comm. 11(1):4059. doi: 10.1038/s41467-020-17892-0). In some embodiments, the neutralization is determined at a specific concentration or within a range of concentrations of reference IgG antibody, e.g., at 0.1 nm, 1 nM, or 0.1 nM to 10 nM.

In certain embodiments, the greater potency of the provided multimeric binding molecule relative to the reference IgG can be measured, e.g., as inhibition of binding of the human coronavirus to its receptor, e.g., the SARS-CoV or SARS-CoV-2 spike protein binding to angiotensin-converting enzyme 2 (ACE2), at a lower 50% effective concentration (EC₅₀) or lower 50% inhibitory concentration (IC₅₀) than that of the bivalent reference IgG antibody. In certain embodiments the provided multimeric binding molecule can inhibit binding of the human coronavirus to its receptor, e.g., SARS-CoV or SARS-CoV-2 spike protein binding to ACE2 under conditions where the bivalent reference IgG antibody cannot inhibit binding. In certain embodiments, the greater potency of the provided multimeric binding molecule relative to the reference IgG can be measured, e.g., as inhibition of binding to the MERS-CoV spike protein binding of dipeptidyl-peptidase 4 (DPP4), at a lower EC₅₀ than the bivalent reference IgG antibody. In certain embodiments the provided multimeric binding molecule can inhibit binding of MERS-CoV-spike protein binding to DPP4 under conditions where the bivalent reference IgG antibody cannot inhibit binding. In certain embodiments, the provided multimeric binding molecule can neutralize the human coronavirus, e.g., SARS-CoV, SARS-CoV-2, or MERS-CoV, at a lower EC₅₀ than the bivalent reference IgG antibody. In certain embodiments, the provided multimeric binding molecule can neutralize the human coronavirus, e.g., SARS-CoV, SARS-CoV-2, or MERS-CoV under conditions where the bivalent reference IgG antibody cannot neutralize the respective human coronavirus. In certain embodiments, the provided multimeric binding molecule can protect infected animals, or prevent infection in uninfected animals in a therapeutic animal model at a lower 50% effective dose (ED₅₀) than the bivalent reference IgG antibody. In certain embodiments, the provided multimeric binding molecule can protect infected animals, or prevent infection in uninfected animals in a therapeutic animal model under conditions where the bivalent reference IgG antibody cannot protect. In certain embodiments, the provided multimeric binding molecule can comprise any combination of the foregoing properties.

In certain embodiments, the provided multimeric binding molecule can neutralize infectivity of a human coronavirus, e.g., SARS-CoV, SARS-CoV-2, or MERS-CoV, at a lower EC₅₀ than the bivalent reference IgG antibody. In certain embodiments, the provided multimeric binding molecule can neutralize infectivity of a human coronavirus, e.g., SARS-CoV, SARS-CoV-2, or MERS-CoV under conditions where the bivalent reference IgG antibody cannot neutralize. In certain embodiments, the EC₅₀ of the provided multimeric binding molecule is at least two-fold, at least five-fold, at least ten-fold, at least fifty-fold, at least 100-fold, at least 500-fold, or at least 1000-fold, or at least 10,000-fold lower than the EC₅₀ of the bivalent reference IgG antibody. The EC₅₀ can be measured either as mass per volume, e.g., μg/ml, or as the number of molecules present, e.g., moles/liter. In certain embodiments, the conditions where the bivalent reference IgG antibody cannot neutralize comprise neutralization of an antibody-resistant variant of a human coronavirus, e.g., SARS-CoV, SARS-CoV-2, or MERS-CoV. In certain embodiments, the antibody resistant variant of the human coronavirus, e.g., SARS-CoV, SARS-CoV-2, or MERS-CoV, comprises an “escape mutant” of a human coronavirus, e.g., SARS-CoV, SARS-CoV-2, or MERS-CoV virus that arose following contact with the bivalent reference IgG antibody. By “the human coronavirus, e.g., a SARS-CoV, SARS-CoV-2, or MERS-CoV virus that arose following contact with the bivalent reference IgG antibody” is meant a variant SARS-CoV-2 or MERS-CoV virus that arises in response to selective pressure. For example, an escape mutant can arise during an in vitro neutralization assay in which SARS-CoV-2 virus is contacted with the bivalent reference IgG antibody and then used to infect ACE2-expressing host cells, or during in in vivo infection of a subject animal, where the subject animal is administered the bivalent reference IgG antibody either prior to or subsequent to the virus infection. During viral replication in the host cells or subject animal mutations may arise that confer resistance to the bivalent reference IgG antibody.

In some embodiments the provided multimeric binding molecule can confer protection against a human coronavirus, e.g., SARS-CoV, SARS-CoV-2, or MERS-CoV, infection in a therapeutic or prophylactic animal model at a lower 50% effective dose (ED₅₀) than the bivalent reference IgG antibody, or wherein the binding molecule can confer protection against the human coronavirus, e.g., SARS-CoV, SARS-CoV-2, or MERS-CoV, infection in a therapeutic or prophylactic animal model under conditions where the bivalent reference IgG antibody cannot protect. As used herein, measurements of “protection” against the human coronavirus, e.g., SARS-CoV, SARS-CoV-2, or MERS-CoV, infection in an animal model can include a reduced human coronavirus, e.g., SARS-CoV, SARS-CoV-2, or MERS-CoV viral load in the subject animals, e.g., in the animals' lungs, survival of the subject animals from an otherwise lethal human coronavirus infection, and/or a reduction of symptoms typical of the human coronavirus infection in the animal model, e.g., weight loss, fever, difficulty breathing, or neurological symptoms. The ED₅₀ can be measured either as mass per volume, e.g., μg/ml, or as the number of molecules present, e.g., moles/liter. In certain embodiments, the conditions where the bivalent reference IgG antibody cannot protect comprises a virus challenge with an antibody-resistant variant of a human coronavirus, e.g., SARS-CoV, SARS-CoV-2, or MERS-CoV. In certain embodiments, the antibody resistant variant of the human coronavirus comprises an “escape mutant” of a human coronavirus that arose following contact with a bivalent reference IgG antibody.

In some embodiments the multimeric binding molecule reduces, inhibits, or blocks the human coronavirus from binding to its receptor at a lower EC₅₀ than the bivalent reference IgG antibody or reduces, inhibits, or blocks the human coronavirus from binding to its receptor under conditions where the bivalent reference IgG antibody cannot reduce, inhibit, or block the human coronavirus from binding to its receptor. In certain embodiments, the receptor is expressed on the surface of a cell, e.g., a cultured host cell, e.g., a Vero cell, or a cell in a susceptible subject, e.g., a human subject. In some embodiments, the binding molecule inhibits human coronavirus binding to its receptor at a lower 50% effective concentration (EC₅₀) than the bivalent reference IgG antibody. In some embodiments, the EC₅₀ is at least two-fold, at least three-fold, at least four-fold, at least five-fold, at least ten-fold, at least twenty-fold, at least thirty-fold, at least forty-fold, or at least fifty-fold lower than the EC₅₀ of the bivalent reference IgG antibody.

In some embodiments, the multimeric binding molecule binds a cell surface receptor involved in the attachment of the human coronavirus to a cell, a prerequisite for viral infection of the cell. In some embodiments, the human coronavirus is SARS-CoV (also referred to herein as SARS-CoV1), SARS-CoV-2, or human coronavirus NL63/HCoV-NL63, and the multimeric binding molecule reduces, inhibits, or blocks the human coronavirus from binding to its receptor, ACE2. An exemplary precursor human ACE2 amino acid sequence (UniprotKB Q9BYF1) is presented as SEQ ID NO: 14. As provided by UniprotKB, the signal peptide comprises amino acids 1 to 17 of SEQ ID NO: 14, the mature protein comprises amino acids 18 to 805 of SEQ ID NO: 2, the extracellular domain comprises amino acids 18 to 740 of SEQ ID NO: 14, the transmembrane domain comprises amino acids 741 to 761 of SEQ ID NO: 14, and the cytoplasmic domain comprises amino acids 762 to 805 of SEQ ID NO: 14. Amino acids 30-41, 82-84, and 353-357 of SEQ ID NO: 14 are reported to interact with the SARS-CoV spike protein. See Zhang, C., et al., EMBO J. 24:1634-1643 (2005).

In some embodiments, the human coronavirus is HCoV-229, and the multimeric binding molecule reduces, inhibits, or blocks the human coronavirus from binding to its receptor, aminopeptidase N (hAPN), also known as CD13.

In some embodiments, the human coronavirus is HCoV-OC43, and the multimeric binding molecule reduces, inhibits, or blocks the human coronavirus from binding to its receptor, n-acetyl-9-O-acetylneuraminic acid.

In some embodiments, the human coronavirus is HCoV-HKU1, and the multimeric binding molecule reduces, inhibits, or blocks the human coronavirus from binding to its receptor, O-acetylated sialic acid.

In some embodiments, the human coronavirus is MERS-CoV, and the multimeric binding molecule reduces, inhibits, or blocks the human coronavirus from binding to its receptor, dipeptidyl-peptidase 4 (DPP4), also known as CD26. An exemplary version of human DPP4 has the UniProtKB accession number P27487.

In some embodiments the multimeric binding molecule reduces, inhibits, or blocks the SARS-CoV or the SARS-CoV-2 S protein from binding to ACE2 at a lower EC₅₀ than the bivalent reference IgG antibody or reduces, inhibits, or blocks the SARS-CoV or the SARS-CoV-2 S protein from binding to ACE2 under conditions where the bivalent reference IgG antibody cannot reduce, inhibit, or block the SARS-CoV or the SARS-CoV-2 S protein from binding to ACE2. In certain embodiments the ACE2 is human ACE2, e.g., amino acids 18 to 805 of SEQ ID NO: 14. In certain embodiments, ACE2 is expressed on the surface of a cell, e.g., a cultured host cell, e.g., a Vero cell, or a cell in a susceptible subject, e.g., a human subject. In some embodiments, the binding molecule inhibits SARS-CoV-2 binding to its receptor, e.g., ACE2, at a lower 50% effective concentration (EC₅₀) than the bivalent reference IgG antibody. In some embodiments, the EC₅₀ is at least two-fold, at least three-fold, at least four-fold, at least five-fold, at least ten-fold, at least twenty-fold, at least thirty-fold, at least forty-fold, or at least fifty-fold lower than the EC₅₀ of the bivalent reference IgG antibody.

In some embodiments the multimeric binding molecule reduces, inhibits, or blocks the MERS-CoV S protein from binding to DPP4/CD26 at a lower EC₅₀ than the bivalent reference IgG antibody or reduces, inhibits, or blocks the MERS-CoV S protein from binding to DPP4/CD26 under conditions where the bivalent reference IgG antibody cannot reduce, inhibit, or block the MERS-CoV S protein from binding to DPP4/CD26. In certain embodiments the DPP4/CD26 is human DPP4/CD26. In certain embodiments, DPP4/CD26 is expressed on the surface of a cell, e.g., a cultured host cell, e.g., a Vero cell, or a cell in a susceptible subject, e.g., a human subject. In some embodiments, the binding molecule inhibits MERS-CoV binding to its receptor, e.g., DPP4/CD26, at a lower 50% effective concentration (EC₅₀) than the bivalent reference IgG antibody. In some embodiments, the EC₅₀ is at least two-fold, at least three-fold, at least four-fold, at least five-fold, at least ten-fold, at least twenty-fold, at least thirty-fold, at least forty-fold, or at least fifty-fold lower than the EC₅₀ of the bivalent reference IgG antibody.

Human coronaviruses are known to develop one or more mutations overtime that may alter the behavior of the virus. For example, SARS-CoV-2 has developed a spike mutant (D614G) that is believed to be more infectious than the original SARS-CoV-2 strain that does not contain this mutation (Korber et al. Cell 2020, DOI: 10.1016/j.cell.2020.06.043). D510G and I528T mutations in the MERS-CoV spike (S) protein can result in reduced binding to its receptor, DPP4, resulting in a reduction in a reduction in levels of viral entry into cells having low levels of DPP4 (Kleine-Weber et al., J. Virol. 2019).

A reference precursor SARS-CoV S protein (UniProtKB—P59594 (SPIKE_SARS)) is presented herein as SEQ ID NO: 16. Myriad variant SARS CoV S proteins have been sequenced and are available in the literature but share the common structure of SEQ ID NO: 16. As reported in UniProtKB, the signal peptide of the precursor SARS-CoV S protein corresponds to amino acids 1 to 13 of SEQ ID NO: 16, the mature SARS-CoV S protein corresponds to amino acids 14 to 1255 of SEQ ID NO: 16, the S1 region of the SARS-CoV S protein corresponds to amino acids 14 to 667 of SEQ ID NO: 16, the S2 region of the SARS-CoV S protein corresponds to amino acids 668 to 1255 of SEQ ID NO: 16, the receptor binding domain (RBD) of the SARS-CoV S protein corresponds to amino acids 306 to 527 of SEQ ID NO: 16 (underlined below), the receptor binding motif of the SARS-CoV S protein corresponds to amino acids 424 to 494 of SEQ ID NO: 16, the extracellular domain of the SARS-CoV S protein corresponds to amino acids 14 to 1195 of SEQ ID NO: 16, the transmembrane domain of the SARS-CoV S protein corresponds to amino acids 1196 to 1216 of SEQ ID NO: 16, and the cytoplasmic domain of the SARS-CoV S protein corresponds to amino 1214 to 1255 of SEQ ID NO: 16. As persons of ordinary skill in the art will recognize, the RBD of various SARS-CoV S proteins present in the environment have mutated so an RBD that “corresponds” to amino acids 306 to 527 of SEQ ID NO: 16 may not be identical to amino acids 306 to 527 of SEQ ID NO: 16. The TMPRSS2 or furin cleavage site between the S1 and S2 subunits is between amino acids 667 and 668 of SEQ ID NO: 16.

### SEQ ID NO: 16: SARS-CoV Spike Protein, UniProt: P59594

```
      1 MEIFLLFLTL TSGSDLDRCT TFDDVQAPNY TQHTSSMRGV YYPDEIFRSD TLYLTQDLFL
    61 PFYSNVTGFH TINHTFGNPV IPFKDGIYFA ATEKSNVVRG WVFGSTMNNK SQSVIIINNS
  121 TNVVIRACNF ELCDNPFFAV SKPMGTQTHT MIFDNAFNCT FEYISDAFSL DVSEKSGNFK
  181 HLREFVFKNK DGFLYVYKGY QPIDVVRDLP SGFNTLKPIF KLPLGINITN FRAILTAFSP
  241 AQDIWGTSAA AYFVGYLKPT TFMLKYDENG TITDAVDCSQ NPLAELKCSV KSFEIDKGIY
  301 QTSNFRVVPS GDVVRFPNIT NLCPFGEVFN ATKFPSVYAW ERKKISNCVA DYSVLYNSTF
  361 FSTFKCYGVS ATKLNDLCFS NVYADSFVVK GDDVRQIAPG QTGVIADYNY KLPDDFMGCV
  421 LAWNTRNIDA TSTGNYNYKY RYLRHGKLRP FERDISNVPF SPDGKPCTPP ALNCYWPLND
  481 YGFYTTTGIG YQPYRVVVLS FELLNAPATV CGPKLSTDLI KNQCVNFNFN GLTGTGVLTP
  541 SSKRFQPFQQ FGRDVSDFTD SVRDPKTSEI LDISPCSFGG VSVITPGTNA SSEVAVLYQD
  601 VNCTDVSTAI HADQLTPAWR IYSTGNNVFQ TQAGCLIGAE HVDTSYECDI PIGAGICASY
  661 HTVSLLRSTS QKSIVAYTMS LGADSSIAYS NNTIAIPTNF SISITTEVMP VSMAKTSVDC
  721 NMYICGDSTE CANLLLQYGS FCTQLNRALS GIAAEQDRNT REVFAQVKQM YKTPTLKYFG
  781 GFNFSQILPD PLKPTKRSFI EDLLFNKVTL ADAGFMKQYG ECLGDINARD LICAQKFNGL
  841 TVLPPLLTDD MIAAYTAALV SGTATAGWTF GAGAALQIPF AMQMAYRFNG IGVTQNVLYE
  901 NQKQIANQFN KAISQIQESL TTTSTALGKL QDVVNQNAQA LNTLVKQLSS NFGAISSVLN
  961 DILSRLDKVE AEVQIDRLIT GRLQSLQTYV TQQLIRAAEI RASANLAATK MSECVLGQSK
1021 RVDFCGKGYH LMSFPQAAPH GVVFLHVTYV PSQERNFTTA PAICHEGKAY FPREGVFVFN
1081 GTSWFITQRN FFSPQIITTD NTFVSGNCDV VIGIINNTVY DPLQPELDSF KEELDKYFKN
1141 HTSPDVDLGD ISGINASVVN IQKEIDRLNE VAKNLNESLI DLQELGKYEQ YIKWPWYVWL
1201 GFIAGLIAIV MVTILLCCMT SCCSCLKGAC SCGSCCKFDE DDSEPVLKGV KLHYT
```

A reference precursor SARS-CoV-2 S protein (UniProtKB—P0DTC2 (SPIKE_SARS2)) is presented herein as SEQ ID NO: 17. Myriad variant SARS CoV-2 S proteins have been sequenced and are available in the literature but share the common structure of SEQ ID NO: 17. As reported in UniProtKB, the signal peptide of the precursor SARS-CoV-2 S protein corresponds to amino acids 1 to 12 of SEQ ID NO: 17, the mature SARS-CoV-2 S protein corresponds to amino acids 13 to 1273 of SEQ ID NO: 17, the S1 region of the SARS-CoV-2 S protein corresponds to amino acids 13 to 685 of SEQ ID NO: 17, the S2 region of the SARS-CoV-2 S protein corresponds to amino acids 686 to 1273 of SEQ ID NO: 17, the receptor binding domain (RBD) of the SARS-CoV-2 S protein corresponds to amino acids 319 to 541 of SEQ ID NO: 17 (underlined below, Yan, R. et al., Science 367:1444-1448 (2020)), the receptor binding motif of the SARS-CoV-2 S protein corresponds to amino acids 437 to 508 of SEQ ID NO: 17, the extracellular domain of the SARS-CoV-2 S protein corresponds to amino acids 13 to 1213 of SEQ ID NO: 17, the transmembrane domain of the SARS-CoV-2 S protein corresponds to amino acids 1214 to 1234 of SEQ ID NO: 17, and the cytoplasmic domain of the SARS-CoV-2 S protein corresponds to amino 1235 to 1273 of SEQ ID NO: 17. As persons of ordinary skill in the art will recognize, the RBD of various SARS-CoV-2 S proteins present in the environment have mutated so an RBD that “corresponds” to amino acids 319 to 541 of SEQ ID NO: 17 may not be identical to amino acids 319 to 541 of SEQ ID NO: 17. The TMPRSS2 or furin cleavage site between the S1 and S2 subunits is between amino acids 685 and 686 of SEQ ID NO: 17 (Hoffmann, M. et al., Cell 181:271-280 (2020)).

### SEQ ID NO: 17: SARS-CoV-2 Spike Protein, UniProt: P0DTC2

```
      1 MFVFLVLLPL VSSQCVNLTT RTQLPPAYTN SFTRGVYYPD KVFRSSVLHS TQDLFLPFFS
    61 NVTWFHAIHV SGTNGTKRFD NPVLPFNDGV YFASTEKSNI IRGWIFGTTL DSKTQSLLIV
  121 NNATNVVIKV CEFQFCNDPF LGVYYHKNNK SWMESEFRVY SSANNCTFEY VSQPFLMDLE
  181 GKQGNFKNLR EFVFKNIDGY FKIYSKHTPI NLVRDLPQGF SALEPLVDLP IGINITRFQT
  241 LLALHRSYLT PGDSSSGWTA GAAAYYVGYL QPRTFLLKYN ENGTITDAVD CALDPLSETK
  301 CTLKSFTVEK GIYQTSNFRV QPTESIVRFP NITNLCPFGE VFNATRFASV YAWNRKRISN
  361 CVADYSVLYN SASFSTFKCY GVSPIKLNDL CFTNVYADSF VIRGDEVRQI APGQTGKIAD
  421 YNYKLPDDFT GCVIAWNSNN LDSKVGGNYN YLYRLFRKSN LKPFERDIST EIYQAGSTPC
  481 NGVEGFNCYF PLQSYGFQPT NGVGYQPYRV VVLSFELLHA PATVCGPKKS TNLVKNKCVN
  541 FNFNGLTGTG VLTESNKKFL PFQQFGRDIA DTTDAVRDPQ TLEILDITPC SFGGVSVITP
  601 GINTSNQVAV LYQDVNCTEV PVAIHADQLT PTWRVYSTGS NVFQTRAGCL IGAEHVNNSY
  661 ECDIPIGAGI CASYQTQTNS PRRAPSVASQ SIIAYTMSLG AENSVAYSNN SIAIPTNFTI
  721 SVTTEILPVS MTKTSVDCTM YICGDSTECS NLLLQYGSFC TQLNRALTGI AVEQDKNTQE
  781 VFAQVKQIYK TPPIKDFGGF NFSQILPDPS KPSKRSFIED LLFNKVTLAD AGFIKQYGDC
  841 LGDIAARDLI CAQKENGLTV LPPLLTDEMI AQYTSALLAG TITSGWTFGA GAALQIPFAM
  901 QMAYRFNGIG VTQNVLYENQ KLIANQFNSA IGKIQDSLSS TASALGKLQD VVNQNAQALN
  961 TLVKQLSSNF GAISSVLNDI LSRLDKVEAE VQIDRLITGR LQSLQTYVTQ QLIRAAEIRA
1021 SANLAATKMS ECVLGQSKRV DFCGKGYHLM SFPQSAPHGV VFLHVTYVPA QEKNFTTAPA
1081 ICHDGKAHFP REGVFVSNGT HWFVTQRNFY EPQIITTDNT FVSGNCDVVI GIVNNTVYDP
1141 LQPELDSFKE ELDKYFKNHT SPDVDLGDIS GINASVVNIQ KEIDRLNEVA KNLNESLIDL
1201 QELGKYEQYI KWPWYIWLGF IAGLIAIVMV TIMLCCMTSC CSCLKGCCSC GSCCKFDEDD
1261 SEPVLKGVKL HYT
```

A reference precursor MERS-CoV S protein (UniProtKB—K9N5Q8 (SPIKE_MERS1)) is presented herein as SEQ ID NO: 18. Myriad variant MERS-CoV S proteins have been sequenced and are available in the literature but share the common structure of SEQ ID NO: 18. As reported in UniProtKB, the signal peptide of the precursor MERS-CoV S protein corresponds to amino acids 1 to 17 of SEQ ID NO: 18, the mature MERS-CoV S protein corresponds to amino acids 18 to 1353 of SEQ ID NO: 18, the S1 region of the MERS-CoV S protein corresponds to amino acids 18 to 751 of SEQ ID NO: 18, the S2 region of the MERS-CoV S protein corresponds to amino acids 752 to 1353 of SEQ ID NO: 18, the receptor binding domain (RBD) of the MERS-CoV S protein corresponds to amino acids 367 to 606 of SEQ ID NO: 18 (underlined below, Wang, et al., Cell Res. 23:986-993 (2013)), the extracellular domain of the MERS-CoV S protein corresponds to amino acids 18 to 1296 of SEQ ID NO: 18, the transmembrane domain of the MERS-CoV S protein corresponds to amino acids 1297 to 1317 of SEQ ID NO: 18, and the cytoplasmic domain of the MERS-CoV S protein corresponds to amino 1318 to 1353 of SEQ ID NO: 18. As persons of ordinary skill in the art will recognize, the RBD of various MERS-CoV S proteins present in the environment have mutated so an RBD that “corresponds” to amino acids 367 to 606 of SEQ ID NO: 18 may not be identical to amino acids 367 to 606 of SEQ ID NO: 18. The furin cleavage site between the S1 and S2 subunits is between amino acids 751 and 752 of SEQ ID NO: 18.

### SEQ ID NO: 18: MERS-CoV Spike Protein, UniProt: K9N5Q8

```
      1 MIHSVFLLMF LLTPTESYVD VGPDSVKSAC IEVDIQQTFF DKTWPRPIDV SKADGIIYPQ
    61 GRTYSNITIT YQGLFPYQGD HGDMYVYSAG HATGTTPQKL FVANYSQDVK QFANGFVVRI
  121 GAAANSTGTV IISPSTSATI RKIYPAFMLG SSVGNFSDGK MGRFFNHTLV LLPDGCGTLL
  181 RAFYCILEPR SGNHCPAGNS YTSFATYHTP ATDCSDGNYN RNASLNSFKE YFNLRNCTFM
  241 YTYNITEDEI LEWFGITQTA QGVHLFSSRY VDLYGGNMFQ FATLPVYDTI KYYSIIPHSI
  301 RSIQSDRKAW AAFYVYKLQP LTFLLDFSVD GYIRRAIDCG FNDLSQLHCS YESFDVESGV
  361 YSVSSFEAKP SGSVVEQAEG VECDFSPLLS GTPPQVYNFK RLVFTNCNYN LTKLLSLFSV
  421 NDFTCSQISP AAIASNCYSS LILDYFSYPL SMKSDLSVSS AGPISQFNYK QSFSNPTCLI
  481 LATVPHNLTT ITKPLKYSYI NKCSRFLSDD RTEVPQLVNA NQYSPCVSIV PSTVWEDGDY
  541 YRKQLSPLEG GGWLVASGST VAMTEQLQMG FGITVQYGTD TNSVCPKLEF ANDTKIASQL
  601 GNCVEYSLYG VEGRGVFQNC TAVGVRQQRF VYDAYQNLVG YYSDDGNYYC LRACVSVPVS
  661 VIYDKETKTH ATLFGSVACE HISSTMSQYS RSTRSMLKRR DSTYGPLQTP VGCVLGLVNS
  721 SLFVEDCKLP LGQSLCALPD TPSTLTPRSV RSVPGEMRLA SIAFNHPIQV DQLNSSYFKL
  781 SIPTNFSFGV TQEYIQTTIQ KVTVDCKQYV CNGFQKCEQL LREYGQFCSK INQALHGANL
  841 RQDDSVRNLF ASVKSSQSSP IIPGFGGDFN LTLLEPVSIS TGSRSARSAI EDLLFDKVTI
  901 ADPGYMQGYD DCMQQGPASA RDLICAQYVA GYKVLPPLMD VNMEAAYTSS LLGSIAGVGW
  961 TAGLSSFAAI PFAQSIFYRL NGVGITQQVL SENQKLIANK FNQALGAMQT GETTTNEAFH
1021 KVQDAVNNNA QALSKLASEL SNTFGAISAS IGDIIQRLDV LEQDAQIDRL INGRLTTLNA
1081 FVAQQLVRSE SAALSAQLAK DKVNECVKAQ SKRSGFCGQG THIVSFVVNA PNGLYFMHVG
1141 YYPSNHIEVV SAYGLCDAAN PTNCIAPVNG YFIKTNNTRI VDEWSYTGSS FYAPEPITSL
1201 NTKYVAPQVT YQNISTNLPP PLLGNSTGID FQDELDEFFK NVSTSIPNFG SLTQINTTLL
1261 DLTYEMLSLQ QVVKALNESY IDLKELGNYT YYNKWPWYIW LGFIAGLVAL ALCVFFILCC
1321 TGCGTNCMGK LKCNRCCDRY EEYDLEPHKV HVH
```

Human coronaviruses are known to develop one or more mutations, particularly in the receptor binding domain (RBD) of the spike (S) protein over time that may alter the behavior of the virus. The SARS-CoV-2 variants are identified, for example, by nomenclature referred to as “pango” lineages (Rambaut, A., et al., Nature Microbiol. 5:1403-1407 (2020)), and have been assigned Greek letter nomenclature by the World Health Organization (who.int/en/activities/tracking-SARS-CoV-2-variants/). SARS CoV-2 variants of clinical relevance are cataloged by the Centers for Disease Control, e.g., at cdc.gov/coronavirus/2019-ncov/variants/variant-info.html (last visited Jul. 5, 2021). The CDC catalog is updated regularly. Those of skill in the art will understand that these lineages do not correspond to exact sequences and are generally characterized by the amino acid variations noted below, particularly in the RBD region, but may be mixtures and may include additional or fewer amino acid deletions, insertions, and/or substitutions relative to SEQ ID NO: 17.

For example, the pango lineage B.1.1.7 or WHO “Alpha” variant first identified in the UK includes an RBD substitution of tyrosine (Y) for asparagine (N) at a position corresponding to amino acid 501 in SEQ ID NO: 17, and can include additional spike protein alterations such as amino acid deletions at positions corresponding to amino acids 69, 70, and 144 of SEQ ID NO: 17, and amino acid substitutions A570D, D614G, P681H, T716I, S982A, D1118H corresponding to the indicated positions in SEQ ID NO: 17. By “an amino acid corresponding to amino acid 501 in SEQ ID NO: 17 (and other spike protein mutations described herein) is meant the amino acid in the sequence of any given SARS-CoV-2 spike protein, which is homologous to N501 in SEQ ID NO: 17. Variant viruses carrying this “N501Y” mutation have been shown to be more highly transmissible than the non-variant virus. See Leung, K., et al., Euro Surveill. 26:2002106. doi: 10.2807/1560-7917.ES.2020.26.1.2002106 (2021).

The pango lineage B.1.351 or WHO “Beta” variant first identified in South Africa includes K417N, E484K and N501Y RBD substitutions corresponding to the indicated positions in SEQ ID NO: 17 and can include additional spike protein substitutions such as D80A, D215G, D614G, and A701V and amino acid deletions corresponding to amino acids 241-243 (all positions corresponding to SEQ ID NO: 17). This variant is likewise believed to be more highly transmissible. See Wibmer, C K et al., Nature Med.: 27:622-625, doi: 10.1038/s41591-021-01285-x (2021).

The pango lineage P.1 or WHO “Gamma” variant first identified in Brazil includes K417T, E484K, and N501Y RBD substitutions corresponding to the indicated positions in SEQ ID NO: 17 and can include additional spike protein substitutions such as L18F, T20N, P26S, D138Y, R190S, D614G, H655Y, and T10271 (all positions corresponding to SEQ ID NO: 17). This variant again is believed to be more highly transmissible. See Faria, Nuno R., et al., Virological (2021) (available at icpcovid.com, visited Feb. 19, 2021). Additionally, a SARS-CoV-2 variant with a D614G mutation is believed to have increased infectivity and transmissibility (Korber B., et al., Cell 182:812-827 (2020)).

The pango lineage B.1.525 or WHO “Eta” variant first identified in Nigeria includes an E484K RBD substitution corresponding to the indicated position in SEQ ID NO: 17 and can include additional spike protein alterations such as amino acid deletions at positions corresponding to amino acids 69, 70, and 144 of SEQ ID NO: 17, and spike protein substitutions Q52R, A67V, D614G, Q677H, F888L (all positions corresponding to SEQ ID NO: 17).

The pango lineage B.1.617.1 or WHO “Kappa” variant first identified in India includes L452R and E484Q RBD substitutions corresponding to the indicated positions in SEQ ID NO: 17 and can include spike protein substitutions such as G142D, E154K, D614G, P681R, Q1071H, and H1101D (all positions corresponding to SEQ ID NO: 17).

The pango lineage B.1.617.2 or WHO “Delta” variant first identified in India includes L452R and T478K RBD substitutions corresponding to the indicated positions in SEQ ID NO: 17 and can include additional spike protein substitutions such as T19R, G142D, D614G, P681R, and D950N (all positions corresponding to SEQ ID NO: 17). A newly-emerging Delta variant, designated “Delta Plus” or Pango lineage B.1.617.2/AY.1 further includes a K417N substitution corresponding to SEQ ID NO: 17.

The pango lineage B.1.617.2.v2 variant first identified in India includes L452R and T478K RBD substitutions corresponding to the indicated positions in SEQ ID NO: 17 and can include additional spike protein alterations such as amino acid deletions at positions corresponding to amino acids 157 and 158 of SEQ ID NO: 17, and spike protein substitutions such as T19R, G142D, E156G, D614G, P681R, and D950N (all positions corresponding to SEQ ID NO: 17).

The pango lineage B.1.618 variant first identified in India includes an E484K RBD substitution corresponding to the indicated position in SEQ ID NO: 17 and can include additional spike protein alterations such as amino acid deletions at positions corresponding to amino acids 145 and 146 of SEQ ID NO: 17, and spike protein substitutions such as H49Y and D614G (all positions corresponding to SEQ ID NO: 17).

Of particular concern is the potential development of escape mutations that can reduce or prevent neutralization by a therapy, such as an antibody therapy. The multimeric binding molecules disclosed herein may be able to maintain the ability to bind and neutralize strains of the human coronavirus that are escape mutants for the corresponding IgG antibody. Additionally, the multimeric binding molecules disclosed herein may be less prone to generating escape mutants. Accordingly, in certain embodiments, the human coronavirus, e.g., SARS-CoV-2, is a reference IgG antibody escape mutant.

Prevalent SARS-CoV-2 escape mutations include, without limitation, N439K, Y453F, S477N, and N501Y, which are four prevalent RBD mutations in circulation and are associated with resistance to several neutralizing mAbs (Thomson, E. C., et al. Cell 184: 1171-1187 (2021); Liu, Z., et al. bioRxiv doi: 10.2139/ssrn.3725763 (2020); Weisblum, Y., et al., eLife 9:e61312 doi: 10.7554/eLife.61312 (Oct. 28, 2020); Hayashi et al. medRxiv doi: 10.1101/2021.01.28.21250577). Other RBD mutations are associated with resistance to three approved mAbs, Bamlanivimab (E484K, F490S, Q493R, S494P), Casivirivimab (REGN-10933, K417E, Y453F, L455F, G476S, F486V, Q493K) and Imdevimab (REGN-10987, K444Q, V445A, G446V) (Fact Sheet for Health Care Providers Emergency Use Authorization (EUA) of Bamlanivimab, (2020); Fact Sheet for Health Care Providers Emergency Use Authorization (EUA) Of Casirivimab and Imdevimab, (2020), both available at fda.gov (visited Jan. 25, 2021).

Exemplary escape mutations in the SARS-CoV S protein corresponding to SEQ ID NO: 16 that block neutralization by selected SARS-CoV antibodies are identified, e.g., in Rockx, B., et al. J. Infect. Dis. 201:946-955 (2010), and Sui, et al., J. Virol. 88:13769-13780 (2014).

Exemplary MERS-CoV escape mutations in the MERS-CoV S protein corresponding to SEQ ID NO: 18 that block neutralization by selected MERS-CoV antibodies are identified, e.g., in Kleine Weber et al., J. Virol. 93(2):e01381-18 (2019).

Antibody-dependent enhancement (ADE) of diseases caused by human coronaviruses is a concern (Houser, K. V., et al., PLoS Pathog. 13: Article e1006565 (2017) (MERS-CoV); Weiss, R. C., and F. W. Scott Comp. Immunol. Microbiol. Infect. Dis 4:175-189 (1981) (feline infectious peritonitis virus); and Kam, Y. W., et al., Vaccine 25:729-740 (2007) (SARS-CoV)). It is believed that Fcγ receptors may mediate antibody dependent entry into cells (Kam et al., supra). Fcγ receptors do not bind IgA or IgM antibodies. Accordingly, the multimeric binding molecules disclosed herein can have a reduced risk of ADE than the reference IgG antibody. In some embodiments, the multimeric binding molecule cannot cause ADE.

As the terms “SARS” and “MERS” imply, infection with SARS-CoV, SARS-CoV-2, and MERS-CoV often leads to severe respiratory symptoms. These respiratory symptoms make the respiratory mucosa an important tissue to target for any molecule developed to treat or prevent SARS, COVID-19, or MERS; however, the epithelium often prevents the transcytosis of therapeutics such as IgG antibodies. By contrast, polymeric immunoglobulin receptors (pIgR) on the epithelium bind the J-chain of IgA and IgM antibodies and transports the molecules across the epithelia to the mucosa. During this process, the process, the extracellular region of pIgR is cleaved and is then termed the secretory component. Accordingly, in some embodiments, the multimeric binding molecule can transport across vascular endothelial cells via J-chain binding to the polymeric Ig receptor (PIgR). It is believed that recombinant secretory component can also facilitate transcytosis of J-chain comprising molecules to the mucosa. Accordingly, in some embodiments, the multimeric binding molecule comprises a secretory component, or fragment or variant thereof. In some embodiments, the multimeric binding molecule comprises a secretory component.

In certain embodiments, the binding molecule is more potent than a bivalent reference IgG antibody comprising two of the human coronavirus, e.g., SARS-CoV, SARS-CoV-2 or MERS-CoV, binding domains. In some embodiments, the binding molecule neutralizes human coronavirus, e.g., SARS-CoV, SARS-CoV-2 or MERS-CoV infectivity at a lower 50% effective concentration (EC₅₀) than the bivalent reference IgG antibody. In some embodiments, the EC₅₀ is at least two-fold, at least three-fold, at least four-fold, at least five-fold, at least ten-fold, at least twenty-fold, at least thirty-fold, at least forty-fold, at least fifty-fold, at least seventy-five-fold, at least 100-fold, at least 200-fold, at least 300-fold, at least 400-fold, at least 500-fold, at least 750-fold or at least 1000-fold lower than the EC₅₀ of the bivalent reference IgG antibody.

In some embodiments, the multimeric binding molecule reduces, inhibits, or blocks the human coronavirus, e.g., SARS-CoV or SARS-CoV-2 from binding to its receptor, e.g., angiotensin-converting enzyme 2 (ACE2). In some embodiments, the multimeric binding molecule reduces, inhibits, or blocks the human coronavirus, e.g., MERS-CoV binding to its receptor, e.g., dipeptidyl peptidase 4 (DPP4). In some embodiments, the multimeric binding molecule reduces, inhibits, or blocks the human coronavirus binding to its receptor more than a bivalent reference IgG antibody comprising two of the human coronavirus, e.g., SARS-CoV, SARS-CoV-2 or MERS-CoV, binding domains. In some embodiments, the multimeric binding molecule reduces, inhibits, or blocks SARS-CoV or SARS-CoV2 binding to ACE2 more than a bivalent reference IgG antibody comprising two of the human coronavirus, e.g., SARS-CoV or SARS-CoV-2, binding domains. In some embodiments, the multimeric binding molecule reduces, inhibits, or blocks MERS-CoV binding to DPP4 more than a bivalent reference IgG antibody comprising two of the human coronavirus, e.g., MERS-CoV, binding domains. In some embodiments, the binding molecule inhibits human coronavirus, e.g., SARS-CoV-2 binding to its receptor, e.g., ACE2, at a lower 50% inhibitory concentration (IC₅₀) than the bivalent reference IgG antibody. In some embodiments, the binding molecule inhibits human coronavirus, e.g., MERS-CoV binding to its receptor, e.g., DPP4, at a lower 50% inhibitory concentration (IC₅₀) than the bivalent reference IgG antibody. In some embodiments, the IC₅₀ is at least two-fold, at least three-fold, at least four-fold, at least five-fold, at least ten-fold, at least twenty-fold, at least thirty-fold, at least forty-fold, at least fifty-fold, at least seventy-five-fold, at least 100-fold, at least 200-fold, at least 300-fold, at least 400-fold, at least 500-fold, at least 750-fold or at least 1000-fold lower than the IC₅₀ of the bivalent reference IgG antibody.

In some embodiments, the multimeric binding molecules are dimeric and comprise two bivalent binding units or variants or fragments thereof. In some embodiments, the multimeric binding molecules are dimeric, comprise two bivalent binding units or variants or fragments thereof, and further comprise a J-chain or functional fragment or variant thereof as described herein. In some embodiments, the multimeric binding molecules are dimeric, comprise two bivalent binding units or variants or fragments thereof, and further comprise a J-chain or functional fragment or variant thereof as described herein, where each binding unit comprises two IgA heavy chain constant regions or multimerizing fragments or variants thereof.

In some embodiments, the multimeric binding molecules are tetrameric and comprise four bivalent binding units or variants or fragments thereof. In some embodiments, the multimeric binding molecules are tetrameric, comprise four bivalent binding units or variants or fragments thereof, and further comprise a J-chain or functional fragment or variant thereof as described herein. In some embodiments, the multimeric binding molecules are tetrameric, comprise four bivalent binding units or variants or fragments thereof, and further comprise a J-chain or functional fragment or variant thereof as described herein, where each binding unit comprises two IgA heavy chain constant regions or multimerizing fragments or variants thereof.

In some embodiments, the multimeric binding molecules are pentameric and comprise five bivalent binding units or variants or fragments thereof. In some embodiments, the multimeric binding molecules are pentameric and comprise five bivalent binding units or variants or fragments thereof, and further comprise a J-chain or functional fragment or variant thereof as described herein. In some embodiments, the multimeric binding molecules are pentameric and comprise five bivalent binding units or variants or fragments thereof, and further comprise a J-chain or functional fragment or variant thereof as described herein, where each binding unit comprises two IgM heavy chain constant regions or multimerizing fragments or variants thereof.

In some embodiments, the multimeric binding molecules are hexameric and comprise six bivalent binding units or variants or fragments thereof. In some embodiments, the multimeric binding molecules are hexameric and comprise six bivalent binding units or variants or fragments thereof, and where each binding unit comprises two IgM heavy chain constant regions or multimerizing fragments or variants thereof.

In certain embodiments, heavy chain constant regions in the provided binding molecules are each associated with a binding domain, e.g., an antibody antigen-binding domain, e.g., a scFv, a VHH or the VH subunit of an antibody antigen-binding domain. The multimeric binding molecule disclosed herein can comprise three to twelve binding domains that are human coronavirus, e.g., SARS-CoV, SARS-CoV-2 or MERS-CoV binding domains. In some embodiments, the multimeric binding molecule, such as an IgA antibody, an IgA-like antibody, or an IgA-derived binding molecule comprises three to eight binding domains that specifically bind to the human coronavirus, e.g., SARS-CoV, SARS-CoV-2 or MERS-CoV. In some embodiments, the multimeric binding molecule, such as an IgA antibody, an IgA-like antibody, or an IgA-derived binding molecule comprises four binding domains that specifically bind to the human coronavirus, e.g., SARS-CoV, SARS-CoV-2 or MERS-CoV. In some embodiments, the multimeric binding molecule, such as an IgA antibody, an IgA-like antibody, or an IgA-derived binding molecule comprises eight binding domains that specifically bind to the human coronavirus, e.g., SARS-CoV, SARS-CoV-2 or MERS-CoV. In some embodiments, the multimeric binding molecule, such as an IgM antibody, an IgM-like antibody, or an IgM-derived binding molecule comprises ten or twelve binding domains that specifically bind to the human coronavirus, e.g., SARS-CoV, SARS-CoV-2 or MERS-CoV.

In certain embodiments, the provided multimeric binding molecule is multispecific, e.g., bispecific, trispecific, or tetraspecific, where two or more binding domains associated with the heavy chain constant regions of the binding molecule specifically bind to different targets. In certain embodiments, the binding domains of the multimeric binding molecule all specifically bind to the human coronavirus, e.g., SARS-CoV, SARS-CoV-2 or MERS-CoV. In certain embodiments, the binding domains of the multimeric binding molecule are identical. In such cases, the multimeric binding molecule can still be bispecific, if, for example, a binding domain with a different specificity is part of a modified J-chain as described elsewhere herein. In certain embodiments, the binding domains are antibody-derived antigen-binding domains, e.g., a scFv associated with the heavy chain constant regions or a VH subunit of an antibody binding domain associated with the heavy chain constant regions.

For example, in some embodiments, the provided multimeric binding molecule binds a conserved, or highly conserved, cryptic receptor binding domain epitope and cross-reacts or binds spike protein epitopes of all human corona viruses. See, e.g., Tortorici et al., Nature, (2021) doi: 10.1038/s41586-021-03817-4. In some embodiments, the multimeric binding protein that binds all human coronaviruses comprise a heavy chain variable region (VH) and a light chain variable region (VL), wherein the VH and VL comprise six immunoglobulin complementarity determining regions HCDR1, HCDR2, HCDR3, LCDR1, LCDR2, and LCDR3 comprise the CDRs of an antibody comprising the VH and VL of SEQ ID NOS:384 and 385. In some embodiments the multimeric binding protein that binds all human coronaviruses is an IgM antibody. In some embodiments the IgM antibody comprises a modified or variant J-chain. In some embodiments the multimeric binding protein that binds all human coronaviruses is an IgA antibody.

In certain embodiments, each binding unit comprises two heavy chains each comprising a VH situated amino terminal to the heavy chain constant region, and two immunoglobulin light chains each comprising a light chain variable domain (VL) situated amino terminal to an immunoglobulin light chain constant region, e.g., a kappa or lambda constant region. The provided VH and VL combine to form an antigen-binding domain that specifically binds to the target. In certain embodiments each antigen-binding domain of each binding molecule binds to the human coronavirus, e.g., SARS-CoV, SARS-CoV-2 or MERS-CoV. In certain embodiments, each antigen-binding domain of each binding molecule is identical.

In certain embodiments, the three to twelve identical binding domains of the multimeric binding molecule that bind to SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of any of SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 104 and SEQ ID NO: 105, SEQ ID NO: 106 and SEQ ID NO: 107, SEQ ID NO: 108 and SEQ ID NO: 109, SEQ ID NO: 110 and SEQ ID NO: 111, SEQ ID NO: 112 and SEQ ID NO: 113, SEQ ID NO: 114 and SEQ ID NO: 115, SEQ ID NO: 116 and SEQ ID NO: 117, SEQ ID NO: 118 and SEQ ID NO: 119, SEQ ID NO: 120 and SEQ ID NO: 121, SEQ ID NO: 122 and SEQ ID NO: 123, SEQ ID NO: 124 and SEQ ID NO: 125, SEQ ID NO: 126 and SEQ ID NO: 127, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 150 and SEQ ID NO: 151, SEQ ID NO: 152 and SEQ ID NO: 153, SEQ ID NO: 154 and SEQ ID NO: 155, SEQ ID NO: 156 and SEQ ID NO: 157, SEQ ID NO: 158 and SEQ ID NO: 159, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 178 and SEQ ID NO: 179, SEQ ID NO: 180 and SEQ ID NO: 181, SEQ ID NO: 182 and SEQ ID NO: 183, SEQ ID NO: 184 and SEQ ID NO: 185, SEQ ID NO: 186 and SEQ ID NO: 187, SEQ ID NO: 188 and SEQ ID NO: 189, SEQ ID NO: 190 and SEQ ID NO: 191, SEQ ID NO: 192 and SEQ ID NO: 193, SEQ ID NO: 194 and SEQ ID NO: 195, SEQ ID NO: 196 and SEQ ID NO: 197, SEQ ID NO: 198 and SEQ ID NO: 199, SEQ ID NO: 200 and SEQ ID NO: 201, SEQ ID NO: 202 and SEQ ID NO: 203, SEQ ID NO: 204 and SEQ ID NO: 205, SEQ ID NO: 206 and SEQ ID NO: 207, SEQ ID NO: 208 and SEQ ID NO: 209, SEQ ID NO: 210 and SEQ ID NO: 211, SEQ ID NO: 212 and SEQ ID NO: 213, SEQ ID NO: 214 and SEQ ID NO: 215, SEQ ID NO: 216 and SEQ ID NO: 217, SEQ ID NO: 218 and SEQ ID NO: 219, SEQ ID NO: 220 and SEQ ID NO: 221, SEQ ID NO: 222 and SEQ ID NO: 223, SEQ ID NO: 224 and SEQ ID NO: 225, SEQ ID NO: 226 and SEQ ID NO: 227, SEQ ID NO: 228 and SEQ ID NO: 229, SEQ ID NO: 230 and SEQ ID NO: 231, SEQ ID NO: 232 and SEQ ID NO: 233, SEQ ID NO: 234 and SEQ ID NO: 235, SEQ ID NO: 236 and SEQ ID NO: 237, SEQ ID NO: 238 and SEQ ID NO: 239, SEQ ID NO: 240 and SEQ ID NO: 241, SEQ ID NO: 242 and SEQ ID NO: 243, SEQ ID NO: 244 and SEQ ID NO: 245, SEQ ID NO: 246 and SEQ ID NO: 247, SEQ ID NO: 248 and SEQ ID NO: 249, SEQ ID NO: 250 and SEQ ID NO: 251, SEQ ID NO: 252 and SEQ ID NO: 253, SEQ ID NO: 254 and SEQ ID NO: 255, SEQ ID NO: 256 and SEQ ID NO: 257, SEQ ID NO: 258 and SEQ ID NO: 259, SEQ ID NO: 260 and SEQ ID NO: 261, SEQ ID NO: 264 and SEQ ID NO: 265, SEQ ID NO: 266 and SEQ ID NO: 267, SEQ ID NO: 268 and SEQ ID NO: 269, SEQ ID NO: 270 and SEQ ID NO: 271, SEQ ID NO: 272 and SEQ ID NO: 273, SEQ ID NO: 274 and SEQ ID NO: 275, SEQ ID NO: 276 and SEQ ID NO: 277, SEQ ID NO: 278 and SEQ ID NO: 279, SEQ ID NO: 280 and SEQ ID NO: 281, SEQ ID NO: 282 and SEQ ID NO: 283, SEQ ID NO: 284 and SEQ ID NO: 285, SEQ ID NO: 286 and SEQ ID NO: 287, SEQ ID NO: 288 and SEQ ID NO: 289, SEQ ID NO: 290 and SEQ ID NO: 291, SEQ ID NO: 292 and SEQ ID NO: 293, SEQ ID NO: 294 and SEQ ID NO: 295, SEQ ID NO: 296 and SEQ ID NO: 297, SEQ ID NO: 298 and SEQ ID NO: 299, SEQ ID NO: 300 and SEQ ID NO: 301, SEQ ID NO: 302 and SEQ ID NO: 303, SEQ ID NO: 304 and SEQ ID NO: 305, SEQ ID NO: 306 and SEQ ID NO: 307, SEQ ID NO: 308 and SEQ ID NO: 309, SEQ ID NO: 310 and SEQ ID NO: 311, SEQ ID NO: 312 and SEQ ID NO: 313, SEQ ID NO: 314 and SEQ ID NO: 315, SEQ ID NO: 316 and SEQ ID NO: 317, SEQ ID NO: 318 and SEQ ID NO: 319, SEQ ID NO: 320 and SEQ ID NO: 321, SEQ ID NO: 322 and SEQ ID NO: 323, SEQ ID NO: 324 and SEQ ID NO: 325, SEQ ID NO: 326 and SEQ ID NO: 327, SEQ ID NO: 328 and SEQ ID NO: 329, SEQ ID NO: 330 and SEQ ID NO: 331, SEQ ID NO: 332 and SEQ ID NO: 333, SEQ ID NO: 336 and SEQ ID NO: 337, SEQ ID NO: 338 and SEQ ID NO: 339, SEQ ID NO: 340 and SEQ ID NO: 341, SEQ ID NO: 342 and SEQ ID NO: 343, SEQ ID NO: 344 and SEQ ID NO: 345, SEQ ID NO: 348 and SEQ ID NO: 349, SEQ ID NO: 350 and SEQ ID NO: 351, SEQ ID NO: 352 and SEQ ID NO: 353, SEQ ID NO: 354 and SEQ ID NO: 355, SEQ ID NO: 356 and SEQ ID NO: 357, SEQ ID NO: 358 and SEQ ID NO: 359, SEQ ID NO: 362 and SEQ ID NO: 363, SEQ ID NO: 364 and SEQ ID NO: 365, SEQ ID NO: 366 and SEQ ID NO: 367, SEQ ID NO: 368 and SEQ ID NO: 369, SEQ ID NO: 370 and SEQ ID NO: 371, SEQ ID NO: 372 and SEQ ID NO: 373, SEQ ID NO: 374 and SEQ ID NO: 375, SEQ ID NO: 376 and SEQ ID NO: 377, SEQ ID NO: 378 and SEQ ID NO: 379, SEQ ID NO: 380 and SEQ ID NO: 381, SEQ ID NO: 382 and SEQ ID NO: 383, SEQ ID NO: 384 and SEQ ID NO: 385, SEQ ID NO: 386 and SEQ ID NO: 387, SEQ ID NO: 388 and SEQ ID NO: 389, SEQ ID NO: 390 and SEQ ID NO: 391, SEQ ID NO: 392 and SEQ ID NO: 393, SEQ ID NO: 394 and SEQ ID NO: 395, SEQ ID NO: 396 and SEQ ID NO: 397, SEQ ID NO: 398 and SEQ ID NO: 399, SEQ ID NO: 400 and SEQ ID NO: 401, SEQ ID NO: 402 and SEQ ID NO: 403, SEQ ID NO: 404 and SEQ ID NO: 405, SEQ ID NO: 406 and SEQ ID NO: 407, SEQ ID NO: 408 and SEQ ID NO: 409, SEQ ID NO: 410 and SEQ ID NO: 411, SEQ ID NO: 412 and SEQ ID NO: 413, SEQ ID NO: 414 and SEQ ID NO: 415, SEQ ID NO: 416 and SEQ ID NO: 417, SEQ ID NO: 418 and SEQ ID NO: 419, SEQ ID NO: 420 and SEQ ID NO: 421, SEQ ID NO: 422 and SEQ ID NO: 423, SEQ ID NO: 424 and SEQ ID NO: 425, SEQ ID NO: 426 and SEQ ID NO: 427, SEQ ID NO: 428 and SEQ ID NO: 429, SEQ ID NO: 430 and SEQ ID NO: 431, SEQ ID NO: 432 and SEQ ID NO: 433, SEQ ID NO: 434 and SEQ ID NO: 435, SEQ ID NO: 436 and SEQ ID NO: 437, SEQ ID NO: 438 and SEQ ID NO: 439, SEQ ID NO: 440 and SEQ ID NO: 441, SEQ ID NO: 442 and SEQ ID NO: 443, SEQ ID NO: 444 and SEQ ID NO: 445, SEQ ID NO: 446 and SEQ ID NO: 447, SEQ ID NO: 448 and SEQ ID NO: 449, SEQ ID NO: 450 and SEQ ID NO: 451, SEQ ID NO: 452 and SEQ ID NO: 453, SEQ ID NO: 454 and SEQ ID NO: 455, SEQ ID NO: 456 and SEQ ID NO: 457, SEQ ID NO: 458 and SEQ ID NO: 459, SEQ ID NO: 460 and SEQ ID NO: 461, SEQ ID NO: 462 and SEQ ID NO: 463, SEQ ID NO: 464 and SEQ ID NO: 465, SEQ ID NO: 466 and SEQ ID NO: 467, SEQ ID NO: 468 and SEQ ID NO: 469, SEQ ID NO: 470 and SEQ ID NO: 471, SEQ ID NO: 472 and SEQ ID NO: 473, SEQ ID NO: 474 and SEQ ID NO: 475, SEQ ID NO: 476 and SEQ ID NO: 477, SEQ ID NO: 478 and SEQ ID NO: 479, SEQ ID NO: 480 and SEQ ID NO: 481, SEQ ID NO: 482 and SEQ ID NO: 483, SEQ ID NO: 484 and SEQ ID NO: 485, SEQ ID NO: 486 and SEQ ID NO: 487, SEQ ID NO: 488 and SEQ ID NO: 489, SEQ ID NO: 490 and SEQ ID NO: 491, SEQ ID NO: 492 and SEQ ID NO: 493, SEQ ID NO: 494 and SEQ ID NO: 495, SEQ ID NO: 496 and SEQ ID NO: 497, SEQ ID NO: 498 and SEQ ID NO: 499, SEQ ID NO: 500 and SEQ ID NO: 501, SEQ ID NO: 502 and SEQ ID NO: 503, SEQ ID NO: 504 and SEQ ID NO: 505, SEQ ID NO: 506 and SEQ ID NO: 507, SEQ ID NO: 508 and SEQ ID NO: 509, SEQ ID NO: 628 and SEQ ID NO: 629, SEQ ID NO: 630 and SEQ ID NO: 631, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, or SEQ ID NO: 644 SEQ ID NO: 645, SEQ ID NO: 646 and SEQ ID NO: 647, SEQ ID NO: 648 and SEQ ID NO: 649, SEQ ID NO: 650 and SEQ ID NO: 651, or SEQ ID NO: 652 and SEQ ID NO: 653, respectively with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 106 and SEQ ID NO: 107, SEQ ID NO: 110 and SEQ ID NO: 111, SEQ ID NO: 118 and SEQ ID NO: 119, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 142 and SEQ ID NO: 143, SEQ ID NO: 146 and SEQ ID NO: 147, SEQ ID NO: 150 and SEQ ID NO: 151, SEQ ID NO: 156 and SEQ ID NO: 157, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 180 and SEQ ID NO: 181, SEQ ID NO: 182 and SEQ ID NO: 183, SEQ ID NO: 186 and SEQ ID NO: 187, SEQ ID NO: 188 and SEQ ID NO: 189, SEQ ID NO: 194 and SEQ ID NO: 195, SEQ ID NO: 200 and SEQ ID NO: 201, SEQ ID NO: 206 and SEQ ID NO: 207, SEQ ID NO: 208 and SEQ ID NO: 209, SEQ ID NO: 218 and SEQ ID NO: 219, SEQ ID NO: 220 and SEQ ID NO: 221, SEQ ID NO: 222 and SEQ ID NO: 223, SEQ ID NO: 224 and SEQ ID NO: 225, SEQ ID NO: 228 and SEQ ID NO: 229, SEQ ID NO: 230 and SEQ ID NO: 231, SEQ ID NO: 240 and SEQ ID NO: 241, SEQ ID NO: 244 and SEQ ID NO: 245, SEQ ID NO: 246 and SEQ ID NO: 247, SEQ ID NO: 256 and SEQ ID NO: 257, SEQ ID NO: 258 and SEQ ID NO: 259, SEQ ID NO: 260 and SEQ ID NO: 261, SEQ ID NO: 264 and SEQ ID NO: 265, SEQ ID NO: 266 and SEQ ID NO: 267, SEQ ID NO: 268 and SEQ ID NO: 269, SEQ ID NO: 270 and SEQ ID NO: 271, SEQ ID NO: 272 and SEQ ID NO: 273, SEQ ID NO: 274 and SEQ ID NO: 275, SEQ ID NO: 276 and SEQ ID NO: 277, SEQ ID NO: 278 and SEQ ID NO: 279, SEQ ID NO: 280 and SEQ ID NO: 281, SEQ ID NO: 282 and SEQ ID NO: 283, SEQ ID NO: 284 and SEQ ID NO: 285, SEQ ID NO: 286 and SEQ ID NO: 287, SEQ ID NO: 288 and SEQ ID NO: 289, SEQ ID NO: 290 and SEQ ID NO: 291, SEQ ID NO: 292 and SEQ ID NO: 293, SEQ ID NO: 294 and SEQ ID NO: 295, SEQ ID NO: 296 and SEQ ID NO: 297, SEQ ID NO: 298 and SEQ ID NO: 299, SEQ ID NO: 300 and SEQ ID NO: 301, SEQ ID NO: 302 and SEQ ID NO: 303, SEQ ID NO: 304 and SEQ ID NO: 305, SEQ ID NO: 306 and SEQ ID NO: 307, SEQ ID NO: 308 and SEQ ID NO: 309, SEQ ID NO: 310 and SEQ ID NO: 311, SEQ ID NO: 312 and SEQ ID NO: 313, SEQ ID NO: 314 and SEQ ID NO: 315, SEQ ID NO: 316 and SEQ ID NO: 317, SEQ ID NO: 318 and SEQ ID NO: 319, SEQ ID NO: 320 and SEQ ID NO: 321, SEQ ID NO: 322 and SEQ ID NO: 323, SEQ ID NO: 324 and SEQ ID NO: 325, SEQ ID NO: 326 and SEQ ID NO: 327, SEQ ID NO: 328 and SEQ ID NO: 329, SEQ ID NO: 330 and SEQ ID NO: 331, SEQ ID NO: 332 and SEQ ID NO: 333, SEQ ID NO: 336 and SEQ ID NO: 337, SEQ ID NO: 338 and SEQ ID NO: 339, SEQ ID NO: 340 and SEQ ID NO: 341, SEQ ID NO: 342 and SEQ ID NO: 343, SEQ ID NO: 344 and SEQ ID NO: 345, SEQ ID NO: 348 and SEQ ID NO: 349, SEQ ID NO: 350 and SEQ ID NO: 351, SEQ ID NO: 352 and SEQ ID NO: 353, SEQ ID NO: 354 and SEQ ID NO: 355, SEQ ID NO: 356 and SEQ ID NO: 357, SEQ ID NO: 358 and SEQ ID NO: 359, SEQ ID NO: 360 and SEQ ID NO: 361, SEQ ID NO: 362 and SEQ ID NO: 363, SEQ ID NO: 364 and SEQ ID NO: 365, SEQ ID NO: 366 and SEQ ID NO: 367, SEQ ID NO: 368 and SEQ ID NO: 369, SEQ ID NO: 370 and SEQ ID NO: 371, SEQ ID NO: 372 and SEQ ID NO: 373, SEQ ID NO: 374 and SEQ ID NO: 375, SEQ ID NO: 376 and SEQ ID NO: 377, SEQ ID NO: 378 and SEQ ID NO: 379, SEQ ID NO: 380 and SEQ ID NO: 381, SEQ ID NO: 382 and SEQ ID NO: 383, SEQ ID NO: 384 and SEQ ID NO: 385, SEQ ID NO: 386 and SEQ ID NO: 387, SEQ ID NO: 388 and SEQ ID NO: 389, SEQ ID NO: 390 and SEQ ID NO: 391, SEQ ID NO: 392 and SEQ ID NO: 393, SEQ ID NO: 394 and SEQ ID NO: 395, SEQ ID NO: 396 and SEQ ID NO: 397, SEQ ID NO: 398 and SEQ ID NO: 399, SEQ ID NO: 400 and SEQ ID NO: 401, SEQ ID NO: 402 and SEQ ID NO: 403, SEQ ID NO: 404 and SEQ ID NO: 405, SEQ ID NO: 406 and SEQ ID NO: 407, SEQ ID NO: 408 and SEQ ID NO: 409, SEQ ID NO: 410 and SEQ ID NO: 411, SEQ ID NO: 412 and SEQ ID NO: 413, SEQ ID NO: 414 and SEQ ID NO: 415, SEQ ID NO: 416 and SEQ ID NO: 417, SEQ ID NO: 418 and SEQ ID NO: 419, SEQ ID NO: 420 and SEQ ID NO: 421, SEQ ID NO: 422 and SEQ ID NO: 423, SEQ ID NO: 424 and SEQ ID NO: 425, SEQ ID NO: 426 and SEQ ID NO: 427, SEQ ID NO: 428 and SEQ ID NO: 429, SEQ ID NO: 430 and SEQ ID NO: 431, SEQ ID NO: 432 and SEQ ID NO: 433, SEQ ID NO: 434 and SEQ ID NO: 435, SEQ ID NO: 436 and SEQ ID NO: 437, SEQ ID NO: 438 and SEQ ID NO: 439, SEQ ID NO: 440 and SEQ ID NO: 441, SEQ ID NO: 442 and SEQ ID NO: 443, SEQ ID NO: 444 and SEQ ID NO: 445, SEQ ID NO: 446 and SEQ ID NO: 447, SEQ ID NO: 448 and SEQ ID NO: 449, SEQ ID NO: 450 and SEQ ID NO: 451, SEQ ID NO: 452 and SEQ ID NO: 453, SEQ ID NO: 454 and SEQ ID NO: 455, SEQ ID NO: 456 and SEQ ID NO: 457, SEQ ID NO: 458 and SEQ ID NO: 459, SEQ ID NO: 460 and SEQ ID NO: 461, SEQ ID NO: 462 and SEQ ID NO: 463, SEQ ID NO: 464 and SEQ ID NO: 465, SEQ ID NO: 466 and SEQ ID NO: 467, SEQ ID NO: 468 and SEQ ID NO: 469, SEQ ID NO: 470 and SEQ ID NO: 471, SEQ ID NO: 472 and SEQ ID NO: 473, SEQ ID NO: 474 and SEQ ID NO: 475, SEQ ID NO: 476 and SEQ ID NO: 477, SEQ ID NO: 478 and SEQ ID NO: 479, SEQ ID NO: 480 and SEQ ID NO: 481, SEQ ID NO: 482 and SEQ ID NO: 483, SEQ ID NO: 484 and SEQ ID NO: 485, SEQ ID NO: 486 and SEQ ID NO: 487, SEQ ID NO: 488 and SEQ ID NO: 489, SEQ ID NO: 490 and SEQ ID NO: 491, SEQ ID NO: 492 and SEQ ID NO: 493, SEQ ID NO: 494 and SEQ ID NO: 495, SEQ ID NO: 496 and SEQ ID NO: 497, SEQ ID NO: 498 and SEQ ID NO: 499, SEQ ID NO: 500 and SEQ ID NO: 501, SEQ ID NO: 502 and SEQ ID NO: 503, SEQ ID NO: 504 and SEQ ID NO: 505, SEQ ID NO: 506 and SEQ ID NO: 507, SEQ ID NO: 508 and SEQ ID NO: 509, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, SEQ ID NO: 646 and SEQ ID NO: 647, SEQ ID NO: 648 and SEQ ID NO: 649, SEQ ID NO: 650 and SEQ ID NO: 651, or SEQ ID NO: 652 and SEQ ID NO: 653, respectively with zero, one, or two single amino acid substitutions in one or more of the HCDRs or LCDRs, wherein the bivalent reference IgG antibody comprising two of the binding domains that specifically bind to the human coronavirus can neutralize SARS-CoV-2.

In certain embodiments, the three to twelve identical binding domains of the multimeric binding molecule that bind to SARS-CoV, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of any of SEQ ID NO: 84 and SEQ ID NO: 85, SEQ ID NO: 118 and SEQ ID NO: 119, SEQ ID NO: 120 and SEQ ID NO: 121, SEQ ID NO: 138 and SEQ ID NO: 139, SEQ ID NO: 162 and SEQ ID NO: 163, SEQ ID NO: 222 and SEQ ID NO: 223, SEQ ID NO: 236 and SEQ ID NO: 237, SEQ ID NO: 252 and SEQ ID NO: 253, SEQ ID NO: 260 and SEQ ID NO: 261, SEQ ID NO: 262 and SEQ ID NO: 263, SEQ ID NO: 296 and SEQ ID NO: 297, SEQ ID NO: 384 and SEQ ID NO: 385, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, or SEQ ID NO: 646 and SEQ ID NO: 647, respectively with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 84 and SEQ ID NO: 85, SEQ ID NO: 260 and SEQ ID NO: 261, SEQ ID NO: 262 and SEQ ID NO: 263, SEQ ID NO: 296 and SEQ ID NO: 297, SEQ ID NO: 384 and SEQ ID NO: 385, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, or SEQ ID NO: 646 and SEQ ID NO: 647, respectively with zero, one, or two single amino acid substitutions in one or more of the HCDRs or LCDRs, wherein the bivalent reference IgG antibody comprising two of the binding domains that specifically bind to the human coronavirus can neutralize SARS-CoV.

In certain embodiments, the three to twelve identical binding domains of the multimeric binding molecule that bind to MERS-CoV, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of any of SEQ ID NO: 510 and SEQ ID NO: 511, SEQ ID NO: 512 and SEQ ID NO: 513, SEQ ID NO: 514 and SEQ ID NO: 515, SEQ ID NO: 516 and SEQ ID NO: 517, SEQ ID NO: 518 and SEQ ID NO: 519, SEQ ID NO: 520 and SEQ ID NO: 521, SEQ ID NO: 522 and SEQ ID NO: 523, SEQ ID NO: 524 and SEQ ID NO: 525, SEQ ID NO: 526 and SEQ ID NO: 527, SEQ ID NO: 528 and SEQ ID NO: 529, SEQ ID NO: 530 and SEQ ID NO: 531, SEQ ID NO: 532 and SEQ ID NO: 533, SEQ ID NO: 534 and SEQ ID NO: 535, SEQ ID NO: 536 and SEQ ID NO: 537, SEQ ID NO: 538 and SEQ ID NO: 539, SEQ ID NO: 540 and SEQ ID NO: 541, SEQ ID NO: 542 and SEQ ID NO: 543, SEQ ID NO: 544 and SEQ ID NO: 545, SEQ ID NO: 546 and SEQ ID NO: 547, SEQ ID NO: 548 and SEQ ID NO: 549, SEQ ID NO: 550 and SEQ ID NO: 551, SEQ ID NO: 552 and SEQ ID NO: 553, SEQ ID NO: 554 and SEQ ID NO: 555, SEQ ID NO: 556 and SEQ ID NO: 557, SEQ ID NO: 558 and SEQ ID NO: 559, SEQ ID NO: 560 and SEQ ID NO: 561, SEQ ID NO: 562 and SEQ ID NO: 563, SEQ ID NO: 564 and SEQ ID NO: 565, SEQ ID NO: 566 and SEQ ID NO: 567, SEQ ID NO: 568 and SEQ ID NO: 569, SEQ ID NO: 570 and SEQ ID NO: 571, SEQ ID NO: 572 and SEQ ID NO: 573, SEQ ID NO: 574 and SEQ ID NO: 575, SEQ ID NO: 576 and SEQ ID NO: 577, SEQ ID NO: 578 and SEQ ID NO: 579, SEQ ID NO: 580 and SEQ ID NO: 581, SEQ ID NO: 582 and SEQ ID NO: 583, SEQ ID NO: 584 and SEQ ID NO: 585, SEQ ID NO: 586 and SEQ ID NO: 587, SEQ ID NO: 588 and SEQ ID NO: 589, SEQ ID NO: 590 and SEQ ID NO: 591, SEQ ID NO: 592 and SEQ ID NO: 593, SEQ ID NO: 594 and SEQ ID NO: 595, SEQ ID NO: 596 and SEQ ID NO: 597, SEQ ID NO: 598 and SEQ ID NO: 599, SEQ ID NO: 600 and SEQ ID NO: 601, SEQ ID NO: 602 and SEQ ID NO: 603, SEQ ID NO: 604 and SEQ ID NO: 605, SEQ ID NO: 606 and SEQ ID NO: 607, SEQ ID NO: 608 and SEQ ID NO: 609, SEQ ID NO: 610 and SEQ ID NO: 611, SEQ ID NO: 612 and SEQ ID NO: 613, SEQ ID NO: 614 and SEQ ID NO: 615, SEQ ID NO: 616 and SEQ ID NO: 617, SEQ ID NO: 618 and SEQ ID NO: 619, SEQ ID NO: 620 and SEQ ID NO: 621, SEQ ID NO: 622 and SEQ ID NO: 623, SEQ ID NO: 624 and SEQ ID NO: 625, SEQ ID NO: 626 and SEQ ID NO: 627, or SEQ ID NO: 630 and SEQ ID NO: 631, respectively with zero, one, or two single amino acid substitutions in one or more of the HCDRs or LCDRs.

In certain embodiments, the three to twelve identical binding domains of the multimeric binding molecule that specifically bind to a human coronavirus, e.g., MERS-CoV, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 510 and SEQ ID NO: 511, SEQ ID NO: 512 and SEQ ID NO: 513, SEQ ID NO: 514 and SEQ ID NO: 515, SEQ ID NO: 516 and SEQ ID NO: 517, SEQ ID NO: 518 and SEQ ID NO: 519, SEQ ID NO: 520 and SEQ ID NO: 521, SEQ ID NO: 522 and SEQ ID NO: 523, SEQ ID NO: 524 and SEQ ID NO: 525, SEQ ID NO: 526 and SEQ ID NO: 527, SEQ ID NO: 528 and SEQ ID NO: 529, SEQ ID NO: 530 and SEQ ID NO: 531, SEQ ID NO: 532 and SEQ ID NO: 533, SEQ ID NO: 534 and SEQ ID NO: 535, SEQ ID NO: 536 and SEQ ID NO: 537, SEQ ID NO: 538 and SEQ ID NO: 539, SEQ ID NO: 540 and SEQ ID NO: 541, SEQ ID NO: 542 and SEQ ID NO: 543, SEQ ID NO: 544 and SEQ ID NO: 545, SEQ ID NO: 546 and SEQ ID NO: 547, SEQ ID NO: 548 and SEQ ID NO: 549, SEQ ID NO: 550 and SEQ ID NO: 551, SEQ ID NO: 552 and SEQ ID NO: 553, SEQ ID NO: 554 and SEQ ID NO: 555, SEQ ID NO: 556 and SEQ ID NO: 557, SEQ ID NO: 558 and SEQ ID NO: 559, SEQ ID NO: 560 and SEQ ID NO: 561, SEQ ID NO: 562 and SEQ ID NO: 563, SEQ ID NO: 564 and SEQ ID NO: 565, SEQ ID NO: 566 and SEQ ID NO: 567, SEQ ID NO: 568 and SEQ ID NO: 569, SEQ ID NO: 574 and SEQ ID NO: 575, SEQ ID NO: 576 and SEQ ID NO: 577, SEQ ID NO: 578 and SEQ ID NO: 579, SEQ ID NO: 580 and SEQ ID NO: 581, SEQ ID NO: 582 and SEQ ID NO: 583, SEQ ID NO: 584 and SEQ ID NO: 585, SEQ ID NO: 586 and SEQ ID NO: 587, SEQ ID NO: 588 and SEQ ID NO: 589, SEQ ID NO: 590 and SEQ ID NO: 591, SEQ ID NO: 592 and SEQ ID NO: 593, SEQ ID NO: 594 and SEQ ID NO: 595, SEQ ID NO: 596 and SEQ ID NO: 597, SEQ ID NO: 598 and SEQ ID NO: 599, SEQ ID NO: 600 and SEQ ID NO: 601, SEQ ID NO: 602 and SEQ ID NO: 603, SEQ ID NO: 604 and SEQ ID NO: 605, SEQ ID NO: 606 and SEQ ID NO: 607, SEQ ID NO: 608 and SEQ ID NO: 609, SEQ ID NO: 610 and SEQ ID NO: 611, SEQ ID NO: 612 and SEQ ID NO: 613, SEQ ID NO: 614 and SEQ ID NO: 615, SEQ ID NO: 616 and SEQ ID NO: 617, SEQ ID NO: 618 and SEQ ID NO: 619, SEQ ID NO: 620 and SEQ ID NO: 621, SEQ ID NO: 622 and SEQ ID NO: 623, SEQ ID NO: 624 and SEQ ID NO: 625, SEQ ID NO: 626 and SEQ ID NO: 627, or SEQ ID NO: 630 and SEQ ID NO: 631, respectively with zero, one, or two single amino acid substitutions in one or more of the HCDRs or LCDRs, wherein the bivalent reference IgG antibody comprising two of the binding domains that specifically bind to the human coronavirus can neutralize MERS-CoV.

In certain embodiments, the three to twelve identical binding domains of the multimeric binding molecule that specifically bind to a human coronavirus, e.g., SARS-CoV or SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 118 and SEQ ID NO: 119, SEQ ID NO: 162 and SEQ ID NO: 163, SEQ ID NO: 222 and SEQ ID NO: 223, SEQ ID NO: 260 and SEQ ID NO: 261, SEQ ID NO: 296 and SEQ ID NO: 297, SEQ ID NO: 384 and SEQ ID NO: 385, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, or SEQ ID NO: 646 and SEQ ID NO: 647, respectively with zero, one, or two single amino acid substitutions in one or more of the HCDRs or LCDRs, wherein the bivalent reference IgG antibody comprising two of the binding domains that specifically bind to the human coronavirus can neutralize SARS-CoV-2 and can bind SARS-CoV.

In certain embodiments, the three to twelve identical binding domains of the multimeric binding molecule that specifically bind to at least two human coronaviruses, e.g., SARS-CoV and SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 260 and SEQ ID NO: 261, SEQ ID NO: 296 and SEQ ID NO: 297, SEQ ID NO: 384 and SEQ ID NO: 385, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, or SEQ ID NO: 646 and SEQ ID NO: 647, respectively with zero, one, or two single amino acid substitutions in one or more of the HCDRs or LCDRs, wherein the bivalent reference IgG antibody comprising two of the binding domains that specifically bind to the human coronavirus can neutralize SARS-CoV and SARS-CoV-2.

In certain embodiments, the three to twelve identical binding domains of the multimeric binding molecule that specifically bind to a human coronavirus, e.g., SARS-CoV-2, comprise an antibody VH and a VL, wherein the VH and VL comprise amino acid sequences at least 80%, at least 85%, at least 90%, at least 95% or 100% identical to SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 104 and SEQ ID NO: 105, SEQ ID NO: 106 and SEQ ID NO: 107, SEQ ID NO: 108 and SEQ ID NO: 109, SEQ ID NO: 110 and SEQ ID NO: 111, SEQ ID NO: 112 and SEQ ID NO: 113, SEQ ID NO: 114 and SEQ ID NO: 115, SEQ ID NO: 116 and SEQ ID NO: 117, SEQ ID NO: 118 and SEQ ID NO: 119, SEQ ID NO: 120 and SEQ ID NO: 121, SEQ ID NO: 122 and SEQ ID NO: 123, SEQ ID NO: 124 and SEQ ID NO: 125, SEQ ID NO: 126 and SEQ ID NO: 127, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 150 and SEQ ID NO: 151, SEQ ID NO: 152 and SEQ ID NO: 153, SEQ ID NO: 154 and SEQ ID NO: 155, SEQ ID NO: 156 and SEQ ID NO: 157, SEQ ID NO: 158 and SEQ ID NO: 159, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 178 and SEQ ID NO: 179, SEQ ID NO: 180 and SEQ ID NO: 181, SEQ ID NO: 182 and SEQ ID NO: 183, SEQ ID NO: 184 and SEQ ID NO: 185, SEQ ID NO: 186 and SEQ ID NO: 187, SEQ ID NO: 188 and SEQ ID NO: 189, SEQ ID NO: 190 and SEQ ID NO: 191, SEQ ID NO: 192 and SEQ ID NO: 193, SEQ ID NO: 194 and SEQ ID NO: 195, SEQ ID NO: 196 and SEQ ID NO: 197, SEQ ID NO: 198 and SEQ ID NO: 199, SEQ ID NO: 200 and SEQ ID NO: 201, SEQ ID NO: 202 and SEQ ID NO: 203, SEQ ID NO: 204 and SEQ ID NO: 205, SEQ ID NO: 206 and SEQ ID NO: 207, SEQ ID NO: 208 and SEQ ID NO: 209, SEQ ID NO: 210 and SEQ ID NO: 211, SEQ ID NO: 212 and SEQ ID NO: 213, SEQ ID NO: 214 and SEQ ID NO: 215, SEQ ID NO: 216 and SEQ ID NO: 217, SEQ ID NO: 218 and SEQ ID NO: 219, SEQ ID NO: 220 and SEQ ID NO: 221, SEQ ID NO: 222 and SEQ ID NO: 223, SEQ ID NO: 224 and SEQ ID NO: 225, SEQ ID NO: 226 and SEQ ID NO: 227, SEQ ID NO: 228 and SEQ ID NO: 229, SEQ ID NO: 230 and SEQ ID NO: 231, SEQ ID NO: 232 and SEQ ID NO: 233, SEQ ID NO: 234 and SEQ ID NO: 235, SEQ ID NO: 236 and SEQ ID NO: 237, SEQ ID NO: 238 and SEQ ID NO: 239, SEQ ID NO: 240 and SEQ ID NO: 241, SEQ ID NO: 242 and SEQ ID NO: 243, SEQ ID NO: 244 and SEQ ID NO: 245, SEQ ID NO: 246 and SEQ ID NO: 247, SEQ ID NO: 248 and SEQ ID NO: 249, SEQ ID NO: 250 and SEQ ID NO: 251, SEQ ID NO: 252 and SEQ ID NO: 253, SEQ ID NO: 254 and SEQ ID NO: 255, SEQ ID NO: 256 and SEQ ID NO: 257, SEQ ID NO: 258 and SEQ ID NO: 259, SEQ ID NO: 260 and SEQ ID NO: 261, SEQ ID NO: 264 and SEQ ID NO: 265, SEQ ID NO: 266 and SEQ ID NO: 267, SEQ ID NO: 268 and SEQ ID NO: 269, SEQ ID NO: 270 and SEQ ID NO: 271, SEQ ID NO: 272 and SEQ ID NO: 273, SEQ ID NO: 274 and SEQ ID NO: 275, SEQ ID NO: 276 and SEQ ID NO: 277, SEQ ID NO: 278 and SEQ ID NO: 279, SEQ ID NO: 280 and SEQ ID NO: 281, SEQ ID NO: 282 and SEQ ID NO: 283, SEQ ID NO: 284 and SEQ ID NO: 285, SEQ ID NO: 286 and SEQ ID NO: 287, SEQ ID NO: 288 and SEQ ID NO: 289, SEQ ID NO: 290 and SEQ ID NO: 291, SEQ ID NO: 292 and SEQ ID NO: 293, SEQ ID NO: 294 and SEQ ID NO: 295, SEQ ID NO: 296 and SEQ ID NO: 297, SEQ ID NO: 298 and SEQ ID NO: 299, SEQ ID NO: 300 and SEQ ID NO: 301, SEQ ID NO: 302 and SEQ ID NO: 303, SEQ ID NO: 304 and SEQ ID NO: 305, SEQ ID NO: 306 and SEQ ID NO: 307, SEQ ID NO: 308 and SEQ ID NO: 309, SEQ ID NO: 310 and SEQ ID NO: 311, SEQ ID NO: 312 and SEQ ID NO: 313, SEQ ID NO: 314 and SEQ ID NO: 315, SEQ ID NO: 316 and SEQ ID NO: 317, SEQ ID NO: 318 and SEQ ID NO: 319, SEQ ID NO: 320 and SEQ ID NO: 321, SEQ ID NO: 322 and SEQ ID NO: 323, SEQ ID NO: 324 and SEQ ID NO: 325, SEQ ID NO: 326 and SEQ ID NO: 327, SEQ ID NO: 328 and SEQ ID NO: 329, SEQ ID NO: 330 and SEQ ID NO: 331, SEQ ID NO: 332 and SEQ ID NO: 333, SEQ ID NO: 336 and SEQ ID NO: 337, SEQ ID NO: 338 and SEQ ID NO: 339, SEQ ID NO: 340 and SEQ ID NO: 341, SEQ ID NO: 342 and SEQ ID NO: 343, SEQ ID NO: 344 and SEQ ID NO: 345, SEQ ID NO: 348 and SEQ ID NO: 349, SEQ ID NO: 350 and SEQ ID NO: 351, SEQ ID NO: 352 and SEQ ID NO: 353, SEQ ID NO: 354 and SEQ ID NO: 355, SEQ ID NO: 356 and SEQ ID NO: 357, SEQ ID NO: 358 and SEQ ID NO: 359, SEQ ID NO: 362 and SEQ ID NO: 363, SEQ ID NO: 364 and SEQ ID NO: 365, SEQ ID NO: 366 and SEQ ID NO: 367, SEQ ID NO: 368 and SEQ ID NO: 369, SEQ ID NO: 370 and SEQ ID NO: 371, SEQ ID NO: 372 and SEQ ID NO: 373, SEQ ID NO: 374 and SEQ ID NO: 375, SEQ ID NO: 376 and SEQ ID NO: 377, SEQ ID NO: 378 and SEQ ID NO: 379, SEQ ID NO: 380 and SEQ ID NO: 381, SEQ ID NO: 382 and SEQ ID NO: 383, SEQ ID NO: 384 and SEQ ID NO: 385, SEQ ID NO: 386 and SEQ ID NO: 387, SEQ ID NO: 388 and SEQ ID NO: 389, SEQ ID NO: 390 and SEQ ID NO: 391, SEQ ID NO: 392 and SEQ ID NO: 393, SEQ ID NO: 394 and SEQ ID NO: 395, SEQ ID NO: 396 and SEQ ID NO: 397, SEQ ID NO: 398 and SEQ ID NO: 399, SEQ ID NO: 400 and SEQ ID NO: 401, SEQ ID NO: 402 and SEQ ID NO: 403, SEQ ID NO: 404 and SEQ ID NO: 405, SEQ ID NO: 406 and SEQ ID NO: 407, SEQ ID NO: 408 and SEQ ID NO: 409, SEQ ID NO: 410 and SEQ ID NO: 411, SEQ ID NO: 412 and SEQ ID NO: 413, SEQ ID NO: 414 and SEQ ID NO: 415, SEQ ID NO: 416 and SEQ ID NO: 417, SEQ ID NO: 418 and SEQ ID NO: 419, SEQ ID NO: 420 and SEQ ID NO: 421, SEQ ID NO: 422 and SEQ ID NO: 423, SEQ ID NO: 424 and SEQ ID NO: 425, SEQ ID NO: 426 and SEQ ID NO: 427, SEQ ID NO: 428 and SEQ ID NO: 429, SEQ ID NO: 430 and SEQ ID NO: 431, SEQ ID NO: 432 and SEQ ID NO: 433, SEQ ID NO: 434 and SEQ ID NO: 435, SEQ ID NO: 436 and SEQ ID NO: 437, SEQ ID NO: 438 and SEQ ID NO: 439, SEQ ID NO: 440 and SEQ ID NO: 441, SEQ ID NO: 442 and SEQ ID NO: 443, SEQ ID NO: 444 and SEQ ID NO: 445, SEQ ID NO: 446 and SEQ ID NO: 447, SEQ ID NO: 448 and SEQ ID NO: 449, SEQ ID NO: 450 and SEQ ID NO: 451, SEQ ID NO: 452 and SEQ ID NO: 453, SEQ ID NO: 454 and SEQ ID NO: 455, SEQ ID NO: 456 and SEQ ID NO: 457, SEQ ID NO: 458 and SEQ ID NO: 459, SEQ ID NO: 460 and SEQ ID NO: 461, SEQ ID NO: 462 and SEQ ID NO: 463, SEQ ID NO: 464 and SEQ ID NO: 465, SEQ ID NO: 466 and SEQ ID NO: 467, SEQ ID NO: 468 and SEQ ID NO: 469, SEQ ID NO: 470 and SEQ ID NO: 471, SEQ ID NO: 472 and SEQ ID NO: 473, SEQ ID NO: 474 and SEQ ID NO: 475, SEQ ID NO: 476 and SEQ ID NO: 477, SEQ ID NO: 478 and SEQ ID NO: 479, SEQ ID NO: 480 and SEQ ID NO: 481, SEQ ID NO: 482 and SEQ ID NO: 483, SEQ ID NO: 484 and SEQ ID NO: 485, SEQ ID NO: 486 and SEQ ID NO: 487, SEQ ID NO: 488 and SEQ ID NO: 489, SEQ ID NO: 490 and SEQ ID NO: 491, SEQ ID NO: 492 and SEQ ID NO: 493, SEQ ID NO: 494 and SEQ ID NO: 495, SEQ ID NO: 496 and SEQ ID NO: 497, SEQ ID NO: 498 and SEQ ID NO: 499, SEQ ID NO: 500 and SEQ ID NO: 501, SEQ ID NO: 502 and SEQ ID NO: 503, SEQ ID NO: 504 and SEQ ID NO: 505, SEQ ID NO: 506 and SEQ ID NO: 507, SEQ ID NO: 508 and SEQ ID NO: 509, SEQ ID NO: 628 and SEQ ID NO: 629, SEQ ID NO: 630 and SEQ ID NO: 631, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, SEQ ID NO: 646 and SEQ ID NO: 647, SEQ ID NO: 648 and SEQ ID NO: 649, SEQ ID NO: 650 and SEQ ID NO: 651, or SEQ ID NO: 652 and SEQ ID NO: 653, respectively.

In certain embodiments, the three to twelve identical binding domains of the multimeric binding molecule that specifically bind to a human coronavirus, e.g., SARS-CoV-2, comprise an antibody VH and a VL, where the VH and VL comprise amino acid sequences at least 80%, at least 85%, at least 90%, at least 95% or 100% identical to SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 106 and SEQ ID NO: 107, SEQ ID NO: 110 and SEQ ID NO: 111, SEQ ID NO: 118 and SEQ ID NO: 119, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 142 and SEQ ID NO: 143, SEQ ID NO: 146 and SEQ ID NO: 147, SEQ ID NO: 150 and SEQ ID NO: 151, SEQ ID NO: 156 and SEQ ID NO: 157, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 180 and SEQ ID NO: 181, SEQ ID NO: 182 and SEQ ID NO: 183, SEQ ID NO: 186 and SEQ ID NO: 187, SEQ ID NO: 188 and SEQ ID NO: 189, SEQ ID NO: 194 and SEQ ID NO: 195, SEQ ID NO: 200 and SEQ ID NO: 201, SEQ ID NO: 206 and SEQ ID NO: 207, SEQ ID NO: 208 and SEQ ID NO: 209, SEQ ID NO: 218 and SEQ ID NO: 219, SEQ ID NO: 220 and SEQ ID NO: 221, SEQ ID NO: 222 and SEQ ID NO: 223, SEQ ID NO: 224 and SEQ ID NO: 225, SEQ ID NO: 228 and SEQ ID NO: 229, SEQ ID NO: 230 and SEQ ID NO: 231, SEQ ID NO: 240 and SEQ ID NO: 241, SEQ ID NO: 244 and SEQ ID NO: 245, SEQ ID NO: 246 and SEQ ID NO: 247, SEQ ID NO: 256 and SEQ ID NO: 257, SEQ ID NO: 258 and SEQ ID NO: 259, SEQ ID NO: 260 and SEQ ID NO: 261, SEQ ID NO: 264 and SEQ ID NO: 265, SEQ ID NO: 266 and SEQ ID NO: 267, SEQ ID NO: 268 and SEQ ID NO: 269, SEQ ID NO: 270 and SEQ ID NO: 271, SEQ ID NO: 272 and SEQ ID NO: 273, SEQ ID NO: 274 and SEQ ID NO: 275, SEQ ID NO: 276 and SEQ ID NO: 277, SEQ ID NO: 278 and SEQ ID NO: 279, SEQ ID NO: 280 and SEQ ID NO: 281, SEQ ID NO: 282 and SEQ ID NO: 283, SEQ ID NO: 284 and SEQ ID NO: 285, SEQ ID NO: 286 and SEQ ID NO: 287, SEQ ID NO: 288 and SEQ ID NO: 289, SEQ ID NO: 290 and SEQ ID NO: 291, SEQ ID NO: 292 and SEQ ID NO: 293, SEQ ID NO: 294 and SEQ ID NO: 295, SEQ ID NO: 296 and SEQ ID NO: 297, SEQ ID NO: 298 and SEQ ID NO: 299, SEQ ID NO: 300 and SEQ ID NO: 301, SEQ ID NO: 302 and SEQ ID NO: 303, SEQ ID NO: 304 and SEQ ID NO: 305, SEQ ID NO: 306 and SEQ ID NO: 307, SEQ ID NO: 308 and SEQ ID NO: 309, SEQ ID NO: 310 and SEQ ID NO: 311, SEQ ID NO: 312 and SEQ ID NO: 313, SEQ ID NO: 314 and SEQ ID NO: 315, SEQ ID NO: 316 and SEQ ID NO: 317, SEQ ID NO: 318 and SEQ ID NO: 319, SEQ ID NO: 320 and SEQ ID NO: 321, SEQ ID NO: 322 and SEQ ID NO: 323, SEQ ID NO: 324 and SEQ ID NO: 325, SEQ ID NO: 326 and SEQ ID NO: 327, SEQ ID NO: 328 and SEQ ID NO: 329, SEQ ID NO: 330 and SEQ ID NO: 331, SEQ ID NO: 332 and SEQ ID NO: 333, SEQ ID NO: 336 and SEQ ID NO: 337, SEQ ID NO: 338 and SEQ ID NO: 339, SEQ ID NO: 340 and SEQ ID NO: 341, SEQ ID NO: 342 and SEQ ID NO: 343, SEQ ID NO: 344 and SEQ ID NO: 345, SEQ ID NO: 348 and SEQ ID NO: 349, SEQ ID NO: 350 and SEQ ID NO: 351, SEQ ID NO: 352 and SEQ ID NO: 353, SEQ ID NO: 354 and SEQ ID NO: 355, SEQ ID NO: 356 and SEQ ID NO: 357, SEQ ID NO: 358 and SEQ ID NO: 359, SEQ ID NO: 360 and SEQ ID NO: 361, SEQ ID NO: 362 and SEQ ID NO: 363, SEQ ID NO: 364 and SEQ ID NO: 365, SEQ ID NO: 366 and SEQ ID NO: 367, SEQ ID NO: 368 and SEQ ID NO: 369, SEQ ID NO: 370 and SEQ ID NO: 371, SEQ ID NO: 372 and SEQ ID NO: 373, SEQ ID NO: 374 and SEQ ID NO: 375, SEQ ID NO: 376 and SEQ ID NO: 377, SEQ ID NO: 378 and SEQ ID NO: 379, SEQ ID NO: 380 and SEQ ID NO: 381, SEQ ID NO: 382 and SEQ ID NO: 383, SEQ ID NO: 384 and SEQ ID NO: 385, SEQ ID NO: 386 and SEQ ID NO: 387, SEQ ID NO: 388 and SEQ ID NO: 389, SEQ ID NO: 390 and SEQ ID NO: 391, SEQ ID NO: 392 and SEQ ID NO: 393, SEQ ID NO: 394 and SEQ ID NO: 395, SEQ ID NO: 396 and SEQ ID NO: 397, SEQ ID NO: 398 and SEQ ID NO: 399, SEQ ID NO: 400 and SEQ ID NO: 401, SEQ ID NO: 402 and SEQ ID NO: 403, SEQ ID NO: 404 and SEQ ID NO: 405, SEQ ID NO: 406 and SEQ ID NO: 407, SEQ ID NO: 408 and SEQ ID NO: 409, SEQ ID NO: 410 and SEQ ID NO: 411, SEQ ID NO: 412 and SEQ ID NO: 413, SEQ ID NO: 414 and SEQ ID NO: 415, SEQ ID NO: 416 and SEQ ID NO: 417, SEQ ID NO: 418 and SEQ ID NO: 419, SEQ ID NO: 420 and SEQ ID NO: 421, SEQ ID NO: 422 and SEQ ID NO: 423, SEQ ID NO: 424 and SEQ ID NO: 425, SEQ ID NO: 426 and SEQ ID NO: 427, SEQ ID NO: 428 and SEQ ID NO: 429, SEQ ID NO: 430 and SEQ ID NO: 431, SEQ ID NO: 432 and SEQ ID NO: 433, SEQ ID NO: 434 and SEQ ID NO: 435, SEQ ID NO: 436 and SEQ ID NO: 437, SEQ ID NO: 438 and SEQ ID NO: 439, SEQ ID NO: 440 and SEQ ID NO: 441, SEQ ID NO: 442 and SEQ ID NO: 443, SEQ ID NO: 444 and SEQ ID NO: 445, SEQ ID NO: 446 and SEQ ID NO: 447, SEQ ID NO: 448 and SEQ ID NO: 449, SEQ ID NO: 450 and SEQ ID NO: 451, SEQ ID NO: 452 and SEQ ID NO: 453, SEQ ID NO: 454 and SEQ ID NO: 455, SEQ ID NO: 456 and SEQ ID NO: 457, SEQ ID NO: 458 and SEQ ID NO: 459, SEQ ID NO: 460 and SEQ ID NO: 461, SEQ ID NO: 462 and SEQ ID NO: 463, SEQ ID NO: 464 and SEQ ID NO: 465, SEQ ID NO: 466 and SEQ ID NO: 467, SEQ ID NO: 468 and SEQ ID NO: 469, SEQ ID NO: 470 and SEQ ID NO: 471, SEQ ID NO: 472 and SEQ ID NO: 473, SEQ ID NO: 474 and SEQ ID NO: 475, SEQ ID NO: 476 and SEQ ID NO: 477, SEQ ID NO: 478 and SEQ ID NO: 479, SEQ ID NO: 480 and SEQ ID NO: 481, SEQ ID NO: 482 and SEQ ID NO: 483, SEQ ID NO: 484 and SEQ ID NO: 485, SEQ ID NO: 486 and SEQ ID NO: 487, SEQ ID NO: 488 and SEQ ID NO: 489, SEQ ID NO: 490 and SEQ ID NO: 491, SEQ ID NO: 492 and SEQ ID NO: 493, SEQ ID NO: 494 and SEQ ID NO: 495, SEQ ID NO: 496 and SEQ ID NO: 497, SEQ ID NO: 498 and SEQ ID NO: 499, SEQ ID NO: 500 and SEQ ID NO: 501, SEQ ID NO: 502 and SEQ ID NO: 503, SEQ ID NO: 504 and SEQ ID NO: 505, SEQ ID NO: 506 and SEQ ID NO: 507, SEQ ID NO: 508 and SEQ ID NO: 509, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, SEQ ID NO: 646 and SEQ ID NO: 647, SEQ ID NO: 648 and SEQ ID NO: 649, SEQ ID NO: 650 and SEQ ID NO: 651, or SEQ ID NO: 652 and SEQ ID NO: 653, respectively, wherein the bivalent reference IgG antibody comprising two of the binding domains that specifically bind to the human coronavirus can neutralize SARS-CoV-2.

In certain embodiments, the three to twelve identical binding domains of the multimeric binding molecule that specifically bind to a human coronavirus, e.g., SARS-CoV, comprise an antibody VH and a VL, where the VH and VL comprise amino acid sequences at least 80%, at least 85%, at least 90%, at least 95% or 100% identical to SEQ ID NO: 84 and SEQ ID NO: 85, SEQ ID NO: 118 and SEQ ID NO: 119, SEQ ID NO: 120 and SEQ ID NO: 121, SEQ ID NO: 138 and SEQ ID NO: 139, SEQ ID NO: 162 and SEQ ID NO: 163, SEQ ID NO: 222 and SEQ ID NO: 223, SEQ ID NO: 236 and SEQ ID NO: 237, SEQ ID NO: 252 and SEQ ID NO: 253, SEQ ID NO: 260 and SEQ ID NO: 261, SEQ ID NO: 262 and SEQ ID NO: 263, SEQ ID NO: 296 and SEQ ID NO: 297, SEQ ID NO: 384 and SEQ ID NO: 385, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, or SEQ ID NO: 646 and SEQ ID NO: 647, respectively.

In certain embodiments, the three to twelve identical binding domains of the multimeric binding molecule that specifically bind to a human coronavirus, e.g., SARS-CoV, comprise an antibody VH and a VL, where the VH and VL comprise amino acid sequences at least 80%, at least 85%, at least 90%, at least 95% or 100% identical to SEQ ID NO: 84 and SEQ ID NO: 85, SEQ ID NO: 260 and SEQ ID NO: 261, SEQ ID NO: 262 and SEQ ID NO: 263, SEQ ID NO: 296 and SEQ ID NO: 297, SEQ ID NO: 384 and SEQ ID NO: 385, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, or SEQ ID NO: 646 and SEQ ID NO: 647, respectively, wherein the bivalent reference IgG antibody comprising two of the binding domains that specifically bind to the human coronavirus can neutralize SARS-CoV.

In certain embodiments, the three to twelve identical binding domains of the multimeric binding molecule that specifically bind to a human coronavirus, e.g., MERS-CoV, comprise an antibody VH and a VL, where the VH and VL comprise amino acid sequences at least 80%, at least 85%, at least 90%, at least 95% or 100% identical to SEQ ID NO: 510 and SEQ ID NO: 511, SEQ ID NO: 512 and SEQ ID NO: 513, SEQ ID NO: 514 and SEQ ID NO: 515, SEQ ID NO: 516 and SEQ ID NO: 517, SEQ ID NO: 518 and SEQ ID NO: 519, SEQ ID NO: 520 and SEQ ID NO: 521, SEQ ID NO: 522 and SEQ ID NO: 523, SEQ ID NO: 524 and SEQ ID NO: 525, SEQ ID NO: 526 and SEQ ID NO: 527, SEQ ID NO: 528 and SEQ ID NO: 529, SEQ ID NO: 530 and SEQ ID NO: 531, SEQ ID NO: 532 and SEQ ID NO: 533, SEQ ID NO: 534 and SEQ ID NO: 535, SEQ ID NO: 536 and SEQ ID NO: 537, SEQ ID NO: 538 and SEQ ID NO: 539, SEQ ID NO: 540 and SEQ ID NO: 541, SEQ ID NO: 542 and SEQ ID NO: 543, SEQ ID NO: 544 and SEQ ID NO: 545, SEQ ID NO: 546 and SEQ ID NO: 547, SEQ ID NO: 548 and SEQ ID NO: 549, SEQ ID NO: 550 and SEQ ID NO: 551, SEQ ID NO: 552 and SEQ ID NO: 553, SEQ ID NO: 554 and SEQ ID NO: 555, SEQ ID NO: 556 and SEQ ID NO: 557, SEQ ID NO: 558 and SEQ ID NO: 559, SEQ ID NO: 560 and SEQ ID NO: 561, SEQ ID NO: 562 and SEQ ID NO: 563, SEQ ID NO: 564 and SEQ ID NO: 565, SEQ ID NO: 566 and SEQ ID NO: 567, SEQ ID NO: 568 and SEQ ID NO: 569, SEQ ID NO: 570 and SEQ ID NO: 571, SEQ ID NO: 572 and SEQ ID NO: 573, SEQ ID NO: 574 and SEQ ID NO: 575, SEQ ID NO: 576 and SEQ ID NO: 577, SEQ ID NO: 578 and SEQ ID NO: 579, SEQ ID NO: 580 and SEQ ID NO: 581, SEQ ID NO: 582 and SEQ ID NO: 583, SEQ ID NO: 584 and SEQ ID NO: 585, SEQ ID NO: 586 and SEQ ID NO: 587, SEQ ID NO: 588 and SEQ ID NO: 589, SEQ ID NO: 590 and SEQ ID NO: 591, SEQ ID NO: 592 and SEQ ID NO: 593, SEQ ID NO: 594 and SEQ ID NO: 595, SEQ ID NO: 596 and SEQ ID NO: 597, SEQ ID NO: 598 and SEQ ID NO: 599, SEQ ID NO: 600 and SEQ ID NO: 601, SEQ ID NO: 602 and SEQ ID NO: 603, SEQ ID NO: 604 and SEQ ID NO: 605, SEQ ID NO: 606 and SEQ ID NO: 607, SEQ ID NO: 608 and SEQ ID NO: 609, SEQ ID NO: 610 and SEQ ID NO: 611, SEQ ID NO: 612 and SEQ ID NO: 613, SEQ ID NO: 614 and SEQ ID NO: 615, SEQ ID NO: 616 and SEQ ID NO: 617, SEQ ID NO: 618 and SEQ ID NO: 619, SEQ ID NO: 620 and SEQ ID NO: 621, SEQ ID NO: 622 and SEQ ID NO: 623, SEQ ID NO: 624 and SEQ ID NO: 625, SEQ ID NO: 626 and SEQ ID NO: 627, or SEQ ID NO: 630 and SEQ ID NO: 631, respectively.

In certain embodiments, the three to twelve identical binding domains of the multimeric binding molecule that specifically bind to a human coronavirus, e.g., MERS-CoV, comprise an antibody VH and a VL, where the VH and VL comprise amino acid sequences at least 80%, at least 85%, at least 90%, at least 95% or 100% identical to SEQ ID NO: 510 and SEQ ID NO: 511, SEQ ID NO: 512 and SEQ ID NO: 513, SEQ ID NO: 514 and SEQ ID NO: 515, SEQ ID NO: 516 and SEQ ID NO: 517, SEQ ID NO: 518 and SEQ ID NO: 519, SEQ ID NO: 520 and SEQ ID NO: 521, SEQ ID NO: 522 and SEQ ID NO: 523, SEQ ID NO: 524 and SEQ ID NO: 525, SEQ ID NO: 526 and SEQ ID NO: 527, SEQ ID NO: 528 and SEQ ID NO: 529, SEQ ID NO: 530 and SEQ ID NO: 531, SEQ ID NO: 532 and SEQ ID NO: 533, SEQ ID NO: 534 and SEQ ID NO: 535, SEQ ID NO: 536 and SEQ ID NO: 537, SEQ ID NO: 538 and SEQ ID NO: 539, SEQ ID NO: 540 and SEQ ID NO: 541, SEQ ID NO: 542 and SEQ ID NO: 543, SEQ ID NO: 544 and SEQ ID NO: 545, SEQ ID NO: 546 and SEQ ID NO: 547, SEQ ID NO: 548 and SEQ ID NO: 549, SEQ ID NO: 550 and SEQ ID NO: 551, SEQ ID NO: 552 and SEQ ID NO: 553, SEQ ID NO: 554 and SEQ ID NO: 555, SEQ ID NO: 556 and SEQ ID NO: 557, SEQ ID NO: 558 and SEQ ID NO: 559, SEQ ID NO: 560 and SEQ ID NO: 561, SEQ ID NO: 562 and SEQ ID NO: 563, SEQ ID NO: 564 and SEQ ID NO: 565, SEQ ID NO: 566 and SEQ ID NO: 567, SEQ ID NO: 568 and SEQ ID NO: 569, SEQ ID NO: 574 and SEQ ID NO: 575, SEQ ID NO: 576 and SEQ ID NO: 577, SEQ ID NO: 578 and SEQ ID NO: 579, SEQ ID NO: 580 and SEQ ID NO: 581, SEQ ID NO: 582 and SEQ ID NO: 583, SEQ ID NO: 584 and SEQ ID NO: 585, SEQ ID NO: 586 and SEQ ID NO: 587, SEQ ID NO: 588 and SEQ ID NO: 589, SEQ ID NO: 590 and SEQ ID NO: 591, SEQ ID NO: 592 and SEQ ID NO: 593, SEQ ID NO: 594 and SEQ ID NO: 595, SEQ ID NO: 596 and SEQ ID NO: 597, SEQ ID NO: 598 and SEQ ID NO: 599, SEQ ID NO: 600 and SEQ ID NO: 601, SEQ ID NO: 602 and SEQ ID NO: 603, SEQ ID NO: 604 and SEQ ID NO: 605, SEQ ID NO: 606 and SEQ ID NO: 607, SEQ ID NO: 608 and SEQ ID NO: 609, SEQ ID NO: 610 and SEQ ID NO: 611, SEQ ID NO: 612 and SEQ ID NO: 613, SEQ ID NO: 614 and SEQ ID NO: 615, SEQ ID NO: 616 and SEQ ID NO: 617, SEQ ID NO: 618 and SEQ ID NO: 619, SEQ ID NO: 620 and SEQ ID NO: 621, SEQ ID NO: 622 and SEQ ID NO: 623, SEQ ID NO: 624 and SEQ ID NO: 625, SEQ ID NO: 626 and SEQ ID NO: 627, or SEQ ID NO: 630 and SEQ ID NO: 631, respectively, wherein the bivalent reference IgG antibody comprising two of the binding domains that specifically bind to the human coronavirus can neutralize MERS-CoV.

In certain embodiments, the three to twelve identical binding domains of the multimeric binding molecule that specifically bind to a human coronavirus, e.g., SARS-CoV or SARS-CoV-2, comprise an antibody VH and a VL, where the VH and VL comprise amino acid sequences at least 80%, at least 85%, at least 90%, at least 95% or 100% identical to SEQ ID NO: 118 and SEQ ID NO: 119, SEQ ID NO: 162 and SEQ ID NO: 163, SEQ ID NO: 222 and SEQ ID NO: 223, SEQ ID NO: 260 and SEQ ID NO: 261, SEQ ID NO: 296 and SEQ ID NO: 297, SEQ ID NO: 384 and SEQ ID NO: 385, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, or SEQ ID NO: 646 and SEQ ID NO: 647, respectively, wherein the bivalent reference IgG antibody comprising two of the binding domains that specifically bind to the human coronavirus can neutralize SARS-CoV-2 and can bind SARS-CoV.

In certain embodiments, the three to twelve identical binding domains of the multimeric binding molecule that specifically bind to a human coronavirus, e.g., SARS-CoV or SARS-CoV-2, comprise an antibody VH and a VL, where the VH and VL comprise amino acid sequences at least 80%, at least 85%, at least 90%, at least 95% or 100% identical to SEQ ID NO: 260 and SEQ ID NO: 261, SEQ ID NO: 296 and SEQ ID NO: 297, SEQ ID NO: 384 and SEQ ID NO: 385, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, or SEQ ID NO: 646 and SEQ ID NO: 647, respectively with zero, one, or two single amino acid substitutions in one or more of the HCDRs or LCDRs, wherein the bivalent reference IgG antibody comprising two of the binding domains that specifically bind to the human coronavirus can neutralize SARS-CoV and SARS-CoV-2.

In certain embodiments, the three to twelve SARS-CoV2-binding domains comprise a single domain variable region (a “nanobody” or VHH), where the VHH comprises three immunoglobulin complementarity determining regions HCDR1, HCDR2, and HCDR3, where the HCDR1, HCDR2, and HCDR3 the CDRs of an antibody comprising the VHH of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, or SEQ ID NO: 83 with zero, one, or two single amino acid substitutions in one or more of the HCDRs. In certain embodiments, the three to twelve SARS-CoV2-binding domains comprise a single domain variable region (VHH), where the VHH comprises three immunoglobulin complementarity determining regions HCDR1, HCDR2, and HCDR3, where the HCDR1, HCDR2, and HCDR3 the CDRs of an antibody comprising the VHH of SEQ ID NO: 83 with zero, one, or two single amino acid substitutions in one or more of the HCDRs.

In certain embodiments, the three to twelve SARS-CoV2-binding domains of the binding molecule comprise an antibody VHH, where the VHH comprises an amino acid sequence at least 80%, at least 85%, at least 90%, at least 95% or 100% identical to SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, or SEQ ID NO: 83 such as the amino acid sequences at least 80%, at least 85%, at least 90%, at least 95% or 100% identical to SEQ ID NO: 83.

In certain embodiments, the three to twelve identical binding domains of the multimeric binding molecule that specifically bind to a human coronavirus, e.g., SARS-CoV or SARS-CoV-2, comprise an extracellular SARS-CoV or SARS-CoV-2 RBD-binding fragment of angiotensin-converting enzyme 2 (ACE2). In certain embodiments, the three to twelve human coronavirus binding domains, e.g., SARS-CoV-2-binding domains of the binding molecule comprise an extracellular SARS-CoV or SARS-CoV-2 RBD-binding fragment of SEQ ID NO: 14. In certain embodiments, the three to twelve human coronavirus binding domains, e.g., SARS-CoV-2-binding domains, of the binding molecule comprise an amino acid sequence at least 80%, at least 85%, at least 90%, at least 95% or 100% identical to amino acids 18 to 740 of SEQ ID NO: 14. In certain embodiments the multimeric binding molecule is a pentameric or hexameric binding molecule comprising three to twelve IgM, IgM-like, or IgM-derived heavy chains each comprising at least the Cμ3, Cμ4, and tailpiece (tp) domains corresponding to an IgM heavy chain, e.g., a human IgM heavy chain fused to an extracellular domain of ACE2, e.g., human ACE2, e.g., an amino acid sequence at least 80%, at least 85%, at least 90%, at least 95% or 100% identical to amino acids 18 to 740 of SEQ ID NO: 14. In certain embodiments the heavy chains comprise the Cμ3, Cμ4, and tailpiece (tp) domains corresponding to an IgM heavy chain, e.g., a human IgM heavy chain with a modified human IgG1 hinge region fused to the N-terminus where the cysteine at position 7 of the human IgG1 hinge region is substituted with serine, e.g., amino acids 724 to 741 of SEQ ID NO: 15 (VEPKSSD KTHTCPPCPA P). In certain embodiments the binding molecule is engineered to reduce or eliminate complement-mediated cytotoxicity, e.g., comprising amino acid substitutions P311A, P313S corresponding to the positions in SEQ ID NO: 1 and SEQ ID NO: 2, or comprising the amino acid substitution K315D corresponding to the position in SEQ ID NO: 1 and SEQ ID NO: 2. In certain embodiments the multimeric binding molecule comprises ten or twelve heavy chains comprising the amino acid sequence SEQ ID NO: 15.

In certain embodiments, the three to twelve identical binding domains of the multimeric binding molecule that specifically bind to a human coronavirus, e.g., SARS-CoV-1, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 84 and SEQ ID NO: 85, or SEQ ID NO: 262 and SEQ ID NO: 263, respectively, with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV-1, comprise an antibody VH and a VL, where the VH and VL comprise amino acid sequences at least 80%, at least 85%, at least 90%, at least 95% or 100% identical to SEQ ID NO: 84 and SEQ ID NO: 85, or SEQ ID NO: 262 and SEQ ID NO: 263, respectively.

In certain embodiments, the three to twelve identical binding domains of the multimeric binding molecule that specifically bind to a human coronavirus, e.g., SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 256 and SEQ ID NO: 257, respectively, with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV-2, comprise an antibody VH and a VL, where the VH and VL comprise amino acid sequences at least 80%, at least 85%, at least 90%, at least 95% or 100% identical to SEQ ID NO: 256 and SEQ ID NO: 257, respectively.

In certain embodiments, the three to twelve identical binding domains of the multimeric binding molecule that specifically bind to a human coronavirus, e.g., SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 260 and SEQ ID NO: 261, respectively, with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV-2, comprise an antibody VH and a VL, where the VH and VL comprise amino acid sequences at least 80%, at least 85%, at least 90%, at least 95% or 100% identical to SEQ ID NO: 260 and SEQ ID NO: 261, respectively.

In certain embodiments, the three to twelve identical binding domains of the multimeric binding molecule that specifically bind to a human coronavirus, e.g., SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 264 and SEQ ID NO: 265, or SEQ ID NO: 266 and SEQ ID NO: 267, respectively, with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV-2, comprise an antibody VH and a VL, where the VH and VL comprise amino acid sequences at least 80%, at least 85%, at least 90%, at least 95% or 100% identical to SEQ ID NO: 264 and SEQ ID NO: 265, or SEQ ID NO: 266 and SEQ ID NO: 267, respectively.

In certain embodiments, the three to twelve identical binding domains of the multimeric binding molecule that specifically bind to a human coronavirus, e.g., SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 88 and SEQ ID NO: 89, respectively, with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV-2, comprise an antibody VH and a VL, where the VH and VL comprise amino acid sequences at least 80%, at least 85%, at least 90%, at least 95% or 100% identical to SEQ ID NO: 88 and SEQ ID NO: 89, respectively.

In certain embodiments, the three to twelve identical binding domains of the multimeric binding molecule that specifically bind to a human coronavirus, e.g., SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 268 and SEQ ID NO: 269, respectively, with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV-2, comprise an antibody VH and a VL, where the VH and VL comprise amino acid sequences at least 80%, at least 85%, at least 90%, at least 95% or 100% identical to SEQ ID NO: 268 and SEQ ID NO: 269, respectively.

In certain embodiments, the three to twelve identical binding domains of the multimeric binding molecule that specifically bind to a human coronavirus, e.g., SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 270 and SEQ ID NO: 271, respectively, with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV-2, comprise an antibody VH and a VL, where the VH and VL comprise amino acid sequences at least 80%, at least 85%, at least 90%, at least 95% or 100% identical to SEQ ID NO: 270 and SEQ ID NO: 271, respectively.

In certain embodiments, the three to twelve identical binding domains of the multimeric binding molecule that specifically bind to a human coronavirus, e.g., SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 272 and SEQ ID NO: 273, SEQ ID NO: 274 and SEQ ID NO: 275, SEQ ID NO: 276 and SEQ ID NO: 277, SEQ ID NO: 278 and SEQ ID NO: 279, SEQ ID NO: 280 and SEQ ID NO: 281, SEQ ID NO: 282 and SEQ ID NO: 283, respectively, with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV-2, comprise an antibody VH and a VL, where the VH and VL comprise amino acid sequences at least 80%, at least 85%, at least 90%, at least 95% or 100% identical to SEQ ID NO: 272 and SEQ ID NO: 273, SEQ ID NO: 274 and SEQ ID NO: 275, SEQ ID NO: 276 and SEQ ID NO: 277, SEQ ID NO: 278 and SEQ ID NO: 279, SEQ ID NO: 280 and SEQ ID NO: 281, SEQ ID NO: 282 and SEQ ID NO: 283, respectively.

In certain embodiments, the three to twelve identical binding domains of the multimeric binding molecule that specifically bind to a human coronavirus, e.g., SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 284 and SEQ ID NO: 285, SEQ ID NO: 286 and SEQ ID NO: 287, SEQ ID NO: 288 and SEQ ID NO: 289, SEQ ID NO: 290 and SEQ ID NO: 291, respectively, with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV-2, comprise an antibody VH and a VL, where the VH and VL comprise amino acid sequences at least 80%, at least 85%, at least 90%, at least 95% or 100% identical to SEQ ID NO: 284 and SEQ ID NO: 285, SEQ ID NO: 286 and SEQ ID NO: 287, SEQ ID NO: 288 and SEQ ID NO: 289, SEQ ID NO: 290 and SEQ ID NO: 291, respectively.

In certain embodiments, the three to twelve identical binding domains of the multimeric binding molecule that specifically bind to a human coronavirus, e.g., SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 292 and SEQ ID NO: 293, respectively, with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV-2, comprise an antibody VH and a VL, where the VH and VL comprise amino acid sequences at least 80%, at least 85%, at least 90%, at least 95% or 100% identical to SEQ ID NO: 292 and SEQ ID NO: 293, respectively.

In certain embodiments, the three to twelve identical binding domains of the multimeric binding molecule that specifically bind to a human coronavirus, e.g., SARS-CoV and SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 384 and SEQ ID NO: 385, respectively, with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV and SARS-CoV-2, comprise an antibody VH and a VL, where the VH and VL comprise amino acid sequences at least 80%, at least 85%, at least 90%, at least 95% or 100% identical to SEQ ID NO: 384 and SEQ ID NO: 385, respectively.

In certain embodiments, the three to twelve identical binding domains of the multimeric binding molecule that specifically bind to a human coronavirus, e.g., SARS-CoV and SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 646 and SEQ ID NO: 647, respectively, with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV and SARS-CoV-2, comprise an antibody VH and a VL, where the VH and VL comprise amino acid sequences at least 80%, at least 85%, at least 90%, at least 95% or 100% identical to SEQ ID NO: 646 and SEQ ID NO: 647, respectively.

In certain embodiments, the three to twelve identical binding domains of the multimeric binding molecule that specifically bind to a human coronavirus, e.g., SARS-CoV-2, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 654 and SEQ ID NO: 655, respectively, with zero, one, or two single amino acid substitutions in one or more of the HCDRs or LCDRs.

In certain embodiments, the three to twelve identical binding domains of the multimeric binding molecule that specifically bind to a human coronavirus, e.g., SARS-CoV-2, comprise an antibody VH and a VL, where the VH and VL comprise amino acid sequences at least 80%, at least 85%, at least 90%, at least 95% or 100% identical to SEQ ID NO: 654 and SEQ ID NO: 655, respectively.

In certain embodiments, the three to twelve identical binding domains of the multimeric binding molecule that specifically bind to a human coronavirus, e.g., MERS-CoV, comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 522 and SEQ ID NO: 523, SEQ ID NO: 576 and SEQ ID NO: 577, SEQ ID NO: 610 and SEQ ID NO: 611, SEQ ID NO: 612 and SEQ ID NO: 613, MERS-4, or SEQ ID NO: 630 and SEQ ID NO: 631, respectively, with zero, one, or two single amino acid substitutions in one or more of the HCDRs or LCDRs.

In certain embodiments, the three to twelve identical binding domains of the multimeric binding molecule that specifically bind to a human coronavirus, e.g., MERS-CoV, comprise an antibody VH and a VL, where the VH and VL comprise amino acid sequences at least 80%, at least 85%, at least 90%, at least 95% or 100% identical to SEQ ID NO: 522 and SEQ ID NO: 523, SEQ ID NO: 576 and SEQ ID NO: 577, SEQ ID NO: 610 and SEQ ID NO: 611, SEQ ID NO: 612 and SEQ ID NO: 613, SEQ ID NO: 614 and SEQ ID NO: 615, or SEQ ID NO: 630 and SEQ ID NO: 631, respectively.

### IgM Antibodies, IgM-Like Antibodies, Other IgM-Derived Binding Molecules

IgM is the first immunoglobulin produced by B cells in response to stimulation by antigen. Naturally occurring IgM is naturally present at around 1.5 mg/ml in serum with a half-life of about 5 days. IgM is a pentameric or hexameric molecule and thus includes five or six binding units. An IgM binding unit typically includes two light and two heavy chains. While an IgG heavy chain constant region contains three heavy chain constant domains (CH1, CH2 and CH3), the heavy (μ) constant region of IgM additionally contains a fourth constant domain (CH4) and includes a C-terminal “tailpiece.” The human IgM constant region typically comprises the amino acid sequence SEQ ID NO: 1 (identical to, e.g., GenBank Accession Nos. pir∥S37768, CAA47708.1, and CAA47714.1, allele IGHM*03) or SEQ ID NO: 2 (identical to, e.g., GenBank Accession No. sp|P01871.4, allele IGHM*04). The human Cμ1 region ranges from about amino acid 5 to about amino acid 102 of SEQ ID NO: 1 or SEQ ID NO: 2; the human Cμ2 region ranges from about amino acid 114 to about amino acid 205 of SEQ ID NO: 1 or SEQ ID NO: 2, the human Cμ3 region ranges from about amino acid 224 to about amino acid 319 of SEQ ID NO: 1 or SEQ ID NO: 2, the Cμ4 region ranges from about amino acid 329 to about amino acid 430 of SEQ ID NO: 1 or SEQ ID NO: 2, and the tailpiece ranges from about amino acid 431 to about amino acid 453 of SEQ ID NO: 1 or SEQ ID NO: 2.

Other forms and alleles of the human IgM constant region with minor sequence variations exist, including, without limitation, GenBank Accession Nos. CAB37838.1, and pir∥HHU. The amino acid substitutions, insertions, and/or deletions at positions corresponding to SEQ ID NO: 1 or SEQ ID NO: 2 described and claimed elsewhere in this disclosure can likewise be incorporated into alternate human IgM sequences, as well as into IgM constant region amino acid sequences of other species.

Each IgM heavy chain constant region can be associated with a binding domain, e.g., an antigen-binding domain, e.g., a scFv or VHH, or a subunit of an antigen-binding domain, e.g., a VH region. Exemplary antigen-binding domains, e.g., binding domains that bind SARS-CoV-2 are described elsewhere herein. In certain embodiments the binding domain can be a non-antibody binding domain, e.g., an ACE-2 ectodomain.

Five IgM binding units can form a complex with an additional small polypeptide chain (the J-chain), or a functional fragment, variant, or derivative thereof, to form a pentameric IgM antibody or IgM-like antibody, as discussed elsewhere herein. The precursor form of the human J-chain is presented as SEQ ID NO: 6. The signal peptide extends from amino acid 1 to about amino acid 22 of SEQ ID NO: 6, and the mature human J-chain extends from about amino acid 23 to amino acid 159 of SEQ ID NO: 6. The mature human J-chain includes the amino acid sequence SEQ ID NO: 7.

Exemplary variant and modified J-chains are provided elsewhere herein. Without the J-chain, an IgM antibody or IgM-like antibody typically assembles into a hexamer, comprising up to twelve antigen-binding domains. With a J-chain, an IgM antibody or IgM-like antibody typically assembles into a pentamer, comprising up to ten antigen-binding domains, or more, if the J-chain is a modified J-chain comprising one or more heterologous polypeptides comprising additional antigen-binding domain(s). The assembly of five or six IgM binding units into a pentameric or hexameric IgM antibody or IgM-like antibody is thought to involve the Cμ4 and tailpiece domains. See, e.g., Braathen, R., et al., J. Biol. Chem. 277:42755-42762 (2002). Accordingly, a pentameric or hexameric IgM antibody provided in this disclosure typically includes at least the Cμ4 and tailpiece domains (also referred to herein collectively as Cμ4-tp). A “multimerizing fragment” of an IgM heavy chain constant region thus includes at least the Cμ4-tp domains. An IgM heavy chain constant region can additionally include a Cμ3 domain or a fragment thereof, a Cμ2 domain or a fragment thereof, a Cμ1 domain or a fragment thereof, and/or other IgM heavy chain domains. In certain embodiments, an IgM-derived binding molecule, e.g., an IgM antibody, IgM-like antibody, or other IgM-derived binding molecule as provided herein can include a complete IgM heavy (μ) chain constant domain, e.g., SEQ ID NO: 1 or SEQ ID NO: 2, or a variant, derivative, or analog thereof, e.g., as provided herein.

In certain embodiments, the disclosure provides a multimeric binding molecule, e.g., a pentameric or hexameric binding molecule, where the binding molecule includes ten or twelve IgM-derived heavy chains, and where the IgM-derived heavy chains comprise IgM heavy chain constant regions each associated with a binding domain that specifically binds to a target. In certain embodiments, the disclosure provides an IgM antibody, IgM-like antibody, or IgM-derived binding molecule that includes five or six bivalent binding units, where each binding unit includes two IgM or IgM-like heavy chain constant regions or multimerizing fragments or variants thereof, each associated with an antigen-binding domain or subunit thereof. In certain embodiments, the two IgM heavy chain constant regions included in each binding unit are human heavy chain constant regions. In some embodiments, the heavy chains are glycosylated. In some embodiments, the heavy chains can be mutated to affect glycosylation.

Where the IgM antibody, IgM-like antibody, or other IgM-derived binding molecule provided in this disclosure is pentameric, the IgM antibody, IgM-like antibody, or other IgM-derived binding molecule typically further includes a J-chain, or functional fragment or variant thereof. In certain embodiments, the J-chain is a modified J-chain or variant thereof that further comprises one or more heterologous moieties attached to the J-chain, as described elsewhere herein. In certain embodiments, the J-chain can be mutated to affect, e.g., enhance, the serum half-life of the IgM antibody, IgM-like antibody, or other IgM-derived binding molecule provided herein, as discussed elsewhere in this disclosure. In certain embodiments the J-chain can be mutated to affect glycosylation and/or serum half-life of the binding molecule, as discussed elsewhere in this disclosure.

An IgM heavy chain constant region can include one or more of a Cμ11 domain or fragment or variant thereof, a Cμ2 domain or fragment or variant thereof, a Cμ3 domain or fragment or variant thereof, a Cμ4 domain or fragment or variant thereof, and/or an IgM tailpiece, provided that the constant region can serve a desired function in the IgM antibody, IgM-like antibody, or other IgM-derived binding molecule, e.g., associate with second IgM constant region to form a binding unit with one, two, or more antigen-binding domain(s), and/or associate with other binding units (and in the case of a pentamer, a J-chain) to form a hexamer or a pentamer. In certain embodiments the two IgM heavy chain constant regions or fragments or variants thereof within an individual binding unit each comprise a Cμ4 domain or fragment or variant thereof, a tailpiece (tp) or fragment or variant thereof, or a combination of a Cμ4 domain and a tp or fragment or variant thereof. In certain embodiments the two IgM heavy chain constant regions or fragments or variants thereof within an individual binding unit each further comprise a Cμ3 domain or fragment or variant thereof, a Cμ2 domain or fragment or variant thereof, a Cμ1 domain or fragment or variant thereof, or any combination thereof.

In some embodiments, the binding units of the IgM antibody, IgM-like antibody, or other IgM-derived binding molecule each comprise two light chains. In some embodiments, the binding units of the IgM antibody, IgM-like antibody, or other IgM-derived binding molecule each comprise two fragments of light chains. In some embodiments, the light chains are kappa light chains. In some embodiments, the light chains are lambda light chains. In some embodiments, the light chains are hybrid kappa-lambda light chains. In some embodiments, each binding unit comprises two immunoglobulin light chains each comprising a VL situated amino terminal to an immunoglobulin light chain constant region.

### IgA Antibodies, IgA-Like Antibodies, Other IgA-Derived Binding Molecules

IgA plays a critical role in mucosal immunity and comprises about 15% of total immunoglobulin produced. IgA can be monomeric or multimeric, forming primarily dimeric molecules, but can also assemble as trimers, tetramers, and/or pentamers. See, e.g., de Sousa-Pereira, P., and J. M. Woof, Antibodies 8:57 (2019). An IgA binding unit typically includes two light and two heavy chains. IgA contains three heavy chain constant region domains (Cα1, Cα2 and Cα3), a hinge region between Cα1 and Cα2, and includes a C-terminal “tailpiece.” Human IgA has two subtypes, IgA1 and IgA2. The human IgA1 constant region typically includes the amino acid sequence SEQ ID NO: 3. The human Cα1 domain extends from about amino acid 6 to about amino acid 98 of SEQ ID NO: 3; the human IgA1 hinge region extends from about amino acid 102 to about amino acid 124 of SEQ ID NO: 3, the human Cα3 domain extends from about amino acid 228 to about amino acid 330 of SEQ ID NO: 3, and the tailpiece extends from about amino acid 331 to about amino acid 352 of SEQ ID NO: 3. The human IgA2 constant region typically includes the amino acid sequence SEQ ID NO: 4. The human Cal domain extends from about amino acid 6 to about amino acid 98 of SEQ ID NO: 4; the human IgA2 hinge region extends from about amino acid 102 to about amino acid 111 of SEQ ID NO: 4, the human Cα2 domain extends from about amino acid 113 to about amino acid 206 of SEQ ID NO: 4, the human Cα3 domain extends from about amino acid 215 to about amino acid 317 of SEQ ID NO: 4, and the tailpiece extends from about amino acid 318 to about amino acid 340 of SEQ ID NO: 4.

Two IgA binding units can form a complex with two additional polypeptide chains, the J-chain (e.g., the mature human J-chain of SEQ ID NO: 7) and the secretory component (precursor, SEQ ID NO: 5, mature: amino acids 19 to 603 of SEQ ID NO: 5) to form a secretory IgA (sIgA) antibody. The assembly of IgA binding units into a dimeric sIgA antibody is thought to involve the Cα3 and tailpiece domains (also referred to herein collectively as the Cα3-tp domain). Accordingly, a dimeric sIgA antibody provided in this disclosure typically includes IgA constant regions that include at least the Cα3 and tailpiece domains. Four IgA binding units can likewise form a tetramer complex with a J-chain. A sIgA antibody can also form as a higher order multimer, e.g., a tetramer or pentamer.

An IgA heavy chain constant region can additionally include a Cα2 domain or a fragment thereof, an IgA hinge region, a Cα1 domain or a fragment thereof, and/or other IgA heavy chain domains. In certain aspects, an IgA antibody or IgA-like binding molecule as provided herein can include a complete IgA heavy (u) chain constant domain (e.g., SEQ ID NO: 3 or SEQ ID NO: 4), or a variant, derivative, or analog thereof. In some embodiments, the IgA heavy chain constant regions or multimerizing fragments thereof are human IgA constant regions.

In some embodiments, each binding unit of an IgA antibody, IgA-like antibody, or other IgA-derived binding molecule comprises two light chains. In some embodiments, each binding unit of an IgA antibody, IgA-like antibody, or other IgA-derived binding molecule comprises two fragments of light chains. In some embodiments, the light chains are kappa light chains. In some embodiments, the light chains are lambda light chains. In some embodiments the light chains are hybrid kappa-lambda light chains. In some embodiments, each binding unit comprises two immunoglobulin light chains each comprising a VL situated amino terminal to an immunoglobulin light chain constant region.

### J-Chains and Functional Fragments or Variants Thereof

In certain embodiments, the multimeric binding molecule provided herein comprises a J-chain or functional fragment or variant thereof. In certain embodiments, the multimeric binding molecule provided herein is pentameric and comprises a J-chain or functional fragment or variant thereof. In certain embodiments, the multimeric binding molecule provided herein is a dimeric IgA molecule or a pentameric IgM molecule and comprises a J-chain or functional fragment or variant thereof. In some embodiments, the multimeric binding molecule can comprise a naturally occurring J-chain sequence, such as a mature human J-chain sequence (e.g., SEQ ID NO: 7). Alternatively, in some embodiments, the multimeric binding molecule can comprise a variant J-chain sequence, such as a variant sequence described herein with reduced glycosylation or reduced binding to one or more polymeric Ig receptors (e.g., pIgR, Fc alpha-mu receptor (FcαμR), or Fc mu receptor (FcμR)). See, e.g., U.S. Pat. No. 10,899,835, which is incorporated herein by reference in its entirety. In some embodiments, the multimeric binding molecule can comprise a functional fragment of a naturally occurring or variant J-chain. As persons of ordinary skill in the art will recognize, “a functional fragment” or a “functional variant” in this context includes those fragments and variants that can associate with binding units, e.g., IgM or IgA heavy chain constant regions, to form a pentameric IgM antibody, IgM-like antibody, or IgM-derived binding molecule or a dimeric IgA antibody, IgA-like antibody, or IgA-derived binding molecule, and/or can associate with certain immunoglobulin receptors, e.g., the polymeric immunoglobulin receptor (pIgR).

In certain embodiments, the J-chain can be modified, e.g., by introduction of a heterologous moiety, or two or more heterologous moieties, e.g., polypeptides, without interfering with the ability of binding molecule to assemble and bind to its binding target(s). See U.S. Pat. Nos. 9,951,134, 10,400,038, 10,618,978, and in U.S. Patent Application Publication No. US-2019-0185570, each of which is incorporated herein by reference in its entirety.

Accordingly, a binding molecule provided by this disclosure, including multispecific IgA, IgA-like, IgM, or IgM-like antibodies as described elsewhere herein, can comprise a modified J-chain or functional fragment or variant thereof comprising a heterologous moiety, e.g., a heterologous polypeptide, introduced, e.g., fused or chemically conjugated, into the J-chain or fragment or variant thereof. In certain embodiments, the heterologous polypeptide can be fused to the N-terminus of the J-chain or functional fragment or variant thereof, the C-terminus of the J-chain or functional fragment or variant thereof, or to both the N-terminus and C-terminus of the J-chain or functional fragment or variant thereof. In certain embodiments the heterologous polypeptide can be fused internally within the J-chain or functional fragment or variant thereof. In some embodiments, the heterologous polypeptide can be introduced into the J-chain at or near a glycosylation site. In some embodiments, the heterologous polypeptide can be introduced into the J-chain within about 10 amino acid residues from the C-terminus, or within about 10 amino acids from the N-terminus. In certain embodiments, the heterologous polypeptide can be introduced into the mature human J-chain of SEQ ID NO: 7 between cysteine residues 92 and 101 of SEQ ID NO: 7, or an equivalent location in a J-chain sequence, e.g., a J-chain variant or functional fragment of a J-chain. In a further embodiment, the heterologous polypeptide can be introduced into the mature human J-chain of SEQ ID NO: 7 at or near a glycosylation site. In a further embodiment, the heterologous polypeptide can be introduced into the mature human J-chain of SEQ ID NO: 7 within about 10 amino acid residues from the C-terminus, or within about 10 amino acids from the N-terminus.

In certain embodiments the heterologous moiety can be a peptide or polypeptide sequence fused in frame to the J-chain or chemically conjugated to the J-chain or fragment or variant thereof. In certain embodiments, the heterologous polypeptide is fused to the J-chain or functional fragment thereof via a peptide linker. Any suitable linker can be used, for example the peptide linker can include at least 5 amino acids, at least ten amino acids, and least 20 amino acids, at least 30 amino acids or more, and so on. In certain embodiments, the peptide linker includes least 5 amino acids, but no more than 25 amino acids. In certain embodiments the peptide linker can consist of 5 amino acids, 10 amino acids, 15 amino acids, 20 amino acids, or 25 amino acids. In certain embodiments, the peptide linker consists of GGGGS (SEQ ID NO: 9), GGGGSGGGGS (SEQ ID NO: 10), GGGGSGGGGSGGGGS (SEQ ID NO: 11), GGGGSGGGGSGGGGSGGGGS (SEQ ID NO: 12), or GGGGSGGGGSGGGGSGGGGSGGGGS (SEQ ID NO: 13).

Heterologous moieties to be attached to a J-chain can include, without limitation, a binding moiety, e.g., an antibody or antigen-binding fragment thereof, e.g., a single chain Fv (scFv) molecule, e.g., an scFv that binds pIgR or mucin, a cytokine, e.g., IL-2 or IL-15 (see, e.g., PCT Application No. PCT US2019/057702, which is incorporated herein by reference in its entirety), a stabilizing peptide that can increase the half-life of the binding molecule, e.g., human serum albumin (HSA) or an HSA binding molecule, a pIgR or mucin binding molecule, or a heterologous chemical moiety such as a polymer.

In some embodiments, a modified J-chain can comprise an antigen-binding domain that can include without limitation a polypeptide capable of specifically binding to a target antigen. In certain embodiments, an antigen-binding domain associated with a modified J-chain can be an antibody or an antigen-binding fragment thereof. In certain embodiments the antigen-binding domain can be a scFv antigen-binding domain or a single-chain antigen-binding domain derived, e.g., from a camelid or condricthoid antibody. In certain embodiments, the target is a target epitope, a target antigen, a target cell, or a target organ. In some embodiments, the antigen binding domain binds Interleukin 6 (IL6), mucin, or pIgR.

In certain embodiments, the binding domain, e.g., scFv fragment can specifically bind to human coronavirus. In certain embodiments, binding domain binds to a different epitope of the human coronavirus than the three to twelve identical binding domains of the multimeric binding molecule that specifically bind to the human coronavirus. In certain embodiments, the binding domain, e.g., scFv fragment can specifically bind to SARS-CoV-2.

In some embodiments, the SARS-CoV-2-specific binding domain, e.g., scFv fragment comprises a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 104 and SEQ ID NO: 105, SEQ ID NO: 106 and SEQ ID NO: 107, SEQ ID NO: 108 and SEQ ID NO: 109, SEQ ID NO: 110 and SEQ ID NO: 111, SEQ ID NO: 112 and SEQ ID NO: 113, SEQ ID NO: 114 and SEQ ID NO: 115, SEQ ID NO: 116 and SEQ ID NO: 117, SEQ ID NO: 118 and SEQ ID NO: 119, SEQ ID NO: 120 and SEQ ID NO: 121, SEQ ID NO: 122 and SEQ ID NO: 123, SEQ ID NO: 124 and SEQ ID NO: 125, SEQ ID NO: 126 and SEQ ID NO: 127, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 150 and SEQ ID NO: 151, SEQ ID NO: 152 and SEQ ID NO: 153, SEQ ID NO: 154 and SEQ ID NO: 155, SEQ ID NO: 156 and SEQ ID NO: 157, SEQ ID NO: 158 and SEQ ID NO: 159, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 178 and SEQ ID NO: 179, SEQ ID NO: 180 and SEQ ID NO: 181, SEQ ID NO: 182 and SEQ ID NO: 183, SEQ ID NO: 184 and SEQ ID NO: 185, SEQ ID NO: 186 and SEQ ID NO: 187, SEQ ID NO: 188 and SEQ ID NO: 189, SEQ ID NO: 190 and SEQ ID NO: 191, SEQ ID NO: 192 and SEQ ID NO: 193, SEQ ID NO: 194 and SEQ ID NO: 195, SEQ ID NO: 196 and SEQ ID NO: 197, SEQ ID NO: 198 and SEQ ID NO: 199, SEQ ID NO: 200 and SEQ ID NO: 201, SEQ ID NO: 202 and SEQ ID NO: 203, SEQ ID NO: 204 and SEQ ID NO: 205, SEQ ID NO: 206 and SEQ ID NO: 207, SEQ ID NO: 208 and SEQ ID NO: 209, SEQ ID NO: 210 and SEQ ID NO: 211, SEQ ID NO: 212 and SEQ ID NO: 213, SEQ ID NO: 214 and SEQ ID NO: 215, SEQ ID NO: 216 and SEQ ID NO: 217, SEQ ID NO: 218 and SEQ ID NO: 219, SEQ ID NO: 220 and SEQ ID NO: 221, SEQ ID NO: 222 and SEQ ID NO: 223, SEQ ID NO: 224 and SEQ ID NO: 225, SEQ ID NO: 226 and SEQ ID NO: 227, SEQ ID NO: 228 and SEQ ID NO: 229, SEQ ID NO: 230 and SEQ ID NO: 231, SEQ ID NO: 232 and SEQ ID NO: 233, SEQ ID NO: 234 and SEQ ID NO: 235, SEQ ID NO: 236 and SEQ ID NO: 237, SEQ ID NO: 238 and SEQ ID NO: 239, SEQ ID NO: 240 and SEQ ID NO: 241, SEQ ID NO: 242 and SEQ ID NO: 243, SEQ ID NO: 244 and SEQ ID NO: 245, SEQ ID NO: 246 and SEQ ID NO: 247, SEQ ID NO: 248 and SEQ ID NO: 249, SEQ ID NO: 250 and SEQ ID NO: 251, SEQ ID NO: 252 and SEQ ID NO: 253, SEQ ID NO: 254 and SEQ ID NO: 255, SEQ ID NO: 256 and SEQ ID NO: 257, SEQ ID NO: 258 and SEQ ID NO: 259, SEQ ID NO: 260 and SEQ ID NO: 261, SEQ ID NO: 264 and SEQ ID NO: 265, SEQ ID NO: 266 and SEQ ID NO: 267, SEQ ID NO: 268 and SEQ ID NO: 269, SEQ ID NO: 270 and SEQ ID NO: 271, SEQ ID NO: 272 and SEQ ID NO: 273, SEQ ID NO: 274 and SEQ ID NO: 275, SEQ ID NO: 276 and SEQ ID NO: 277, SEQ ID NO: 278 and SEQ ID NO: 279, SEQ ID NO: 280 and SEQ ID NO: 281, SEQ ID NO: 282 and SEQ ID NO: 283, SEQ ID NO: 284 and SEQ ID NO: 285, SEQ ID NO: 286 and SEQ ID NO: 287, SEQ ID NO: 288 and SEQ ID NO: 289, SEQ ID NO: 290 and SEQ ID NO: 291, SEQ ID NO: 292 and SEQ ID NO: 293, SEQ ID NO: 294 and SEQ ID NO: 295, SEQ ID NO: 296 and SEQ ID NO: 297, SEQ ID NO: 298 and SEQ ID NO: 299, SEQ ID NO: 300 and SEQ ID NO: 301, SEQ ID NO: 302 and SEQ ID NO: 303, SEQ ID NO: 304 and SEQ ID NO: 305, SEQ ID NO: 306 and SEQ ID NO: 307, SEQ ID NO: 308 and SEQ ID NO: 309, SEQ ID NO: 310 and SEQ ID NO: 311, SEQ ID NO: 312 and SEQ ID NO: 313, SEQ ID NO: 314 and SEQ ID NO: 315, SEQ ID NO: 316 and SEQ ID NO: 317, SEQ ID NO: 318 and SEQ ID NO: 319, SEQ ID NO: 320 and SEQ ID NO: 321, SEQ ID NO: 322 and SEQ ID NO: 323, SEQ ID NO: 324 and SEQ ID NO: 325, SEQ ID NO: 326 and SEQ ID NO: 327, SEQ ID NO: 328 and SEQ ID NO: 329, SEQ ID NO: 330 and SEQ ID NO: 331, SEQ ID NO: 332 and SEQ ID NO: 333, SEQ ID NO: 336 and SEQ ID NO: 337, SEQ ID NO: 338 and SEQ ID NO: 339, SEQ ID NO: 340 and SEQ ID NO: 341, SEQ ID NO: 342 and SEQ ID NO: 343, SEQ ID NO: 344 and SEQ ID NO: 345, SEQ ID NO: 348 and SEQ ID NO: 349, SEQ ID NO: 350 and SEQ ID NO: 351, SEQ ID NO: 352 and SEQ ID NO: 353, SEQ ID NO: 354 and SEQ ID NO: 355, SEQ ID NO: 356 and SEQ ID NO: 357, SEQ ID NO: 358 and SEQ ID NO: 359, SEQ ID NO: 362 and SEQ ID NO: 363, SEQ ID NO: 364 and SEQ ID NO: 365, SEQ ID NO: 366 and SEQ ID NO: 367, SEQ ID NO: 368 and SEQ ID NO: 369, SEQ ID NO: 370 and SEQ ID NO: 371, SEQ ID NO: 372 and SEQ ID NO: 373, SEQ ID NO: 374 and SEQ ID NO: 375, SEQ ID NO: 376 and SEQ ID NO: 377, SEQ ID NO: 378 and SEQ ID NO: 379, SEQ ID NO: 380 and SEQ ID NO: 381, SEQ ID NO: 382 and SEQ ID NO: 383, SEQ ID NO: 384 and SEQ ID NO: 385, SEQ ID NO: 386 and SEQ ID NO: 387, SEQ ID NO: 388 and SEQ ID NO: 389, SEQ ID NO: 390 and SEQ ID NO: 391, SEQ ID NO: 392 and SEQ ID NO: 393, SEQ ID NO: 394 and SEQ ID NO: 395, SEQ ID NO: 396 and SEQ ID NO: 397, SEQ ID NO: 398 and SEQ ID NO: 399, SEQ ID NO: 400 and SEQ ID NO: 401, SEQ ID NO: 402 and SEQ ID NO: 403, SEQ ID NO: 404 and SEQ ID NO: 405, SEQ ID NO: 406 and SEQ ID NO: 407, SEQ ID NO: 408 and SEQ ID NO: 409, SEQ ID NO: 410 and SEQ ID NO: 411, SEQ ID NO: 412 and SEQ ID NO: 413, SEQ ID NO: 414 and SEQ ID NO: 415, SEQ ID NO: 416 and SEQ ID NO: 417, SEQ ID NO: 418 and SEQ ID NO: 419, SEQ ID NO: 420 and SEQ ID NO: 421, SEQ ID NO: 422 and SEQ ID NO: 423, SEQ ID NO: 424 and SEQ ID NO: 425, SEQ ID NO: 426 and SEQ ID NO: 427, SEQ ID NO: 428 and SEQ ID NO: 429, SEQ ID NO: 430 and SEQ ID NO: 431, SEQ ID NO: 432 and SEQ ID NO: 433, SEQ ID NO: 434 and SEQ ID NO: 435, SEQ ID NO: 436 and SEQ ID NO: 437, SEQ ID NO: 438 and SEQ ID NO: 439, SEQ ID NO: 440 and SEQ ID NO: 441, SEQ ID NO: 442 and SEQ ID NO: 443, SEQ ID NO: 444 and SEQ ID NO: 445, SEQ ID NO: 446 and SEQ ID NO: 447, SEQ ID NO: 448 and SEQ ID NO: 449, SEQ ID NO: 450 and SEQ ID NO: 451, SEQ ID NO: 452 and SEQ ID NO: 453, SEQ ID NO: 454 and SEQ ID NO: 455, SEQ ID NO: 456 and SEQ ID NO: 457, SEQ ID NO: 458 and SEQ ID NO: 459, SEQ ID NO: 460 and SEQ ID NO: 461, SEQ ID NO: 462 and SEQ ID NO: 463, SEQ ID NO: 464 and SEQ ID NO: 465, SEQ ID NO: 466 and SEQ ID NO: 467, SEQ ID NO: 468 and SEQ ID NO: 469, SEQ ID NO: 470 and SEQ ID NO: 471, SEQ ID NO: 472 and SEQ ID NO: 473, SEQ ID NO: 474 and SEQ ID NO: 475, SEQ ID NO: 476 and SEQ ID NO: 477, SEQ ID NO: 478 and SEQ ID NO: 479, SEQ ID NO: 480 and SEQ ID NO: 481, SEQ ID NO: 482 and SEQ ID NO: 483, SEQ ID NO: 484 and SEQ ID NO: 485, SEQ ID NO: 486 and SEQ ID NO: 487, SEQ ID NO: 488 and SEQ ID NO: 489, SEQ ID NO: 490 and SEQ ID NO: 491, SEQ ID NO: 492 and SEQ ID NO: 493, SEQ ID NO: 494 and SEQ ID NO: 495, SEQ ID NO: 496 and SEQ ID NO: 497, SEQ ID NO: 498 and SEQ ID NO: 499, SEQ ID NO: 500 and SEQ ID NO: 501, SEQ ID NO: 502 and SEQ ID NO: 503, SEQ ID NO: 504 and SEQ ID NO: 505, SEQ ID NO: 506 and SEQ ID NO: 507, SEQ ID NO: 508 and SEQ ID NO: 509, SEQ ID NO: 628 and SEQ ID NO: 629, SEQ ID NO: 630 and SEQ ID NO: 631, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, SEQ ID NO: 646 and SEQ ID NO: 647, SEQ ID NO: 648 and SEQ ID NO: 649, SEQ ID NO: 650 and SEQ ID NO: 651, or SEQ ID NO: 652 and SEQ ID NO: 653, respectively with zero, one, or two single amino acid substitutions in one or more of the HCDRs or LCDRs.

In some embodiments, the SARS-CoV-2-specific binding domain, e.g., scFv fragment comprises an antibody VH and a VL, where the VH and VL comprise amino acid sequences at least 80%, at least 85%, at least 90%, at least 95% or 100% identical to SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 104 and SEQ ID NO: 105, SEQ ID NO: 106 and SEQ ID NO: 107, SEQ ID NO: 108 and SEQ ID NO: 109, SEQ ID NO: 110 and SEQ ID NO: 111, SEQ ID NO: 112 and SEQ ID NO: 113, SEQ ID NO: 114 and SEQ ID NO: 115, SEQ ID NO: 116 and SEQ ID NO: 117, SEQ ID NO: 118 and SEQ ID NO: 119, SEQ ID NO: 120 and SEQ ID NO: 121, SEQ ID NO: 122 and SEQ ID NO: 123, SEQ ID NO: 124 and SEQ ID NO: 125, SEQ ID NO: 126 and SEQ ID NO: 127, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 150 and SEQ ID NO: 151, SEQ ID NO: 152 and SEQ ID NO: 153, SEQ ID NO: 154 and SEQ ID NO: 155, SEQ ID NO: 156 and SEQ ID NO: 157, SEQ ID NO: 158 and SEQ ID NO: 159, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 178 and SEQ ID NO: 179, SEQ ID NO: 180 and SEQ ID NO: 181, SEQ ID NO: 182 and SEQ ID NO: 183, SEQ ID NO: 184 and SEQ ID NO: 185, SEQ ID NO: 186 and SEQ ID NO: 187, SEQ ID NO: 188 and SEQ ID NO: 189, SEQ ID NO: 190 and SEQ ID NO: 191, SEQ ID NO: 192 and SEQ ID NO: 193, SEQ ID NO: 194 and SEQ ID NO: 195, SEQ ID NO: 196 and SEQ ID NO: 197, SEQ ID NO: 198 and SEQ ID NO: 199, SEQ ID NO: 200 and SEQ ID NO: 201, SEQ ID NO: 202 and SEQ ID NO: 203, SEQ ID NO: 204 and SEQ ID NO: 205, SEQ ID NO: 206 and SEQ ID NO: 207, SEQ ID NO: 208 and SEQ ID NO: 209, SEQ ID NO: 210 and SEQ ID NO: 211, SEQ ID NO: 212 and SEQ ID NO: 213, SEQ ID NO: 214 and SEQ ID NO: 215, SEQ ID NO: 216 and SEQ ID NO: 217, SEQ ID NO: 218 and SEQ ID NO: 219, SEQ ID NO: 220 and SEQ ID NO: 221, SEQ ID NO: 222 and SEQ ID NO: 223, SEQ ID NO: 224 and SEQ ID NO: 225, SEQ ID NO: 226 and SEQ ID NO: 227, SEQ ID NO: 228 and SEQ ID NO: 229, SEQ ID NO: 230 and SEQ ID NO: 231, SEQ ID NO: 232 and SEQ ID NO: 233, SEQ ID NO: 234 and SEQ ID NO: 235, SEQ ID NO: 236 and SEQ ID NO: 237, SEQ ID NO: 238 and SEQ ID NO: 239, SEQ ID NO: 240 and SEQ ID NO: 241, SEQ ID NO: 242 and SEQ ID NO: 243, SEQ ID NO: 244 and SEQ ID NO: 245, SEQ ID NO: 246 and SEQ ID NO: 247, SEQ ID NO: 248 and SEQ ID NO: 249, SEQ ID NO: 250 and SEQ ID NO: 251, SEQ ID NO: 252 and SEQ ID NO: 253, SEQ ID NO: 254 and SEQ ID NO: 255, SEQ ID NO: 256 and SEQ ID NO: 257, SEQ ID NO: 258 and SEQ ID NO: 259, SEQ ID NO: 260 and SEQ ID NO: 261, SEQ ID NO: 264 and SEQ ID NO: 265, SEQ ID NO: 266 and SEQ ID NO: 267, SEQ ID NO: 268 and SEQ ID NO: 269, SEQ ID NO: 270 and SEQ ID NO: 271, SEQ ID NO: 272 and SEQ ID NO: 273, SEQ ID NO: 274 and SEQ ID NO: 275, SEQ ID NO: 276 and SEQ ID NO: 277, SEQ ID NO: 278 and SEQ ID NO: 279, SEQ ID NO: 280 and SEQ ID NO: 281, SEQ ID NO: 282 and SEQ ID NO: 283, SEQ ID NO: 284 and SEQ ID NO: 285, SEQ ID NO: 286 and SEQ ID NO: 287, SEQ ID NO: 288 and SEQ ID NO: 289, SEQ ID NO: 290 and SEQ ID NO: 291, SEQ ID NO: 292 and SEQ ID NO: 293, SEQ ID NO: 294 and SEQ ID NO: 295, SEQ ID NO: 296 and SEQ ID NO: 297, SEQ ID NO: 298 and SEQ ID NO: 299, SEQ ID NO: 300 and SEQ ID NO: 301, SEQ ID NO: 302 and SEQ ID NO: 303, SEQ ID NO: 304 and SEQ ID NO: 305, SEQ ID NO: 306 and SEQ ID NO: 307, SEQ ID NO: 308 and SEQ ID NO: 309, SEQ ID NO: 310 and SEQ ID NO: 311, SEQ ID NO: 312 and SEQ ID NO: 313, SEQ ID NO: 314 and SEQ ID NO: 315, SEQ ID NO: 316 and SEQ ID NO: 317, SEQ ID NO: 318 and SEQ ID NO: 319, SEQ ID NO: 320 and SEQ ID NO: 321, SEQ ID NO: 322 and SEQ ID NO: 323, SEQ ID NO: 324 and SEQ ID NO: 325, SEQ ID NO: 326 and SEQ ID NO: 327, SEQ ID NO: 328 and SEQ ID NO: 329, SEQ ID NO: 330 and SEQ ID NO: 331, SEQ ID NO: 332 and SEQ ID NO: 333, SEQ ID NO: 336 and SEQ ID NO: 337, SEQ ID NO: 338 and SEQ ID NO: 339, SEQ ID NO: 340 and SEQ ID NO: 341, SEQ ID NO: 342 and SEQ ID NO: 343, SEQ ID NO: 344 and SEQ ID NO: 345, SEQ ID NO: 348 and SEQ ID NO: 349, SEQ ID NO: 350 and SEQ ID NO: 351, SEQ ID NO: 352 and SEQ ID NO: 353, SEQ ID NO: 354 and SEQ ID NO: 355, SEQ ID NO: 356 and SEQ ID NO: 357, SEQ ID NO: 358 and SEQ ID NO: 359, SEQ ID NO: 362 and SEQ ID NO: 363, SEQ ID NO: 364 and SEQ ID NO: 365, SEQ ID NO: 366 and SEQ ID NO: 367, SEQ ID NO: 368 and SEQ ID NO: 369, SEQ ID NO: 370 and SEQ ID NO: 371, SEQ ID NO: 372 and SEQ ID NO: 373, SEQ ID NO: 374 and SEQ ID NO: 375, SEQ ID NO: 376 and SEQ ID NO: 377, SEQ ID NO: 378 and SEQ ID NO: 379, SEQ ID NO: 380 and SEQ ID NO: 381, SEQ ID NO: 382 and SEQ ID NO: 383, SEQ ID NO: 384 and SEQ ID NO: 385, SEQ ID NO: 386 and SEQ ID NO: 387, SEQ ID NO: 388 and SEQ ID NO: 389, SEQ ID NO: 390 and SEQ ID NO: 391, SEQ ID NO: 392 and SEQ ID NO: 393, SEQ ID NO: 394 and SEQ ID NO: 395, SEQ ID NO: 396 and SEQ ID NO: 397, SEQ ID NO: 398 and SEQ ID NO: 399, SEQ ID NO: 400 and SEQ ID NO: 401, SEQ ID NO: 402 and SEQ ID NO: 403, SEQ ID NO: 404 and SEQ ID NO: 405, SEQ ID NO: 406 and SEQ ID NO: 407, SEQ ID NO: 408 and SEQ ID NO: 409, SEQ ID NO: 410 and SEQ ID NO: 411, SEQ ID NO: 412 and SEQ ID NO: 413, SEQ ID NO: 414 and SEQ ID NO: 415, SEQ ID NO: 416 and SEQ ID NO: 417, SEQ ID NO: 418 and SEQ ID NO: 419, SEQ ID NO: 420 and SEQ ID NO: 421, SEQ ID NO: 422 and SEQ ID NO: 423, SEQ ID NO: 424 and SEQ ID NO: 425, SEQ ID NO: 426 and SEQ ID NO: 427, SEQ ID NO: 428 and SEQ ID NO: 429, SEQ ID NO: 430 and SEQ ID NO: 431, SEQ ID NO: 432 and SEQ ID NO: 433, SEQ ID NO: 434 and SEQ ID NO: 435, SEQ ID NO: 436 and SEQ ID NO: 437, SEQ ID NO: 438 and SEQ ID NO: 439, SEQ ID NO: 440 and SEQ ID NO: 441, SEQ ID NO: 442 and SEQ ID NO: 443, SEQ ID NO: 444 and SEQ ID NO: 445, SEQ ID NO: 446 and SEQ ID NO: 447, SEQ ID NO: 448 and SEQ ID NO: 449, SEQ ID NO: 450 and SEQ ID NO: 451, SEQ ID NO: 452 and SEQ ID NO: 453, SEQ ID NO: 454 and SEQ ID NO: 455, SEQ ID NO: 456 and SEQ ID NO: 457, SEQ ID NO: 458 and SEQ ID NO: 459, SEQ ID NO: 460 and SEQ ID NO: 461, SEQ ID NO: 462 and SEQ ID NO: 463, SEQ ID NO: 464 and SEQ ID NO: 465, SEQ ID NO: 466 and SEQ ID NO: 467, SEQ ID NO: 468 and SEQ ID NO: 469, SEQ ID NO: 470 and SEQ ID NO: 471, SEQ ID NO: 472 and SEQ ID NO: 473, SEQ ID NO: 474 and SEQ ID NO: 475, SEQ ID NO: 476 and SEQ ID NO: 477, SEQ ID NO: 478 and SEQ ID NO: 479, SEQ ID NO: 480 and SEQ ID NO: 481, SEQ ID NO: 482 and SEQ ID NO: 483, SEQ ID NO: 484 and SEQ ID NO: 485, SEQ ID NO: 486 and SEQ ID NO: 487, SEQ ID NO: 488 and SEQ ID NO: 489, SEQ ID NO: 490 and SEQ ID NO: 491, SEQ ID NO: 492 and SEQ ID NO: 493, SEQ ID NO: 494 and SEQ ID NO: 495, SEQ ID NO: 496 and SEQ ID NO: 497, SEQ ID NO: 498 and SEQ ID NO: 499, SEQ ID NO: 500 and SEQ ID NO: 501, SEQ ID NO: 502 and SEQ ID NO: 503, SEQ ID NO: 504 and SEQ ID NO: 505, SEQ ID NO: 506 and SEQ ID NO: 507, SEQ ID NO: 508 and SEQ ID NO: 509, SEQ ID NO: 628 and SEQ ID NO: 629, SEQ ID NO: 630 and SEQ ID NO: 631, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, SEQ ID NO: 646 and SEQ ID NO: 647, SEQ ID NO: 648 and SEQ ID NO: 649, SEQ ID NO: 650 and SEQ ID NO: 651, or SEQ ID NO: 652 and SEQ ID NO: 653, respectively.

In certain embodiments, the binding domain, e.g., scFv fragment can specifically bind to human coronavirus. In certain embodiments, binding domain binds to a different epitope of the human coronavirus than the three to twelve identical binding domains of the multimeric binding molecule that specifically bind to the human coronavirus. In certain embodiments, the binding domain, e.g., scFv fragment can specifically bind to SARS-CoV.

In some embodiments, the SARS-CoV-specific binding domain, e.g., scFv fragment comprises a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 84 and SEQ ID NO: 85, SEQ ID NO: 118 and SEQ ID NO: 119, SEQ ID NO: 120 and SEQ ID NO: 121, SEQ ID NO: 138 and SEQ ID NO: 139, SEQ ID NO: 162 and SEQ ID NO: 163, SEQ ID NO: 222 and SEQ ID NO: 223, SEQ ID NO: 236 and SEQ ID NO: 237, SEQ ID NO: 252 and SEQ ID NO: 253, SEQ ID NO: 260 and SEQ ID NO: 261, SEQ ID NO: 262 and SEQ ID NO: 263, SEQ ID NO: 296 and SEQ ID NO: 297, SEQ ID NO: 384 and SEQ ID NO: 385, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, or SEQ ID NO: 646 and SEQ ID NO: 647, respectively with zero, one, or two single amino acid substitutions in one or more of the HCDRs or LCDRs.

In some embodiments, the SARS-CoV-specific binding domain, e.g., scFv fragment comprises an antibody VH and a VL, where the VH and VL comprise amino acid sequences at least 80%, at least 85%, at least 90%, at least 95% or 100% identical to SEQ ID NO: 84 and SEQ ID NO: 85, SEQ ID NO: 118 and SEQ ID NO: 119, SEQ ID NO: 120 and SEQ ID NO: 121, SEQ ID NO: 138 and SEQ ID NO: 139, SEQ ID NO: 162 and SEQ ID NO: 163, SEQ ID NO: 222 and SEQ ID NO: 223, SEQ ID NO: 236 and SEQ ID NO: 237, SEQ ID NO: 252 and SEQ ID NO: 253, SEQ ID NO: 260 and SEQ ID NO: 261, SEQ ID NO: 262 and SEQ ID NO: 263, SEQ ID NO: 296 and SEQ ID NO: 297, SEQ ID NO: 384 and SEQ ID NO: 385, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, or SEQ ID NO: 646 and SEQ ID NO: 647, respectively.

In certain embodiments, the binding domain, e.g., scFv fragment can specifically bind to human coronavirus. In certain embodiments, binding domain binds to a different epitope of the human coronavirus than the three to twelve identical binding domains of the multimeric binding molecule that specifically bind to the human coronavirus. In certain embodiments, the binding domain, e.g., scFv fragment can specifically bind to MERS-CoV.

In some embodiments, the MERS-CoV-specific binding domain, e.g., scFv fragment comprises a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 510 and SEQ ID NO: 511, SEQ ID NO: 512 and SEQ ID NO: 513, SEQ ID NO: 514 and SEQ ID NO: 515, SEQ ID NO: 516 and SEQ ID NO: 517, SEQ ID NO: 518 and SEQ ID NO: 519, SEQ ID NO: 520 and SEQ ID NO: 521, SEQ ID NO: 522 and SEQ ID NO: 523, SEQ ID NO: 524 and SEQ ID NO: 525, SEQ ID NO: 526 and SEQ ID NO: 527, SEQ ID NO: 528 and SEQ ID NO: 529, SEQ ID NO: 530 and SEQ ID NO: 531, SEQ ID NO: 532 and SEQ ID NO: 533, SEQ ID NO: 534 and SEQ ID NO: 535, SEQ ID NO: 536 and SEQ ID NO: 537, SEQ ID NO: 538 and SEQ ID NO: 539, SEQ ID NO: 540 and SEQ ID NO: 541, SEQ ID NO: 542 and SEQ ID NO: 543, SEQ ID NO: 544 and SEQ ID NO: 545, SEQ ID NO: 546 and SEQ ID NO: 547, SEQ ID NO: 548 and SEQ ID NO: 549, SEQ ID NO: 550 and SEQ ID NO: 551, SEQ ID NO: 552 and SEQ ID NO: 553, SEQ ID NO: 554 and SEQ ID NO: 555, SEQ ID NO: 556 and SEQ ID NO: 557, SEQ ID NO: 558 and SEQ ID NO: 559, SEQ ID NO: 560 and SEQ ID NO: 561, SEQ ID NO: 562 and SEQ ID NO: 563, SEQ ID NO: 564 and SEQ ID NO: 565, SEQ ID NO: 566 and SEQ ID NO: 567, SEQ ID NO: 568 and SEQ ID NO: 569, SEQ ID NO: 570 and SEQ ID NO: 571, SEQ ID NO: 572 and SEQ ID NO: 573, SEQ ID NO: 574 and SEQ ID NO: 575, SEQ ID NO: 576 and SEQ ID NO: 577, SEQ ID NO: 578 and SEQ ID NO: 579, SEQ ID NO: 580 and SEQ ID NO: 581, SEQ ID NO: 582 and SEQ ID NO: 583, SEQ ID NO: 584 and SEQ ID NO: 585, SEQ ID NO: 586 and SEQ ID NO: 587, SEQ ID NO: 588 and SEQ ID NO: 589, SEQ ID NO: 590 and SEQ ID NO: 591, SEQ ID NO: 592 and SEQ ID NO: 593, SEQ ID NO: 594 and SEQ ID NO: 595, SEQ ID NO: 596 and SEQ ID NO: 597, SEQ ID NO: 598 and SEQ ID NO: 599, SEQ ID NO: 600 and SEQ ID NO: 601, SEQ ID NO: 602 and SEQ ID NO: 603, SEQ ID NO: 604 and SEQ ID NO: 605, SEQ ID NO: 606 and SEQ ID NO: 607, SEQ ID NO: 608 and SEQ ID NO: 609, SEQ ID NO: 610 and SEQ ID NO: 611, SEQ ID NO: 612 and SEQ ID NO: 613, SEQ ID NO: 614 and SEQ ID NO: 615, SEQ ID NO: 616 and SEQ ID NO: 617, SEQ ID NO: 618 and SEQ ID NO: 619, SEQ ID NO: 620 and SEQ ID NO: 621, SEQ ID NO: 622 and SEQ ID NO: 623, SEQ ID NO: 624 and SEQ ID NO: 625, SEQ ID NO: 626 and SEQ ID NO: 627, or SEQ ID NO: 630 and SEQ ID NO: 631, respectively with zero, one, or two single amino acid substitutions in one or more of the HCDRs or LCDRs.

In some embodiments, the MERS-CoV-specific binding domain, e.g., scFv fragment comprises an antibody VH and a VL, where the VH and VL comprise amino acid sequences at least 80%, at least 85%, at least 90%, at least 95% or 100% identical to SEQ ID NO: 510 and SEQ ID NO: 511, SEQ ID NO: 512 and SEQ ID NO: 513, SEQ ID NO: 514 and SEQ ID NO: 515, SEQ ID NO: 516 and SEQ ID NO: 517, SEQ ID NO: 518 and SEQ ID NO: 519, SEQ ID NO: 520 and SEQ ID NO: 521, SEQ ID NO: 522 and SEQ ID NO: 523, SEQ ID NO: 524 and SEQ ID NO: 525, SEQ ID NO: 526 and SEQ ID NO: 527, SEQ ID NO: 528 and SEQ ID NO: 529, SEQ ID NO: 530 and SEQ ID NO: 531, SEQ ID NO: 532 and SEQ ID NO: 533, SEQ ID NO: 534 and SEQ ID NO: 535, SEQ ID NO: 536 and SEQ ID NO: 537, SEQ ID NO: 538 and SEQ ID NO: 539, SEQ ID NO: 540 and SEQ ID NO: 541, SEQ ID NO: 542 and SEQ ID NO: 543, SEQ ID NO: 544 and SEQ ID NO: 545, SEQ ID NO: 546 and SEQ ID NO: 547, SEQ ID NO: 548 and SEQ ID NO: 549, SEQ ID NO: 550 and SEQ ID NO: 551, SEQ ID NO: 552 and SEQ ID NO: 553, SEQ ID NO: 554 and SEQ ID NO: 555, SEQ ID NO: 556 and SEQ ID NO: 557, SEQ ID NO: 558 and SEQ ID NO: 559, SEQ ID NO: 560 and SEQ ID NO: 561, SEQ ID NO: 562 and SEQ ID NO: 563, SEQ ID NO: 564 and SEQ ID NO: 565, SEQ ID NO: 566 and SEQ ID NO: 567, SEQ ID NO: 568 and SEQ ID NO: 569, SEQ ID NO: 570 and SEQ ID NO: 571, SEQ ID NO: 572 and SEQ ID NO: 573, SEQ ID NO: 574 and SEQ ID NO: 575, SEQ ID NO: 576 and SEQ ID NO: 577, SEQ ID NO: 578 and SEQ ID NO: 579, SEQ ID NO: 580 and SEQ ID NO: 581, SEQ ID NO: 582 and SEQ ID NO: 583, SEQ ID NO: 584 and SEQ ID NO: 585, SEQ ID NO: 586 and SEQ ID NO: 587, SEQ ID NO: 588 and SEQ ID NO: 589, SEQ ID NO: 590 and SEQ ID NO: 591, SEQ ID NO: 592 and SEQ ID NO: 593, SEQ ID NO: 594 and SEQ ID NO: 595, SEQ ID NO: 596 and SEQ ID NO: 597, SEQ ID NO: 598 and SEQ ID NO: 599, SEQ ID NO: 600 and SEQ ID NO: 601, SEQ ID NO: 602 and SEQ ID NO: 603, SEQ ID NO: 604 and SEQ ID NO: 605, SEQ ID NO: 606 and SEQ ID NO: 607, SEQ ID NO: 608 and SEQ ID NO: 609, SEQ ID NO: 610 and SEQ ID NO: 611, SEQ ID NO: 612 and SEQ ID NO: 613, SEQ ID NO: 614 and SEQ ID NO: 615, SEQ ID NO: 616 and SEQ ID NO: 617, SEQ ID NO: 618 and SEQ ID NO: 619, SEQ ID NO: 620 and SEQ ID NO: 621, SEQ ID NO: 622 and SEQ ID NO: 623, SEQ ID NO: 624 and SEQ ID NO: 625, SEQ ID NO: 626 and SEQ ID NO: 627, or SEQ ID NO: 630 and SEQ ID NO: 631, respectively.

In some embodiments, the binding domain comprises a single domain variable region (VHH), where the VHH comprises three immunoglobulin complementarity determining regions HCDR1, HCDR2, and HCDR3, where the HCDR1, HCDR2, and HCDR3 the CDRs of an antibody comprising the VHH of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, or SEQ ID NO: 83, with zero, one, or two single amino acid substitutions in one or more of the HCDRs.

In some embodiments, the binding domain comprises a VHH amino acid sequence at least 80%, at least 85%, at least 90%, at least 95% or 100% identical to SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, or SEQ ID NO: 83.

In some embodiments, the binding domain is an extracellular SARS-CoV-2 RBD-binding fragment of angiotensin-converting enzyme 2 (ACE2). In some embodiments, the binding domain is an extracellular SARS-CoV-2 RBD-binding fragment comprising amino acids 18 to 740 of SEQ ID NO: 14. In some embodiments, the binding domain comprises an amino acid sequence at least 80%, at least 85%, at least 90%, at least 95% or 100% identical to amino acids 18 to 740 of SEQ ID NO: 14.

The antigen-binding domain can be introduced into the J-chain at any location that allows the binding of the antigen-binding domain to its binding target without interfering with J-chain function or the function of an associated multimeric binding molecule, e.g., a pentameric IgM or a dimeric or tetrameric IgA antibody. Insertion locations include but are not limited to at or near the C-terminus, at or near the N-terminus or at an internal location that, based on the three-dimensional structure of the J-chain, is accessible.

Variant J-Chains that Confer Increased Serum Half-Life

In certain embodiments, the J-chain is a functional variant J-chain that includes one or more single amino acid substitutions, deletions, or insertions relative to a reference J-chain identical to the variant J-chain except for the one or more single amino acid substitutions, deletions, or insertions. For example, certain amino acid substitutions, deletions, or insertions can result in the IgM-derived binding molecule exhibiting an increased serum half-life upon administration to a subject animal relative to a reference IgM-derived binding molecule that is identical except for the one or more single amino acid substitutions, deletions, or insertions in the variant J-chain, and is administered using the same method to the same animal species. In certain embodiments the variant J-chain can include one, two, three, or four single amino acid substitutions, deletions, or insertions relative to the reference J-chain. Exemplary J-chains that confer increased serum half-life can be found, e.g., in U.S. Pat. No. 10,899,935, which is incorporated herein by reference in its entirety.

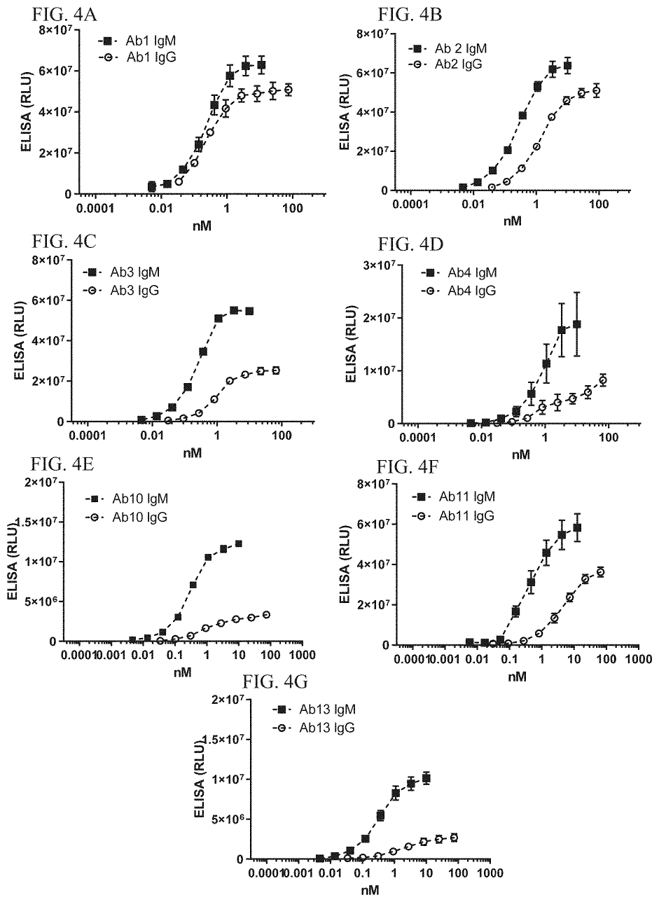

In certain embodiments, the J-chain, such as a modified J-chain, comprises an amino acid substitution at the amino acid position corresponding to amino acid Y102 of the mature wild-type human J-chain (SEQ ID NO: 7). By “an amino acid corresponding to amino acid Y102 of the mature wild-type human J-chain” is meant the amino acid in the sequence of the J-chain, which is homologous to Y102 in the human J-chain. For example, see U.S. Pat. No. 10,899,835, which is incorporated herein by reference in its entirety. The position corresponding to Y102 in SEQ ID NO: 7 is conserved in the J-chain amino acid sequences of at least 43 other species. See FIG. 4 of U.S. Pat. No. 9,951,134, which is incorporated by reference herein. Certain mutations at the position corresponding to Y102 of SEQ ID NO: 7 can inhibit the binding of IgM pentamers comprising the variant J-chain to certain immunoglobulin receptors, e.g., the human or murine Fcαμ receptor, the murine Fcμ receptor, and/or the human or murine polymeric Ig receptor (pIgR).

A multimeric binding molecule comprising a mutation at the amino acid corresponding to Y102 of SEQ ID NO: 7 has an improved serum half-life when administered to an animal than a corresponding multimeric binding molecule that is identical except for the substitution, and which is administered to the same species in the same manner. In certain embodiments, the amino acid corresponding to Y102 of SEQ ID NO: 7 can be substituted with any amino acid. In certain embodiments, the amino acid corresponding to Y102 of SEQ ID NO: 7 can be substituted with alanine (A), serine (S) or arginine (R). In a particular embodiment, the amino acid corresponding to Y102 of SEQ ID NO: 7 can be substituted with alanine. In a particular embodiment the J-chain or functional fragment or variant thereof is a variant human J-chain referred to herein as “J*,” and comprises the amino acid sequence SEQ ID NO: 8.

Wild-type J-chains typically include one N-linked glycosylation site. In certain embodiments, a variant J-chain or functional fragment thereof of a multimeric binding molecule as provided herein includes a mutation within the asparagine(N)-linked glycosylation motif N-X₁-S/T, e.g., starting at the amino acid position corresponding to amino acid 49 (motif N6) of the mature human J-chain (SEQ ID NO: 7) or J* (SEQ ID NO: 8), where N is asparagine, X₁ is any amino acid except proline, and S/T is serine or threonine, and where the mutation prevents glycosylation at that motif. As demonstrated in U.S. Pat. No. 10,899,835, mutations preventing glycosylation at this site can result in the multimeric binding molecule as provided herein, exhibiting an increased serum half-life upon administration to a subject animal relative to a reference multimeric binding molecule that is identical except for the mutation or mutations preventing glycosylation in the variant J-chain, and is administered in the same way to the same animal species.

For example, in certain embodiments the variant J-chain or functional fragment thereof of a pentameric IgM-derived or dimeric IgA-derived binding molecule as provided herein can include an amino acid substitution at the amino acid position corresponding to amino acid N49 or amino acid S51 of SEQ ID NO: 7 or SEQ ID NO: 8, provided that the amino acid corresponding to S51 is not substituted with threonine (T), or where the variant J-chain comprises amino acid substitutions at the amino acid positions corresponding to both amino acids N49 and S51 of SEQ ID NO: 7 or SEQ ID NO: 8. In certain embodiments, the position corresponding to N49 of SEQ ID NO: 7 or SEQ ID NO: 8 is substituted with any amino acid, e.g., alanine (A), glycine (G), threonine (T), serine (S) or aspartic acid (D). In a particular embodiment, the position corresponding to N49 of SEQ ID NO: 7 or SEQ ID NO: 8 can be substituted with alanine (A). In another particular embodiment, the position corresponding to N49 of SEQ ID NO: 7 or SEQ ID NO: 8 can be substituted with aspartic acid (D). In some embodiments, the position corresponding to S51 of SEQ ID NO: 7 or SEQ ID NO: 8 is substituted with alanine (A) or glycine (G). In some embodiments, the position corresponding to S51 of SEQ ID NO: 7 or SEQ ID NO: 8 is substituted with alanine (A).

### Variant IgM Constant Regions

IgM heavy chain constant regions of a multimeric binding molecule as provided herein can be engineered to confer certain desirable properties to the multimeric binding molecules provided herein. For example, in certain embodiments, IgM heavy chain constant regions can be engineered to confer enhanced serum half-life to multimeric binding molecules as provided herein. Exemplary IgM heavy chain constant region mutations that can enhance serum half-life of an IgM-derived binding molecule are disclosed in U.S. Pat. No. 10,899,835, which is incorporated by reference herein in its entirety. For example, a variant IgM heavy chain constant region of the IgM antibody, IgM-like antibody, or IgM-derived binding molecule as provided herein can include an amino acid substitution at a position corresponding to amino acid S401, E402, E403, R344, and/or E345 of a wild-type human IgM constant region (e.g., SEQ ID NO: 1 or SEQ ID NO: 2). By “an amino acid corresponding to amino acid S401, E402, E403, R344, and/or E345 of a wild-type human IgM constant region” is meant the amino acid in the sequence of the IgM constant region of any species which is homologous to 5401, E402, E403, R344, and/or E345 in the human IgM constant region. In certain embodiments, the amino acid corresponding to 5401, E402, E403, R344, and/or E345 of SEQ ID NO: 1 or SEQ ID NO: 2 can be substituted with any amino acid, e.g., alanine.

In certain embodiments, an IgM antibody, IgM-like antibody, or other IgM-derived binding molecule as provided herein, can be engineered to exhibit reduced complement-dependent cytotoxicity (CDC) activity to cells in the presence of complement, relative to a reference IgM antibody, IgM-like antibody, or other IgM-derived binding molecule with corresponding reference human IgM constant regions identical, except for the mutations conferring reduced CDC activity. These CDC mutations can be combined with any of the mutations to confer increased serum half-life as provided herein. By “corresponding reference human IgM constant region” is meant a human IgM constant region that is identical to the variant IgM constant region except for the modification or modifications in the constant region affecting CDC activity. In certain embodiments, the variant human IgM constant region includes one or more amino acid substitutions, e.g., in the Cμ3 domain, relative to a wild-type human IgM constant region as described, e.g., in U.S. Patent Application Publication No. US 2021-0147567, which is incorporated herein by reference in its entirety. Assays for measuring CDC are well known to those of ordinary skill in the art, and exemplary assays are described e.g., in U.S. Patent Application Publication No. US 2021-0147567.

In certain embodiments, a variant human IgM constant region conferring reduced CDC activity includes an amino acid substitution corresponding to the wild-type human IgM constant region at position L310, P311, P313, and/or K315 of SEQ ID NO: 1 (human IgM constant region allele IGHM*03) or SEQ ID NO: 2 (human IgM constant region allele IGHM*04). In certain embodiments, a variant human IgM constant region conferring reduced CDC activity includes an amino acid substitution corresponding to the wild-type human IgM constant region at position P311 of SEQ ID NO: 1 or SEQ ID NO: 2. In other embodiments the variant IgM constant region as provided herein contains an amino acid substitution corresponding to the wild-type human IgM constant region at position P313 of SEQ ID NO: 1 or SEQ ID NO: 2. In other embodiments the variant IgM constant region as provided herein contains a combination of substitutions corresponding to the wild-type human IgM constant region at positions P311 of SEQ ID NO: 1 or SEQ ID NO: 2 and P313 of SEQ ID NO: 1 or SEQ ID NO: 2. These proline residues can be independently substituted with any amino acid, e.g., with alanine, serine, or glycine. In certain embodiments, a variant human IgM constant region conferring reduced CDC activity includes an amino acid substitution corresponding to the wild-type human IgM constant region at position K315 of SEQ ID NO: 1 or SEQ ID NO: 2. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position K315 of SEQ ID NO: 1 or SEQ ID NO: 2 with aspartic acid.

Human and certain non-human primate IgM constant regions typically include five (5) naturally occurring asparagine (N)-linked glycosylation motifs or sites. As used herein “an N-linked glycosylation motif” comprises or consists of the amino acid sequence N-X₁-S/T, where N is asparagine, X₁ is any amino acid except proline (P), and S/T is serine (S) or threonine (T). The glycan is attached to the nitrogen atom of the asparagine residue. See, e.g., Drickamer K, Taylor M E (2006), Introduction to Glycobiology (2nd ed.). Oxford University Press, USA. N-linked glycosylation motifs occur in the human IgM heavy chain constant regions of SEQ ID NO: 1 or SEQ ID NO: 2 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Accordingly, in some embodiments, IgM heavy chain constant regions of a multimeric binding molecule as provided herein comprise 5 N-linked glycosylation motifs: N1, N2, N3, N4, and N5. In some embodiments, at least three of the N-linked glycosylation motifs (e.g., N1, N2, and N3) on each IgM heavy chain constant region are occupied by a complex glycan.

In certain embodiments, at least one, at least two, at least three, or at least four of the N-X₁-S/T motifs can include an amino acid insertion, deletion, or substitution that prevents glycosylation at that motif. In certain embodiments, the IgM-derived multimeric binding molecule can include an amino acid insertion, deletion, or substitution at motif N1, motif N2, motif N3, motif N5, or any combination of two or more, three or more, or all four of motifs N1, N2, N3, or N5, where the amino acid insertion, deletion, or substitution prevents glycosylation at that motif. In some embodiment, the IgM constant region comprises one or more substitutions relative to a wild-type human IgM constant region at positions 46, 209, 272, or 440 of SEQ ID NO: 1 (human IgM constant region allele IGHM*03) or SEQ ID NO: 2 (human IgM constant region allele IGHM*04). See, e.g., PCT Application No. PCT/US2020/047495, which is incorporated herein by reference in its entirety.

### Polynucleotides and Vectors

In certain embodiments, this disclosure provides a polynucleotide comprising a nucleic acid sequence that encodes a polypeptide subunit of a multimeric binding molecule described herein. In some embodiments, the polynucleotide encodes a polypeptide subunit comprising a heavy chain constant region and at least an antibody VH or VHH portion of the SARS-CoV-2-binding domain of the multimeric binding molecule. In some embodiments, the polynucleotide encodes a polypeptide subunit comprising the heavy chain of the multimeric binding molecule. In some embodiments, the polynucleotide encodes a polypeptide subunit comprising a human IgM constant region or fragment thereof fused to the C-terminal end of a VH or VHH comprising HCDR1, HCDR2, and HCDR3 regions comprising the CDRs contained in the VH or VHH amino acid sequences SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 336, SEQ ID NO: 338, SEQ ID NO: 340, SEQ ID NO: 342, SEQ ID NO: 344, SEQ ID NO: 348, SEQ ID NO: 350, SEQ ID NO: 352, SEQ ID NO: 354, SEQ ID NO: 356, SEQ ID NO: 358, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 628, SEQ ID NO: 630, SEQ ID NO: 632, SEQ ID NO: 634, SEQ ID NO: 636, SEQ ID NO: 638, SEQ ID NO: 640, SEQ ID NO: 642, SEQ ID NO: 644, SEQ ID NO: 646, SEQ ID NO: 648, SEQ ID NO: 650, or SEQ ID NO: 652, with zero, one, or two single amino acid substitutions in one or more of the HCDRs. In some embodiments, the polynucleotide encodes a polypeptide subunit comprising a human IgM constant region or fragment thereof fused to the C-terminal end of a VH or VHH comprising an amino acid sequence at least 80%, at least 85%, at least 90%, at least 95% or 100% identical to SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 336, SEQ ID NO: 338, SEQ ID NO: 340, SEQ ID NO: 342, SEQ ID NO: 344, SEQ ID NO: 348, SEQ ID NO: 350, SEQ ID NO: 352, SEQ ID NO: 354, SEQ ID NO: 356, SEQ ID NO: 358, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 628, SEQ ID NO: 630, SEQ ID NO: 632, SEQ ID NO: 634, SEQ ID NO: 636, SEQ ID NO: 638, SEQ ID NO: 640, SEQ ID NO: 642, SEQ ID NO: 644, SEQ ID NO: 646, SEQ ID NO: 648, SEQ ID NO: 650, or SEQ ID NO: 652.

In certain embodiments, this disclosure provides a polynucleotide comprising a nucleic acid sequence that encodes a polypeptide subunit of a multimeric binding molecule described herein. In some embodiments, the polynucleotide encodes a polypeptide subunit comprising a heavy chain constant region and at least an antibody VH portion of the SARS-CoV-binding domain of the multimeric binding molecule. In some embodiments, the polynucleotide encodes a polypeptide subunit comprising the heavy chain of the multimeric binding molecule. In some embodiments, the polynucleotide encodes a polypeptide subunit comprising a human IgM constant region or fragment thereof fused to the C-terminal end of a VH comprising HCDR1, HCDR2, and HCDR3 regions comprising the CDRs contained in the VH amino acid sequences SEQ ID NO: 84, SEQ ID NO: 118, SEQ ID NO: 120, SEQ ID NO: 138, SEQ ID NO: 162, SEQ ID NO: 222, SEQ ID NO: 236, SEQ ID NO: 252, SEQ ID NO: 260, SEQ ID NO: 262, SEQ ID NO: 296, SEQ ID NO: 384, SEQ ID NO: 628, SEQ ID NO: 632, SEQ ID NO: 634, SEQ ID NO: 636, SEQ ID NO: 638, SEQ ID NO: 640, SEQ ID NO: 642, SEQ ID NO: 644, or SEQ ID NO: 646, with zero, one, or two single amino acid substitutions in one or more of the HCDRs. In some embodiments, the polynucleotide encodes a polypeptide subunit comprising a human IgM constant region or fragment thereof fused to the C-terminal end of a VH comprising an amino acid sequence at least 80%, at least 85%, at least 90%, at least 95% or 100% identical to SEQ ID NO: 84, SEQ ID NO: 118, SEQ ID NO: 120, SEQ ID NO: 138, SEQ ID NO: 162, SEQ ID NO: 222, SEQ ID NO: 236, SEQ ID NO: 252, SEQ ID NO: 260, SEQ ID NO: 262, SEQ ID NO: 296, SEQ ID NO: 384, SEQ ID NO: 628, SEQ ID NO: 632, SEQ ID NO: 634, SEQ ID NO: 636, SEQ ID NO: 638, SEQ ID NO: 640, SEQ ID NO: 642, SEQ ID NO: 644, or SEQ ID NO: 646.

In certain embodiments, this disclosure provides a polynucleotide comprising a nucleic acid sequence that encodes a polypeptide subunit of a multimeric binding molecule described herein. In some embodiments, the polynucleotide encodes a polypeptide subunit comprising a heavy chain constant region and at least an antibody VH portion of the MERS-CoV-binding domain of the multimeric binding molecule. In some embodiments, the polynucleotide encodes a polypeptide subunit comprising the heavy chain of the multimeric binding molecule. In some embodiments, the polynucleotide encodes a polypeptide subunit comprising a human IgM constant region or fragment thereof fused to the C-terminal end of a VH comprising HCDR1, HCDR2, and HCDR3 regions comprising the CDRs contained in the VH amino acid sequence SEQ ID NO: 510, SEQ ID NO: 512, SEQ ID NO: 514, SEQ ID NO: 516, SEQ ID NO: 518, SEQ ID NO: 520, SEQ ID NO: 522, SEQ ID NO: 524, SEQ ID NO: 526, SEQ ID NO: 528, SEQ ID NO: 530, SEQ ID NO: 532, SEQ ID NO: 534, SEQ ID NO: 536, SEQ ID NO: 538, SEQ ID NO: 540, SEQ ID NO: 542, SEQ ID NO: 544, SEQ ID NO: 546, SEQ ID NO: 548, SEQ ID NO: 550, SEQ ID NO: 552, SEQ ID NO: 554, SEQ ID NO: 556, SEQ ID NO: 558, SEQ ID NO: 560, SEQ ID NO: 562, SEQ ID NO: 564, SEQ ID NO: 566, SEQ ID NO: 568, SEQ ID NO: 570, SEQ ID NO: 572, SEQ ID NO: 574, SEQ ID NO: 576, SEQ ID NO: 578, SEQ ID NO: 580, SEQ ID NO: 582, SEQ ID NO: 584, SEQ ID NO: 586, SEQ ID NO: 588, SEQ ID NO: 590, SEQ ID NO: 592, SEQ ID NO: 594, SEQ ID NO: 596, SEQ ID NO: 598, SEQ ID NO: 600, SEQ ID NO: 602, SEQ ID NO: 604, SEQ ID NO: 606, SEQ ID NO: 608, SEQ ID NO: 610, SEQ ID NO: 612, SEQ ID NO: 614, SEQ ID NO: 616, SEQ ID NO: 618, SEQ ID NO: 620, SEQ ID NO: 622, SEQ ID NO: 624, SEQ ID NO: 626, or SEQ ID NO: 630, with zero, one, or two single amino acid substitutions in one or more of the HCDRs. In some embodiments, the polynucleotide encodes a polypeptide subunit comprising a human IgM constant region or fragment thereof fused to the C-terminal end of a VH comprising an amino acid sequence at least 80%, at least 85%, at least 90%, at least 95% or 100% identical to SEQ ID NO: 510, SEQ ID NO: 512, SEQ ID NO: 514, SEQ ID NO: 516, SEQ ID NO: 518, SEQ ID NO: 520, SEQ ID NO: 522, SEQ ID NO: 524, SEQ ID NO: 526, SEQ ID NO: 528, SEQ ID NO: 530, SEQ ID NO: 532, SEQ ID NO: 534, SEQ ID NO: 536, SEQ ID NO: 538, SEQ ID NO: 540, SEQ ID NO: 542, SEQ ID NO: 544, SEQ ID NO: 546, SEQ ID NO: 548, SEQ ID NO: 550, SEQ ID NO: 552, SEQ ID NO: 554, SEQ ID NO: 556, SEQ ID NO: 558, SEQ ID NO: 560, SEQ ID NO: 562, SEQ ID NO: 564, SEQ ID NO: 566, SEQ ID NO: 568, SEQ ID NO: 570, SEQ ID NO: 572, SEQ ID NO: 574, SEQ ID NO: 576, SEQ ID NO: 578, SEQ ID NO: 580, SEQ ID NO: 582, SEQ ID NO: 584, SEQ ID NO: 586, SEQ ID NO: 588, SEQ ID NO: 590, SEQ ID NO: 592, SEQ ID NO: 594, SEQ ID NO: 596, SEQ ID NO: 598, SEQ ID NO: 600, SEQ ID NO: 602, SEQ ID NO: 604, SEQ ID NO: 606, SEQ ID NO: 608, SEQ ID NO: 610, SEQ ID NO: 612, SEQ ID NO: 614, SEQ ID NO: 616, SEQ ID NO: 618, SEQ ID NO: 620, SEQ ID NO: 622, SEQ ID NO: 624, SEQ ID NO: 626, or SEQ ID NO: 630.

In some embodiments, the polynucleotide encodes a polypeptide subunit comprising a light chain constant region and an antibody VL portion of the SARS-CoV-2-binding domain of the multimeric binding molecule. In some embodiments, the polynucleotide encodes a polypeptide subunit comprising the light chain of the multimeric binding molecule. In some embodiments, the polynucleotide encodes a polypeptide subunit comprises a human kappa or lambda light chain constant region or fragment thereof fused to the C-terminal end of a VL comprising LCDR1, LCDR2, and LCDR3 regions comprising the CDRs contained in the VL amino acid sequences SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 337, SEQ ID NO: 339, SEQ ID NO: 341, SEQ ID NO: 343, SEQ ID NO: 345, SEQ ID NO: 349, SEQ ID NO: 351, SEQ ID NO: 353, SEQ ID NO: 355, SEQ ID NO: 357, SEQ ID NO: 359, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509, SEQ ID NO: 629, SEQ ID NO: 631, SEQ ID NO: 633, SEQ ID NO: 635, SEQ ID NO: 637, SEQ ID NO: 639, SEQ ID NO: 641, SEQ ID NO: 643, SEQ ID NO: 645, SEQ ID NO: 647, SEQ ID NO: 649, SEQ ID NO: 651, or SEQ ID NO: 653, with zero, one, or two single amino acid substitutions in one or more of the LCDRs. In some embodiments, the polynucleotide encodes a polypeptide subunit comprises a human kappa or lambda light chain constant region or fragment thereof fused to the C-terminal end of a VL comprising an amino acid sequence at least 80%, at least 85%, at least 90%, at least 95% or 100% identical to SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 337, SEQ ID NO: 339, SEQ ID NO: 341, SEQ ID NO: 343, SEQ ID NO: 345, SEQ ID NO: 349, SEQ ID NO: 351, SEQ ID NO: 353, SEQ ID NO: 355, SEQ ID NO: 357, SEQ ID NO: 359, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509, SEQ ID NO: 629, SEQ ID NO: 631, SEQ ID NO: 633, SEQ ID NO: 635, SEQ ID NO: 637, SEQ ID NO: 639, SEQ ID NO: 641, SEQ ID NO: 643, SEQ ID NO: 645, SEQ ID NO: 647, SEQ ID NO: 649, SEQ ID NO: 651, or SEQ ID NO: 653.

In some embodiments, the polynucleotide encodes a polypeptide subunit comprising a light chain constant region and an antibody VL portion of the SARS-CoV-binding domain of the multimeric binding molecule. In some embodiments, the polynucleotide encodes a polypeptide subunit comprising the light chain of the multimeric binding molecule. In some embodiments, the polynucleotide encodes a polypeptide subunit comprises a human kappa or lambda light chain constant region or fragment thereof fused to the C-terminal end of a VL comprising LCDR1, LCDR2, and LCDR3 regions comprising the CDRs contained in the VL amino acid sequences SEQ ID NO: 85, SEQ ID NO: 119, SEQ ID NO: 121, SEQ ID NO: 139, SEQ ID NO: 163, SEQ ID NO: 223, SEQ ID NO: 237, SEQ ID NO: 253, SEQ ID NO: 261, SEQ ID NO: 263, SEQ ID NO: 297, SEQ ID NO: 345, SEQ ID NO: 629, SEQ ID NO: 633, SEQ ID NO: 635, SEQ ID NO: 637, SEQ ID NO: 639, SEQ ID NO: 641, SEQ ID NO: 643, SEQ ID NO: 645, or SEQ ID NO: 647, with zero, one, or two single amino acid substitutions in one or more of the LCDRs. In some embodiments, the polynucleotide encodes a polypeptide subunit comprises a human kappa or lambda light chain constant region or fragment thereof fused to the C-terminal end of a VL comprising an amino acid sequence at least 80%, at least 85%, at least 90%, at least 95% or 100% identical to SEQ ID NO: 85, SEQ ID NO: 119, SEQ ID NO: 121, SEQ ID NO: 139, SEQ ID NO: 163, SEQ ID NO: 223, SEQ ID NO: 237, SEQ ID NO: 253, SEQ ID NO: 261, SEQ ID NO: 263, SEQ ID NO: 297, SEQ ID NO: 345, SEQ ID NO: 629, SEQ ID NO: 633, SEQ ID NO: 635, SEQ ID NO: 637, SEQ ID NO: 639, SEQ ID NO: 641, SEQ ID NO: 643, SEQ ID NO: 645, or SEQ ID NO: 647.

In some embodiments, the polynucleotide encodes a polypeptide subunit comprising a light chain constant region and an antibody VL portion of the MERS-CoV-binding domain of the multimeric binding molecule. In some embodiments, the polynucleotide encodes a polypeptide subunit comprising the light chain of the multimeric binding molecule. In some embodiments, the polynucleotide encodes a polypeptide subunit comprises a human kappa or lambda light chain constant region or fragment thereof fused to the C-terminal end of a VL comprising LCDR1, LCDR2, and LCDR3 regions comprising the CDRs contained in the VL amino acid sequences SEQ ID NO: 511, SEQ ID NO: 513, SEQ ID NO: 515, SEQ ID NO: 517, SEQ ID NO: 519, SEQ ID NO: 521, SEQ ID NO: 523, SEQ ID NO: 525, SEQ ID NO: 527, SEQ ID NO: 529, SEQ ID NO: 531, SEQ ID NO: 533, SEQ ID NO: 535, SEQ ID NO: 537, SEQ ID NO: 539, SEQ ID NO: 541, SEQ ID NO: 543, SEQ ID NO: 545, SEQ ID NO: 547, SEQ ID NO: 549, SEQ ID NO: 551, SEQ ID NO: 553, SEQ ID NO: 555, SEQ ID NO: 557, SEQ ID NO: 559, SEQ ID NO: 561, SEQ ID NO: 563, SEQ ID NO: 565, SEQ ID NO: 567, SEQ ID NO: 569, SEQ ID NO: 571, SEQ ID NO: 573, SEQ ID NO: 575, SEQ ID NO: 577, SEQ ID NO: 579, SEQ ID NO: 581, SEQ ID NO: 583, SEQ ID NO: 585, SEQ ID NO: 587, SEQ ID NO: 589, SEQ ID NO: 591, SEQ ID NO: 593, SEQ ID NO: 595, SEQ ID NO: 597, SEQ ID NO: 599, SEQ ID NO: 601, SEQ ID NO: 603, SEQ ID NO: 605, SEQ ID NO: 607, SEQ ID NO: 609, SEQ ID NO: 611, SEQ ID NO: 613, SEQ ID NO: 615, SEQ ID NO: 617, SEQ ID NO: 619, SEQ ID NO: 621, SEQ ID NO: 623, SEQ ID NO: 625, SEQ ID NO: 627, or SEQ ID NO: 631, with zero, one, or two single amino acid substitutions in one or more of the LCDRs. In some embodiments, the polynucleotide encodes a polypeptide subunit comprises a human kappa or lambda light chain constant region or fragment thereof fused to the C-terminal end of a VL comprising an amino acid sequence at least 80%, at least 85%, at least 90%, at least 95% or 100% identical to SEQ ID NO: 511, SEQ ID NO: 513, SEQ ID NO: 515, SEQ ID NO: 517, SEQ ID NO: 519, SEQ ID NO: 521, SEQ ID NO: 523, SEQ ID NO: 525, SEQ ID NO: 527, SEQ ID NO: 529, SEQ ID NO: 531, SEQ ID NO: 533, SEQ ID NO: 535, SEQ ID NO: 537, SEQ ID NO: 539, SEQ ID NO: 541, SEQ ID NO: 543, SEQ ID NO: 545, SEQ ID NO: 547, SEQ ID NO: 549, SEQ ID NO: 551, SEQ ID NO: 553, SEQ ID NO: 555, SEQ ID NO: 557, SEQ ID NO: 559, SEQ ID NO: 561, SEQ ID NO: 563, SEQ ID NO: 565, SEQ ID NO: 567, SEQ ID NO: 569, SEQ ID NO: 571, SEQ ID NO: 573, SEQ ID NO: 575, SEQ ID NO: 577, SEQ ID NO: 579, SEQ ID NO: 581, SEQ ID NO: 583, SEQ ID NO: 585, SEQ ID NO: 587, SEQ ID NO: 589, SEQ ID NO: 591, SEQ ID NO: 593, SEQ ID NO: 595, SEQ ID NO: 597, SEQ ID NO: 599, SEQ ID NO: 601, SEQ ID NO: 603, SEQ ID NO: 605, SEQ ID NO: 607, SEQ ID NO: 609, SEQ ID NO: 611, SEQ ID NO: 613, SEQ ID NO: 615, SEQ ID NO: 617, SEQ ID NO: 619, SEQ ID NO: 621, SEQ ID NO: 623, SEQ ID NO: 625, SEQ ID NO: 627, or SEQ ID NO: 631.

In certain embodiments, this disclosure provides a vector comprising one or more polynucleotides described herein. In some embodiments, the vector further comprises a polynucleotide comprising a nucleic acid sequence that encodes a J-chain or a functional fragment or variant thereof, such as a J-chain, functional fragment or variant thereof described herein.

In some embodiments, the vector is a viral vector, such as an adenoviral or adeno-associated viral (AAV) vector. In some embodiments, this disclosure provides a viral particle comprising a viral vector disclosed herein. In some embodiments, the viral particle is an adenoviral particle or an AAV particle.

In certain embodiments, this disclosure provides a composition comprising a first vector and a second vector, where: a) the first vector comprises a polynucleotide comprising a nucleic acid sequence that encodes the heavy chain of the multimeric binding molecule and the second vector comprises a polynucleotide comprising a nucleic acid sequence that encodes the light chain of the multimeric binding molecule, b) the first vector comprises a polynucleotide comprising a nucleic acid sequence that encodes the heavy chain of the multimeric binding molecule and a polynucleotide comprising a nucleic acid sequence that encodes the light chain of the multimeric binding molecule and the second vector comprises a polynucleotide comprising a nucleic acid sequence that encodes a J-chain or a functional fragment or variant thereof, c) the first vector comprises a polynucleotide comprising a nucleic acid sequence that encodes the heavy chain of the multimeric binding molecule and a polynucleotide comprising a nucleic acid sequence that encodes a J-chain or a functional fragment or variant thereof and the second vector comprises a polynucleotide comprising a nucleic acid sequence that encodes the light chain of the multimeric binding molecule, or d) the first vector comprises a polynucleotide comprising a nucleic acid sequence that encodes the light chain of the multimeric binding molecule and a polynucleotide comprising a nucleic acid sequence that encodes a J-chain or a functional fragment or variant thereof and the second vector comprises a polynucleotide comprising a nucleic acid sequence that encodes the heavy chain of the multimeric binding molecule. In certain embodiments, this disclosure provides a composition comprising a first vector, a second vector, and a third vector, where the first vector comprises a polynucleotide comprising a nucleic acid sequence that encodes the heavy chain of the multimeric binding molecule, the second vector comprises a polynucleotide comprising a nucleic acid sequence that encodes the light chain of the multimeric binding molecule, and the third vector comprises a polynucleotide comprising a nucleic acid sequence that encodes a J-chain or a functional fragment or variant thereof.

### Host Cells

In certain embodiments, this disclosure provides a host cell that is capable of producing the multimeric binding molecule as provided herein. In certain embodiments, the host cell comprises one or more vectors, a composition comprising multiple vectors, or polynucleotides disclosed herein. The disclosure also provides a method of producing the multimeric binding molecule as provided herein, where the method comprises culturing the provided host cell, and recovering the multimeric binding molecule.

### Methods of Use

The disclosure further provides a method of treating a disease or disorder in a subject in need of treatment, where the method includes administering to the subject a therapeutically effective amount of a multimeric binding molecule as provided herein. By “therapeutically effective dose or amount” or “effective amount” is intended an amount of a multimeric binding molecule that when administered brings about a positive therapeutic response with respect to treatment of subject. Examples of positive therapeutic responses include, without limitation, prevention of respiratory tract colonization or infection by a human coronavirus, e.g., SARS-CoV, SARS-CoV-2, or MERS-CoV, prevention of human coronavirus, e.g., SARS-CoV, SARS-CoV-2, or MERS-CoV attachment, penetration, and/or replication upon exposure to the virus, prevention of human coronavirus, e.g., SARS-CoV, SARS-CoV-2, or MERS-CoV symptoms, alleviation of human coronavirus, e.g., SARS-CoV, SARS-CoV-2, or MERS-CoV symptoms, reduction of the number of human coronavirus, e.g., SARS-CoV, SARS-CoV-2, or MERS-CoV symptoms, or reduction in the severity of human coronavirus, e.g., SARS-CoV, SARS-CoV-2, or MERS-CoV symptoms. “Symptoms” include, without limitation, one or more of fever, chills, muscle or body aches, fatigue, headache, sore throat, coughing, shortness of breath, difficulty breathing, loss of taste and/or the ability to smell, pneumonia, congestion, nausea, or diarrhea.

Effective doses of compositions for treatment of a disease or disorder vary depending upon many different factors, including means of administration, target site, physiological state of the subject, whether the subject is human or an animal, other medications administered, and whether treatment is prophylactic or therapeutic. Usually, the subject is a human, but non-human mammals including transgenic mammals can also be treated. Treatment dosages can be titrated using routine methods known to those of skill in the art to optimize safety and efficacy.

In certain embodiments, the disclosure provides a method for treating a human coronavirus disease, e.g., Corona Virus Disease 2019 (COVID-19) in a subject in need of treatment, where the method includes administering to the subject an effective amount of a multimeric binding molecule as provided herein. In certain embodiments, administration of a multimeric binding molecule as provided herein to a subject results in greater potency, e.g., greater efficacy at an equivalent dose or the ability to administer a lower dose and achieve equivalent efficacy, than administration of an equivalent amount of a monomeric binding molecule, such as an IgG, binding to the same binding partner. By “efficacy” is meant the ability of the treatment to, for example, reduce symptoms in an infected subject, reduce the severity of symptoms in an infected subject, prevent symptoms in an infected but asymptomatic subject, reduce the need for auxiliary oxygen in an infected subject or reduce time on a ventilator, reduce the need or the dosage of concomitant medications, reduce the time in intensive care, spare hospital resources, or prevent or reduce transmission from an infected subject to non-infected persons. In certain embodiments the multimeric binding molecule as provided herein can also treat the subject more safely, e.g., by preventing antibody-dependent enhancement of infection, and by effectively neutralizing “escape mutant” viruses. In certain embodiments the monomeric binding molecule includes identical binding polypeptides to the multimeric binding molecule as provided herein. By “an equivalent amount” is meant, e.g., an amount measured by molecular weight, e.g., in total milligrams, or alternatively, a molar equivalent, e.g., where equivalent numbers of molecules are administered.

In other embodiments, the disclosure provides a method for preventing a human coronavirus disease, e.g., Corona Virus Disease 2019 (COVID-19) in a subject in need thereof, e.g., a subject susceptible to human coronavirus, e.g., SARS-CoV-2 infection or a subject susceptible to more severe human coronavirus infection-associated disease or disorder, e.g., COVID-19 symptoms due to proximity to human coronavirus infection-associated disease or disorder, e.g., COVID-19 patients, e.g., healthcare providers and/or family members, or due to secondary conditions such as advanced age, diabetes, heart disease, or obesity, where the method includes administering to the subject an effective amount of a multimeric binding molecule as provided herein. In certain embodiments, administration of a multimeric binding molecule as provided herein to a subject results in greater potency, e.g., as noted above, than administration of an equivalent amount of a monomeric binding polypeptide, such as an IgG, binding to the same binding partner. In certain embodiments the monomeric binding molecule includes identical antigen binding domains to the multimeric binding molecule as provided herein. By “an equivalent amount” is meant, e.g., an amount measured by molecular weight, e.g., in total milligrams, or alternative, a molar equivalent, e.g., where equivalent numbers of molecules are administered.

The subject can be any animal, e.g., a mammal, in need of treatment or prevention, in certain embodiments, the subject is a human subject.

In its simplest form, a preparation to be administered to a subject is multimeric binding molecule as provided herein administered in a conventional dosage form, which can be combined with a pharmaceutical excipient, carrier or diluent as described elsewhere herein.

A multimeric binding molecule of the disclosure can be administered by any suitable method, e.g., parenterally, intraventricularly, orally, by inhalation spray, topically, rectally, intranasally, buccally, vaginally, via an implanted reservoir, or a combination thereof. The term “parenteral” as used herein includes subcutaneous, intravenous, intramuscular, intra-articular, intra-synovial, intrasternal, intrathecal, intrahepatic, intralesional and intracranial injection or infusion techniques.

In certain embodiments the multimeric binding molecule is delivered intranasally, e.g., in an atomized form produced by a suitable spray delivery device, e.g., a MAD NASAL™ Intranasal Mucosal Atomization Device, produced by Teleflex. In certain embodiments, the multimeric binding molecule is delivered orally, e.g., in an atomized form produced by a suitable spray delivery device, e.g., a MAD NASAL™ Intranasal Mucosal Atomization Device, produced by Teleflex. In certain embodiments, the multimeric binding molecule is delivered intranasally and orally, e.g., in an atomized form produced by a suitable spray delivery device, e.g., a MAD NASAL™ Intranasal Mucosal Atomization Device, produced by Teleflex.

In certain embodiments the multimeric binding molecule is delivered via inhalation, e.g., in a nebulized form.

In certain embodiments the multimeric binding molecule is delivered intravenously. In certain embodiments the multimeric binding molecule is delivered intranasally, e.g., in an atomized form produced by a suitable spray delivery device, e.g., a MAD NASAL™ Intranasal Mucosal Atomization Device, produced by Teleflex and is delivered intravenously. In certain embodiments, the multimeric binding molecule is delivered orally, e.g., in an atomized form produced by a suitable spray delivery device, e.g., a MAD NASAL™ Intranasal Mucosal Atomization Device, produced by Teleflex and is delivered intravenously. In certain embodiments, the multimeric binding molecule is delivered intranasally and orally, e.g., in an atomized form produced by a suitable spray delivery device, e.g., a MAD NASAL™ Intranasal Mucosal Atomization Device, produced by Teleflex and is delivered intravenously.

### Pharmaceutical Compositions and Administration Methods

The disclosure further provides a composition, e.g., a pharmaceutical composition, comprising a multimeric binding molecule, or two or more multimeric binding molecules, as provided herein. In certain embodiments the composition includes a cocktail of two or more different multimeric binding molecules as described here, that bind to different epitopes on a single human coronavirus, e.g., SARS-CoV-2 or MERS-CoV. In certain aspects the different epitopes can be on the same coronavirus protein, e.g., structural protein, for example the spike (S) protein. A composition as provided herein can further include a pharmaceutically acceptable carrier and/or excipient and can be formulated so as to be suitable for a desired mode of administration.

Methods of preparing and administering a multimeric binding molecule as provided herein to a subject in need thereof can be determined by a skilled person in view of this disclosure. The route of administration of can be, for example, oral, parenteral, intranasally, by inhalation, by aerosol, or topical. The term parenteral as used herein includes, e.g., intravenous, intraarterial, intraperitoneal, intramuscular, subcutaneous, rectal, or vaginal administration. While these forms of administration are contemplated as suitable forms, another example of a form for administration would be a solution for injection, in particular for intravenous, or intraarterial injection or drip. A suitable pharmaceutical composition can include a buffer (e.g., acetate, phosphate, or citrate buffer), a surfactant (e.g., polysorbate), optionally a stabilizer agent (e.g., human albumin), etc.

As discussed herein, a multimeric binding molecule as provided herein can be administered in a pharmaceutically effective amount for the treatment of a subject in need thereof. In this regard, it will be appreciated that the disclosed multimeric binding molecule can be formulated so as to facilitate administration and promote stability of the active agent. Pharmaceutical compositions accordingly can include a pharmaceutically acceptable, non-toxic, sterile carrier such as physiological saline, non-toxic buffers, preservatives, and the like. A pharmaceutically effective amount of a multimeric binding molecule as provided herein means an amount sufficient to achieve effective binding to a target and to achieve a therapeutic benefit. Suitable formulations are described in Remington's Pharmaceutical Sciences (Mack Publishing Co.) 16th ed. (1980).

Certain pharmaceutical compositions provided herein can be orally administered in an acceptable dosage form including, e.g., capsules, tablets, aqueous suspensions, or solutions.

Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents. In some embodiments, the pharmaceutical composition is administered by nasal aerosol. In some embodiments, the pharmaceutical composition is for administration by nasal aerosol. In some embodiments, the pharmaceutical composition, such as a pharmaceutical composition for administration by nasal aerosol, comprises a pH adjuster, such as HCl; a buffer; an emulsifier, such as polysorbate or carbomer; sugar or mono- or polyol, such as a monosaccharide (e.g., glucose, dextrose, or fructose), disaccharide (e.g., sucrose, lactose, or maltose), ribose, glycerin, sorbitol, xylitol, inositol, propylene glycol, galactose, mannose, xylose, rhamnose, glutaraldehyde, ethanol, mannitol, polyethylene glycol, glycerol, chitosal, phenylethyl alcohol; a preservative; cellulose, such as microcrystalline cellulose or carboxymethylcellulose; or mixtures thereof.

In some embodiments, the pharmaceutical composition is administered by inhalation. In some embodiments, the pharmaceutical composition is for administration by inhalation. In some embodiments, the pharmaceutical composition, such as a pharmaceutical composition for administration by inhalation, is a dry powder, such as for a dry powder inhaler, or a liquid, such as for a nebulizer, such as an airjet-compressor nebulizer or a mesh-based nebulizer. In some embodiments, the pharmaceutical composition, such as a pharmaceutical composition for administration by inhalation, comprises sugar or mono- or polyol, such as lactose, trelose, mannitol, sorbitol; buffer, such as histidine, proline, or arginine buffer; saline; polysorbate; or mixtures thereof.

The amount of a multimeric binding molecule that can be combined with carrier materials to produce a single dosage form will vary depending, e.g., upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, a multimeric binding molecule as provided herein can be administered to a subject in need of therapy in an amount sufficient to produce a therapeutic effect or a prophylactic effect. A multimeric binding molecule as provided herein can be administered to the subject in a conventional dosage form prepared by combining the multimeric binding molecule of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

This disclosure also provides for the use of a multimeric binding molecule as provided herein in the manufacture of a medicament for treating, preventing, or managing a human coronavirus infection, e.g., COVID-19.

In some embodiments, the compositions and methods provided herein can be used for the treatment of infections that have not been effectively treated using existing or established treatments.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All of the references cited above, as well as all references cited herein, are incorporated herein by reference in their entireties.

The following examples are offered by way of illustration and not by way of limitation.

### EXEMPLARY EMBODIMENTS

Among the provided embodiments are:

Embodiment 1. A multimeric binding molecule comprising two to six bivalent binding units or variants or fragments thereof, wherein each binding unit comprises two IgM or IgA heavy chain constant regions or multimerizing fragments or variants thereof, each associated with a binding domain, wherein three to twelve of the binding domains are identical and specifically bind to a human coronavirus, and wherein the binding molecule is more potent than a bivalent reference IgG antibody comprising two of the binding domains that specifically bind to the human coronavirus.

Embodiment 2. The multimeric binding molecule of embodiment 1, wherein the human coronavirus is SARS-CoV, MERS-CoV, SARS-CoV-2, variants thereof, derivatives thereof, or any combination thereof.

Embodiment 3. The multimeric binding molecule of embodiment 1 or embodiment 2, wherein the binding molecule can neutralize infectivity of the human coronavirus at a greater potency than the bivalent reference IgG antibody comprising two of the binding domains that specifically bind to the human coronavirus.

Embodiment 4. The multimeric binding molecule of embodiment 3, wherein the binding molecule can neutralize infectivity of the human coronavirus at a lower 50% effective concentration (EC50) than the bivalent reference IgG antibody.

Embodiment 5. The multimeric binding molecule of embodiment 4, wherein the EC50 is at least two-fold, at least five-fold, at least ten-fold, at least fifty-fold, at least 100-fold, at least 500-fold, or at least 1000-fold lower than the EC50 of the bivalent reference IgG antibody.

Embodiment 6. The multimeric binding molecule of embodiment 1 or embodiment 2, wherein the binding molecule can inhibit binding of the human coronavirus to its receptor at a lower 50% inhibitory concentration (IC50) than the bivalent reference IgG antibody.

Embodiment 7. The multimeric binding molecule of embodiment 6, wherein the human coronavirus is SARS-CoV or SARS-CoV-2 and the receptor is human angiotensin-converting enzyme 2 (ACE2), or wherein the human coronavirus is MERS-CoV and the receptor is human dipeptidyl-peptidase 4 (DPP4).

Embodiment 8. The multimeric binding molecule of any one of embodiments 1 to 7, wherein the three to twelve binding domains that specifically bind to a human coronavirus bind a human coronavirus structural protein or fragment thereof.

Embodiment 9. The multimeric binding molecule of embodiment 8, wherein the human coronavirus structural protein comprises a nucleocapsid (N) protein, a membrane (M) protein, an envelope (E) protein, a spike (S) protein, any fragment thereof, any subunit thereof, or any combination thereof.

Embodiment 10. The multimeric binding molecule of embodiment 9, wherein the human coronavirus structural protein comprises the S protein, a fragment thereof, or a subunit thereof.

Embodiment 11. The multimeric binding molecule of embodiment 10, wherein the three to twelve binding domains that specifically bind to the human coronavirus S protein bind the S protein subunit 1 (S1), the S protein receptor binding domain (RBD), the S protein subunit 2 (S2), the S protein furin cleavage site, or any combination thereof.

Embodiment 12. The multimeric binding molecule of embodiment 11, wherein the three to twelve binding domains that specifically bind to the human coronavirus S protein bind the S protein RBD.

Embodiment 13. The multimeric binding molecule of any one of embodiments 1 to 12, wherein the three to twelve identical binding domains are immunoglobulin antigen binding domains comprising a heavy chain variable region (VH) and a light chain variable region (VL).

Embodiment 14. The multimeric binding molecule of embodiment 13, wherein each binding unit comprises two heavy chains each comprising a VH and two light chains each comprising a VL.

Embodiment 15. The multimeric binding molecule of embodiment 13 or embodiment 14, wherein the immunoglobulin antigen-binding domains are human or humanized antigen binding domains.

Embodiment 16. The multimeric binding molecule of any one of embodiments 1 to 12, wherein the three to twelve identical binding domains are single-domain variable regions (VHH), and wherein each binding unit comprises two heavy chains each comprising the VHH.

Embodiment 17. The multimeric binding molecule of any one of embodiments 1 to 16, wherein the human coronavirus is SARS-CoV-2.

Embodiment 18. The multimeric binding molecule of embodiment 17, wherein the three to twelve identical binding domains each specifically bind to SARS-CoV-2 and comprise a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, and wherein the HCDR1, HCDR2, HCDR3, LCDR1, LCDR2, and LCDR3 comprise the CDRs of an antibody comprising the VH and VL of SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 104 and SEQ ID NO: 105, SEQ ID NO: 106 and SEQ ID NO: 107, SEQ ID NO: 108 and SEQ ID NO: 109, SEQ ID NO: 110 and SEQ ID NO: 111, SEQ ID NO: 112 and SEQ ID NO: 113, SEQ ID NO: 114 and SEQ ID NO: 115, SEQ ID NO: 116 and SEQ ID NO: 117, SEQ ID NO: 118 and SEQ ID NO: 119, SEQ ID NO: 120 and SEQ ID NO: 121, SEQ ID NO: 122 and SEQ ID NO: 123, SEQ ID NO: 124 and SEQ ID NO: 125, SEQ ID NO: 126 and SEQ ID NO: 127, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 150 and SEQ ID NO: 151, SEQ ID NO: 152 and SEQ ID NO: 153, SEQ ID NO: 154 and SEQ ID NO: 155, SEQ ID NO: 156 and SEQ ID NO: 157, SEQ ID NO: 158 and SEQ ID NO: 159, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 178 and SEQ ID NO: 179, SEQ ID NO: 180 and SEQ ID NO: 181, SEQ ID NO: 182 and SEQ ID NO: 183, SEQ ID NO: 184 and SEQ ID NO: 185, SEQ ID NO: 186 and SEQ ID NO: 187, SEQ ID NO: 188 and SEQ ID NO: 189, SEQ ID NO: 190 and SEQ ID NO: 191, SEQ ID NO: 192 and SEQ ID NO: 193, SEQ ID NO: 194 and SEQ ID NO: 195, SEQ ID NO: 196 and SEQ ID NO: 197, SEQ ID NO: 198 and SEQ ID NO: 199, SEQ ID NO: 200 and SEQ ID NO: 201, SEQ ID NO: 202 and SEQ ID NO: 203, SEQ ID NO: 204 and SEQ ID NO: 205, SEQ ID NO: 206 and SEQ ID NO: 207, SEQ ID NO: 208 and SEQ ID NO: 209, SEQ ID NO: 210 and SEQ ID NO: 211, SEQ ID NO: 212 and SEQ ID NO: 213, SEQ ID NO: 214 and SEQ ID NO: 215, SEQ ID NO: 216 and SEQ ID NO: 217, SEQ ID NO: 218 and SEQ ID NO: 219, SEQ ID NO: 220 and SEQ ID NO: 221, SEQ ID NO: 222 and SEQ ID NO: 223, SEQ ID NO: 224 and SEQ ID NO: 225, SEQ ID NO: 226 and SEQ ID NO: 227, SEQ ID NO: 228 and SEQ ID NO: 229, SEQ ID NO: 230 and SEQ ID NO: 231, SEQ ID NO: 232 and SEQ ID NO: 233, SEQ ID NO: 234 and SEQ ID NO: 235, SEQ ID NO: 236 and SEQ ID NO: 237, SEQ ID NO: 238 and SEQ ID NO: 239, SEQ ID NO: 240 and SEQ ID NO: 241, SEQ ID NO: 242 and SEQ ID NO: 243, SEQ ID NO: 244 and SEQ ID NO: 245, SEQ ID NO: 246 and SEQ ID NO: 247, SEQ ID NO: 248 and SEQ ID NO: 249, SEQ ID NO: 250 and SEQ ID NO: 251, SEQ ID NO: 252 and SEQ ID NO: 253, SEQ ID NO: 254 and SEQ ID NO: 255, SEQ ID NO: 256 and SEQ ID NO: 257, SEQ ID NO: 258 and SEQ ID NO: 259, SEQ ID NO: 260 and SEQ ID NO: 261, SEQ ID NO: 264 and SEQ ID NO: 265, SEQ ID NO: 266 and SEQ ID NO: 267, SEQ ID NO: 268 and SEQ ID NO: 269, SEQ ID NO: 270 and SEQ ID NO: 271, SEQ ID NO: 272 and SEQ ID NO: 273, SEQ ID NO: 274 and SEQ ID NO: 275, SEQ ID NO: 276 and SEQ ID NO: 277, SEQ ID NO: 278 and SEQ ID NO: 279, SEQ ID NO: 280 and SEQ ID NO: 281, SEQ ID NO: 282 and SEQ ID NO: 283, SEQ ID NO: 292 and SEQ ID NO: 293, SEQ ID NO: 294 and SEQ ID NO: 295, SEQ ID NO: 296 and SEQ ID NO: 297, SEQ ID NO: 298 and SEQ ID NO: 299, SEQ ID NO: 300 and SEQ ID NO: 301, SEQ ID NO: 302 and SEQ ID NO: 303, SEQ ID NO: 304 and SEQ ID NO: 305, SEQ ID NO: 306 and SEQ ID NO: 307, SEQ ID NO: 308 and SEQ ID NO: 309, SEQ ID NO: 310 and SEQ ID NO: 311, SEQ ID NO: 312 and SEQ ID NO: 313, SEQ ID NO: 314 and SEQ ID NO: 315, SEQ ID NO: 316 and SEQ ID NO: 317, SEQ ID NO: 318 and SEQ ID NO: 319, SEQ ID NO: 320 and SEQ ID NO: 321, SEQ ID NO: 322 and SEQ ID NO: 323, SEQ ID NO: 324 and SEQ ID NO: 325, SEQ ID NO: 326 and SEQ ID NO: 327, SEQ ID NO: 328 and SEQ ID NO: 329, SEQ ID NO: 330 and SEQ ID NO: 331, SEQ ID NO: 332 and SEQ ID NO: 333, SEQ ID NO: 336 and SEQ ID NO: 337, SEQ ID NO: 338 and SEQ ID NO: 339, SEQ ID NO: 340 and SEQ ID NO: 341, SEQ ID NO: 342 and SEQ ID NO: 343, SEQ ID NO: 344 and SEQ ID NO: 345, SEQ ID NO: 348 and SEQ ID NO: 349, SEQ ID NO: 350 and SEQ ID NO: 351, SEQ ID NO: 352 and SEQ ID NO: 353, SEQ ID NO: 354 and SEQ ID NO: 355, SEQ ID NO: 356 and SEQ ID NO: 357, SEQ ID NO: 358 and SEQ ID NO: 359, SEQ ID NO: 362 and SEQ ID NO: 363, SEQ ID NO: 364 and SEQ ID NO: 365, SEQ ID NO: 366 and SEQ ID NO: 367, SEQ ID NO: 368 and SEQ ID NO: 369, SEQ ID NO: 370 and SEQ ID NO: 371, SEQ ID NO: 372 and SEQ ID NO: 373, SEQ ID NO: 374 and SEQ ID NO: 375, SEQ ID NO: 376 and SEQ ID NO: 377, SEQ ID NO: 378 and SEQ ID NO: 379, SEQ ID NO: 380 and SEQ ID NO: 381, SEQ ID NO: 382 and SEQ ID NO: 383, SEQ ID NO: 384 and SEQ ID NO: 385, SEQ ID NO: 386 and SEQ ID NO: 387, SEQ ID NO: 388 and SEQ ID NO: 389, SEQ ID NO: 390 and SEQ ID NO: 391, SEQ ID NO: 392 and SEQ ID NO: 393, SEQ ID NO: 394 and SEQ ID NO: 395, SEQ ID NO: 396 and SEQ ID NO: 397, SEQ ID NO: 398 and SEQ ID NO: 399, SEQ ID NO: 400 and SEQ ID NO: 401, SEQ ID NO: 402 and SEQ ID NO: 403, SEQ ID NO: 404 and SEQ ID NO: 405, SEQ ID NO: 406 and SEQ ID NO: 407, SEQ ID NO: 408 and SEQ ID NO: 409, SEQ ID NO: 410 and SEQ ID NO: 411, SEQ ID NO: 412 and SEQ ID NO: 413, SEQ ID NO: 414 and SEQ ID NO: 415, SEQ ID NO: 416 and SEQ ID NO: 417, SEQ ID NO: 418 and SEQ ID NO: 419, SEQ ID NO: 420 and SEQ ID NO: 421, SEQ ID NO: 422 and SEQ ID NO: 423, SEQ ID NO: 424 and SEQ ID NO: 425, SEQ ID NO: 426 and SEQ ID NO: 427, SEQ ID NO: 428 and SEQ ID NO: 429, SEQ ID NO: 430 and SEQ ID NO: 431, SEQ ID NO: 432 and SEQ ID NO: 433, SEQ ID NO: 434 and SEQ ID NO: 435, SEQ ID NO: 436 and SEQ ID NO: 437, SEQ ID NO: 438 and SEQ ID NO: 439, SEQ ID NO: 440 and SEQ ID NO: 441, SEQ ID NO: 442 and SEQ ID NO: 443, SEQ ID NO: 444 and SEQ ID NO: 445, SEQ ID NO: 446 and SEQ ID NO: 447, SEQ ID NO: 448 and SEQ ID NO: 449, SEQ ID NO: 450 and SEQ ID NO: 451, SEQ ID NO: 452 and SEQ ID NO: 453, SEQ ID NO: 454 and SEQ ID NO: 455, SEQ ID NO: 456 and SEQ ID NO: 457, SEQ ID NO: 458 and SEQ ID NO: 459, SEQ ID NO: 460 and SEQ ID NO: 461, SEQ ID NO: 462 and SEQ ID NO: 463, SEQ ID NO: 464 and SEQ ID NO: 465, SEQ ID NO: 466 and SEQ ID NO: 467, SEQ ID NO: 468 and SEQ ID NO: 469, SEQ ID NO: 470 and SEQ ID NO: 471, SEQ ID NO: 472 and SEQ ID NO: 473, SEQ ID NO: 474 and SEQ ID NO: 475, SEQ ID NO: 476 and SEQ ID NO: 477, SEQ ID NO: 478 and SEQ ID NO: 479, SEQ ID NO: 480 and SEQ ID NO: 481, SEQ ID NO: 482 and SEQ ID NO: 483, SEQ ID NO: 484 and SEQ ID NO: 485, SEQ ID NO: 486 and SEQ ID NO: 487, SEQ ID NO: 488 and SEQ ID NO: 489, SEQ ID NO: 490 and SEQ ID NO: 491, SEQ ID NO: 492 and SEQ ID NO: 493, SEQ ID NO: 494 and SEQ ID NO: 495, SEQ ID NO: 496 and SEQ ID NO: 497, SEQ ID NO: 498 and SEQ ID NO: 499, SEQ ID NO: 500 and SEQ ID NO: 501, SEQ ID NO: 502 and SEQ ID NO: 503, SEQ ID NO: 504 and SEQ ID NO: 505, SEQ ID NO: 506 and SEQ ID NO: 507, SEQ ID NO: 508 and SEQ ID NO: 509, SEQ ID NO: 628 and SEQ ID NO: 629, SEQ ID NO: 630 and SEQ ID NO: 631, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, SEQ ID NO: 646 and SEQ ID NO: 647, SEQ ID NO: 648 and SEQ ID NO: 649, SEQ ID NO: 650 and SEQ ID NO: 651, or SEQ ID NO: 652 and SEQ ID NO: 653, respectively with zero, one, or two single amino acid substitutions in one or more of the HCDRs or LCDRs.

Embodiment 19. The multimeric binding molecule of embodiment 18, wherein the VH and VL comprise amino acid sequences at least 80%, at least 85%, at least 90%, at least 95% or 100% identical to the recited VH and VL amino acid sequences, respectively.

Embodiment 20. The multimeric binding molecule of embodiment 18, wherein the VH and VL comprise six immunoglobulin complementarity determining regions HCDR1, HCDR2, HCDR3, LCDR1, LCDR2, and LCDR3, and wherein the HCDR1, HCDR2, HCDR3, LCDR1, LCDR2, and LCDR3 comprise the CDRs of an antibody comprising the VH and VL of SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 106 and SEQ ID NO: 107, SEQ ID NO: 110 and SEQ ID NO: 111, SEQ ID NO: 118 and SEQ ID NO: 119, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 142 and SEQ ID NO: 143, SEQ ID NO: 146 and SEQ ID NO: 147, SEQ ID NO: 150 and SEQ ID NO: 151, SEQ ID NO: 156 and SEQ ID NO: 157, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 180 and SEQ ID NO: 181, SEQ ID NO: 182 and SEQ ID NO: 183, SEQ ID NO: 186 and SEQ ID NO: 187, SEQ ID NO: 188 and SEQ ID NO: 189, SEQ ID NO: 194 and SEQ ID NO: 195, SEQ ID NO: 200 and SEQ ID NO: 201, SEQ ID NO: 206 and SEQ ID NO: 207, SEQ ID NO: 208 and SEQ ID NO: 209, SEQ ID NO: 218 and SEQ ID NO: 219, SEQ ID NO: 220 and SEQ ID NO: 221, SEQ ID NO: 222 and SEQ ID NO: 223, SEQ ID NO: 224 and SEQ ID NO: 225, SEQ ID NO: 228 and SEQ ID NO: 229, SEQ ID NO: 230 and SEQ ID NO: 231, SEQ ID NO: 240 and SEQ ID NO: 241, SEQ ID NO: 244 and SEQ ID NO: 245, SEQ ID NO: 246 and SEQ ID NO: 247, SEQ ID NO: 256 and SEQ ID NO: 257, SEQ ID NO: 258 and SEQ ID NO: 259, SEQ ID NO: 260 and SEQ ID NO: 261, SEQ ID NO: 264 and SEQ ID NO: 265, SEQ ID NO: 266 and SEQ ID NO: 267, SEQ ID NO: 268 and SEQ ID NO: 269, SEQ ID NO: 270 and SEQ ID NO: 271, SEQ ID NO: 272 and SEQ ID NO: 273, SEQ ID NO: 274 and SEQ ID NO: 275, SEQ ID NO: 276 and SEQ ID NO: 277, SEQ ID NO: 278 and SEQ ID NO: 279, SEQ ID NO: 280 and SEQ ID NO: 281, SEQ ID NO: 282 and SEQ ID NO: 283, SEQ ID NO: 292 and SEQ ID NO: 293, SEQ ID NO: 294 and SEQ ID NO: 295, SEQ ID NO: 296 and SEQ ID NO: 297, SEQ ID NO: 298 and SEQ ID NO: 299, SEQ ID NO: 300 and SEQ ID NO: 301, SEQ ID NO: 302 and SEQ ID NO: 303, SEQ ID NO: 304 and SEQ ID NO: 305, SEQ ID NO: 306 and SEQ ID NO: 307, SEQ ID NO: 308 and SEQ ID NO: 309, SEQ ID NO: 310 and SEQ ID NO: 311, SEQ ID NO: 312 and SEQ ID NO: 313, SEQ ID NO: 314 and SEQ ID NO: 315, SEQ ID NO: 316 and SEQ ID NO: 317, SEQ ID NO: 318 and SEQ ID NO: 319, SEQ ID NO: 320 and SEQ ID NO: 321, SEQ ID NO: 322 and SEQ ID NO: 323, SEQ ID NO: 324 and SEQ ID NO: 325, SEQ ID NO: 326 and SEQ ID NO: 327, SEQ ID NO: 328 and SEQ ID NO: 329, SEQ ID NO: 330 and SEQ ID NO: 331, SEQ ID NO: 332 and SEQ ID NO: 333, SEQ ID NO: 336 and SEQ ID NO: 337, SEQ ID NO: 338 and SEQ ID NO: 339, SEQ ID NO: 340 and SEQ ID NO: 341, SEQ ID NO: 342 and SEQ ID NO: 343, SEQ ID NO: 344 and SEQ ID NO: 345, SEQ ID NO: 348 and SEQ ID NO: 349, SEQ ID NO: 350 and SEQ ID NO: 351, SEQ ID NO: 352 and SEQ ID NO: 353, SEQ ID NO: 354 and SEQ ID NO: 355, SEQ ID NO: 356 and SEQ ID NO: 357, SEQ ID NO: 358 and SEQ ID NO: 359, SEQ ID NO: 360 and SEQ ID NO: 361, SEQ ID NO: 362 and SEQ ID NO: 363, SEQ ID NO: 364 and SEQ ID NO: 365, SEQ ID NO: 366 and SEQ ID NO: 367, SEQ ID NO: 368 and SEQ ID NO: 369, SEQ ID NO: 370 and SEQ ID NO: 371, SEQ ID NO: 372 and SEQ ID NO: 373, SEQ ID NO: 374 and SEQ ID NO: 375, SEQ ID NO: 376 and SEQ ID NO: 377, SEQ ID NO: 378 and SEQ ID NO: 379, SEQ ID NO: 380 and SEQ ID NO: 381, SEQ ID NO: 382 and SEQ ID NO: 383, SEQ ID NO: 384 and SEQ ID NO: 385, SEQ ID NO: 386 and SEQ ID NO: 387, SEQ ID NO: 388 and SEQ ID NO: 389, SEQ ID NO: 390 and SEQ ID NO: 391, SEQ ID NO: 392 and SEQ ID NO: 393, SEQ ID NO: 394 and SEQ ID NO: 395, SEQ ID NO: 396 and SEQ ID NO: 397, SEQ ID NO: 398 and SEQ ID NO: 399, SEQ ID NO: 400 and SEQ ID NO: 401, SEQ ID NO: 402 and SEQ ID NO: 403, SEQ ID NO: 404 and SEQ ID NO: 405, SEQ ID NO: 406 and SEQ ID NO: 407, SEQ ID NO: 408 and SEQ ID NO: 409, SEQ ID NO: 410 and SEQ ID NO: 411, SEQ ID NO: 412 and SEQ ID NO: 413, SEQ ID NO: 414 and SEQ ID NO: 415, SEQ ID NO: 416 and SEQ ID NO: 417, SEQ ID NO: 418 and SEQ ID NO: 419, SEQ ID NO: 420 and SEQ ID NO: 421, SEQ ID NO: 422 and SEQ ID NO: 423, SEQ ID NO: 424 and SEQ ID NO: 425, SEQ ID NO: 426 and SEQ ID NO: 427, SEQ ID NO: 428 and SEQ ID NO: 429, SEQ ID NO: 430 and SEQ ID NO: 431, SEQ ID NO: 432 and SEQ ID NO: 433, SEQ ID NO: 434 and SEQ ID NO: 435, SEQ ID NO: 436 and SEQ ID NO: 437, SEQ ID NO: 438 and SEQ ID NO: 439, SEQ ID NO: 440 and SEQ ID NO: 441, SEQ ID NO: 442 and SEQ ID NO: 443, SEQ ID NO: 444 and SEQ ID NO: 445, SEQ ID NO: 446 and SEQ ID NO: 447, SEQ ID NO: 448 and SEQ ID NO: 449, SEQ ID NO: 450 and SEQ ID NO: 451, SEQ ID NO: 452 and SEQ ID NO: 453, SEQ ID NO: 454 and SEQ ID NO: 455, SEQ ID NO: 456 and SEQ ID NO: 457, SEQ ID NO: 458 and SEQ ID NO: 459, SEQ ID NO: 460 and SEQ ID NO: 461, SEQ ID NO: 462 and SEQ ID NO: 463, SEQ ID NO: 464 and SEQ ID NO: 465, SEQ ID NO: 466 and SEQ ID NO: 467, SEQ ID NO: 468 and SEQ ID NO: 469, SEQ ID NO: 470 and SEQ ID NO: 471, SEQ ID NO: 472 and SEQ ID NO: 473, SEQ ID NO: 474 and SEQ ID NO: 475, SEQ ID NO: 476 and SEQ ID NO: 477, SEQ ID NO: 478 and SEQ ID NO: 479, SEQ ID NO: 480 and SEQ ID NO: 481, SEQ ID NO: 482 and SEQ ID NO: 483, SEQ ID NO: 484 and SEQ ID NO: 485, SEQ ID NO: 486 and SEQ ID NO: 487, SEQ ID NO: 488 and SEQ ID NO: 489, SEQ ID NO: 490 and SEQ ID NO: 491, SEQ ID NO: 492 and SEQ ID NO: 493, SEQ ID NO: 494 and SEQ ID NO: 495, SEQ ID NO: 496 and SEQ ID NO: 497, SEQ ID NO: 498 and SEQ ID NO: 499, SEQ ID NO: 500 and SEQ ID NO: 501, SEQ ID NO: 502 and SEQ ID NO: 503, SEQ ID NO: 504 and SEQ ID NO: 505, SEQ ID NO: 506 and SEQ ID NO: 507, SEQ ID NO: 508 and SEQ ID NO: 509, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, SEQ ID NO: 646 and SEQ ID NO: 647, SEQ ID NO: 648 and SEQ ID NO: 649, SEQ ID NO: 650 and SEQ ID NO: 651, or SEQ ID NO: 652 and SEQ ID NO: 653, respectively, with zero, one, or two single amino acid substitutions in one or more HCDRs or LCDRs.

Embodiment 21. The multimeric binding molecule of embodiment 20, wherein the HCDR1, HCDR2, HCDR3, LCDR1, LCDR2, and LCDR3 comprise the CDRs of an antibody comprising the VH and VL of SEQ ID NO: 88 and SEQ ID NO: 89.

Embodiment 22. The multimeric binding molecule of embodiment 20, wherein the HCDR1, HCDR2, HCDR3, LCDR1, LCDR2, and LCDR3 comprise the CDRs of an antibody comprising the VH and VL of SEQ ID NO: 260 and SEQ ID NO: 261.

Embodiment 23. The multimeric binding molecule of embodiment 20, wherein the HCDR1, HCDR2, HCDR3, LCDR1, LCDR2, and LCDR3 comprise the CDRs of an antibody comprising the VH and VL of SEQ ID NO: 264 and SEQ ID NO: 265.

Embodiment 24. The multimeric binding molecule of embodiment 20, wherein the HCDR1, HCDR2, HCDR3, LCDR1, LCDR2, and LCDR3 comprise the CDRs of an antibody comprising the VH and VL of SEQ ID NO: 266 and SEQ ID NO: 267.

Embodiment 25. The multimeric binding molecule of embodiment 20, wherein the HCDR1, HCDR2, HCDR3, LCDR1, LCDR2, and LCDR3 comprise the CDRs of an antibody comprising the VH and VL of SEQ ID NO: 274 and SEQ ID NO: 275, SEQ ID NO: 278, and SEQ ID NO: 279, SEQ ID NO: 280 and SEQ ID NO: 281, or SEQ ID NO: 282 and SEQ ID NO: 283.

Embodiment 26. The multimeric binding molecule of embodiment 20, wherein the HCDR1, HCDR2, HCDR3, LCDR1, LCDR2, and LCDR3 comprise the CDRs of an antibody comprising the VH and VL of SEQ ID NO: 292 and SEQ ID NO: 293.

Embodiment 27. The multimeric binding molecule of any one of embodiments 20 to 26, wherein the VH and VL comprise amino acid sequences at least 80%, at least 85%, at least 90%, at least 95% or 100% identical to the recited VH and VL amino acid sequences, respectively.

Embodiment 28. The multimeric binding molecule of any one of embodiments 20 to 27, wherein the bivalent reference IgG antibody comprising two of the binding domains can neutralize SARS-CoV-2.

Embodiment 29. The multimeric binding molecule of embodiment 20, wherein the VH and VL comprise six immunoglobulin complementarity determining regions HCDR1, HCDR2, HCDR3, LCDR1, LCDR2, and LCDR3, and wherein the HCDR1, HCDR2, HCDR3, LCDR1, LCDR2, and LCDR3 comprise the CDRs of an antibody comprising the VH and VL of SEQ ID NO: 118 and SEQ ID NO: 119, SEQ ID NO: 162 and SEQ ID NO: 163, SEQ ID NO: 222 and SEQ ID NO: 223, SEQ ID NO: 260 and SEQ ID NO: 261, SEQ ID NO: 296 and SEQ ID NO: 297, SEQ ID NO: 384 and SEQ ID NO: 385, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, or SEQ ID NO: 646 and SEQ ID NO: 647, respectively.

Embodiment 30. The multimeric binding molecule of embodiment 29, wherein the VH and VL comprise amino acid sequences at least 80%, at least 85%, at least 90%, at least 95% or 100% identical to the recited VH and VL amino acid sequences, respectively.

Embodiment 31. The multimeric binding molecule of embodiment 29 or embodiment 30, wherein the bivalent reference IgG antibody comprising two of the binding domains can neutralize SARS-CoV-2 and specifically binds to SARS-CoV.

Embodiment 32. The multimeric binding molecule of embodiment 29, wherein the VH and VL comprise six immunoglobulin complementarity determining regions HCDR1, HCDR2, HCDR3, LCDR1, LCDR2, and LCDR3, and wherein the HCDR1, HCDR2, HCDR3, LCDR1, LCDR2, and LCDR3 comprise the CDRs of an antibody comprising the VH and VL of SEQ ID NO: 260 and SEQ ID NO: 261, SEQ ID NO: 296 and SEQ ID NO: 297, SEQ ID NO: 384 and SEQ ID NO: 385, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, SEQ ID NO: 644 and SEQ ID NO: 645, or SEQ ID NO: 646 and SEQ ID NO: 647, respectively.

Embodiment 33. The multimeric binding protein of embodiment 32, wherein the VH and VL comprise the CDRs of an antibody comprising the VH and VL of SEQ ID NO:384 and SEQ ID NO: 385.

Embodiment 34. The multimeric binding protein of embodiment 32, wherein the VH and VL comprise the CDRs of an antibody comprising the VH and VL of SEQ ID NO: 646 and SEQ ID NO: 647.

Embodiment 35. The multimeric binding molecule of any one of embodiments 32 to 34, wherein the VH and VL comprise amino acid sequences at least 80%, at least 85%, at least 90%, at least 95% or 100% identical to the recited VH and VL amino acid sequences, respectively.

Embodiment 36. The multimeric binding molecule of any one of embodiments 32 to 35, wherein the bivalent reference IgG antibody comprising two of the binding domains can neutralize SARS-CoV and SARS-CoV-2.

Embodiment 37. The multimeric binding molecule of embodiment 17, wherein the three to twelve identical binding domains each specifically bind to SARS-CoV-2 and comprise a single domain variable region (VHH), wherein the VHH comprises three immunoglobulin complementarity determining regions HCDR1, HCDR2, and HCDR3, wherein the HCDR1, HCDR2, and HCDR3 comprise the CDRs of an antibody comprising the VHH of SEQ ID NO: SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, or SEQ ID NO:83, with zero, one, or two single amino acid substitutions in one or more of the HCDRs.

Embodiment 38. The multimeric binding molecule of embodiment 37, wherein the VHH comprises an amino acid sequence at least 80%, at least 85%, at least 90%, at least 95% or 100% identical to the recited VHH amino acid sequence.

Embodiment 39. The multimeric binding molecule of any one of embodiments 1 to 11, wherein the three to twelve identical binding domains of the multimeric binding molecule that specifically bind to a human coronavirus comprise an extracellular SARS-CoV-2 RBD-binding fragment of angiotensin-converting enzyme 2 (ACE2).

Embodiment 40. The multimeric binding molecule of any one of embodiments 1 to 16, wherein the human coronavirus is SARS-CoV.

Embodiment 41. The multimeric binding molecule of embodiment 40, wherein the three to twelve identical binding domains each specifically bind to SARS-CoV and comprise a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, and wherein the HCDR1, HCDR2, HCDR3, LCDR1, LCDR2, and LCDR3 comprise the CDRs of an antibody comprising the VH and VL SEQ ID NO: 84 and SEQ ID NO: 85, SEQ ID NO: 118 and SEQ ID NO: 119, SEQ ID NO: 120 and SEQ ID NO: 121, SEQ ID NO: 138 and SEQ ID NO: 139, SEQ ID NO: 162 and SEQ ID NO: 163, SEQ ID NO: 222 and SEQ ID NO: 223, SEQ ID NO: 236 and SEQ ID NO: 237, SEQ ID NO: 252 and SEQ ID NO: 253, SEQ ID NO: 260 and SEQ ID NO: 261, SEQ ID NO: 262 and SEQ ID NO: 263, SEQ ID NO: 296 and SEQ ID NO: 297, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, or SEQ ID NO: 644 and SEQ ID NO: 645.

Embodiment 42. The multimeric binding molecule of embodiment 41, wherein the VH and VL comprise amino acid sequences at least 80%, at least 85%, at least 90%, at least 95% or 100% identical to the recited VH and VL amino acid sequences, respectively.

Embodiment 43. The multimeric binding molecule of embodiment 41, wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL of SEQ ID NO: 84 and SEQ ID NO: 85, SEQ ID NO: 260 and SEQ ID NO: 261, SEQ ID NO: 262 and SEQ ID NO: 263, SEQ ID NO: 296 and SEQ ID NO: 297, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, or SEQ ID NO: 644 and SEQ ID NO: 645.

Embodiment 44. The multimeric binding molecule of embodiment 43, wherein the VH and VL comprise amino acid sequences at least 80%, at least 85%, at least 90%, at least 95% or 100% identical to the recited VH and VL amino acid sequences, respectively.

Embodiment 45. The multimeric binding molecule of embodiment 43 or embodiment 44, wherein the bivalent reference IgG antibody comprising two of the binding domains can neutralize SARS-CoV.

Embodiment 46. The multimeric binding molecule of any one of embodiments 1 to 16, wherein the human coronavirus is MERS-CoV.

Embodiment 47. The multimeric binding molecule of embodiment 46, wherein the three to twelve identical binding domains each specifically bind to MERS-CoV and comprise a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, and wherein the HCDR1, HCDR2, HCDR3, LCDR1, LCDR2, and LCDR3 comprise the CDRs of an antibody comprising the VH and VL SEQ ID NO:510 and SEQ ID NO:511, SEQ ID NO:512 and SEQ ID NO:513, SEQ ID NO:514 and SEQ ID NO:515, SEQ ID NO:516 and SEQ ID NO:517, SEQ ID NO:518 and SEQ ID NO:519, SEQ ID NO:520 and SEQ ID NO:521, SEQ ID NO:522 and SEQ ID NO:523, SEQ ID NO:524 and SEQ ID NO:525, SEQ ID NO:526 and SEQ ID NO:527, SEQ ID NO:528 and SEQ ID NO:529, SEQ ID NO:530 and SEQ ID NO:531, SEQ ID NO:532 and SEQ ID NO:533, SEQ ID NO:534 and SEQ ID NO:535, SEQ ID NO:536 and SEQ ID NO:537, SEQ ID NO:538 and SEQ ID NO:539, SEQ ID NO:540 and SEQ ID NO:541, SEQ ID NO:542 and SEQ ID NO:543, SEQ ID NO:544 and SEQ ID NO:545, SEQ ID NO:546 and SEQ ID NO:547, SEQ ID NO:548 and SEQ ID NO:549, SEQ ID NO:550 and SEQ ID NO:551, SEQ ID NO:552 and SEQ ID NO:553, SEQ ID NO:554 and SEQ ID NO:555, SEQ ID NO:556 and SEQ ID NO:557, SEQ ID NO:558 and SEQ ID NO:559, SEQ ID NO:560 and SEQ ID NO:561, SEQ ID NO:562 and SEQ ID NO:563, SEQ ID NO:564 and SEQ ID NO:565, SEQ ID NO:566 and SEQ ID NO:567, SEQ ID NO:568 and SEQ ID NO:569, SEQ ID NO:570 and SEQ ID NO:571, SEQ ID NO:572 and SEQ ID NO:573, SEQ ID NO:574 and SEQ ID NO:575, SEQ ID NO:576 and SEQ ID NO:577, SEQ ID NO:578 and SEQ ID NO:579, SEQ ID NO:580 and SEQ ID NO:581, SEQ ID NO:582 and SEQ ID NO:583, SEQ ID NO:584 and SEQ ID NO:585, SEQ ID NO:586 and SEQ ID NO:587, SEQ ID NO:588 and SEQ ID NO:589, SEQ ID NO:590 and SEQ ID NO:591, SEQ ID NO:592 and SEQ ID NO:593, SEQ ID NO:594 and SEQ ID NO:595, SEQ ID NO:596 and SEQ ID NO:597, SEQ ID NO:598 and SEQ ID NO:599, SEQ ID NO:600 and SEQ ID NO:601, SEQ ID NO:602 and SEQ ID NO:603, SEQ ID NO:604 and SEQ ID NO:605, SEQ ID NO:606 and SEQ ID NO:607, SEQ ID NO:608 and SEQ ID NO:609, SEQ ID NO:610 and SEQ ID NO:611, SEQ ID NO:612 and SEQ ID NO:613, SEQ ID NO:614 and SEQ ID NO:615, SEQ ID NO:616 and SEQ ID NO:617, SEQ ID NO:618 and SEQ ID NO:619, SEQ ID NO:620 and SEQ ID NO:621, SEQ ID NO:622 and SEQ ID NO:623, SEQ ID NO:624 and SEQ ID NO:625, SEQ ID NO:626 and SEQ ID NO:627, or SEQ ID NO:630 and SQ ID NO:631, respectively, with zero, one, or two single amino acid substitutions in one or more of the HCDRs or LCDRs.

Embodiment 48. The multimeric binding molecule of embodiment 47, wherein the VH and VL comprise amino acid sequences at least 80%, at least 85%, at least 90%, at least 95% or 100% identical to the recited VH and VL amino acid sequences, respectively.

Embodiment 49. The multimeric binding molecule of any of embodiment 47, wherein the HCDR1, HCDR2, HCDR3, LCDR1, LCDR2, and LCDR3 comprise the CDRs of an antibody comprising the VH and VL of SEQ ID NO: 510 and SEQ ID NO: 511, SEQ ID NO: 512 and SEQ ID NO: 513, SEQ ID NO: 514 and SEQ ID NO: 515, SEQ ID NO: 516 and SEQ ID NO: 517, SEQ ID NO: 518 and SEQ ID NO: 519, SEQ ID NO: 520 and SEQ ID NO: 521, SEQ ID NO: 522 and SEQ ID NO: 523, SEQ ID NO: 524 and SEQ ID NO: 525, SEQ ID NO: 526 and SEQ ID NO: 527, SEQ ID NO: 528 and SEQ ID NO: 529, SEQ ID NO: 530 and SEQ ID NO: 531, SEQ ID NO: 532 and SEQ ID NO: 533, SEQ ID NO: 534 and SEQ ID NO: 535, SEQ ID NO: 536 and SEQ ID NO: 537, SEQ ID NO: 538 and SEQ ID NO: 539, SEQ ID NO: 540 and SEQ ID NO: 541, SEQ ID NO: 542 and SEQ ID NO: 543, SEQ ID NO: 544 and SEQ ID NO: 545, SEQ ID NO: 546 and SEQ ID NO: 547, SEQ ID NO: 548 and SEQ ID NO: 549, SEQ ID NO: 550 and SEQ ID NO: 551, SEQ ID NO: 552 and SEQ ID NO: 553, SEQ ID NO: 554 and SEQ ID NO: 555, SEQ ID NO: 556 and SEQ ID NO: 557, SEQ ID NO: 558 and SEQ ID NO: 559, SEQ ID NO: 560 and SEQ ID NO: 561, SEQ ID NO: 562 and SEQ ID NO: 563, SEQ ID NO: 564 and SEQ ID NO: 565, SEQ ID NO: 566 and SEQ ID NO: 567, SEQ ID NO: 568 and SEQ ID NO: 569, SEQ ID NO: 574 and SEQ ID NO: 575, SEQ ID NO: 576 and SEQ ID NO: 577, SEQ ID NO: 578 and SEQ ID NO: 579, SEQ ID NO: 580 and SEQ ID NO: 581, SEQ ID NO: 582 and SEQ ID NO: 583, SEQ ID NO: 584 and SEQ ID NO: 585, SEQ ID NO: 586 and SEQ ID NO: 587, SEQ ID NO: 588 and SEQ ID NO: 589, SEQ ID NO: 590 and SEQ ID NO: 591, SEQ ID NO: 592 and SEQ ID NO: 593, SEQ ID NO: 594 and SEQ ID NO: 595, SEQ ID NO: 596 and SEQ ID NO: 597, SEQ ID NO: 598 and SEQ ID NO: 599, SEQ ID NO: 600 and SEQ ID NO: 601, SEQ ID NO: 602 and SEQ ID NO: 603, SEQ ID NO: 604 and SEQ ID NO: 605, SEQ ID NO: 606 and SEQ ID NO: 607, SEQ ID NO: 608 and SEQ ID NO: 609, SEQ ID NO: 610 and SEQ ID NO: 611, SEQ ID NO: 612 and SEQ ID NO: 613, SEQ ID NO: 614 and SEQ ID NO: 615, SEQ ID NO: 616 and SEQ ID NO: 617, SEQ ID NO: 618 and SEQ ID NO: 619, SEQ ID NO: 620 and SEQ ID NO: 621, SEQ ID NO: 622 and SEQ ID NO: 623, SEQ ID NO: 624 and SEQ ID NO: 625, SEQ ID NO: 626 and SEQ ID NO: 627, or SEQ ID NO: 630 and SEQ ID NO: 631, respectively.

Embodiment 50. The multimeric binding molecule of any of embodiment 49, wherein the HCDR1, HCDR2, HCDR3, LCDR1, LCDR2, and LCDR3 comprise the CDRs of an antibody comprising the VH and VL of SEQ ID NO: 522 and SEQ ID NO: 523, SEQ ID NO: 576 and SEQ ID NO: 577, SEQ ID NO: 610 and SEQ ID NO: 611, SEQ ID NO: 612 and SEQ ID NO: 613, SEQ ID NO: 614 and SEQ ID NO: 615, or SEQ ID NO: 630 and SEQ ID NO: 631, respectively.

Embodiment 51. The multimeric binding molecule of embodiment 49 or embodiment 50, wherein the VH and VL comprise amino acid sequences at least 80%, at least 85%, at least 90%, at least 95% or 100% identical to the recited VH and VL amino acid sequences, respectively.

Embodiment 52. The multimeric binding molecule of any one of embodiments 49 to 51, wherein the bivalent reference IgG antibody comprising two of the binding domains can neutralize MERS-CoV.

Embodiment 53. The multimeric binding molecule of embodiment 46, wherein the three to twelve identical binding domains of the multimeric binding molecule comprise an extracellular MERS-CoV RBD-binding fragment of dipeptidyl peptidase 4 (DPP4).

Embodiment 54. The multimeric binding molecule of any one of embodiments 1 to 53, which can neutralize escape mutants of the bivalent reference IgG antibody comprising two of the binding domains.

Embodiment 55. The multimeric binding molecule of any one of embodiments 1 to 54, comprising two or four bivalent IgA or IgA-like binding units and a J chain or functional fragment or variant thereof, wherein each binding unit comprises two IgA heavy chain constant regions or multimerizing fragments or variants thereof, each comprising an IgA Cα3 domain and an IgA tailpiece domain.

Embodiment 56. The multimeric binding molecule of embodiment 55, which is a dimeric binding molecule comprising two bivalent IgA or IgA-like binding units.

Embodiment 57. The multimeric binding molecule of embodiment 55 or embodiment 56, wherein each IgA heavy chain constant region or multimerizing fragment or variant thereof further comprises a Cα1 domain, a Cα2 domain, an IgA hinge region, or any combination thereof.

Embodiment 58. The multimeric binding molecule of any one of embodiments 55 to 57, wherein the IgA heavy chain constant regions or multimerizing fragments or variants thereof are human IgA constant regions.

Embodiment 59. The multimeric binding molecule of any one of embodiments 55 to 58, wherein each binding unit comprises two IgA heavy chains each comprising a VH situated amino terminal to the IgA constant region or multimerizing fragment thereof, and two immunoglobulin light chains each comprising a VL situated amino terminal to an immunoglobulin light chain constant region.

Embodiment 60. The multimeric binding molecule of any one of embodiments 1 to 54, comprising five or six bivalent IgM or IgM-like binding units, wherein each binding unit comprises two IgM heavy chain constant regions or multimerizing fragments or variants thereof, each comprising an IgM Cμ4 and IgM tailpiece domain.

Embodiment 61. The multimeric binding molecule of embodiment 60, wherein each IgM heavy chain constant region or multimerizing fragment or variant thereof further comprises a Cμ1 domain, a Cμ2 domain, a Cμ3 domain, or any combination thereof.

Embodiment 62. The multimeric binding molecule of embodiment 60 or embodiment 61, wherein the IgM heavy chain constant regions or multimerizing fragments or variants thereof are human IgM constant regions.

Embodiment 63. The multimeric binding molecule of embodiment 62, wherein the IgM heavy chain constant regions each comprise the amino acid sequence SEQ ID NO: 1, SEQ ID NO: 2, or a multimerizing fragment or variant thereof.

Embodiment 64. The multimeric binding molecule of any one of embodiments 60 to 63, wherein each binding unit comprises two IgM heavy chains each comprising a VH situated amino terminal to the IgM constant region or fragment thereof, and two immunoglobulin light chains each comprising a VL situated amino terminal to an immunoglobulin light chain constant region.

Embodiment 65. The multimeric binding molecule of any one of embodiments 60 to 63, wherein the IgM constant regions each comprise one or more amino acid substitutions corresponding to a wild-type human IgM constant region at position 310, 311, 313, and/or 315 of SEQ ID NO: 1 or SEQ ID NO: 2, and wherein the multimeric binding molecule exhibits reduced complement-dependent cytotoxicity (CDC) activity to cells in the presence of complement, relative to a reference binding molecule that is identical except for the one or more amino acid substitutions.

Embodiment 66. The multimeric binding molecule of any one of embodiments 60 to 64, wherein the IgM constant regions each comprise one or more substitutions corresponding to a wild-type human IgM constant region at positions 46, 209, 272, or 440 of SEQ ID NO: 1 or SEQ ID NO: 2, wherein the one or more amino acid substitutions prevent asparagine (N)-linked glycosylation.

Embodiment 67. The multimeric binding molecule of any one of embodiments 60 to 66 which is pentameric, and further comprises a J-chain or functional fragment or variant thereof.

Embodiment 68. The multimeric binding molecule of any one of embodiments 55 to 59 or 67, which can transport across vascular endothelial cells via J-chain binding to the polymeric Ig receptor (PIgR).

Embodiment 69. The multimeric binding molecule of any one of embodiments 55 to 59 or 67, further comprising a secretory component, or fragment or variant thereof.

Embodiment 70. The multimeric binding molecule of any one of embodiments 55 to 59 or 67 to 69, wherein the J-chain or functional fragment or variant thereof further comprises a heterologous polypeptide, wherein the heterologous polypeptide is directly or indirectly fused to the J-chain or functional fragment or variant thereof.

Embodiment 71. The multimeric binding molecule of embodiment 70, wherein the heterologous polypeptide is fused to the J-chain or fragment thereof via a peptide linker.

Embodiment 72. The multimeric binding molecule of embodiment 71, wherein the peptide linker comprises at least 5 amino acids, but no more than 25 amino acids.

Embodiment 73. The multimeric binding molecule of embodiment 71 or 72, wherein the peptide linker consists of GGGGS (SEQ ID NO: 9), GGGGSGGGGS (SEQ ID NO: 10), GGGGSGGGGSGGGGS (SEQ ID NO: 11), GGGGSGGGGSGGGGSGGGGS (SEQ ID NO: 12), or GGGGSGGGGSGGGGSGGGGSGGGGS (SEQ ID NO: 13).

Embodiment 74. The multimeric binding molecule of any one of embodiments 70 to 73, wherein the heterologous polypeptide is fused to the N-terminus of the J-chain or fragment or variant thereof, the C-terminus of the J-chain or fragment or variant thereof, or to both the N-terminus and C-terminus of the J-chain or fragment or variant thereof.

Embodiment 75. The multimeric binding molecule of any one of embodiments 70 to 74, wherein the heterologous polypeptide can influence the absorption, distribution, metabolism and/or excretion (ADME) of the multimeric binding molecule.

Embodiment 76. The multimeric binding molecule of any one of embodiments 70 to 75, wherein the heterologous polypeptide comprises an albumin or an albumin binding domain.

Embodiment 77. The multimeric binding molecule of any one of embodiments 70 to 76, wherein the heterologous polypeptide comprises human serum albumin.

Embodiment 78. The multimeric binding molecule of any one of embodiments 70 to 77, wherein the heterologous polypeptide comprises an antigen binding domain.

Embodiment 79. The multimeric binding molecule of embodiment 78, wherein the antigen binding domain binds to the human coronavirus.

Embodiment 80. The multimeric binding molecule of embodiment 79, wherein the antigen binding domain binds to a different epitope of the human coronavirus than the three to twelve identical binding domains of the multimeric binding molecule that specifically bind to the human coronavirus.

Embodiment 81. The multimeric binding molecule of any one of embodiments 78 to 80, wherein the antigen binding domain of the heterologous polypeptide is an antibody or antigen-binding fragment thereof.

Embodiment 82. The multimeric binding molecule of embodiment 81, wherein the antigen-binding fragment comprises a Fab fragment, a Fab′ fragment, a F(ab′)2 fragment, a Fd fragment, a Fv fragment, a single-chain Fv (scFv) fragment, a disulfide-linked Fv (sdFv) fragment, or any combination thereof.

Embodiment 83. The multimeric binding molecule of embodiment 81 or embodiment 82, wherein the antigen-binding fragment is a scFv fragment.

Embodiment 84. The multimeric binding molecule of any one of embodiments 70 to 74, wherein the heterologous polypeptide comprises an extracellular SARS-CoV-2 RBD-binding fragment of angiotensin-converting enzyme 2 (ACE2).

Embodiment 85. The multimeric binding molecule of any one of embodiments 70 to 74, wherein the heterologous polypeptide comprises an extracellular MERS-COV RBD-binding fragment of dipeptidyl peptidase 4 (DPP4).

Embodiment 86. The multimeric binding molecule of any one of embodiments 74 to 85, wherein the J-chain or functional fragment or variant thereof further comprises an additional heterologous polypeptide, wherein the additional heterologous polypeptide is directly or indirectly fused to the J-chain or functional fragment or variant thereof.

Embodiment 87. The multimeric binding molecule of embodiment 86, wherein the additional heterologous polypeptide can influence the absorption, distribution, metabolism and/or excretion (ADME) of the multimeric binding molecule.

Embodiment 88. The multimeric binding molecule of embodiment 86 or embodiment 87, wherein the additional heterologous polypeptide comprises an albumin or an albumin binding protein.

Embodiment 89. The multimeric binding molecule of embodiment 88, wherein the additional heterologous polypeptide comprises human serum albumin.

Embodiment 90. A composition comprising the multimeric binding molecule of any one of embodiments 1 to 89.

Embodiment 91. A composition comprising two or more nonidentical multimeric binding molecules according to any one of embodiments 1 to 89, wherein the two or more multimeric binding molecules bind to different epitopes of a single human coronavirus.

Embodiment 92. A polynucleotide comprising a nucleic acid sequence that encodes a polypeptide subunit of the binding molecule of any one of embodiments 1 to 89.

Embodiment 93. A vector comprising the polynucleotide of embodiment 92.

Embodiment 94. A host cell comprising the polynucleotide of embodiment 92, or the vector of embodiment 93, wherein the host cell can express the multimeric binding molecule of any one of embodiments 1 to 89, or a subunit thereof.

Embodiment 95. A method of producing the multimeric binding molecule of any one of embodiments 1 to 89, comprising culturing the host cell of embodiment 94, and recovering the multimeric binding molecule.

Embodiment 96. The method of embodiment 95, further comprising contacting the multimeric binding molecule with a secretory component, or fragment or variant thereof.

Embodiment 97. A method for treating a human coronavirus disease in a subject in need of treatment comprising administering to the subject an effective amount of the multimeric binding molecule of any one of embodiments 1 to 89, wherein the multimeric binding molecule exhibits greater potency than an equivalent amount of a bivalent reference IgG antibody comprising two of the binding domains that specifically bind to the human coronavirus.

Embodiment 98. A method for preventing a human coronavirus disease in a subject, comprising administering to the subject an effective amount of the multimeric binding molecule of any one of embodiments 1 to 89, wherein the multimeric binding molecule exhibits greater potency than an equivalent amount of a bivalent reference IgG antibody comprising two of the binding domains that specifically bind to the human coronavirus.

Embodiment 99. The method of embodiment 97 or embodiment 98, wherein the human coronavirus disease is severe acute respiratory syndrome (SARS).

Embodiment 100. The method of embodiment 97 or embodiment 98, wherein the human coronavirus disease is coronavirus disease 2019 (COVID-19).

Embodiment 101. The method of embodiment 97 or embodiment 98, wherein the human coronavirus disease is Middle East Respiratory Syndrome (MERS).

Embodiment 102. The method of any one of embodiments 97 to 101, wherein the subject is human.

Embodiment 103. The method of any one of embodiments 97 to 102, wherein the administering comprises intravenous, subcutaneous, intramuscular, intranasal, and/or inhalation administration.

### EXAMPLES

### Example 1: Generation of Antibodies to SARS-CoV-1 and SARS-CoV-2

The VH and VL regions of two anti-SARS-CoV-1 antibodies, CR3022 and CR3014 (US20060121580), were incorporated into IgM, IgA1, and IgA2m2 formats (each with an exemplary J-chain, SEQ ID NO: 7) and IgG format according to standard cloning protocols. CR3022 IgG is known to bind SARS-CoV-2 and CR3014 IgG does not. CR3022 constructs included the VH and VL amino acid sequences SEQ ID NO: 84 and SEQ ID NO: 85, respectively, and CR3014 constructs included the VH and VL amino acid sequences SEQ ID NO: 262 and SEQ ID NO: 263, respectively.

The IgM and IgG antibody constructs were expressed and purified according to methods described in WO2017196867. The IgA antibody constructs were expressed using the same methods described in WO2017196867 as the IgM antibody constructs, except that the appropriate IgA heavy chain was used. The IgM antibodies assembled as pentamers with a J-chain, and the IgA antibodies assembled as dimers and/or tetramers with a J-chain (data not shown).

### Example 2: Antibody Binding Measured by ELISA

CR3022 was originally developed as an anti-SARS-CoV-1 IgG antibody (US20060121580) and CR3022 IgG has recently been demonstrated to also bind SARS-CoV-2 (Tian et al. Emerging Microbes & Infections, 2020, doi: 10.1080/22221751.2020.1729069). ELISA was used to determine how the IgM, IgA1, IgA2m2, and IgG1 formats affect binding to SARS-CoV-1 and SARS-CoV-2.

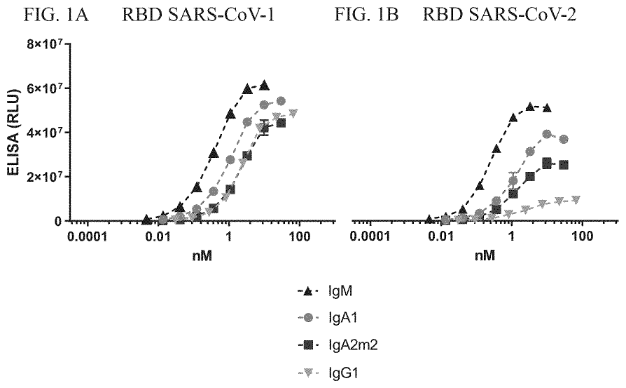

Binding of CR3022 IgM, IgA1, IgA2m2, and IgG (described in Example 1) to SARS-CoV-1 and SARS-CoV-2 was measured in ELISA assays as follows. 96-well white polystyrene ELISA plates (Pierce 15042) were coated with 100 μL per well of 0.5 μg/mL recombinant SARS-CoV-1 Receptor Binding Domain (RBD) with a his tag, recombinant SARS-CoV-2 RBD with a his tag, recombinant SARS-CoV-2 RBD with a Fc tag, or recombinant SARS-CoV-2 spike (S) protein trimer, overnight at 4° C. Plates were then washed 5 times with 0.05% PBS-Tween and blocked with 2% BSA-PBS. After blocking, 100 μL of serial dilutions of CR3022 IgM, IgA1, IgA2m2, or IgG; standards; or controls were added to the wells and incubated at room temperature for 2 hours. The plates were then washed 10 times and incubated with HRP conjugated mouse anti-human kappa (Southern Biotech, 9230-05; 1:6000 diluted in 2% BSA-PBS) for 30 min. After 10 final washes using 0.05% PBS-Tween, the plates were read out using SuperSignal chemiluminescent substrate (ThermoFisher, 37070). Luminescent data were collected on an EnVision plate reader (Perkin-Elmer) and analyzed with GraphPad Prism using a 4-parameter logistic model. Binding to SARS-CoV-1 RBD is shown in FIG. 1A, and binding to SARS-CoV-2 RBD is shown in FIG. 1B. Similar binding profiles were seen with SARS-CoV-2 RBD his tag, SARS-CoV-2 Fc, and SARS-CoV-2 S trimer.

### Example 3: Pseudovirus Neutralization Assay

CR3022 IgG and CR3014 IgG have both been reported to neutralize SARS-CoV-1 (US20060121580). CR3014 IgG does not bind SARS-CoV-2 RBD whereas CR3022 IgG does bind SARS-CoV-2 RBD (Tian et al., Emerging Microbes & Infections, doi: 10.1080/22221751.2020.1729069, 2020). However, there has been disagreement whether CR3022 IgG is capable of neutralizing SARS-CoV-2 (Yuan et al., Science 10.1126/science.abb7269; 2020), Huo et al., Cell Host Microbe, doi: 10.1016/j.chom.2020.06.010, 2020). A pseudovirus neutralization assay was used to determine how the IgM, IgA1, IgA2m2, and IgG1 formats affect neutralization of pseudoviruses comprising the SARS-CoV-1 or SARS-CoV-2 spike protein.

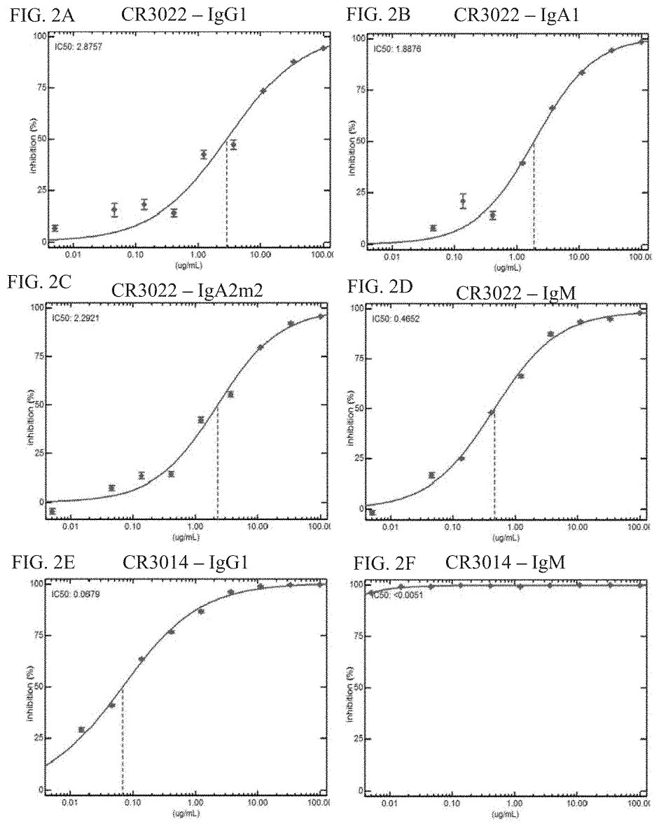

A SARS-CoV pseudovirus neutralization assay was performed generally as described in Richman et al. (PNAS, 2003, 100(7): 4144-4149), wherein host cells that have been infected with an HIV-based pseudovirus express luciferase. In the SARS-CoV-1 or SARS-CoV-2 pseudovirus neutralization assay, the pseudovirus was engineered to express SARS-CoV-1 or SARS-CoV-2 spike proteins in place of HIV gp160. Various concentrations of CR3022 IgG1, IgA1, IgA2m2, or IgM or CR3014 IgG1 and IgM were incubated with the SARS-CoV-1 or SARS-CoV-2 pseudovirus for 1 hr. at 37° C. HEK-293 cells expressing human ACE-2 were then inoculated with the antibody/pseudovirus solution. The amount of luciferase activity in the infected cells was measured 72 h post-inoculation to determine the neutralizing activity. A decrease in luciferase activity indicated neutralizing activity by the antibody assayed. Neutralizing activity was calculated as the percent inhibition of viral replication (luciferase activity) at each antibody dilution compared with an antibody-negative control. The percent inhibitions of the SARS-CoV-1 pseudovirus for CR3022 IgG1, CR3022 IgA1, CR3022 IgA2m2, CR3022 IgM, CR3014 IgG1, and CR3014 IgM are shown in FIGS. 2A-2F, respectively. Neither CR3022 nor CR3014, in any format, neutralized SARS-CoV-2 pseudovirus infectivity. In contrast, all forms of CR3022 and CR3014 neutralized SARS-CoV-1, with the IgM and IgA forms being the most potent. The IC₅₀ and fold improvement over IgG1 for each IgA and IgM antibody are shown in Table 2. CR3022 is ˜6-fold more potent as an IgM than as an IgG1 in neutralizing SARS-CoV-1 pseudovirus on a weight basis, and CR3014 is at least 13-fold more potent as an IgM than as an IgG.

TABLE 2
Pseudovirus Neutralization Results

| | IC₅₀ (μg/mL) | | Fold Over IgG1 (CoV-1) | |
|---|---|---|---|---|
| | SARS- | SARS- | By | By |
| Antibody | CoV-1 | CoV-2 | Weight | Molar |
| CR3022-IgG1 | 2.9 | >100,000 | 1.0 | 1.0 |
| CR3022-IgA1 + J | 1.9 | >100,000 | 1.5 | 3.4 |
| CR3022-IgA2m2 + J | 2.3 | >100,000 | 1.3 | 2.8 |
| CR3022-IgM + J | 0.47 | >100,000 | 6.2 | 37 |
| CR3014-IgG1 | 0.068 | >100,000 | 1.0 | 1.0 |
| CR3014-IgM + J | <0.0051 | >100,000 | >13 | >80 |

### Example 4: Antibody Transcytosis

To determine if CR3022 IgA1, IgA2, and IgM antibodies are capable of functionally interacting with the polymeric immunoglobulin receptor (pIgR) and being transcytosed across a cell barrier, an in vitro transcytosis assay was developed, modified from the transcytosis assay describe by Chung et al. (2019, mAbs, 11(5): 942-955).

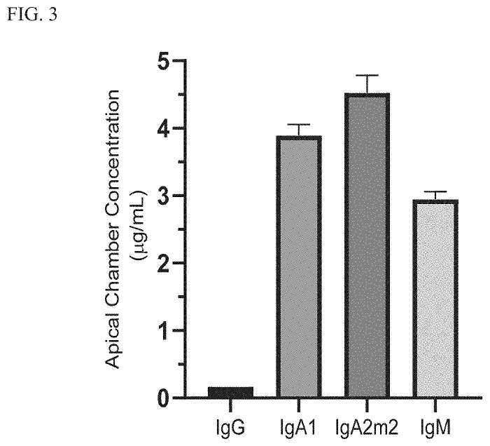

MDCK.2 (Sigma) cells were transfected to express human pIgR. The hpIgR-expressing MDCK.2 cells were seeded at a density of 1×10⁵ cells/well in growth medium (DMEM High Glucose supplemented with 10% FBS) in a 96-well trans-well plate and were incubated for 48 hours at 37° C. The transwell insert and corresponding monolayer of cells were transferred to a receiver plate in which the basolateral chamber contained 11.1, 33.3, or 100 μg/mL of CR3022 IgA1, IgA2, or IgM antibodies in growth medium. A human IgG antibody was also included as a control. The cells were incubated at 37° C. for another 24 hours. The media from the apical chamber were then collected and the amount of transcytosed molecules was determined by ELISA. The resulting concentrations for each antibody format are shown in FIG. 3. CR3022 IgA1, IgA2 and IgM antibodies were efficiently transcytosed across the cell monolayer, whereas the IgG antibody was not.

### Example 5: Additional SARS-CoV-2 Antibody Generation

The VH and VL regions of eight anti-SARS-CoV-2 antibodies were incorporated into IgM with an exemplary J-chain, SEQ ID NO: 7 and IgG format according to standard cloning protocols. The VH and VL amino acid sequences used for each antibodies construct are shown in Table 3.

TABLE 3
Antibody Constructs Generated

SEQ ID NO

| | VH | VL |
|---|---|---|
| Ab1 | 88 | 89 |
| Ab2 | 260 | 261 |
| Ab3 | 264 | 265 |
| Ab4 | 266 | 267 |
| Ab5 | 284 | 285 |
| Ab6 | 286 | 287 |
| Ab7 | 288 | 289 |
| Ab8 | 290 | 291 |
| Ab9 | 292 | 293 |
| Ab10 | 274 | 275 |
| Ab11 | 278 | 279 |
| Ab12 | 280 | 281 |
| Ab13 | 282 | 283 |

The IgM antibodies were purified according to methods described in Keyt, B., et al. Antibodies: 9:53, doi: 10.3390/antib9040053 (2020), or, e.g., as described in WO2017196867. The IgA and IgG antibodies were purified by affinity chromatography. The IgM antibodies assembled as pentamers with a J-chain and the IgG antibodies properly assembled as monomers (data not shown).

### Example 6: Antibody Binding to SARS-CoV-2 Measured by ELISA

ELISA was used to determine how the IgM and IgG1 formats affect binding to SARS-CoV-2. Binding of Ab1-Ab4 IgM and IgG (described in Example 5) to SARS-CoV-2 was measured in ELISA assays as follows. 96-well white polystyrene ELISA plates (Pierce 15042) were coated with 100 μL per well of 0.5 μg/mL recombinant SARS-CoV-2 RBD with a HIS tag (for Ab1-Ab4) or S protein trimer (for Ab10-Ab13) overnight at 4° C. Plates were then washed 5 times with 0.05% PBS-Tween and blocked with 2% BSA-PBS. After blocking, 100 μL of serial dilutions of IgM or IgG antibodies, standards, or controls were added to the wells and incubated at room temperature for 2 hours. The plates were then washed 10 times and incubated with HRP conjugated mouse anti-human kappa (Southern Biotech, 9230-05; 1:6000 diluted in 2% BSA-PBS) or mouse anti-human lambda (clone JDC-12, Southern Biotech, 9180-05; 1:6000 diluted in 2% BSA-PBS) for 30 min. After 10 final washes using 0.05% PBS-Tween, the plates were read out using SuperSignal chemiluminescent substrate (ThermoFisher, 37070). Luminescent data were collected on an EnVision plate reader (PerkinElmer) and analyzed with GraphPad Prism using a 4-parameter logistic model. Binding of Ab1-Ab4 to SARS-CoV-2 RBD is shown in FIGS. 4A-4D, respectively. Binding of Ab10, Ab11, and Ab13 to SARS-CoV-2 S protein trimer is shown in FIGS. 4E-4G, respectively.

For Ab1-Ab4, Ab10, Ab11, and Ab13, both the IgG and IgM versions bound well to the SARS-CoV-2 RBD or S trimer but, in each case, the IgM bound much better than the IgG. Ab12 poorly bound the S trimer in both the IgG and IgM formats (data not shown).

### Example 7: SARS-CoV-2 Neutralization Assay

A SARS-CoV-2 live virus microneutralization assay was performed generally as described in Graham et al. (Clin Transl Immunology, 2020, 9(10):e1189, doi: 10.1002/cti2.1189.) using a focus reduction microneutralization test (FRNT) with various concentrations of antibodies. Specifically, the neutralizing activity of IgG1 and IgM versions of Ab1-Ab9 were assessed in vitro. Antibodies were combined with focus forming units of SARS-CoV-2 and incubated for 60 minutes at 37° C. and then assayed undiluted and in serial dilutions until reaching an endpoint of 1:3200. Samples were applied to confluent cell monolayers in 96 well plates and were incubated for 60 minutes at 37° C., followed by overlaying the wells with 1.2% methylcellulose in cDMEM and incubation at 37° C., 5% CO₂ for 24 hours. Infected cells were fixed in 25% formaldehyde in 3×PBS, then permeabilized with 0.1% Triton in 1×PBS for 15 minutes and then incubated with a primary anti-SARS-CoV monoclonal antibody, and then a secondary detectable antibody. Cell levels were counted using established methods. IC₅₀ determinations were made using a non-linear regression curve fit (log [inhibitor] vs. normalized response−variable slope) in GraphPad Prism. The IC₅₀ and fold improvement over IgG1 for each antibody are shown in Table 4.

TABLE 4
Neutralization Results

| | IgM IC₅₀ | IgG IC₅₀ | IgM Fold Improvement |
|---|---|---|---|
| | (ng/mL) | (ng/mL) | Over IgG1 |
| Ab1 | 13 | 130 | 10 |
| Ab2 | 50 | 92 | 1.8 |
| Ab3 | 3.8 | 11 | 2.9 |
| Ab4 | 3.1 | 180 | 59 |
| Ab5 | 11 | 70 | 6.2 |
| Ab6 | 41 | 2800 | 68 |
| Ab7 | 1.6 | 30 | 19 |
| Ab8 | 28 | 93 | 3.3 |
| Ab9 | 1.0 | 3.2 | 3.2 |

All antibodies tested neutralized SARS-CoV-2 and all IgM formatted antibodies were more potent at neutralizing SARS-CoV-2 relative to the IgGs.

### Example 8: In Vivo Assays

A variety of animal models are contemplated for testing of the disclosed binding molecule, e.g., antibodies to the spike protein of SARS-CoV-2. Such animal models included models described in Munoz-Fontela et al., Nature, 2020, 586: 509-515, which is herein incorporated by reference in its entirety. The most common animal models involve studies in mice, hamsters, and ferrets, as well as non-human primates such as rhesus macaques, cynomolgus macaques, and African green monkeys. Inoculation of virus in all animal models is done via intranasal administration—primate studies may also include intratracheal inoculation. For these studies, authentic SARS-CoV-2 virus, variant strains of SARS-CoV-2, or pseudovirus (e.g., VSV, HIV, Lentivirus, etc.) expressing the SARS-CoV-2 spike protein are used for infection, and dosing of experimental agents is done prophylactically (dosed before infection) or therapeutically (dosed after infection).

Since mice are not normally susceptible to infection by SARS-CoV-2 due to differences in their ACE2 protein, several methods have been developed to allow reproducible virus replication in this species. These include (i) the use of mice transgenic for human ACE2, (ii) the transduction of human ACE2 into mice via adenovirus or adeno-associated viral vectors, and (iii) the use of mouse-adapted SARS-CoV-2 virus that has been adapted to bind mouse ACE2 via repeated passage in mice. Some examples of mouse-adapted SARS-CoV-2 strains have been described by Li et al., Proc. Natl. Acad. Sci. USA, 2020, 117(47):29832-29838. and Gu et al., Science, 2020, 369 (6511): 1603-1607.

Similarly, multiple routes of administration for the test antibodies are contemplated for use herein in animal studies of SARS-CoV-2 infection. These include intravenous (IV), intraperitoneal (IP), intranasal (IN), intramuscular (IM), subcutaneous (SC) and inhalation (INH). Antibodies to be tested can be administered before (prophylaxis) or after (treatment) viral infection at doses ranging from for example 0.01 to 50 mg/kg. For mouse studies, typical times for prophylaxis or treatment range from 4 to 48 hrs. before or after infection. Control groups receive isotype control antibodies. Two days after infection, lung samples are harvested from all mice and homogenized in 1 mL PBS and analyzed for infectious virus by plaque assay or by RT-qPCR. In some studies, lung tissues are also collected and examined for changes in morphology, cytokines, and other markers of inflammation. Exemplary protocols are discussed below for studying antibodies to SARS-CoV-2 in vivo.

Studies with Mouse-Adapted SARS-CoV-2

BALB/c mice (10 to 12 weeks old, 5 to 10 per group) are anesthetized with isoflurane and infected intranasally (IN) with 10⁴ pfu of a mouse-adapted SARS-CoV-2 strain in 50 μL of phosphate-buffered saline (PBS). Antibodies to be tested are administered IP or IN before (prophylaxis) or after (treatment) viral infection, and viral loads in the lung are assessed 48 hrs. post infection.

Studies with Transgenic Mice (e.g., Zhang et al., bioRxiv, 2020, doi: 10.1101/2020.12.08.416677)

hACE2 transgenic mice (e.g., K18-hACE2, 6- to 9-wk-old, 5 to 6 per group) are treated IP with 0.3 mg (15 mg/kg) of antibody or negative controls 15 h prior to IN infection with 10⁵ pfu of wild-type SARS-CoV-2. Lung tissue is homogenized in phosphate-buffered saline (PBS) and virus replication assessed by plaque assay.

Alternatively, antibodies of various concentrations are administered IN (20 μL per nostril). Ten hours later, mice are infected IN with SARS-CoV-2 spike protein-expressing pseudotype lentivirus (20 μL per nostril). Seven days after intranasal administration of virus, bioluminescent imaging is performed for each mouse.

Studies with Transduced Mice (e.g., Zost et al., Nature, 2020, 584: 443-449)

BalbC mice (10-11 weeks old) are given a single intraperitoneal injection of 2 mg of anti-IFNAR1 monoclonal antibody (MAR1-5A355, Leinco) one day before intranasal administration of 2.5×10⁸ PFU of AdV-hACE2. Five days after AdV transduction, mice are inoculated with 4×10⁵ PFU of SARS-CoV-2 via the IN route. Anti-SARS-CoV-2 human monoclonal antibodies or isotype control monoclonal antibodies are administered 24 h before (prophylaxis) or 12 h after (treatment) SARS-CoV-2 inoculation. Weights are monitored on a daily basis, and mice are euthanized at 2 or 7 dpi and tissues are collected for analysis of PFU.

### Studies in Syrian Hamsters (e.g., Tortorici et al., Science, 2020, 370: 950-957)

Female hamsters of 6-10 weeks old are anesthetized with ketamine/xylazine/atropine and inoculated intranasally with 50 μL containing 2×10⁶ TCID₅₀ SARS-CoV-2. Antibodies to be tested are administered IP up to 48 hours before infection. Hamsters are monitored for appearance, behavior, and weight. At day 4 post infection (pi), hamsters are euthanized by intraperitoneal injection of 500 μL Dolethal (200 mg/mL sodium pentobarbital, Vetoquinol SA). Lungs are collected, and viral RNA and infectious virus were quantified by RT-qPCR and plaque assays, respectively.

### Studies in Rhesus Macaques (e.g., Baum et al., Science, 2020, 370: 1110-1115)

Antibodies or saline are administered through intravenous infusion. Animals (4 per group) are challenged with 1.1×10⁵ PFU SARS-CoV-2 (USA-WA1/2020 (NR-52281; BEI Resources) total dose divided between IN and intratracheal routes. Virus is administered using a 3 mL syringe to drop-wise instill 1 mL by the intranasal (IN) route (0.5 mL in each nare) and using a French rubber tube, is administered 1 mL via the intratracheal (IT) route. Viral titers are collected by nasal swabs (2× Copan flocked per animal, placed into one vial each with 1 mL PBS) and bronchioalveolar lavage (BAL) using 10 mL saline via a rubber feeding tube. Collected swabs and BAL aliquots are stored at −80° C. until viral load analysis.

### Example 9: Neutralization Assay

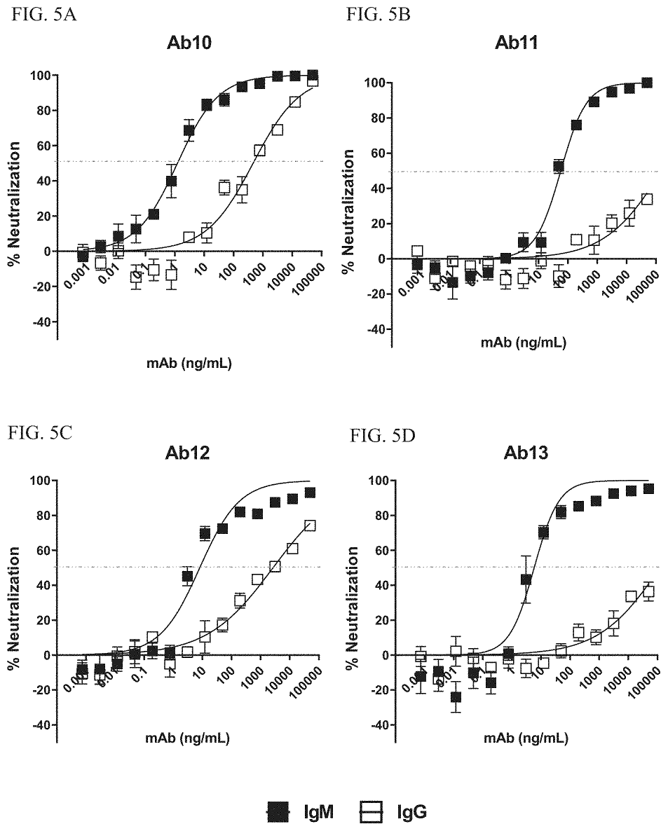

A SARS-CoV-2 live virus microneutralization assay was performed generally as described in Graham et al. (Clin Transl Immunology, 2020, 9(10):e1189, doi: 10.1002/cti2.1189.) as described in Example 7, using various concentrations of IgG1 and IgM versions of Ab10-Ab13. Data were analyzed with using a non-linear regression curve fit (log [inhibitor] vs. normalized response−variable slope) in GraphPad Prism. The resulting % Neutralization of Ab10-Ab13 are shown in FIGS. 5A-5D, respectively. The IC₅₀ and fold improvement over IgG1 for each antibody are shown in Table 5.

TABLE 5
Neutralization Results

| | IgM IC₅₀ | IgG IC₅₀ | IgM Fold Improvement |
|---|---|---|---|
| | (ng/mL) | (ng/mL) | Over IgG1 |
| Ab10 | 1.2 | 470 | 390 |
| Ab11 | 57 | >50,000 | >870 |
| Ab12 | 8.1 | 2700 | 330 |
| Ab13 | 5.8 | >50,000 | >8,600 |

All antibodies tested neutralized SARS-CoV-2 and all IgM formatted antibodies were more potent at neutralizing SARS-CoV-2 relative to the IgGs.

### Example 10: Pseudovirus Neutralization Assay

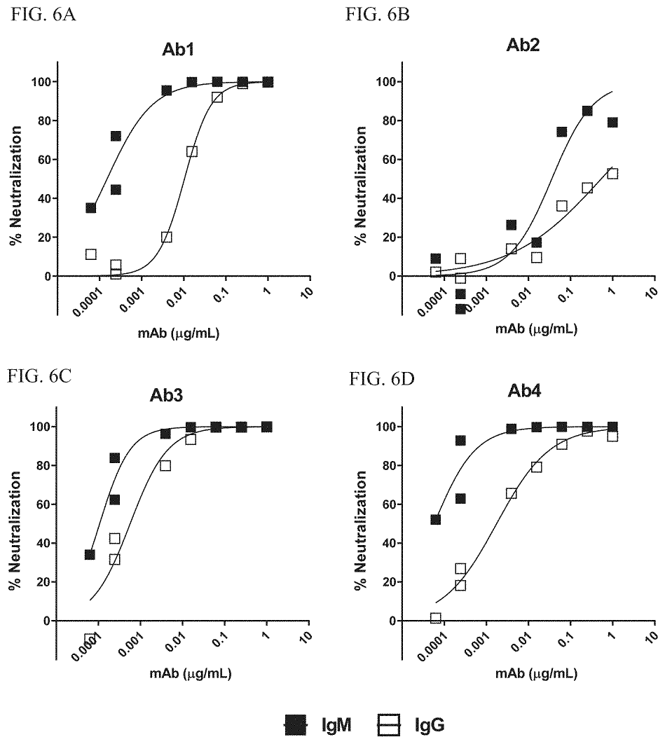

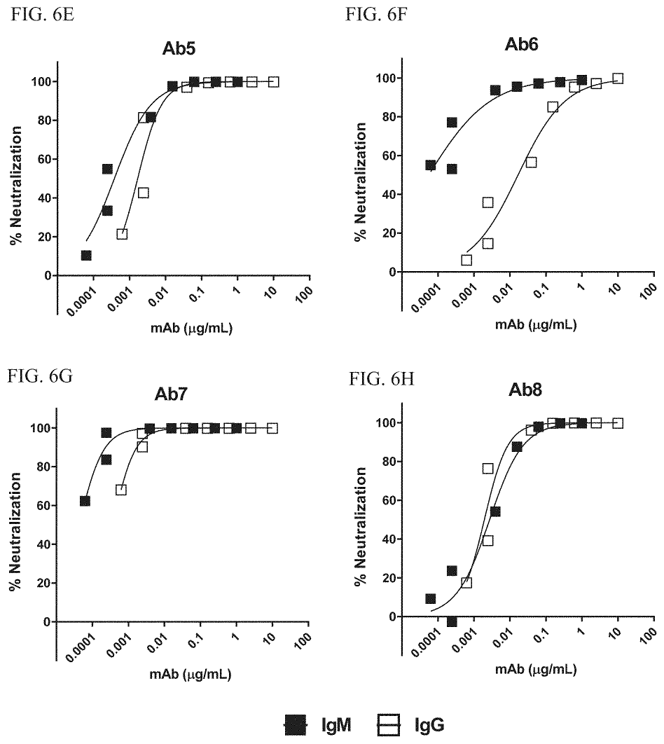

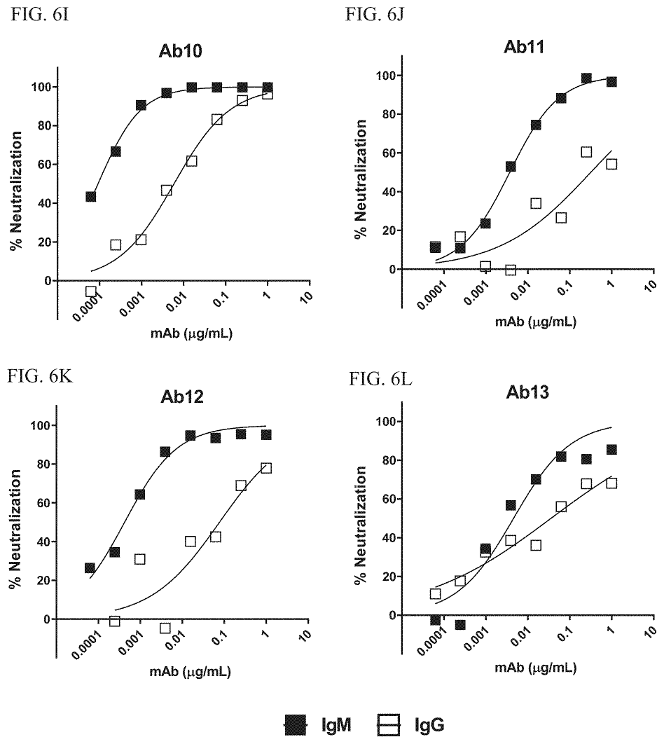

A lentivirus-based SARS-CoV-2 pseudovirus particle was generated expressing spike protein on the surface (Accession number: MN908947.3). The pseudovirus neutralization assay is based on previously described methodologies using luciferase-expressing HIV-1 pseudovirions (Richman et al. (Proc. Natl. Acad. Sci. USA, 2003, 100(7): 4144-4149), Folegatti et al., Lancet, 2020, 396: 467-478). Briefly, neutralizing antibody (Nab) titers were determined by creating 9 serial four-fold dilutions of Ab1-Ab13 IgM or IgG antibodies, which were mixed with ˜10⁵ relative light units (RLU) of SARS-CoV-2 pseudotyped virus and incubated at 37° C. for one hour. Separately, irrelevant pseudotyped control virus was also mixed with test samples. Following the 1-hour incubation, HEK 293 ACE2-transfected cells were added to the well. The plates were incubated for 60-80 hours at 37° C. and then assayed for luciferase expression. Neutralization titers are reported as the reciprocal of the serum dilution conferring 50% inhibition (ID₅₀) of pseudovirus infection. % Inhibition=100%−(((RLU_{(vector+sample+Diluent)}−RLU_{(Background)})/(RLU_{(Vector+Diluent)}−RLU_{(Background)}))×100%). SARS CoV-2 nAb Assay Positive and Negative Control Sera were included on each 96-well assay plate. The data for Ab9 did not meet quality control standards, and therefore are not included in the analysis. Data were analyzed using a non-linear regression curve fit (log [inhibitor] vs. normalized response−variable slope) in GraphPad Prism. The resulting % Neutralization of Ab1-Ab8, and Ab10-Ab13 are shown in FIGS. 6A-6L, respectively. The IC₅₀ and fold improvement over IgG1 for each antibody are shown in Table 6.

TABLE 6
Pseudovirus Neutralization Results

| | IgM IC₅₀ | IgG IC₅₀ | IgM Fold Improvement |
|---|---|---|---|
| | (ng/mL) | (ng/mL) | Over IgG1 |
| Ab1 | 0.15 | 10 | 67 |
| Ab2 | 36 | 530 | 15 |
| Ab3 | 0.11 | 0.57 | 5.2 |
| Ab4 | 0.058 | 1.8 | 31 |
| Ab5 | 0.38 | 1.7 | 4.5 |
| Ab6 | 0.055 | 16 | 290 |
| Ab7 | 0.042 | 0.37 | 8.8 |
| Ab8 | 2.7 | 1.9 | — |
| Ab10 | 0.092 | 5.9 | 64 |
| Ab11 | 3.7 | 320 | 86 |
| Ab12 | 0.42 | 77 | 180 |
| Ab13 | 4.6 | 36 | 7.8 |

### Example 11: Biolayer Interferometry Affinity Measurement Assay

The binding affinities & avidity of Ab7, Ab8, or Ab10 IgG or IgM antibodies with SARS-CoV-2 RBD (MW 26.5 KDa) or variant RBDs were determined by biolayer interferometry (BLI) on an Octet-384 (Sartorius/ForteBio, NY, USA) using Anti-Penta-His biosensors(Sartorius/Fortebio Cat #18-5120). PBST (1×PBS+1% BSA+0.05% Tween-20) buffer was used as Antibody/RBD/ACE-2 dilution and sensor hydration buffer. The experiment followed a six-step sequential assay at 24° C. First, biosensors were hydrated for 10 minutes. Samples and buffer were applied in 384-well plate. After initial baseline of 30 s, sensors were loaded with various RBDs for 240 s, then moved into buffer for a baseline for 30 s. Next, various concentrations of antibody were associated for 240 s and dissociated for 500 s in PBST. Results were analyzed by ForteBio Data Analysis software 9.0 using global fit model. The K_{D} values are shown in Tables 7-9. Underline indicates significant difference between IgM and IgG formats. WB indicates weak binding.

### Example 12: ACE2 Blocking Assay

Analysis of the ability of IgG1 and IgM versions of Ab7, Ab8, or Ab10 to block ACE2-Fc (Acrobiosystems, Cat #AC2-H5257) binding of SARS-CoV-2 WT and various variant RBDs was performed by BLI on an Octet-384 (Sartorius/Fortebio, NY, USA) using anti-Penta His biosensors (Sartorius/Fortebio Cat #18-5120). PBST (1×PBS+1% BSA+0.05% Tween-20) buffer was used as Antibody/RBD/ACE-2 dilution and sensor hydration buffer. The experiment followed a six-step sequential assay at 24° C. First, biosensors were hydrated for 10 minutes. Samples and buffer were applied in 384-well plate. After initial baseline of 30 s, sensors were loaded with RBD for 400 s, then moved into buffer for a baseline for 30 s. Next, various concentrations of antibody were associated for 600 s followed by baseline for 20 s. Then, 100 nM of ACE-2 was associated for 600 s. Results were analyzed by ForteBio Data Analysis software 9.0. as (nm) rise of ACE2 binding. % RBD-ACE2 blocking was plotted & IC₅₀ values were calculated using GraphPad Prism 8.0. The IC₅₀ values in nM and M are shown in Tables 7-9. Underline indicates significant difference between IgM and IgG formats. WB indicates weak binding.

TABLE 7
Binding Kinetics and ACE2 Blocking Assay Results for Ab7

| | | RBD | | | | | |
|---|---|---|---|---|---|---|---|
| Ab7 | Ab Format | WT | K417E | G446V | Y453F | E484K | Q493K |
| Avidity | IgM | <0.001 | <0.001 | <0.001 | <0.001 | 0.007 | <0.001 |
| KD (nM) | IgG | <0.001 | <0.001 | <0.001 | <0.001 | WB | <0.001 |
| ACE2 Blocking | IgM | 0.11 | 0.36 | 0.32 | 0.42 | 0.87 | 0.30 |
| IC₅₀ (nM) | IgG | 0.88 | 2.4 | 2.1 | 3.2 | >69 | 2.2 |
| ACE2 Blocking | IgM | 0.098 | 0.31 | 0.28 | 0.36 | 0.76 | 0.26 |
| IC₅₀ (μg/mL) | IgG | 0.13 | 0.35 | 0.30 | 0.46 | >10 | 0.32 |

TABLE 8
Binding Kinetics and ACE2 Blocking Assay Results for Ab8

| | | RBD | | | | | |
|---|---|---|---|---|---|---|---|
| Ab8 | Ab Format | WT | K417E | G446V | Y453F | E484K | Q493K |
| Avidity | IgM | <0.001 | <0.001 | <0.001 | <0.001 | <0.001 | <0.001 |
| KD (nM) | IgG | <0.001 | 2.0 | <0.001 | <0.001 | <0.001 | <0.001 |
| ACE2 Blocking | IgM | 0.30 | 1.2 | 0.90 | 1.1 | 1.5 | 1.3 |
| IC₅₀ (nM) | IgG | 2.8 | 7.2 | 6.1 | 7.2 | 19 | 24 |
| ACE2 Blocking | IgM | 0.26 | 1.0 | 0.78 | 0.98 | 1.3 | 1.1 |
| IC₅₀ (μg/mL) | IgG | 0.41 | 1.0 | 0.88 | 1.0 | 2.8 | 3.5 |

TABLE 9
Binding Kinetics and ACE2 Blocking Assay Results for Ab10

| | | RBD | | | | | |
|---|---|---|---|---|---|---|---|
| Ab10 | Ab Format | WT | K417E | G446V | Y453F | E484K | Q493K |
| Avidity | IgM | <0.001 | <0.001 | 0.11 | <0.001 | <0.001 | <0.001 |
| KD (nM) | IgG | <0.001 | <0.001 | 120 | <0.001 | <0.001 | 0.60 |
| ACE2 Blocking | IgM | 0.27 | 0.32 | 5.0 | 0.49 | 0.47 | 0.35 |
| IC₅₀ (nM) | IgG | 3.2 | 3.8 | >69 | 8.6 | 10 | 4.4 |
| ACE2 Blocking | IgM | 0.23 | 0.28 | 4.3 | 0.42 | 0.41 | 0.30 |
| IC₅₀ (μg/mL) | IgG | 0.46 | 0.55 | >10 | 1.2 | 1.5 | 0.64 |

### Example 13: Biolayer Interferometry Affinity Measurement Assay

The binding affinities & avidities of Ab3 and Ab4 IgG or IgM antibodies were determined as described in Example 11 with SARS-CoV-2 RBD (MW 26.5 KDa) or a larger number of variant RBDs. The KD values (in nM) are shown in Table 10. Underline indicates significant difference between IgM and IgG formats. NB indicates no binding.

TABLE 10
Binding Kinetics for Ab3 and Ab4

| | | Ab3 | | Ab4 | |
|---|---|---|---|---|---|
| | Ab | Avidity KD (nM) | | Avidity KD (nM) | |
| | Format | IgM | IgG | IgM | IgG |
| RBD | WT | <0.001 | <0.001 | <0.001 | <0.001 |
| | K417E | <0.001 | 1.4 | <0.001 | <0.001 |
| | G446V | <0.001 | <0.001 | <0.001 | <0.001 |
| | Y453F | 0.017 | 64 | <0.001 | <0.001 |
| | E484K | 0.004 | 0.67 | <0.001 | <0.001 |
| | N501Y | <0.001 | <0.001 | <0.001 | <0.001 |
| | S494P | <0.001 | 0.0098 | <0.001 | <0.001 |
| | G446V | <0.001 | <0.001 | 2.3 | NB |
| | F490S | <0.001 | <0.001 | <0.001 | <0.001 |
| | N439K | <0.001 | <0.001 | <0.001 | 0.48 |
| | G476S | <0.001 | <0.001 | 0.0096 | 0.020 |
| | L455F | <0.001 | 1.1 | <0.001 | <0.001 |

### Example 14: Generation of Anti-MERS-CoV Antibodies

The VH and VL regions of human anti-MERS antibodies, e.g., those listed in Table 13, e.g., the VH and VL of SEQ ID NO: 522 and SEQ ID NO: 523, SEQ ID NO: 576 and SEQ ID NO: 577, SEQ ID NO: 610 and SEQ ID NO: 611, SEQ ID NO: 612 and SEQ ID NO: 613, SEQ ID NO: 614 and SEQ ID NO: 615, or SEQ ID NO: 630 and SEQ ID NO: 631, respectively, are incorporated into IgM, IgA1, and IgA2m2 formats (which may comprise an exemplary J-chain, e.g., a human J-chain, a modified J-chain, or a functional fragment or variant thereof) and IgG format according to standard cloning protocols.

The IgM, IgA, and IgG antibody constructs are expressed in Expi293 or CHO cells. The IgM antibodies are purified according to methods described in Keyt, B., et al. Antibodies: 9:53, doi: 10.3390/antib9040053 (2020). The IgA and IgG antibodies are purified by affinity chromatography. The IgM antibodies assemble as hexamers or pentamers with a J-chain, and the IgA antibodies assembled as dimers or tetramers with a J-chain.

### Example 15: MERS-CoV Antibody Binding Measured by ELISA

Binding of anti-MERS-CoV IgM, IgA1, IgA2m2, and IgG antibodies (produced as described in Example 15) to MERS-CoV is measured in ELISA assays as follows: 96-well white polystyrene ELISA plates (Pierce 15042) are coated with 100 μL per well of 0.5 μg/mL recombinant MERS-CoV Receptor Binding Domain (RBD) with a his tag, recombinant MERS-CoV RBD with a Fc tag, or recombinant MERS-CoV spike (S) protein trimer, overnight at 4° C. Plates are then washed 5 times with 0.05% PBS-Tween and blocked with 2% BSA-PBS. After blocking, 100 μL of serial dilutions of anti-MERS-CoV IgM, IgA1, IgA2m2, or IgG antibodies; standards; or controls are added to the wells and are incubated at room temperature for 2 hours. The plates are then washed 10 times and are incubated with HRP conjugated mouse anti-human kappa (Southern Biotech, 9230-05; 1:6000 diluted in 2% BSA-PBS) for 30 min. After 10 final washes using 0.05% PBS-Tween, the plates are read using SuperSignal chemiluminescent substrate (ThermoFisher, 37070). Luminescent data is collected on an EnVision plate reader (Perkin-Elmer) and is analyzed with GraphPad Prism using a 4-parameter logistic model.

### Example 16: MERS-CoV Pseudovirus Neutralization Assay

A MERS-CoV pseudovirus neutralization assay is performed generally as described in Example 10 using recombinant pseudoviruses expressing the MERS-CoV spike protein, and various concentrations of anti-MERS-CoV antibodies are produced as described in Example 15.

Briefly, lentivirus-based MERS-CoV pseudovirus particles are generated expressing the MERS-CoV spike protein (e.g., SEQ ID NO: 18) on the surface. The pseudovirus neutralization assay is based on previously described methodologies using luciferase-expressing HIV-1 pseudovirions (Richman et al. (Proc. Natl. Acad. Sci. USA, 2003, 100(7): 4144-4149), Folegatti et al., Lancet, 2020, 396: 467-478). Briefly, neutralizing antibody (Nab) titers are determined by creating 9 serial four-fold dilutions of anti-MERS-CoV IgM, IgG, or IgA antibodies that are produced as described in Example 15, which are mixed with ˜10⁵ relative light units (RLU) of MERS-CoV pseudotyped virus and are incubated at 37° C. for one hour. Separately, irrelevant pseudotyped control virus is also mixed with test samples. Following a 1-hour incubation, human DPP4-expressing cells, e.g., HEK 293 human DPP4-transfected cells are added to the well. The plates are incubated for 60-80 hours at 37° C. and then are assayed for luciferase expression. Neutralization titers are reported as the reciprocal of the serum dilution conferring 50% inhibition (ID₅₀) of pseudovirus infection. % Inhibition=100%−(((RLU_{(Vector+Sample+Diluent)}−RLU_{(Background)})/(RLU_{(Vector+Dluent)}−RLU_{(Background)}))×100%). MERS-CoV nAb Assay Positive and Negative Control Sera are included on each 96-well assay plate. Data are analyzed using a non-linear regression curve fit (log [inhibitor] vs. normalized response−variable slope) in GraphPad Prism.

TABLE 11
Sequences of the Disclosure

| SEQ | | |
|---|---|---|
| ID | Nickname (source) | Sequence |
| 1 | Human IgM Constant | GSASAPTLFPLVSCENSPSDTSSVAVGCLAQDFLPDSITFSWKYKNNSDISSTRGFPSVLRGGKYAATSQVL |
| | region IMGT allele | LPSKDVMQGTDEHVVCKVQHPNGNKEKNVPLPVIAELPPKVSVFVPPRDGFFGNPRKSKLICQATGFSPRQI |
| | IGHM*03 (GenBank: | QVSWLREGKQVGSGVTTDQVQAEAKESGPTTYKVTSTLTIKESDWLSQSMFTCRVDHRGLTFQQNASSMCVP |
| | pir\|S37768\|) | DQDTAIRVFAIPPSFASIFLTKSTKLTCLVTDLTTYDSVTISWTRQNGEAVKTHTNISESHPNATFSAVGEA |
| | | SICEDDWNSGERFTCTVTHTDLPSPLKQTISRPKGVALHRPDVYLLPPAREQLNLRESATITCLVTGFSPAD |
| | | VFVQWMQRGQPLSPEKYVTSAPMPEPQAPGRYFAHSILTVSEEEWNTGETYTCVVAHEALPNRVTERTVDKS |
| | | TGKPTLYNVSLVMSDTAGTCY |
| 2 | Human IgM Constant | GSASAPTLFPLVSCENSPSDTSSVAVGCLAQDFLPDSITFSWKYKNNSDISSTRGFPSVLRGGKYAATSQVL |
| | region IMGT allele | LPSKDVMQGTDEHVVCKVQHPNGNKEKNVPLPVIAELPPKVSVFVPPRDGFFGNPRKSKLICQATGFSPRQI |
| | IGHM*04 (GenBank: | QVSWLREGKQVGSGVTTDQVQAEAKESGPTTYKVTSTLTIKESDWLGQSMFTCRVDHRGLTFQQNASSMCVP |
| | sp\|P01871.4\|) | DQDTAIRVFAIPPSFASIFLTKSTKLTCLVTDLTTYDSVTISWTRQNGEAVKTHTNISESHPNATFSAVGEA |
| | | SICEDDWNSGERFTCTVTHTDLPSPLKQTISRPKGVALHRPDVYLLPPAREQLNLRESATITCLVTGFSPAD |
| | | VFVQWMQRGQPLSPEKYVTSAPMPEPQAPGRYFAHSILTVSEEEWNTGETYTCVVAHEALPNRVTERTVDKS |
| | | TGKPTLYNVSLVMSDTAGTCY |
| 3 | Human IgA1 heavy chain | ASPTSPKVFPLSLCSTQPDGNVVIACLVQGFFPQEPLSVTWSESGQGVTARNFPPSQDASGDLYTTSSQLTL |
| | constant region, e.g., | PATQCLAGKSVTCHVKHYTNPSQDVTVPCPVPSTPPTPSPSTPPTPSPSCCHPRLSLHRPALEDLLLGSEAN |
| | amino acids 144 to 496 | LTCTLTGLRDASGVTFTWTPSSGKSAVQGPPERDLCGCYSVSSVLPGCAEPWNHGKTFTCTAAYPESKTPLT |
| | of GenBank AIC59035.1 | ATLSKSGNTFRPEVHLLPPPSEELALNELVTLTCLARGFSPKDVLVRWLQGSQELPREKYLTWASRQEPSQG |
| | | TTTFAVTSILRVAAEDWKKGDTFSCMVGHEALPLAFTQKTIDRLAGKPTHVNVSVVMAEVDGTCY |
| 4 | Human IgA2 heavy chain | ASPTSPKVFPLSLDSTPQDGNVVVACLVQGFFPQEPLSVTWSESGQNVTARNFPPSQDASGDLYTTSSQLTL |
| | constant region, e.g., | PATQCPDGKSVTCHVKHYTNSSQDVTVPCRVPPPPPCCHPRLSLHRPALEDLLLGSEANLTCTLTGLRDASG |
| | amino acids 1 to 340 | ATFTWTPSSGKSAVQGPPERDLCGCYSVSSVLPGCAQPWNHGETFTCTAAHPELKTPLTANITKSGNTFRPE |
| | of GenBank P01877.4 | VHLLPPPSEELALNELVTLTCLARGFSPKDVLVRWLQGSQELPREKYLTWASRQEPSQGTTTYAVTSILRVA |
| | | AEDWKKGETFSCMVGHEALPLAFTQKTIDRMAGKPTHINVSVVMAEADGTCY |
| 5 | Precursor Human | MLLFVLTCLLAVFPAISTKSPIFGPEEVNSVEGNSVSITCYYPPTSVNRHTRKYWCRQGARGGCITLISSEG |
| | Secretory Component | YVSSKYAGRANLTNFPENGTFVVNIAQLSQDDSGRYKCGLGINSRGLSFDVSLEVSQGPGLLNDTKVYTVDL |
| | | GRTVTINCPFKTENAQKRKSLYKQIGLYPVLVIDSSGYVNPNYTGRIRLDIQGTGQLLFSVVINQLRLSDAG |
| | | QYLCQAGDDSNSNKKNADLQVLKPEPELVYEDLRGSVTFHCALGPEVANVAKFLCRQSSGENCDVVVNTLGK |
| | | RAPAFEGRILLNPQDKDGSFSVVITGLRKEDAGRYLCGAHSDGQLQEGSPIQAWQLFVNEESTIPRSPTVVK |
| | | GVAGGSVAVLCPYNRKESKSIKYWCLWEGAQNGRCPLLVDSEGWVKAQYEGRLSLLEEPGNGTFTVILNQLT |
| | | SRDAGFYWCLTNGDTLWRTTVEIKIIEGEPNLKVPGNVTAVLGETLKVPCHFPCKFSSYEKYWCKWNNTGCQ |
| | | ALPSQDEGPSKAFVNCDENSRLVSLTLNLVTRADEGWYWCGVKQGHFYGETAAVYVAVEERKAAGSRDVSLA |
| | | KADAAPDEKVLDSGFREIENKAIQDPRLFAEEKAVADTRDQADGSRASVDSGSSEEQGGSSRALVSTLVPLG |
| | | LVLAVGAVAVGVARARHRKNVDRVSIRSYRTDISMSDFENSREFGANDNMGASSITQETSLGGKEEFVATTE |
| | | STTETKEPKKAKRSSKEEAEMAYKDFLLQSSTVAAEAQDGPQEA |
| 6 | Precursor Human J | MKNHLLFWGVLAVFIKAVHVKAQEDERIVLVDNKCKCARITSRIIRSSEDPNEDIVERNIRIIVPLNNRENI |
| | Chain | SDPTSPLRTRFVYHLSDLCKKCDPTEVELDNQIVTATQSNICDEDSATETCYTYDRNKCYTAVVPLVYGGET |
| | | KMVETALTPDACYPD |
| 7 | Mature Human J Chain | QEDERIVLVDNKCKCARITSRIIRSSEDPNEDIVERNIRIIVPLNNRENISDPTSPLRTRFVYHLSDLCKKC |
| | | DPTEVELDNQIVTATQSNICDEDSATETCYTYDRNKCYTAVVPLVYGGETKMVETALTPDACYPD |
| 8 | J Chain Y102A mutation | QEDERIVLVDNKCKCARITSRIIRSSEDPNEDIVERNIRIIVPLNNRENISDPTSPLRTRFVYHLSDLCKKC |
| | | DPTEVELDNQIVTATQSNICDEDSATETCATYDRNKCYTAVVPLVYGGETKMVETALTPDACYPD |
| 9 | “5” Peptide linker | GGGGS |
| 10 | “10” Peptide linker | GGGGSGGGGS |
| 11 | “15” Peptide linker | GGGGSGGGGSGGGGS |
| 12 | “20” Peptide linker | GGGGSGGGGSGGGGSGGGGS |
| 13 | “25” Peptide linker | GGGGSGGGGSGGGGSGGGGSGGGGS |
| 14 | human ACE2 UniprotKB | >sp\|Q9BYF1\|ACE2_HUMAN Angiotensin-converting enzyme 2 |
| | Q9BYF1 | OS = Homo sapiens OX = 9606 GN = ACE2 PE = 1 SV = 2 |
| | | MSSSSWLLLSLVAVTAAQSTIEEQAKTFLDKFNHEAEDLFYQSSLASWNYNTNITEENVQ |
| | | NMNNAGDKWSAFLKEQSTLAQMYPLQEIQNLTVKLQLQALQQNGSSVLSEDKSKRLNTIL |
| | | NTMSTIYSTGKVCNPDNPQECLLLEPGLNEIMANSLDYNERLWAWESWRSEVGKQLRPLY |
| | | EEYVVLKNEMARANHYEDYGDYWRGDYEVNGVDGYDYSRGQLIEDVEHTFEEIKPLYEHL |
| | | HAYVRAKLMNAYPSYISPIGCLPAHLLGDMWGRFWINLYSLTVPFGQKPNIDVTDAMVDQ |
| | | AWDAQRIFKEAEKFFVSVGLPNMTQGFWENSMLTDPGNVQKAVCHPTAWDLGKGDFRILM |
| | | CTKVTMDDFLTAHHEMGHIQYDMAYAAQPFLLRNGANEGFHEAVGEIMSLSAATPKHLKS |
| | | IGLLSPDFQEDNETEINFLLKQALTIVGILPFTYMLEKWRWMVFKGEIPKDQWMKKWWEM |
| | | KREIVGVVEPVPHDETYCDPASLFHVSNDYSFIRYYTRTLYQFQFQEALCQAAKHEGPLH |
| | | KCDISNSTEAGQKLFNMLRLGKSEPWILALENVVGAKNMNVRPLLNYFEPLFTWLKDQNK |
| | | NSFVGWSTDWSPYADQSIKVRISLKSALGDKAYEWNDNEMYLFRSSVAYAMRQYFLKVKN |
| | | QMILFGEEDVRVANLKPRISFNFFVTAPKNVSDIIPRTEVEKAIRMSRSRINDAFRLNDN |
| | | SLEFLGIQPTLGPPNQPPVSIWLIVFGVVMGVIVVGIVILIFTGIRDRKKKNKARSGENP |
| | | YASIDISKGENNPGFQNTDDVQTSF |
| 15 | human ACE2 | QSTIEEQAKTFLDKFNHEAEDLFYQSSLASWNYNTNITEENVQNMNNAGDKWSAFLKEQS |
| | Extracellular | TLAQMYPLQEIQNLTVKLQLQALQQNGSSVLSEDKSKRLNTILNTMSTIYSTGKVCNPDN |
| | domain-variant human | PQECLLLEPGLNEIMANSLDYNERLWAWESWRSEVGKQLRPLYEEYVVLKNEMARANHYE |
| | IgG1 hinge region- | DYGDYWRGDYEVNGVDGYDYSRGQLIEDVEHTFEEIKPLYEHLHAYVRAKLMNAYPSYIS |
| | Cmu3, 4tp human P311A, | PIGCLPAHLLGDMWGRFWTNLYSLTVPFGQKPNIDVTDAMVDQAWDAQRIFKEAEKFFVS |
| | P313S | VGLPNMTQGFWENSMLTDPGNVQKAVCHPTAWDLGKGDFRILMCTKVTMDDFLTAHHEMG |
| | | HIQYDMAYAAQPFLLRNGANEGFHEAVGEIMSLSAATPKHLKSIGLLSPDFQEDNETEIN |
| | | FLLKQALTIVGTLPFTYMLEKWRWMVFKGEIPKDQWMKKWWEMKREIVGVVEPVPHDETY |
| | | CDPASLFHVSNDYSFIRYYTRTLYQFQFQEALCQAAKHEGPLHKCDISNSTEAGQKLFNM |
| | | LRLGKSEPWTLALENVVGAKNMNVRPLLNYFEPLFTWLKDQNKNSFVGWSTDWSPYADQS |
| | | IKVRISLKSALGDKAYEWNDNEMYLFRSSVAYAMRQYFLKVKNQMILFGEEDVRVANLKP |
| | | RISFNFFVTAPKNVSDIIPRTEVEKAIRMSRSRINDAFRLNDNSLEFLGIQPILGPPNQP |
| | | PVSVEPKSSDKTHTCPPCPAPDQDTAIRVFAIPPSFASIFLTKSTKLTCLVTDLTTYDSV |
| | | TISWTRQNGEAVKTHTNISESHPNATFSAVGEASICEDDWNSGERFTCTVTHTDLASSLK |
| | | QTISRPKGVALHRPDVYLLPPAREQLNLRESATITCLVTGFSPADVFVQWMQRGQPLSPE |
| | | KYVTSAPMPEPQAPGRYFAHSILTVSEEEWNTGETYTCVVAHEALPNRVTERTVDKSTGK |
| | | PTLYNVSLVMSDTAGTCY |
| 16 | SARS CoV Spike | >sp\|P59594\|SPIKE_SARS Spike glycoprotein OS = Severe acute |
| | Protein | respiratory syndrome coronavirus OX=694009 GN = S PE = 1 SV = 1 |
| | | MFIFLLFLTLTSGSDLDRCTTFDDVQAPNYTQHTSSMRGVYYPDEIFRSDTLYLTQDLFL |
| | | PFYSNVTGFHTINHTFGNPVIPFKDGIYFAATEKSNVVRGWVFGSTMNNKSQSVIIINNS |
| | | TNVVIRACNFELCDNPFFAVSKPMGTQTHTMIFDNAFNCTFEYISDAFSLDVSEKSGNFK |
| | | HLREFVFKNKDGFLYVYKGYQPIDVVRDLPSGFNTLKPIFKLPLGINITNFRAILTAFSP |
| | | AQDIWGTSAAAYFVGYLKPTTFMLKYDENGTITDAVDCSQNPLAELKCSVKSFEIDKGIY |
| | | QTSNFRVVPSGDVVRFPNITNLCPFGEVFNATKFPSVYAWERKKISNCVADYSVLYNSTF |
| | | FSTFKCYGVSATKLNDLCFSNVYADSFVVKGDDVRQIAPGQTGVIADYNYKLPDDFMGCV |
| | | LAWNTRNIDATSTGNYNYKYRYLRHGKLRPFERDISNVPFSPDGKPCTPPALNCYWPLND |
| | | YGFYTTTGIGYQPYRVVVLSFELLNAPATVCGPKLSTDLIKNQCVNFNFNGLTGTGVLTP |
| | | SSKRFQPFQQFGRDVSDFTDSVRDPKTSEILDISPCSFGGVSVITPGTNASSEVAVLYQD |
| | | VNCTDVSTAIHADQLTPAWRIYSTGNNVFQTQAGCLIGAEHVDTSYECDIPIGAGICASY |
| | | HTVSLLRSTSQKSIVAYTMSLGADSSIAYSNNTIAIPTNFSISITTEVMPVSMAKTSVDC |
| | | NMYICGDSTECANLLLQYGSFCTQLNRALSGIAAEQDRNTREVFAQVKQMYKTPTLKYFG |
| | | GFNFSQILPDPLKPTKRSFIEDLLFNKVTLADAGFMKQYGECLGDINARDLICAQKFNGL |
| | | TVLPPLLTDDMIAAYTAALVSGTATAGWTFGAGAALQIPFAMQMAYRFNGIGVTQNVLYE |
| | | NQKQIANQFNKAISQIQESLTTTSTALGKLQDVVNQNAQALNTLVKQLSSNFGAISSVLN |
| | | DILSRLDKVEAEVQIDRLITGRLQSLQTYVTQQLIRAAEIRASANLAATKMSECVLGQSK |
| | | RVDFCGKGYHLMSFPQAAPHGVVFLHVTYVPSQERNFTTAPAICHEGKAYFPREGVFVFN |
| | | GTSWFITQRNFFSPQIITTDNTFVSGNCDVVIGIINNTVYDPLQPELDSFKEELDKYFKN |
| | | HTSPDVDLGDISGINASVVNIQKEIDRLNEVAKNLNESLIDLQELGKYEQYIKWPWYVWL |
| | | GFIAGLIAIVMVTILLCCMTSCCSCLKGACSCGSCCKFDEDDSEPVLKGVKLHYT |
| 17 | SARS-CoV-2 Spike (S) | MFVFLVLLPLVSSQCVNLTTRTQLPPAYTNSFTRGVYYPDKVFRSSVLHSTQDLFLPFFS |
| | Protein, UniProt | NVTWFHAIHVSGTNGTKRFDNPVLPFNDGVYFASTEKSNIIRGWIFGTTLDSKTQSLLIV |
| | P0DTC2 | NNATNVVIKVCEFQFCNDPFLGVYYHKNNKSWMESEFRVYSSANNCTFEYVSQPFLMDLE |
| | | GKQGNFKNLREFVFKNIDGYFKIYSKHTPINLVRDLPQGFSALEPLVDLPIGINITRFQT |
| | | LLALHRSYLTPGDSSSGWTAGAAAYYVGYLQPRTFLLKYNENGTITDAVDCALDPLSETK |
| | | CTLKSFTVEKGIYQTSNFRVQPTESIVRFPNITNLCPFGEVFNATRFASVYAWNRKRISN |
| | | CVADYSVLYNSASFSTFKCYGVSPTKLNDLCFTNVYADSFVIRGDEVRQIAPGQTGKIAD |
| | | YNYKLPDDFTGCVIAWNSNNLDSKVGGNYNYLYRLFRKSNLKPFERDISTEIYQAGSTPC |
| | | NGVEGFNCYFPLQSYGFQPTNGVGYQPYRVVVLSFELLHAPATVCGPKKSTNLVKNKCVN |
| | | FNFNGLTGTGVLTESNKKFLPFQQFGRDIADTTDAVRDPQTLEILDITPCSFGGVSVITP |
| | | GTNTSNQVAVLYQDVNCTEVPVAIHADQLTPTWRVYSIGSNVFQTRAGCLIGAEHVNNSY |
| | | ECDIPIGAGICASYQTQTNSPRRARSVASQSIIAYTMSLGAENSVAYSNNSIAIPTNFTI |
| | | SVTTEILPVSMTKTSVDCTMYICGDSTECSNLLLQYGSFCTQLNRALTGIAVEQDKNTQE |
| | | VFAQVKQIYKTPPIKDFGGFNFSQILPDPSKPSKRSFIEDLLFNKVTLADAGFIKQYGDC |
| | | LGDIAARDLICAQKFNGLTVLPPLLTDEMIAQYTSALLAGTITSGWTFGAGAALQIPFAM |
| | | QMAYRFNGIGVTQNVLYENQKLIANQFNSAIGKIQDSLSSTASALGKLQDVVNQNAQALN |
| | | TLVKQLSSNFGAISSVLNDILSRLDKVEAEVQIDRLITGRLQSLQTYVTQQLIRAAEIRA |
| | | SANLAATKMSECVLGQSKRVDFCGKGYHLMSFPQSAPHGVVFLHVTYVPAQEKNFTTAPA |
| | | ICHDGKAHFPREGVFVSNGTHWFVTQRNFYEPQIITTDNTFVSGNCDVVIGIVNNTVYDP |
| | | LQPELDSFKEELDKYFKNHTSPDVDLGDISGINASVVNIQKEIDRLNEVAKNLNESLIDL |
| | | QELGKYEQYIKWPWYIWLGFIAGLIAIVMVTIMLCCMTSCCSCLKGCCSCGSCCKFDEDD |
| | | SEPVLKGVKLHYT |
| 18 | MERS Spike (S) | >sp\|K9N5Q8\|SPIKE_MERS1 Spike glycoprotein OS = Middle East |
| | Protein, | respiratory syndrome-related coronavirus (isolate United |
| | UniProtKB-K9N5Q8 | Kingdom/H123990006/2012) OX=1263720 GN = S PE = 1 SV = 1 |
| | (SPIKE_MERS1) | MIHSVFLLMFLLTPTESYVDVGPDSVKSACIEVDIQQTFFDKTWPRPIDVSKADGIIYPQ |
| | | GRTYSNITITYQGLFPYQGDHGDMYVYSAGHATGTTPQKLFVANYSQDVKQFANGFVVRI |
| | | GAAANSTGTVIISPSTSATIRKIYPAFMLGSSVGNFSDGKMGRFFNHTLVLLPDGCGTLL |
| | | RAFYCILEPRSGNHCPAGNSYTSFATYHTPATDCSDGNYNRNASLNSFKEYFNLRNCTFM |
| | | YTYNITEDEILEWFGITQTAQGVHLFSSRYVDLYGGNMFQFATLPVYDTIKYYSIIPHSI |
| | | RSIQSDRKAWAAFYVYKLQPLTFLLDFSVDGYIRRAIDCGFNDLSQLHCSYESFDVESGV |
| | | YSVSSFEAKPSGSVVEQAEGVECDFSPLLSGTPPQVYNFKRLVFTNCNYNLTKLLSLFSV |
| | | NDFTCSQISPAAIASNCYSSLILDYFSYPLSMKSDLSVSSAGPISQFNYKQSFSNPTCLI |
| | | LATVPHNLTTITKPLKYSYINKCSRFLSDDRTEVPQLVNANQYSPCVSIVPSTVWEDGDY |
| | | YRKQLSPLEGGGWLVASGSTVAMTEQLQMGFGITVQYGTDTNSVCPKLEFANDTKIASQL |
| | | GNCVEYSLYGVSGRGVFQNCTAVGVRQQRFVYDAYQNLVGYYSDDGNYYCLRACVSVPVS |
| | | VIYDKETKTHATLFGSVACEHISSTMSQYSRSTRSMLKRRDSTYGPLQTPVGCVLGLVNS |
| | | SLFVEDCKLPLGQSLCALPDTPSTLTPRSVRSVPGEMRLASIAFNHPIQVDQLNSSYFKL |
| | | SIPTNFSFGVTQEYIQTTIQKVTVDCKQYVCNGFQKCEQLLREYGQFCSKINQALHGANL |
| | | RQDDSVRNLFASVKSSQSSPIIPGFGGDFNLTLLEPVSISTGSRSARSAIEDLLFDKVTI |
| | | ADPGYMQGYDDCMQQGPASARDLICAQYVAGYKVLPPLMDVNMEAAYTSSLLGSIAGVGW |
| | | TAGLSSFAAIPFAQSIFYRLNGVGITQQVLSENQKLIANKFNQALGAMQTGFTTTNEAFH |
| | | KVQDAVNNNAQALSKLASELSNTFGAISASIGDIIQRLDVLEQDAQIDRLINGRLTTLNA |
| | | FVAQQLVRSESAALSAQLAKDKVNECVKAQSKRSGFCGQGTHIVSFVVNAPNGLYFMHVG |
| | | YYPSNHIEVVSAYGLCDAANPTNCIAPVNGYFIKTNNTRIVDEWSYTGSSFYAPEPITSL |
| | | NTKYVAPQVTYQNISTNLPPPLLGNSTGIDFQDELDEFFKNVSTSIPNFGSLTQINTTLL |
| | | DLTYEMLSLQQVVKALNESYIDLKELGNYTYYNKWPWYIWLGFIAGLVALALCVFFILCC |
| | | TGCGTNCMGKLKCNRCCDRYEEYDLEPHKVHVH |

TABLE 12
Antibody VHH Sequences*

| SEQ | | | | Neutral- |
|---|---|---|---|---|
| ID | VH | Source | Binds to | izes |
| 19 | QVQLVESGGGLVQAGGSLRLSCAASGFPVRKANMHWYRQAPGK | Walter, J., et al., (2020), 18 | SARS-CoV2 | |
| | EREWVAAIMSKGEQTVYADSVEGRFTISRDNAKNTVYLQMNSL | pages, bioRxiv preprint doi: | | |
| | KPEDTAVYYCRVFVGWHYFGQGTQVTVS | 10.1101/2020.04.16.045419 | | |
| 20 | QVQLVESGGGLVQAGGSLRLSCATSGFPVYQANMHWYRQAPGK | Walter, J., et al., (2020), 18 | SARS-CoV2 | |
| | EREWVAAIQSYGDGTHYADSVKGRFTISRDNAKNTVYLQMNSL | pages, bioRxiv preprint doi: | | |
| | KPEDTAVYYCRAVYVGMHYFGQGTQVTVS | 10.1101/2020.04.16.045419 | | |
| 21 | QVQLVESGGGLVQAGGSLRLSCAASGFPVNYKTMWWYRQAPGK | Walter, J., et al., (2020), 18 | SARS-CoV2 | |
| | EREWVAAIWSYGHTTHYADSVKGRFTISRDNAKNTVYLQMNSL | pages, bioRxiv preprint doi: | | |
| | KPEDTAVYYCVVWVGHNYEGQGTQVTVS | 10.1101/2020.04.16.045419 | | |
| 22 | QVQLVESGGGLVQAGGSLRLSCAASGFPVYAQNMHWYRQAPGK | Walter, J., et al., (2020), 18 | SARS-CoV2 | |
| | EREWVAAIYSHGYWTLYADSVKGRFTISRDNAKNTVYLQMNSL | pages, bioRxiv preprint doi: | | |
| | KPEDTAVYYCEVQVGAWYTGQGTQVTVS | 10.1101/2020.04.16.045419 | | |
| 23 | QVQLVESGGGLVQAGGSLRLSCAASGFPVFSGHMHWYRQAPGK | Walter, J., et al., (2020), 18 | SARS-CoV2 | |
| | EREWVAAILSNGDSTHYADSVKGRFTISRDNAKNTVYLQMNSL | pages, bioRxiv preprint doi: | | |
| | KPEDTAVYYCRVHVGAHYFGQGTQVTVS | 10.1101/2020.04.16.045419 | | |
| 24 | QVQLVESGGGLVQAGGSLRLSCAASGFPVEQGRMYWYRQAPGK | Walter, J., et al., (2020), 18 | SARS-CoV2 | |
| | EREWVAAIISHGTVTVYADSVKGRFTISRDNAKNTVYLQMNSL | pages, bioRxiv preprint doi: | | |
| | KPEDTAVYYCYVYVGAQYWGQGTQVTVS | 10.1101/2020.04.16.045419 | | |
| 25 | QVQLVESGGGLVQAGGSLRLSCAASGFPVLFTYMHWYRQAPGK | Walter, J., et al., (2020), 18 | SARS-CoV2 | SARS-CoV2 |
| | EREWVAAIWSSGNSTWYADSVKGRFTISRDNAKNTVYLQMNSL | pages, bioRxiv preprint doi: | | |
| | KPEDTAVYYCFVKVGNWYAGQGTQVTVS | 10.1101/2020.04.16.045419 | | |
| 26 | QVQLVESGGGLVQAGGSLRLSCAASGFPVNAGNMHWYRQAPGK | Walter, J., et al., (2020), 18 | SARS-CoV2 | |
| | EREWVAAIQSYGRTTYYADSVKGRFTISRDNAKNTVYLQMNSL | pages, bioRxiv preprint doi: | | |
| | KPEDTAVYYCRVFVGMHYFGQGTQVTVS | 10.1101/2020.04.16.045419 | | |
| 27 | QVQLVESGGGLVQAGGSLRLSCAASGFPVSSSTMTWYRQAPGK | Walter, J., et al., (2020), 18 | SARS-CoV2 | |
| | EREWVAAINSYGWETHYADSVKGRFTISRDNAKNTVYLQMNSL | pages, bioRxiv preprint doi: | | |
| | KPEDTAVYYCYVYVGGSYIGQGTQVTVS | 10.1101/2020.04.16.045419 | | |
| 28 | QVQLVESGGGLVQAGGSLRLSCAASGFPVQSHYMRWYRQAPGK | Walter, J., et al., (2020), 18 | SARS-CoV2 | |
| | EREWVAAIESTGHHTAYADSVKGRFTISRDNAKNTVYLQMNSL | pages, bioRxiv preprint doi: | | |
| | KPEDTAVYYCTVYVGYEYHGQGTQVTVS | 10.1101/2020.04.16.045419 | | |
| 29 | QVQLVESGGGLVQAGGSLRLSCAASGFPVETENMHWYRQAPGK | Walter, J., et al., (2020), 18 | SARS-CoV2 | |
| | EREWVAAIYSHGMWTAYADSVKGRFTISRDNTKNTVYLQMNSL | pages, bioRxiv preprint doi: | | |
| | KPEDTAVYYCEVEVGKWYFGQGTQVTVS | 10.1101/2020.04.16.045419 | | |
| 30 | QVQLVESGGGLVQAGGSLRLSCAASGFPVKASRMYWYRQAPGK | Walter, J., et al., (2020), 18 | SARS-CoV2 | SARS-CoV2 |
| | EREWVAAIQSFGEVTWYADSVKGRFTISRDNAKNTVYLQMNSL | pages, bioRxiv preprint doi: | | |
| | KPEDTAVYYCYVWVGQEYWGQGTQVTVS | 10.1101/2020.04.16.045419 | | |
| 31 | QVQLVESGGGLVQAGGSLRLSCAASGFPVYASNMHWYRQAPGK | Walter, J., et al., (2020), 18 | SARS-CoV2 | |
| | EREWVAAIESQGYMTAYADSVKGRFTISRDNAKNTVYLQMNSL | pages, bioRxiv preprint doi: | | |
| | KPEDTAVYYCWVIVGEYYVGQGTQVTVS | 10.1101/2020.04.16.045419 | | |
| 32 | QVQLVESGGGLVQAGGSLRLSCAASGFPVQAREMEWYRQAPGK | Walter, J., et al., (2020), 18 | SARS-CoV2 | SARS-CoV2 |
| | EREWVAAIKSTGTYTAYAYSVKGRFTISRDNAKNTVYLQMNSL | pages, bioRxiv preprint doi: | | |
| | KPEDTAVYYCYVYVGSSYIGQGTQVTVS | 10.1101/2020.04.16.045419 | | |
| 33 | QVQLVESGGGLVQAGGSLRLSCAASGFPVKNFEMEWYRKAPGK | Walter, J., et al., (2020), 18 | SARS-CoV2 | SARS-CoV2 |
| | EREWVAAIQSGGVETYYADSVKGRFTISRDNAKNTVYLQMNSL | pages, bioRxiv preprint doi: | | |
| | KPEDTAVYYCEVYVGRSYIGQGTQVTVS | 10.1101/2020.04.16.045419 | | |
| 34 | QVQLVESGGGLVQAGGSLRLSCAASGFPVAYKTMWWYRQAPGK | Walter, J., et al., (2020), 18 | SARS-CoV2 | SARS-CoV2 |
| | EREWVAAIESYGIKWTRYADSVKGRFTISRDNAKNTVYLQMNS | pages, bioRxiv preprint doi: | | |
| | LKPEDTAVYYCIVWVGAQYHGQGTQVTVS | 10.1101/2020.04.16.045419 | | |
| 35 | QVQLVESGGGLVQAGGSLRLSCAASGFPVAGRNMWWYRQAPGK | Walter, J., et al., (2020), 18 | SARS-CoV2 | SARS-CoV2 |
| | EREWVAAIYSSGTYTEYADSVKGRFTISRDNAKNTVYLQMNSL | pages, bioRxiv preprint doi: | | |
| | KPEDTAVYYCHVWVGSLYKGQGTQVTVS | 10.1101/2020.04.16.045419 | | |
| 36 | QVQLVESGGGLVQAGGSLRLSCAASGFPVKHARMWWYRQAPGK | Walter, J., et al., (2020), 18 | SARS-CoV2 | |
| | EREWVAAIDSHGDTTWYADSVKGRFTISRDNAKNTVYLQMNSL | pages, bioRxiv preprint doi: | | |
| | KPEDTAVYYCYVYVGASYWGQGTQVTVS | 10.1101/2020.04.16.045419 | | |
| 37 | QVQLVESGGGLVQAGGSLRLSCAASGFPVNSHEMTWYRQAPGK | Walter, J., et al., (2020), 18 | SARS-CoV2 | |
| | EREWVAAIQSTGTVTEYADSVKGRFTISRDNAKNTVYLQMNSL | pages, bioRxiv preprint doi: | | |
| | KPEDTAVYYCYVYVGSSYLGQGTQVTVS | 10.1101/2020.04.16.045419 | | |
| 38 | QVQLVESGGGLVQAGGSLRLSCAASGFPVEQREMEWYRQAPGK | Walter, J., et al., (2020), 18 | SARS-CoV2 | |
| | EREWVAAIDSNGNYTFYADSVKGRFTISRDNAKNTVYLQMNSL | pages, bioRxiv preprint doi: | | |
| | KPEDTAVYYCYVYVGKSYIGQGTQVTVS | 10.1101/2020.04.16.045419 | | |
| 39 | QVQLVESGGGLVQAGGSLRLSCAASGFPVKHHWMFWYRQAPGK | Walter, J., et al., (2020), 18 | SARS-CoV2 | |
| | EREWVAAIKSYGYGTEYADSVKGRFTISRDNAKNTVYLQMNSL | pages, bioRxiv preprint doi: | | |
| | KPEDTAVYYCFVGVGTHYAGQGTQVTVS | 10.1101/2020.04.16.045419 | | |
| 40 | QVQLVESGGGLVQAGGSLRLSCAASGFPVYAAEMEWYRQAPGK | Walter, J., et al., (2020), 18 | SARS-CoV2 | SARS-CoV2 |
| | EREWVAAISSQGTITYYADSVKGRFTISRDNAKNTVYLQMNSL | pages, bioRxiv preprint doi: | | |
| | KPEDTAVYYCEVYVGKSYIGQGTQVSVS | 10.1101/2020.04.16.045419 | | |
| 41 | QVQLVESGGGLVQAGGSLRLSCAASGFPVYAAEMEWYRQAPGK | Walter, J., et al., (2020), 18 | SARS-CoV2 | SARS-CoV2 |
| | EREWVAAISSQGTITYYADSVKGRFTISRDNAKNTVYLQMNSL | pages, bioRxiv preprint doi: | | |
| | KPEDTAVYYCEVYVGKSYIGQGTQVSVS | 10.1101/2020.04.16.045419 | | |
| 42 | QVQLVESGGGLVQAGGSLRLSCAASGFPVHAWEMAWYRQAPGK | Walter, J., et al., (2020), 18 | SARS-CoV2 | |
| | EREWVAAIRSFGSSTHYADSVKGRFTISRDNAKNTVYLQMNSL | pages, bioRxiv preprint doi: | | |
| | KPEDTAVYYCNVKDFGTHHYAYDYWGQGTQVTVS | 10.1101/2020.04.16.045419 | | |
| 43 | QVQLVESGGGLVQAGGSLRLSCAASGFPVNTWWMHWYRQAPGK | Walter, J., et al., (2020), 18 | SARS-CoV2 | |
| | EREWVAAITSWGFRTYYADSVKGRFTISRDNAKNTVYLQMNSL | pages, bioRxiv preprint doi: | | |
| | KPEDTAVYYCNVKDKGMAVQWYDYWGQGTQVTVS | 10.1101/2020.04.16.045419 | | |
| 44 | QVQLVESGGGLVQAGGSLRLSCAASGFPVYNTWMEWYRQAPGK | Walter, J., et al., (2020), 18 | SARS-CoV2 | |
| | EREWVAAITSHGYKTYYADSVKGRFTISRDNAKNTVYLQMNSL | pages, bioRxiv preprint doi: | | |
| | KPEDTAVYYCNVKDEGDMFTAYDYWGQGTQVTVS | 10.1101/2020.04.16.045419 | | |
| 45 | QVQLVESGGGLVQAGGSLRLSCAASGFPVYHSTMFWYRQAPGK | Walter, J., et al., (2020), 18 | SARS-CoV2 | |
| | EREWVAAIYSSGQHTYYADSVKGRFTISRDNAKNTVYLQMNSL | pages, bioRxiv preprint doi: | | |
| | KPEDTAVYYCNVKDSGQWRQEYDYWGQGTQVTVS | 10.1101/2020.04.16.045419 | | |
| 46 | QVQLVESGGGLVQAGGSLRLSCAASGFPVEHEMAWYRQAPGKE | Walter, J., et al., (2020), 18 | SARS-CoV2 | |
| | REWVAAIRSMGRKTLYADSVKGRFTISRDNAKNTVYLQMNSLK | pages, bioRxiv preprint doi: | | |
| | PEDTAVYYCNVKDFGYTWHEYDYWGQGTQVTVS | 10.1101/2020.04.16.045419 | | |
| 47 | QVQLVESGGGLVQAGGSLRLSCAASGFPVTMAWMWWYRQAPGK | Walter, J., et al., (2020), 18 | SARS-CoV2 | |
| | EREWVAAIRSEGVRTYYADSVKGRFTISRDNAKNTVYLQMNSL | pages, bioRxiv preprint doi: | | |
| | KPEDTAVYYCNVKDYGQAHAYYDYWGQGTQVTVS | 10.1101/2020.04.16.045419 | | |
| 48 | QVQLVESGGGLVQAGGSLRLSCAASGFPVNSHFMEWYRQAPGK | Walter, J., et al., (2020), 18 | SARS-CoV2 | |
| | EREWVAAIQHSSGFHTYYADSVKGRFTISRDNAKNTVYLQMNS | pages, bioRxiv preprint doi: | | |
| | LKPEDTAVYYCNVKDTGTTEDYDYWGQGTQVTVS | 10.1101/2020.04.16.045419 | | |
| 49 | QVQLDESGGGLVQAGGSLRLSCAASGFPVYHAWMEWYRQAPGK | Walter, J., et al., (2020), 18 | SARS-CoV2 | |
| | EREWVAAITSSGRHTYYADSVKGRFTISRDNAKNTVYLQMNSL | pages, bioRxiv preprint doi: | | |
| | KPEDTAVYYCNVKDAGRVYNSYDYWGQGTQVTVS | 10.1101/2020.04.16.045419 | | |
| 50 | QVQLVESGGGLVQAGGSLRLSCAASGFPVAHAWMEWYRQAPGK | Walter, J., et al., (2020), 18 | SARS-CoV2 | |
| | EREWVAAITSYGYKTYYADSVKGRFTISRDNAKNTVYLQMNSL | pages, bioRxiv preprint doi: | | |
| | KPEDTAVYYCNVKDTGTYRFYYDYWGQGTQVTVS | 10.1101/2020.04.16.045419 | | |
| 51 | QVQLVESGGGLVQAGGSLRLSCAASGFPVWNQTMVWYRQAPGK | Walter, J., et al., (2020), 18 | SARS-CoV2 | |
| | EREWVAAIWSMGHTYYADSVKGRFTISRDNAKNTVYLQMNSLK | pages, bioRxiv preprint doi: | | |
| | PEDTAVYYCNVKDAGVYNRYYDYWGQGTQVTVS | 10.1101/2020.04.16.045419 | | |
| 52 | QVQLVESGGGLVQAGGSLRLSCAASGFPVEHYWMEWYRQAPGK | Walter, J., et al., (2020), 18 | SARS-CoV2 | |
| | EREWVAAITSFGYRTYYADSVKGRFTISRDNAKNTVYLQMNSL | pages, bioRxiv preprint doi: | | |
| | KPEDTAVYYCNVKDWGFASHAYDYWGQGIQVTVS | 10.1101/2020.04.16.045419 | | |
| 53 | QVQLVESGGGLVQAGGSLRLSCAASGFPEIAWEMAWYRQAPGK | Walter, J., et al., (2020), 18 | SARS-CoV2 | |
| | EREWVAAIRSFGERTLYADSVKGRFTISRDNAKNTVYLQMNSL | pages, bioRxiv preprint doi: | | |
| | KPEDTAVYYCNVKDFGWQHQEYDYWGQGTQVTVS | 10.1101/2020.04.16.045419 | | |
| 54 | QVQLVESGGGLVQAGGSLRLSCAASGFPVYHAYMEWYRQAPGK | Walter, J., et al., (2020), 18 | SARS-CoV2 | |
| | EREWVAAIYSNGEHTYYADSVKGRFTISRDNAKNTVYLQMNSL | pages, bioRxiv preprint doi: | | |
| | KPEDTAVYYCNVKDSGSFNQAYDYWGQGTQVTVS | 10.1101/2020.04.16.045419 | | |
| 55 | QVQLVESGGGLVQAGGSLRLSCAASGFPVEWSHMHWYRQAPGK | Walter, J., et al., (2020), 18 | SARS-CoV2 | |
| | EREWVAAIVSKGGYTLYADSVKGRFTISRDNAKNTVYLQMNSL | pages, bioRxiv preprint doi: | | |
| | KPEDTAVYYCNVKDYGVHFKRYDYWGQGTQVTVI | 10.1101/2020.04.16.045419 | | |
| 56 | QVQLVESGGGLVQAGGSLRLSCAASGFPVFHVWMEWYRQAPGK | Walter, J., et al., (2020), 18 | SARS-CoV2 | |
| | EREWVAAIDSAGWHTYYADSVKGRFTISRDNAKNTVYLQMNSL | pages, bioRxiv preprint doi: | | |
| | KPEDTAVYYCNVKDAGNTTSAYDYWGQGTQVTVS | 10.1101/2020.04.16.045419 | | |
| 57 | QVQLVESGGGLVQAGGSLRLSCAASGFPVYYNWMEWYRQAPGK | Walter, J., et al., (2020), 18 | SARS-CoV2 | |
| | EREWVAAIHSNGDETFYADSVKGRFTISRDNAKNTVYLQMNSL | pages, bioRxiv preprint doi: | | |
| | KPEDTAVYYCNVKDIDAEAYAYDYWGQGTQVTVS | 10.1101/2020.04.16.045419 | | |
| 58 | QVQLVESGGGLVQAGGSLRLSCAASGFPVYHVWMEWYRQAPGK | Walter, J., et al., (2020), 18 | SARS-CoV2 | |
| | EREWVAAITSSGSHTYYADSVKGRFTISRDNAKNTVYLQMNSL | pages, bioRxiv preprint doi: | | |
| | KPEDTAVYYCNVKDSGQWRVQYDYWGQGTQVTVS | 10.1101/2020.04.16.045419 | | |
| 59 | QVQLVESGGGLVQAGGSLRLSCAASGFPVYWHHMHWYRQAPGK | Walter, J., et al., (2020), 18 | SARS-CoV2 | SARS-CoV2 |
| | EREWVAAIISWGWYTTYADSVKGRFTISRDNAKNTVYLQMNSL | pages, bioRxiv preprint doi: | | |
| | KPEDTAVYYCNVKDHGAQNQMYDYWGQGTQVTVS | 10.1101/2020.04.16.045419 | | |
| 60 | QVQLVESGGGLVQAGGSLRLSCAASGFPVYRDRMAWYRQAPGK | Walter, J., et al., (2020), 18 | SARS-CoV2 | SARS-CoV2 |
| | EREWVAAIYSAGQQTRYADSVKGRFTISRDNAKNTVYLQMNSL | pages, bioRxiv preprint doi: | | |
| | KPEDTAVYYCNVKDVGHHYEYYDYWGQGTQVTVS | 10.1101/2020.04.16.045419 | | |
| 61 | QVQLVESGGGLVQAGGSLRLSCAASGFPVDNGYMHWYRQAPGK | Walter, J., et al., (2020), 18 | SARS-CoV2 | SARS-CoV2 |
| | EREWVAAIDSYGWHTIYADSVKGRFTISRDNAKNTVYLQMNSL | pages, bioRxiv preprint doi: | | |
| | KPEDTAVYYCNVKDKGQMRAAYDYWGQGTQVTVS | 10.1101/2020.04.16.045419 | | |
| 62 | QVQLVESGGGLVQAGGSLRLSCAASGFPVSWHSMYWYRQAPGK | Walter, J., et al., (2020), 18 | SARS-CoV2 | |
| | EREWVAAIFSEGDWTYYADSVKGRFTISRDNAKNTVYLQMNSL | pages, bioRxiv preprint doi: | | |
| | KPEDTAVYYCNVKDYGSSYYKYDYWGQGTQVTVS | 10.1101/2020.04.16.045419 | | |
| 63 | QVQLVESGGGLVQAGGSLRLSCAASGFPVSQSVMAWYRQAPGK | Walter, J., et al., (2020), 18 | SARS-CoV2 | |
| | EREWVAAIYSKGQYTHYADSVKGRFTISRDNAKNTVYLQMNSL | pages, bioRxiv preprint doi: | | |
| | KPEDTAVYYCNVKDAGSSYWDYDYWGQGTQVTVS | 10.1101/2020.04.16.045419 | | |
| 64 | QVQLVESGGGSVQAGGSLRLSCAASGSIGQIEYLGWFRQAPGK | Walter, J., et al., (2020), 18 | SARS-CoV2 | |
| | EREGVAALNTWTGRTYYADSVKGRFTVSLDNAKNTVYLQMNSL | pages, bioRxiv preprint doi: | | |
| | KPEDTALYYCAAARWGRTKPLNTYYYSYWGQGTPVTVS | 10.1101/2020.04.16.045419 | | |
| 65 | QVQLVESGGGSVQAGGSLRLSCAASGYIDKIVYLGWFRQAPGK | Walter, J., et al., (2020), 18 | SARS-CoV2 | |
| | EREGVAALYTLSGHTYYADSVKGRFTVSLDNAKNTVYLQMNSL | pages, bioRxiv preprint doi: | | |
| | KPEDTALYYCAAATEGHAHALYRLHYYWGQGTQVTVS | 10.1101/2020.04.16.045419 | | |
| 66 | QVQLVESGGGLVQAGGSLRLSCAASGFPVYQGEMHWYRQAPGK | Walter, J., et al., (2020), 18 | SARS-CoV2 | |
| | EREWVAAIRSTGVQTWYADSVKGRFTISRDNAKNTVYLQMNSL | pages, bioRxiv preprint doi: | | |
| | KPEDTAVYYCRVWVGTHYFGQGTQVTVS | 10.1101/2020.04.16.045419 | | |
| 67 | QVQLVESGGGSVQAGGSLRLSCAASGNIQRIYYLGWFRQAPGK | Walter, J., et al., (2020), 18 | SARS-CoV2 | |
| | EREGVAALMTYTGHTYYADSVKGRFTVSLDNAKNTVYLQMNSL | pages, bioRxiv preprint doi: | | |
| | KPEDTALYYCAAAYVGAENPLPYSMYGYWGQGTQVTVS | 10.1101/2020.04.16.045419 | | |
| 68 | QVQLVESGGGSVQAGGSLRLSCAASGQISHIKYLGWFRQAPGK | Walter, J., et al., (2020), 18 | SARS-CoV2 | |
| | EREGVAALITRWGQTYYADSVKGRFTVSLDNAKNTVYLQMNSL | pages, bioRxiv preprint doi: | | |
| | KPEDTALYYCAAADYGASDPLWFIHYLYWGQGTQVTVS | 10.1101/2020.04.16.045419 | | |
| 69 | QVQLVESGGGSVQAGGSLRLSCAASGKIWTIKYLGWFRQAPGK | Walter, J., et al., (2020), 18 | SARS-CoV2 | |
| | EREGVAALMTRWGYTYYADSVKGRFTVSLDNAKNTVYLQMNSL | pages, bioRxiv preprint doi: | | |
| | KPEDTALYYCAAANYGSNFPLAEEDYWYWGQGTQVTVS | 10.1101/2020.04.16.045419 | | |
| 70 | QVQLVESGGGSVQAGGSLRLSCAASGNISQIHYLGWFRQAPGK | Walter, J., et al., (2020), 18 | SARS-CoV2 | |
| | EREGVAALNTDYGYTYYADSVKGRFTVSLDNAKNTVYLQMNSL | pages, bioRxiv preprint doi: | | |
| | KPEDTALYYCAAAYYFGDDIPLWWEAYSYWGQGTQVTVS | 10.1101/2020.04.16.045419 | | |
| 71 | QVQLVESGGGSVQAGGSLRLSCAASGNISTIEYLGWFRQAPGK | Walter, J., et al., (2020), 18 | SARS-CoV2 | |
| | EREGVAALYTWHGQTYYADSVKGRFTVSLDNAKNTVYLQMNSL | pages, bioRxiv preprint doi: | | |
| | KPEDTALYYCAAARWGRHMPLSATEYSYWGQGTQVTVS | 10.1101/2020.04.16.045419 | | |
| 72 | QVQLVESGGGSVQAGGSLRLSCAASGNIESIYYLGWFRQAPGK | Walter, J., et al., (2020), 18 | SARS-CoV2 | |
| | EREGVAALWTGDGETYYADSVKGRFTVSLDNAKNTVYLQMNSL | pages, bioRxiv preprint doi: | | |
| | KPEDTALYYCAAAAWGNSAPLTTYRYYYWGQGTQVTVS | 10.1101/2020.04.16.045419 | | |
| 73 | QVQLVESGGGSVQAGGSLRLSCAASGFIYGITYLGWFRQAPGK | Walter, J., et al., (2020), 18 | SARS-CoV2 | |
| | EREGVAALVTWNGQTYYADSVKGRFTVSLDNAKNTVYLQMNSL | pages, bioRxiv preprint doi: | | |
| | KPEDTALYYCAAADWGYDWPLWDEWYWYWGQGTQVTVS | 10.1101/2020.04.16.045419 | | |
| 74 | QVQLVESGGGSVQAGGSLRLSCAASGTIADIKYLGWFRQAPGK | Walter, J., et al., (2020), 18 | SARS-CoV2 | SARS-CoV2 |
| | EREGVAALMTRWGSTYYADSVKGRFTVSLDNAKNTVYLQMNSL | pages, bioRxiv preprint doi: | | |
| | KPEDTALYYCAAANYGANYPLYSQQYSYWGQGTQVTVS | 10.1101/2020.04.16.045419 | | |
| 75 | QVQLVESGGGSVQAGGSLRLSCAASGSISSIKYLGWFRQAPGK | Walter, J., et al., (2020), 18 | SARS-CoV2 | |
| | EREGVAALMTRWGMTYYADSVKGRFTVSLDNAKNTVYLQMNSL | pages, bioRxiv preprint doi: | | |
| | KPEDTALYYCAAANYGANEPLQYTHYNYWGQGTQVTVS | 10.1101/2020.04.16.045419 | | |
| 76 | QVQLVESGGGSVQAGGSLRLSCAASGEIESIFYLGWFRQAPGK | Walter, J., et al., (2020), 18 | SARS-CoV2 | |
| | EREGVAALYTYVGQTYYADSVKGRFTVSLDNAKNTVYLQMNSL | pages, bioRxiv preprint doi: | | |
| | KPEDTALYYCAAASYGAAHPLSIMRYYYWGQGTQVTVS | 10.1101/2020.04.16.045419 | | |
| 77 | QVQLVESGGGSVQAGGSLRLSCAASGTIAHIKYLGWFRQAPGK | Walter, J., et al., (2020), 18 | SARS-CoV2 | |
| | EREGVAALMTKWGQTYYADSVKGRFTVSLDNAKNTVYLQMNSL | pages, bioRxiv preprint doi: | | |
| | KPEDTALYYCAAASYGANFPLKASDYSYWGQGTQVTVS | 10.1101/2020.04.16.045419 | | |
| 78 | QVQLVESGGGSVQAGGSLRLSCAASGSIQAITYLGWFRQAPGK | Walter, J., et al., (2020), 18 | SARS-CoV2 | |
| | EREGVAALVTWNGQTYYADSVKGRFTVSLDNAKNTVYLQMNSL | pages, bioRxiv preprint doi: | | |
| | KPEDTALYYCAAADWGYDWPLWDEWYWYWGQGTQVTVS | 10.1101/2020.04.16.045419 | | |
| 79 | QVQLVESGGGSVQAGGSLRLSCAASGSISSITYLGWFRQAPGK | Walter, J., et al., (2020), 18 | SARS-CoV2 | |
| | EREGVAALVTYSGNTYYADSVKGRFTVSLDNAKNTVYLQMNSL | pages, bioRxiv preprint doi: | | |
| | KPEDTALYYCAAATWGHSWPLYNDEYWYWGQGSQVTVS | 10.1101/2020.04.16.045419 | | |
| 80 | QVQLVESGGGSVQAGGSLRLSCAASGSISSITYLGWFRQAPGK | Walter, J., et al., (2020), 18 | SARS-CoV2 | SARS-CoV2 |
| | EREGVAALITVNGHTYYADSVKGRFTVSLDNAKNTVYLQMNSL | pages, bioRxiv preprint doi: | | |
| | KPEDTALYYCAAAAWGYAWPLHQDDYWYWGQGTQVTVS | 10.1101/2020.04.16.045419 | | |
| 81 | QVQLVESGGGSVQAGGSLRLSCAASGSISSITYLGWFRQAPGK | Walter, J., et al., (2020), 18 | SARS-CoV2 | |
| | EREGVAALNTFNGTTYYADSVKGRFTVSLDNAKNTVYLQMNSL | pages, bioRxiv preprint doi: | | |
| | KPEDTALYYCAAATWGYSWPLIAEYNWYWGQGTQVTVS | 10.1101/2020.04.16.045419 | | |
| 82 | QVQLVESGGGSVQAGGSLRLSCAASGSISSITYLGWFRQAPGK | Walter, J., et al., (2020), 18 | SARS-CoV2 | |
| | EREGVAALKTQAGFTYYADSVKGRFTVSLDNAKNTVYLQMNSL | pages, bioRxiv preprint doi: | | |
| | KPEDTALYYCAAANWGYSWPLYEADDWYWGQGTQVTVS | 10.1101/2020.04.16.045419 | | |
| 83 | QVQLQESGGGLVQAGGSLRLSCAASGRTFSEYAMGWFRQAPGK | Daniel Wrapp et al., 2020 | SARS- | SARS-CoV2 |
| | EREFVATISWSGGSTYYTDSVKGRFTISRDNAKNTVYLQMNSL | (www.sciencedirect.com/ | CoV1, | and SARS- |
| | KPDDTAVYYCAAAGLGTVVSEWDYDYDYWGQGTQVTVSS | science/article/pii/ | SARS-CoV2 | CoV1 |
| | | S0092867420304943) | | |

*Sequence, binding, and neutralization data derived from the CoV-AbDab database: opig.stats.ox.ac.uk/webapps/covabdab/(last visited July 5, 2021) (Raybould, MIJ, et al., Bioinformatics doi = 10.1093/bioinformatics/btaa739 (2020)).

TABLE 13
Antibody VH and VL Sequences*

| SEQ | | SEQ | | | Neutral- | |
|---|---|---|---|---|---|---|
| ID | VH | ID | VL | Binds to | izes | Source |
| 84 | QMQLVQSGTEVKKPGESLKISCKG | 85 | DIQLTQSPDSLAVSLGERATIN | SARS- | SARS- | Meulen, J., et al., |
| | SGYGFITYWIGWVRQMPGKGLEW | | CKSSQSVLYSSINKNYLAWYQ | CoV1, | CoV1 | (2006), PLoS |
| | MGIIYPGDSETRYSPSFQGQVTISA | | QKPGQPPKLLIYWASTRESGV | SARS- | | Medicine, Vol. 3(7): |
| | DKSINTAYLQWSSLKASDTAIYYC | | PDRFSGSGSGTDFTLTISSLQAE | CoV2 | | e237, 1071-1079 |
| | AGGSGISTPMDVWGQGTTVTVSS | | DVAVYYCQQYYSTPYTFGQG | | | |
| | | | TKVEIK | | | |
| 86 | EVQLVQSGAEVKKPGASVKVSCK | 87 | DIVMTQTPATLSLSPGERATLS | SARS- | SARS- | Shi, R., et al., Nature |
| | ASGYTFTSYGISWVRQAPGQGLE | | CRASQSVSSYLAWYQQKPGQ | CoV2 | CoV2 | 584: 120-124 (2020) |
| | WMGWISAYNGNTNYAQKLQGRV | | APRLLIYDASNRATGIPARFSG | | | |
| | TMTTDTSTSTAYMELRSLRSDDTA | | SGSGTDFTLTISSLEPEDFAVY | | | |
| | VYYCAREGYCSGGSCYSGYYYYY | | YCQQRRNWGTFGPGTKVDIK | | | |
| | GMDVWGQGTTVTVSS | | | | | |
| 88 | EVQLVESGGGLVQPGGSLRLSCAA | 89 | DIVMTQSPSSLSASVGDRVTIT | SARS- | SARS- | Shi, R., et al., Nature |
| | SGFTVSSNYMSWVRQAPGKGLEW | | CRASQSISRYLNWYQQKPGKA | CoV2 | CoV2 | 584: 120-124 (2020) |
| | VSVIYSGGSTFYADSVKGRFTISRD | | PKLLIYAASSLQSGVPSRFSGS | | | |
| | NSMNTLFLQMNSLRAEDTAVYYC | | GSGTDFTLTISSLQPEDFATYY | | | |
| | ARVLPMYGDYLDYWGQGTLVTV | | CQQSYSTPPEYTFGQGTKLEIK | | | |
| | SS | | | | | |
| 90 | EVQLVESGGGVVQPGRSLRLSCAA | 91 | DIQLTQSPSSLSASVGDRVTITC | SARS- | SARS- | Robbiani, D., et al., |
| | SGFTFSIYGMHWVRQAPGKGLEW | | RASQSISSYLNWYQQKPGKAP | CoV2 | CoV2 | Nature 584: 437-442 |
| | VAVISYDGSNKYYADSVKGRFTIS | | KLLIYAASSLQSGVPSRFSGSG | | | (2020) |
| | RDNSKNTLYLQMNSLRAEDTAVY | | SGTDFTLTISSLQPEDFATYYC | | | |
| | YCAKEGRPSDIVVVVAFDYWGQG | | QQSYSTPRTFGQGTKVEIK | | | |
| | TLVTVSS | | | | | |
| 92 | EVQLVESGGGLIQPGGSLRLSCAA | 93 | EIVLTQSPGTLSLSPGERATLSC | SARS- | SARS- | Robbiani, D., et al., |
| | SGFTVSSNYMSWVRQAPGKGLEW | | RASQSVSSTYLAWYQQKPGQ | CoV2 | CoV2 | Nature 584: 437-442 |
| | VSVIYSGGSTYYADSVKGRFTISRD | | APRLLIYGASSRATGIPDRFSGS | | (weak) | (2020) |
| | NSKNTLYLQMNSLRAGDTAVYYC | | GSGTDFTLTISRLEPEDFAVYY | | | |
| | ARDYGDFYFDYWGQGTLVTVSS | | CQQYGSSPRTFGQGTKLEIK | | | |
| 94 | QVQLVQSGAEVKKPGASVKVSCK | 95 | AIRMTQSPSSLSASVGDRVTIT | SARS- | SARS- | Robbiani, D., et al., |
| | ASGYTFTGYYMHWVRQAPGQGLE | | CQASQDISNYLNWYQQKPGK | CoV2 | CoV2 | Nature 584: 437-442 |
| | WMGWINPISGGTNYAQKFQGRVT | | APKLLIYDASNLETGVPSRFSG | | | (2020) |
| | MTRDTSISTAYMELSRLRSDDTAV | | SGSGTDFTFTISSLQPEDIATYY | | | |
| | YYCASPASRGYSGYDHGYYYYMD | | CQQYDNLPITFGQGTRLEIK | | | |
| | VWGKGTTVTVSS | | | | | |
| 96 | QVQLVQSGPEVKKPGTSVKVSCK | 97 | EIVLTQSPGTLSLSPGERATLSC | SARS- | SARS- | Robbiani, D., et al., |
| | ASGFTFTSSAVQWVRQARGQRLE | | RASQSVRSSYLAWYQQKPGQ | CoV2 | CoV2 | Nature 584: 437-442 |
| | WIGWIVVGSGNTNYAQKFQERVTI | | APRLLIYGASSRATGIPDRFSGS | | | (2020) |
| | TRDMSTSTAYMELSSLRSEDTAVY | | GSGTDFTLTISRLEPEDFAVYY | | | |
| | YCAAPHCSGGSCLDAFDIWGQGT | | CQQYGSSPWTFGQGTKVEIK | | | |
| | MVTVSS | | | | | |
| 98 | QVQLVESGGGLVKPGGSLRLSCAA | 99 | QSVLTQPPSASGTPGQRVTVSC | SARS- | SARS- | Robbiani, D., et al., |
| | SGFIFSDYCMSWIRRAPGKGLEWL | | SGSSSNIGSNTVNWYQQLPGT | CoV2 | CoV2 | Nature 584: 437-442 |
| | SYISNSGTTRYYADSVKGRFTISRD | | APKLLIYSNNQRPSGVPDRFSG | | (weak) | (2020) |
| | NGRNSLYLQMDSLSAEDTAVYYC | | SKSGTSASLAISGLQSEDEADY | | | |
| | ARRGDGSSSIYYYNYMDVWGKGT | | FCAAWDDSLNGPVFGGGTKL | | | |
| | TVTVSS | | TVL | | | |
| 100 | EVQLVESGGGVVQPGRSLRLSCAA | 101 | DIQMTQSPSTLSASVGDRVTIT | SARS- | SARS- | Robbiani, D., et al., |
| | SGFTFSSYGMHWVRQAPGKGLEW | | CRANQSISSWLAWYQQKPGK | CoV2 | CoV2 | Nature 584: 437-442 |
| | VTVISYDGRNKYYADSVKGRFTIS | | APKLLIYKASSLESGVPSRFSG | | (weak) | (2020) |
| | RDNSKNTLYLQMNSLRAEDTAVY | | SGSGTEFTLTISSLQPDDFATY | | | |
| | YCAREFGDPEWYFDYWGQGTLVT | | YCQQYNSYWTFGQGTKVEIK | | | |
| | VSS | | | | | |
| 102 | QVQLVQSGAEVKKPGASVKVSCM | 103 | QSALTQPPSASGSPGQSVTISC | SARS- | SARS- | Robbiani, D., et al., |
| | ASGYTFTGYYMHWVRQAPGQGLE | | TGTSSDVGGYNYVSWYQQHP | CoV2 | CoV2 | Nature 584: 437-442 |
| | WMGWINPNSGGTNYAQKFQGRV | | GKAPKLMIYEVSKRPSGVPDR | | | (2020) |
| | TMTRDTSISTAYMELSRLRSDDTA | | FSGSKSGNTASLTVSGLQAED | | | |
| | VYYCARDSPFSALGASNDYWGQG | | EAEYYCSSDAGSNNVVFGGGT | | | |
| | TLVTVSS | | KLTVL | | | |
| 104 | EVQLVESGGGVVQPGRSLRLSCAA | 105 | DIQLTQSPSSLSASVGDRVTITC | SARS- | | Robbiani, D., et al., |
| | SGFTFSSYAMHWVRQAPAKGLEW | | RASQSISTYLNWYQQKPGKAP | CoV2 | | Nature 584: 437-442 |
| | VAVILYDGSGKYYADSVKGRFTIS | | KLLIYAASSLQSGVPSRFSGSG | | | (2020) |
| | RDNSKNTLYLQMNSLRAEDTAVY | | SGTDFTLTISSLQPEDFATYYC | | | |
| | YCARDGIVDTALVTWFDYWGQGT | | QQSYSTPPWTFGQGTKVEIK | | | |
| | LVTVSS | | | | | |
| 106 | QVQLVQSGAEVKKPGSSVKVSCK | 107 | EIVLTQSPATLSLSPGERATLSC | SARS- | SARS- | Robbiani, D., et al., |
| | ASGGTFSSYAISWVRQAPGQGLEW | | RASQSVSSYLAWYQQKPGQA | CoV2 | CoV2 | Nature 584: 437-442 |
| | MGGIIPIFGTANYAQKFQGRVTITA | | PRLLIYDASNRATGIPARFSGS | | (weak) | (2020) |
| | DESTSTAYMELSSLRSEDTAVYYC | | GSGTDFTLTISSLEPEDFAVYY | | | |
| | ARGNRLLYCSSTSCYLDAVRQGY | | CQQRSNWPLTFGGGTKVEIK | | | |
| | YYYYYMDVWGKGTTVTVSS | | | | | |
| 108 | EVQLVESGGGVVQPGRSLRLSCAA | 109 | AIRMTQSPSSLSASVGDRVTIT | SARS- | | Robbiani, D., et al., |
| | SGFTFSRYGMHWVRQAPGKGLEW | | CQASQDISNYLNWYQQKPGK | CoV2 | | Nature 584: 437-442 |
| | VAVISYDGSNKYYADSVKGRFTIS | | APKLLIYDASNLETGVPSRFSG | | | (2020) |
| | RDNSKNTLYLQMNSLRAEDTAVY | | SGSGTDFTFTINSLQPEDIATYY | | | |
| | YCAKVTAPYCSGGSCYGGNFDYW | | CQQYDNLPPTFGGGTKVEIK | | | |
| | GQGTLVTVSS | | | | | |
| 110 | EVQLVESGGGLVQPGRSLRLSCAA | 111 | EIVLTQSPATLSLSPGERATLSC | SARS- | SARS- | Robbiani, D., et al., |
| | SGFTFDDYAMHWVRQAPGKGLE | | RASQSVSSYLAWYQQKPGQA | CoV2 | CoV2 | Nature 584: 437-442 |
| | WVSGISWNSGTIGYADSVKGRFTI | | PRLLIYDASNRATGIPARFSGS | | | (2020) |
| | SRDNAKNSLYLQMNSLRAEDTAF | | GSGTDFTLTISSLEPEDFAVYY | | | |
| | YYCAKAGVRGIAAAGPDLNFDHW | | CQQRITFGQGTRLEIK | | | |
| | GQGTLVTVSS | | | | | |
| 112 | EVQLVESGGGVVQPGRSLRLSCAA | 113 | DIQLTQSPSSLSASVGDRVTITC | SARS- | | Robbiani, D., et al., |
| | SGFTFSNYAIHWVRQAPGKGLEW | | RASQSIRSYLNWYQQKPGKAP | CoV2 | | Nature 584: 437-442 |
| | VAVISYDGSNKYYADSVKGRFTIS | | KLLIYAASSLQSGVPSRFSGSG | | | (2020) |
| | RDNSKNTLYLQMNSLRAEDTAVY | | SGTDFTLTISSLQPDDFATYYC | | | |
| | YCARDFDDSSFWAFDYWGQGTLV | | QQSYSTPPATFGQGTKLEIK | | | |
| | TVSS | | | | | |
| 114 | QVQLVQSGAEVKKPGASVKVSCK | 115 | SYELTQPPSVSVAPGKTARITC | SARS- | | Robbiani, D., et al., |
| | ASGYTFTSYYMHWVRQAPGQGLE | | GENNIGSKSVHWYQQKPGQA | CoV2 | | Nature 584: 437-442 |
| | WMGIINPSGGSTSYAQKFQGRVTM | | PVLVIYYDSDRPSGIPERFSGSN | | | (2020) |
| | TRDTSTSTVYMELSSLRSEDTAVY | | SGNTATLTINRVEAGDEADYY | | | |
| | YCARVPREGTPGFDPWGQGTLVT | | CQVWDSSSDHVVFGGGTKLT | | | |
| | VSS | | VL | | | |
| 116 | QVQLQESGPGLVKPSQTLSLTCTV | 117 | DIVMTQSPLSLPVTPGEPASISC | SARS- | | Robbiani, D., et al., |
| | SGGSISSGGYYWSWIRQHPGKGLE | | RSSQSLLHSNGYNYLDWYLQ | CoV2 | | Nature 584: 437-442 |
| | WIGYIYYSGSTYYNPSLKSRVTISV | | KPGQSPQLLIYLGSNRASGVPD | | | (2020) |
| | DTSKNQFSLKLSSVTAADTAVYYC | | RFSGSGSGTDFTLKISRVEAED | | | |
| | ARVWQYYDSSGSFDYWGQGTLVT | | VGVYYCMQALQTPFTFGPGTK | | | |
| | VSS | | VDIK | | | |
| 118 | QVQLQESGPGLVKPSETLSVTCTV | 119 | DIQMTQSPSTLSASVGDSVTIT | SARS- | SARS- | Robbiani, D., et al., |
| | SGGSISSSRYYWGWIRQPPGKGLE | | CRASQSISSWLAWYQQKPGKA | CoV1, | CoV2 | Nature 584: 437-442 |
| | WIGSIYYSGSTYYNPSLKSRVTISV | | PKLLIYKASSLESGVPSRFSGS | SARS- | | (2020) |
| | DTSKNQFSLKLSSVTAADTAVYYC | | GSGTEFTLTISSLQPDDFATYY | CoV2 | | |
| | ARHAAAYYDRSGYYFIEYFQHWG | | CQQYNNYRYTFGQGTKLEIK | | | |
| | QGTLVTVSS | | | | | |
| 120 | EVQLVESGGGVVQPGRSLRLSCAA | 121 | DIQMTQSPSTLSASVGDRVTIT | SARS- | | Robbiani, D., et al., |
| | SGFTFSSYGMHWVRQAPGKGLEW | | CRASQSISSWLAWYQQKPGKA | CoV1 | | Nature 584: 437-442 |
| | VAVISYDGSNKYYADSVKGRFTIS | | PKLLIYKASSLESGVPSRFSGS | (weak), | | (2020) |
| | RDNSKNTLYLQMNSLRAEDTAVY | | GSGTEFTLTISSLQPDDFATYY | SARS- | | |
| | YCAKASGIYCSGGDCYSYYFDYW | | CQQYNSYSTFGQGTKVEIK | CoV2 | | |
| | GQGTLVTVSS | | | | | |
| 122 | QVQLQESGPGLVKPSQTLSLTCTV | 123 | DIVMTQSPLSLPVTPGEPASISC | SARS- | | Robbiani, D., et al., |
| | SGGSISSGGYYWSWIRQHPGKGLE | | RSSQSLLHSNGYNYLDWYLQ | CoV2 | | Nature 584: 437-442 |
| | WIGYIYYSGSTYYNPSLKSRVTISV | | KPGQSPQLLIYLGSNRASGVPD | | | (2020) |
| | DTSKNQFSLKLSSVTAADTAVYYC | | RFSGSGSGTDFTLKISRVEAED | | | |
| | ARTMYYYDSSGSFDYWGQGTLVT | | VGVYYCMQALQTPHTFGGGT | | | |
| | VSS | | KVEIK | | | |
| 124 | EVQLVESGGGVVQPGRSLRLSCAA | 125 | DIQMTQSPSTLSASVGDRVTIT | SARS- | | Robbiani, D., et al., |
| | SGFTFSSYGMHWVRQAPGKGLEW | | CRASQSISSWLAWYQQKPGKA | CoV2 | | Nature 584: 437-442 |
| | VAVISYDGSNKYYADSVKGRFTIS | | PKLLIYKASSLESGVPSRFSGS | | | (2020) |
| | RDNSKNTLYLQMNSLRAEDTAVY | | GSGTEFTLTISSLQPDDFATYY | | | |
| | YCAKASGIYCSGGNCYSYYFDYW | | CQQYNSYSTFGQGTKVEIK | | | |
| | GQGTLVTVSS | | | | | |
| 126 | EVQLVESGGGLVQPGGSLRLSCAA | 127 | DIQMTQSPSSLSASVGDRVTIT | SARS- | | Robbiani, D., et al., |
| | SGFTFSSYDMHWVRQATGKGLEW | | CRASQSISSYLNWYQQKPGKA | CoV2 | | Nature 584: 437-442 |
| | VSAIGTAGDTYYPGSVKGRFTISRE | | PKVLIYAASSLQSGVPSRFSGS | | | (2020) |
| | NAKNSLYLQMNSLRAGDTAVYYC | | GSGTDFTLTISSLQPEDFATYY | | | |
| | ARVGYDSSGYSGWYFDLWGRGTL | | CQQSYSTPPLTFGGGTKVEIK | | | |
| | VTVSS | | | | | |
| 128 | QVQLVESGGGLIQPGGSLRLSCAA | 129 | EIVLTQSPGTLSLSPGERATLSC | SARS- | SARS- | Robbiani, D., et al., |
| | SGFIVSSNYMSWVRQAPGKGLEW | | RASQSVSSSYLAWYQQKPGQ | CoV2 | CoV2 | Nature 584: 437-442 |
| | VSVIYSGGSTFYTDSVKGRFTISRD | | APRLLIYGASSRATGIPDRFSG | | | (2020) |
| | NSKNTLYLQMNSLRAEDTAVYYC | | GGSETDFTLTISRLEPEDCAVY | | | |
| | VRDYGDFYFDYWGQGTLVTVSS | | YCQQYGSSPRTFGQGTKVEIK | | | |
| 130 | QVQLVESGGGLIQPGGSLRLSCAA | 131 | EIVLTQSPGTLSLSPGERATLSC | SARS- | SARS- | Robbiani, D., et al., |
| | SGFIVSSNYMSWVRQAPGKGLEW | | RASQSVSSSYLAWYQQKPGQ | CoV2 | CoV2 | Nature 584: 437-442 |
| | VSVIYSGGSTFYADSVKGRFTISRD | | APRLLIYGASSRATGIPDRFSGS | | | (2020) |
| | NSKNTLYLQMNSLRAEDTAVYYC | | GSGTDFTLTISRLEPEDFAVYY | | | |
| | ARDYGDYYFDYWGQGTLVTVSS | | CQQYGSSPRTFGQGTKVEIK | | | |
| 132 | QVQLQQWGAGLLKPSETLSLTCA | 133 | EIVLTQSPGTLSLSPGERATLSC | SARS- | SARS- | Robbiani, D., et al., |
| | VSGGSLSGFYWTWIRQPPGKGLE | | RASQTVTANYLAWYQQKPGQ | CoV2 | CoV2 | Nature 584: 437-442 |
| | WIGETNHFGSTGYKPSLKSRVTISV | | APRLLIYGASKRATGIPDRFSG | | | (2020) |
| | DMSRNQFSLKVTSVTAADTAVYY | | SGSGTDFTLSISRLEPEDFAVY | | | |
| | CARKPLLYSDFSPGAFDIWGQGTM | | YCQQYTTTPRTFGGGTKVEIK | | | |
| | VTVSS | | | | | |
| 134 | QVQLQQWGAGLLKPSETLSLSCA | 135 | EIVLTQSPGTVSLSPGERATLS | SARS- | SARS- | Robbiani, D., et al., |
| | VYGGSLSGYYWSWIRQPPGKGLE | | CWASQSVSASYLAWYQQKPG | CoV2 | CoV2 | Nature 584: 437-442 |
| | WIGEINHFGSTGYNPSLKSRVTISV | | QAPRLLIYGASSRATGIPDRFS | | | (2020) |
| | DTSKSQFSVKLSSVTAADTAVYYC | | GSGSGTDFTLTISRLEPEDFAV | | | |
| | ARKPLLYSNLSPGAFDIWGQGTMV | | YYCQQYGTTPRTFGGGTKVEI | | | |
| | TVSS | | K | | | |
| 136 | QVQLVESGGGLIQPGGSLRLSCAA | 137 | QSALTQPPSASGSPGQSVTISC | SARS- | SARS- | Robbiani, D., et al., |
| | SGFTVSSNYMSWVRQAPGKGLEW | | TGTSSDVGGYKYVSWYQQHP | CoV2 | CoV2 | Nature 584: 437-442 |
| | VSVIYSGGSTYYADSVKGRFTISRD | | GKAPKLMIYEVSKRPSGVPDR | | | (2020) |
| | NSKNTLYLQMNSLRAEDTAVYYC | | FSGSKSGNTASLTVSGLQAED | | | |
| | ARGEGWELPYDYWGQGTLVTVSS | | EADYYCSSYEGSNNFVVFGGG | | | |
| | | | TKLTVL | | | |
| 138 | QLQLQESGPGLVKPSETLSLTCTVS | 139 | SYELTQPPSVSVAPGKTARITC | SARS- | | Robbiani, D., et al., |
| | GASVSSGSYYWSWIRQPPGKGLE | | GGNNIGSKSVHWYQQKPGQA | CoV1, | | Nature 584: 437-442 |
| | WIGYIYYSGSTNYNPSLKSRVTISV | | PVLVIYFDSDRPSGIPERFSGSN | SARS- | | (2020) |
| | DTSKNQFSLKLSSVTAADTAVYYC | | SGNTATLTISRVEAGDEADYY | CoV2 | | |
| | ARERPGGTYSNTWYTPTDTNWFD | | CQVWDSSRDHVVFGGGTKLT | | | |
| | TWGQGTLVTVSS | | VL | | | |
| 140 | QVQLVQSGAEVKKPGASVRVSCK | 141 | QSVLTQPPSASGTPGQRVTISC | SARS- | | Robbiani, D., et al., |
| | ASGYTFTSYGFSWVRQAPGQGLE | | SGSSSNIGSNYVYWYQQLPGT | CoV2 | | Nature 584: 437-442 |
| | WMGWISAYNGNTNFAQKLQGRV | | APKLLIYRNNQRPSGVPDRFSG | | | (2020) |
| | TMTTDTSTSTAYMELRSLRSDDTA | | SKSGTSASLAISGLRSEDEADY | | | |
| | VYYCARGEAVAGTTGFFDYWGQ | | YCAAWDDSLSGFVVFGGGTK | | | |
| | GTLVTVSS | | LTVL | | | |
| 142 | QVQLQESGPGLVKPSGTLSLTCAV | 143 | QSALTQPASVSGSPGQSITISCT | SARS- | SARS- | Robbiani, D., et al., |
| | SGGSISSTNWWSWVRQPPGKGLE | | GTSSDVGGYNYVSWYQQHPG | CoV2 | CoV2 | Nature 584: 437-442 |
| | WIGEIYHTGSTNYNPSLKSRVTISV | | KAPKLMIYDVSNRPSGVSNRF | | (weak) | (2020) |
| | DKSKNQFSLKLSSVTAADTAVYYC | | SGSKSGNTASLTISGLQAEDEA | | | |
| | VRDGGRPGDAFDIWGQGTMVTVS | | DYYCNSYTSSSTRVFGTGTKV | | | |
| | S | | TVL | | | |
| 144 | EVQLVESGGGLVQPGGSLRLSCAA | 145 | QSALTQPPSASGSPGQSVTISC | SARS- | | Robbiani, D., et al., |
| | SGFTFSSYWMSWVRQAPGKGLEW | | TGTSSDVGGYNYVSWYQQHP | CoV2 | | Nature 584: 437-442 |
| | VANIKQDGSEKYYVDSVKGRFTIS | | GKAPKLMIYEVTKRPSGVPDR | | | (2020) |
| | GDNAKNSLYLHMNSLRAEDTAVY | | FSGSKSGNTASLTVSGLQAED | | | |
| | YCAIQLWLRGGYDYWGQGTLVTV | | EADYYCSSYAGSNNYVVFGG | | | |
| | SS | | GTKLTVL | | | |
| 146 | QVQLQQSGAEVKKPGESLKISCKG | 147 | DIQMTQSPSTLSASVGDRVTIT | SARS- | SARS- | Robbiani, D., et al., |
| | SGYSFTSYWIGWVRQMPGKGLEW | | CRASQSISYWLAWYQQKPGK | CoV2 | CoV2 | Nature 584: 437-442 |
| | MGIIYPGDSDTRYSPSFQGQVTISA | | APKLLIYQASSLESGVPSRFSG | | | (2020) |
| | DKSISTAYMQWSSLKASDTAMYY | | SESGTEFTLTISSLQPDDFATYY | | | |
| | CARSFRDDPRIAVAGPADAFDIWG | | CQQYNSYPYTFGQGTKLEIK | | | |
| | QGTMVTVSS | | | | | |
| 148 | QLQLQESGPGLVKPSETLSLTCTVS | 149 | QSVLTQPPSVSEAPRQRVTISC | SARS- | | Robbiani, D., et al., |
| | GGSISSYYWSWIRQPPGKGLEWIG | | SGSSSNIGNNAVNWYQQVPGK | CoV2 | | Nature 584: 437-442 |
| | YIYYSGSTNYNPSLKSRVTISVDTS | | APKLLIYYDDLLPSGVSDRFSG | | | (2020) |
| | KNQFSLKLSSVTAADTAVYYCAR | | SKSGTSASLAISGLQSEDEADY | | | |
| | VEDWGYCSSTNCYSGAFDIWGQG | | YCAAWDDSLNGAWVFGGGT | | | |
| | TMVTVSS | | KLTVL | | | |
| 150 | QVQLVESGGGVVQPGRSLRLSCA | 151 | QSALTQPASVSGSPGQSITISCT | SARS- | SARS- | Robbiani, D., et al., |
| | ASGFTFSSHAMHWVRQAPGKGLE | | GTSSDVGGYNYVSWYQQHPG | CoV2 | CoV2 | Nature 584: 437-442 |
| | WVAVISYDGSNKYYADSVKGRFTI | | KAPKLMIYDVSNRPSGVSNRF | | (weak) | (2020) |
| | SRDNSKNTLYLQMNSLRAEDTAV | | SGSKSGNTASLTISGLQAEDEA | | | |
| | YYCAREDYYDSSGSFDYWGQGTL | | DYYCSSYTSSSTWVFGGGTKL | | | |
| | VTVSS | | TVL | | | |
| 152 | QVQLVESGGGVVQPGRSLRLSCA | 153 | DIQMTQSPSTLSASVGDRVTIT | SARS- | | Robbiani, D., et al., |
| | ASGFTFSNFGMHWVRQAPGKGLE | | CRASQSMSSWLAWYQQKPGN | CoV2 | | Nature 584: 437-442 |
| | WVAVIWYDGSNKYYADSVKGRFT | | APKLLIYKASSLESGVPSRFSG | | | (2020) |
| | ISRDNSKNTLYLQMNSLRAEDTAV | | SGSGTEFTLTISSLQPDDFATY | | | |
| | YYCARGVNPDDILTGVDAFDIWG | | YCQQHNSSPLTFGGGTKVEIK | | | |
| | QGTMVTVSS | | | | | |
| 154 | QVQLVESGGGLIQPGGSLKLSCVV | 155 | QSVLTQPPSVSGAPGQRVTISC | SARS- | | Robbiani, D., et al., |
| | SGFTVSKNYISWVRQAPGKGLEW | | TGTSSNIGAGYDVHWYQQLPG | CoV2 | | Nature 584: 437-442 |
| | VSVIFAGGSTFYADSVKGRFAISRD | | RAPKVLISGNNIRPSEVPDRFS | | | (2020) |
| | NSNNTLFLQMNSLRVEDTAIYYCA | | GSRSGTSASLAITSLQPEDEAQ | | | |
| | RGDGELFFDQWGQGTLVTVSS | | YYCQSYDSSLYAVFGGGTKLT | | | |
| | | | VL | | | |
| 156 | QVQLVESGGGLIKPGRSLRLSCTAS | 157 | DIVMTQSPLSLSVTPGEPASISC | SARS- | SARS- | Robbiani, D., et al., |
| | GFTFGDYAMTWFRQAPGKGLEW | | RSSQSLLHSNGNNYFDWYLQK | CoV2 | CoV2 | Nature 584: 437-442 |
| | VGFIRSKAYGGTTGYAASVKYRFT | | PGQSPQLLIYLGSNRASGVPDR | | (weak) | (2020) |
| | ISRDDSKSIAYLQMDSLKTEDTAV | | FSGSGSGTDFTLKISRVEAEDV | | | |
| | YYCTRWDGWSQHDYWGQGTLVT | | GVYYCMQVLQIPYTFGQGTKL | | | |
| | VSS | | EIK | | | |
| 158 | QVQLVESGGGVVQPGRSLRLSCA | 159 | NFMLTQPHSVSESPGKTVTISC | SARS- | | Robbiani, D., et al., |
| | ASGFTYSTYAMHWVRQAPGKGLE | | TGSSGSIASNYVQWYQQRPGS | CoV2 | | Nature 584: 437-442 |
| | WVAFISYDGSNKYYADSVKGRFTI | | APTTVIYEDNQRPSGVPDRFSG | | | (2020) |
| | SRDNSKNTLYLQMNSLRAEDTAV | | SIDRSSNSASLTISGLKTEDEAD | | | |
| | YYCARDFYHNWFDPWGQGTLVT | | YYCQSYDSGNHWVVFGGGTR | | | |
| | VSS | | LTVL | | | |
| 160 | QVQLVESGGGVVQPGRSLRLSCA | 161 | QSVLTQPPSVSAAPGQKVTISC | SARS- | SARS- | Robbiani, D., et al., |
| | ASGFTFSTYAMHWVRQAPGEGLE | | SGSSSNIGNNLVSWYQQLPGT | CoV2 | CoV2 | Nature 584: 437-442 |
| | WVAVISYDGSNTYYADSVKGRFTI | | APKLLIYENNKRPSGIPDRFSG | | (weak) | (2020) |
| | SRDNSKNTLYLQMNSLRAEDTAV | | SKSGTSATLGITGLQTGDEAD | | | |
| | YYCARDPIWFGELLSPPFVHFDYW | | YYCGAWDSSLSAGGVYVFGT | | | |
| | GQGTLVTVSS | | GTKVTVL | | | |
| 162 | QVQLVESGGGVVQPGRSLRLSCA | 163 | QPVLTQSPSASASLGASVKLTC | SARS- | SARS- | Robbiani, D., et al., |
| | ASGFTFSNYAMHWVRQAPGKGLE | | TLSSGHSSYAIAWHQQQPEKG | CoV1, | CoV2 | Nature 584: 437-442 |
| | WVAVISYDGSNKYYADSVKGRFTI | | PRYLMKLNTDGSHSKGDGIPD | SARS- | (weak) | (2020) |
| | SRDNSKNTLYLQMNSLRAEDTAIY | | RFSGSSSGAERYLTISSLQSEDE | CoV2 | | |
| | YCASGYTGYDYFVRGDYYGLDV | | ADYYCQTWGTGILVFGGGTK | | | |
| | WGQGTTVTVSS | | LTVL | | | |
| 164 | QVQLVQSGAEVKKPGASVKVSCK | 165 | QSALTQPASVSGSPGQSITISCT | SARS- | SARS- | Robbiani, D., et al., |
| | ASGYTFTSYYMHWVRQAPGQGLE | | GTSSDVGGYKYVSWYQRHPG | CoV2 | CoV2 | Nature 584: 437-442 |
| | WMGIINPSGGSTSYAQKLQGRVT | | KAPKLMIYDVSNRPSGVSNRF | | | (2020) |
| | MTRDTSTSTVYMELSSLRSEDTAV | | SGSKSGNTASLTISGLQAEDEA | | | |
| | YYCARANHETTMDTYYYYYYMD | | DYYCSSYTSSSTSVVFGGGTQ | | | |
| | VWGKGTTVTVSS | | LTVL | | | |
| 166 | EVQLVESGGGLIQPGGSLRLSCAA | 167 | AIRMTQSPSSLSASVGDTVTIT | SARS- | SARS- | Robbiani, D., et al., |
| | SGFTVSSNYMTWVRQAPGKGLEW | | CQASQDISKYLNWYQQKPGK | CoV2 | CoV2 | Nature 584: 437-442 |
| | VSLIYPGGSTYYADSVKGRFTISRD | | APKLLIYDASNLETGVPSRFSG | | | (2020) |
| | NSKNTLYLQMNSLRAEDTAVYYC | | SGSGTDFTFTISSLQPEDIATYY | | | |
| | AREGMGMAAAGTWGQGTLVTVSS | | CQQYDNLPQTFGGGTKVEIK | | | |
| 168 | QVQLVQSGAEVKKPGASVKVSCK | 169 | QSALTQPASVSGSPGQSITISCT | SARS- | SARS- | Robbiani, D., et al., |
| | ASGYTFTGYYMHWVRQAPGQGLE | | GTSSDVGSYNLVSWYQQHPG | CoV2 | CoV2 | Nature 584: 437-442 |
| | WMGWISPVSGGTNYAQKFQGRVT | | KAPKLMIYEGSKRPSGVSNRFS | | | (2020) |
| | MTRDTSISTAYMELSRLRSDDTAV | | GSKSGNTASLTISGLQAEDEAD | | | |
| | YYCARAPLFPTGVLAGDYYYYGM | | YYCCSYAGSSTLVFGGGTKLT | | | |
| | DVWGQGTTVTVSS | | VL | | | |
| 170 | EVQLVESGGGLIQPGGSLRLSCAA | 171 | DIQLTQSPSFLSASVGDRVTITC | SARS- | SARS- | Robbiani, D., et al., |
| | SGLTVSSNYMSWVRQAPGKGLEW | | RASQGISSYLAWYQQKPGKAP | CoV2 | CoV2 | Nature 584: 437-442 |
| | VSVLYSGGSSFYADSVKGRFTISRD | | KLLIYAASTLQSGVPSRFSGSG | | | (2020) |
| | NSKNTLYLQMNSLRAEDTAVYYC | | SGTEFTLTISSLQPEDFATYYC | | | |
| | ARESGDTTMAFDYWGQGTLVTVSS | | QQLNSDSYTFGQGTKLEIK | | | |
| 172 | EVQLVESGGGLIQPGGSLRLSCAA | 173 | DIQLTQSPSFLSASVGDRVTITC | SARS- | SARS- | Robbiani, D., et al., |
| | SGVTVSRNYMSWVRQAPGKGLE | | RASQGISSYLAWYQQKPGKAP | CoV2 | CoV2 | Nature 584: 437-442 |
| | WVSVIYSGGSTYYADSVKGRFTIS | | KLLIYAASTLQSGVPSRFSGSG | | | (2020) |
| | RDNSKNTLYLQMNSLRAEDTAVY | | SGTEFTLTISSLQPEDFATYYC | | | |
| | YCARDLSAAFDIWGQGTMVTVSS | | QQLNSYPPAFGQGTRLEIK | | | |
| 174 | EVQLVESGGGLVQPGGSLRLSCAA | 175 | EIVLTQSPATLSLSPGERATLSC | SARS- | SARS- | Robbiani, D., et al., |
| | SGFTFSGYSMNWVRQAPGKGPEW | | RASQSFSSYLAWYQQKPGQAP | CoV2 | CoV2 | Nature 584: 437-442 |
| | VSYISRSSSTIYYADSVKGRFTISRD | | RLLIYDASNRATGIPARFSGSG | | (weak) | (2020) |
| | NAKNSLYLQMNSLRDEDTAVYYC | | SGTDFTLTISSLEPEDFAVYYC | | | |
| | AREGARVGATYDTYYFDYWGQG | | QQRNNWPPEWTFGQGTKVEI | | | |
| | TLVTVSS | | K | | | |
| 176 | QVQLVQSGPEVKKPGTSVKVSCK | 177 | EIVLTQSPGTLSLSPGERATLSC | SARS- | SARS- | Robbiani, D., et al., |
| | ASGFTFTSSAVQWVRQARGQRLE | | RASQSVSSSYLAWYQQKPGQ | CoV2 | CoV2 | Nature 584: 437-442 |
| | WIGWIVVGSGNTNYAQKFQERVTI | | APRLLIYGASSRATGIPDRFSGS | | | (2020) |
| | TRDMSTSTAYMELSSLRSEDTAVY | | GSGTDFTLTISRLEPEDFAVYY | | | |
| | YCAAPYCSGGSCSDAFDIWGQGT | | CQQYGSSPWTFGQGTKVEIK | | | |
| | MVTVSS | | | | | |
| 178 | QVQLQESGPGLVKPSETLSLSCAV | 179 | NFMLTQPHSVSESPGKTVTISC | SARS- | | Robbiani, D., et al., |
| | SGGSIGSYFWSWIRQPPGKGLEWI | | TGSSGSIASNYVQWYQQRPGS | CoV2 | | Nature 584: 437-442 |
| | GYLHYSGSTNYNPSLKSRVTISVD | | APTTVINEDNQRPSGVPDRFSG | | | (2020) |
| | TSKNQFSLKLSSVTAADTAVYYCA | | SIDSSSNSASLTISGLKTEDEAD | | | |
| | RLQWLRGAFDIWGQGTMVTVSS | | YYCQSYDSSNLVFGGGTKLTV | | | |
| | | | L | | | |
| 180 | QVQLVQSGAEVKKPGASVKVSCK | 181 | QSVLTQPPSASGTPGQRVTISC | SARS- | SARS- | Robbiani, D., et al., |
| | ASGYTFTGYYMHWVRQAPGQGLE | | SGSSSNIGSNTVNWYQQLPGT | CoV2 | CoV2 | Nature 584: 437-442 |
| | WMGWINPNSGGTNYAQKFQGRV | | APKLLIYSNNQRPSGVPDRFSG | | | (2020) |
| | TMTRDTSISTAYMELSRLRSDDTA | | SKSGTSASLAISGLQSEDEADY | | | |
| | VYYCATAHPRRIQGVFFLGPGVW | | YCAAWDDSLNGVVFGGGTKL | | | |
| | GQGTTVTVSS | | TVL | | | |
| 182 | EVQLLESGGGLVQPGGSLRLSCAA | 183 | EIVLTQSPGTLSLSPGERATLSC | SARS- | SARS- | Robbiani, D., et al., |
| | SGFTFSTYAMSWVRQAPGKGLEW | | RASQSVNSRQLAWYQQKPGQ | CoV2 | CoV2 | Nature 584: 437-442 |
| | VSTITGSGRDTYYADSVKGRFTISR | | APRLLIYGASSRATGIPERFSGS | | | (2020) |
| | DNSKNTLFLQLNSLRAEDAAVYSC | | GSGTDFTLTISRLESEDFAVYH | | | |
| | ANHPLASGDDYYHYYMDVWGKG | | CQQYGSSRALTFGGGTKVEIK | | | |
| | TTVTVSS | | | | | |
| 184 | QVQLVQSGAEVKKPGASVKVSCK | 185 | SYELTQPPSVSVAPGKTARITC | SARS- | | Robbiani, D., et al., |
| | ASGYTFTNYYMHWVRQAPGQGLE | | GGNNIGSKSVHWYQQKPGQA | CoV2 | | Nature 584: 437-442 |
| | WMGIINPSGGSTGYAQKFQGRVT | | PVLVIYYDSDRPSGIPERFSGSN | | | (2020) |
| | MTRDTSTSTVYMELSSLRSEDTAV | | SGNTATLTISRVEAGDEADYY | | | |
| | YYCARSRPTPDWYFDLWGRGTLV | | CQVWDSSSDHPGVVFGGGTK | | | |
| | TVSS | | LTVL | | | |
| 186 | QVQLVQSGSEVKKPGSSVKVSCK | 187 | EIVMTQSPATLSVSPGERATLS | SARS- | SARS- | Robbiani, D., et al., |
| | ASGGTFSSYAFSWVRQAPGQGLE | | CRASQSVSSNLAWYQQKPGQ | CoV2 | CoV2 | Nature 584: 437-442 |
| | WMGRIIPILALANYAQKFQGRVTIT | | APRLLIYGASTRATGIPARFSG | | | (2020) |
| | ADKSTSTAYMELSSLRSEDTAVYY | | SGSGTEFTLTISSLQSEDFAVY | | | |
| | CARVNQAVTTPFSMDVWGQGTTV | | YCQQYNNWPITFGQGTRLEIK | | | |
| | TVSS | | | | | |
| 188 | QVQLQESGPGLVKPSGTLSLTCAV | 189 | QSALTQPASVSGSPGQSITISCT | SARS- | SARS- | Robbiani, D., et al., |
| | SGGSISSNNWWSCVRQPPGKGLE | | GTSSDVGGYNYVSWYQQHPG | CoV2 | CoV2 | Nature 584: 437-442 |
| | WIGEIYHSGSTNYNPSLKSRVTISV | | KAPKLMIYDVSNRPSGVSNRF | | (weak) | (2020) |
| | DKSKNQFSLKLSSVTAADTAVYYC | | SGSKSGNTASLTISGLQAEDEA | | | |
| | ARGGDTAMGPEYFDYWGQGTLV | | DYYCSSYTSSSTLLFGGGTKLT | | | |
| | TVSS | | VL | | | |
| 190 | QVQLVESGGGVVQPGRSLRLSCA | 191 | DIQMTQSPSSLSASVGDRVTIT | SARS- | | Robbiani, D., et al., |
| | ASGFTFSSYAMHWVRQAPGKGLE | | CRASQSISSYLNWYQQKPGKA | CoV2 | | Nature 584: 437-442 |
| | WVAVILYDGSNKYYADSVKGRFTI | | PKLLIYAASSLQSGVPSRFSGS | | | (2020) |
| | SRDNSKNTLYLQMNSLRAEDTAV | | GSGTDFTLTISSLQPEDFATYY | | | |
| | YYCARDSDVDTSMVTWFDYWGQ | | CQQSYSTPPWTFGQGTKVEIK | | | |
| | GTLVTVSS | | | | | |
| 192 | EVQLLESGGGLVQPGGSLRLSCAA | 193 | SYELTQPPSVSVAPGKTARITC | SARS- | | Robbiani, D., et al., |
| | SGFTFSNYAMSWVRQAPGKGLEW | | GGNNIGSKSVHWYQQKPGQA | CoV2 | | Nature 584: 437-442 |
| | VSAISGSDGSTYYAGSVKGRFTISR | | PVLVIYYDSDRPSGIPERFSGSN | | | (2020) |
| | DNSKNTLYLQMNSLRAEDTAVYY | | SGNTATLTISRVEAGDEAEYH | | | |
| | CAKDPLITGPTYQYFHYWGQGTL | | CQVWDSSSDRPGVVFGGGTK | | | |
| | VTVSS | | LTVL | | | |
| 194 | QVQLVESGGGVVQPGRSLRLSCA | 195 | DIQMTQSPSTLSASVGDRVTIT | SARS- | SARS- | Robbiani, D., et al., |
| | ASGFTFSSYAMHWVRQAPGKGLE | | CRASQSISNWLAWFQQKPGKA | CoV2 | CoV2 | Nature 584: 437-442 |
| | WVAVIPFDGRNKYYADSVTGRFTI | | PKLLIYEASSLESGVPSRFSGSG | | | (2020) |
| | SRDNSKNTLYLQMNSLRAEDTAV | | SGTEFTLTISSLQPDDFATYYC | | | |
| | YYCASSSGYLFHSDYWGQGTLVT | | QQYNSYPWTFGQGTKVEIK | | | |
| | VSS | | | | | |
| 196 | EVQLVESGGGLVQPGGSLRLSCAA | 197 | NFMLTQPHSVSESPGKTVTISC | SARS- | | Robbiani, D., et al., |
| | SGFTFSTYWMSWVRQPPGKGLEW | | TGSSGSIASNYVQWYQQRPGS | CoV2 | | Nature 584: 437-442 |
| | VANIKQDGSEKYYVDSVKGRFTIS | | APTTVIYEDNQRPSGVPDRFSG | | | (2020) |
| | RDNAKNSLYLQMNSLRADDTAVY | | SIDSSSNSASLTISGLKTEDEAD | | | |
| | YCAGGTWLRSSFDYWGQGTLVTV | | YYCQSYDSSNWVFGGGTKLT | | | |
| | SS | | VL | | | |
| 198 | EVQLVESGGGVVQPGRSLRLSCAA | 199 | DIQMTQSPSSLSASVGDRVTIT | SARS- | | Robbiani, D., et al., |
| | SGFTFSSYAMHWVRQAPGKGLEW | | CQASQDISNYLNWYQQKPGK | CoV2 | | Nature 584: 437-442 |
| | VAVISYDGSNKYSADSVKGRFTIS | | APKLLIYDASNLETGVPSRFSG | | | (2020) |
| | RDNSKNTLYLQMNSLRAEDTAVY | | SGSGTDFTFTISSLQPEDIATYY | | | |
| | YCAKGGAYSYYYYMDVWGKGTT | | CQQYDNLPLTFGGGTKVEIK | | | |
| | VTVSS | | | | | |
| 200 | EVQLVESGGGLVQPGGSLRLSCAA | 201 | DIQLTQSPSFLSASVGDRVTITC | SARS- | SARS- | Robbiani, D., et al., |
| | SGVTVSSNYMSWVRQAPGKGLEW | | RASQGISSYLAWYQQKPGKAP | CoV2 | CoV2 | Nature 584: 437-442 |
| | VSLIYSGGSTFYADSVKGRFTISRD | | KLLIYAASTLQSGVPSRFSGSG | | | (2020) |
| | NSENTLYLQMNTLRAEDTAVYYC | | SGTEFTLTISSLQPEDFATYYC | | | |
| | ARDLYYYGMDVWGQGTTVTVSS | | QQLNSYSYTFGQGTKLEIK | | | |
| 202 | EVQLVESGGGVVQPGRSLRLSCAA | 203 | NFMLTQPHSVSESPGKTVTISC | SARS- | | Robbiani, D., et al., |
| | SGFTFSSYAMFWVRQAPGKGLEW | | TGSSGSIASNYVQWYQQRPGS | CoV2 | | Nature 584: 437-442 |
| | VAVISYDGSNKYYADSVKGRFTIS | | APTTVIYEDNQRPSGVPDRFSG | | | (2020) |
| | RDNSKNTLYLQMNSLRAEDTAVY | | SIDSSSNSASLTISGLKTEDEAD | | | |
| | YCARADLGYCTNGVCYVDYWGQ | | YYCQSYDSSNWVFGGGTKLT | | | |
| | GTLVTVSS | | VL | | | |
| 204 | EVQLVESGGGLVQPGGSLRLSCAA | 205 | QSALTQPASVSGSPGQSITISCT | SARS- | | Robbiani, D., et al., |
| | SGFSVSTKYMTWVRQAPGKGLEW | | GTSNDVGSYTLVSWYQQYPG | CoV2 | | Nature 584: 437-442 |
| | VSVLYSGGSDYYADSVKGRFTISR | | KAPKLLIFEGTKRSSGISNRFSG | | | (2020) |
| | DNSKNALYLQMNSLRVEDTGVYY | | SKSGNTASLTISGLQGEDEADY | | | |
| | CARDSSEVRDHPGHPGRSVGAFDI | | YCCSYAGASTFVFGGGTKLTV | | | |
| | WGQGTMVTVSS | | L | | | |
| 206 | EVQLVESGGGLIQPGGSLRLSCAA | 207 | QSALTQPASVSGSPGQSITISCT | SARS- | SARS- | Robbiani, D., et al., |
| | SGFTVSNNYMSWVRQAPGKGLEW | | GTSSDVGGYNYVSWYQQHPG | CoV2 | CoV2 | Nature 584: 437-442 |
| | VSVIYSGGSTYYADSVKGRFTISRD | | KAPKLMIYDVSNRPSGVSNRF | | | (2020) |
| | KSKNTLYLQMNRLRAEDTAVYYC | | SGSKSGNTASLTISGLQAEDEA | | | |
| | AREGEVEGYNDFWSGYSRDRYYF | | DYYCSSYTSSSTRVFGTGTKV | | | |
| | DYWGQGTLVTVSS | | TVL | | | |
| 208 | EVQLVESGGGLIQPGGSLRLSCAA | 209 | QSALTQPASVSGSPGQSITISCT | SARS- | SARS- | Robbiani, D., et al., |
| | SGFSVSSNYMSWVRQAPGKGLEW | | GTSSDVGGYNYVSWYQQHPG | CoV2 | CoV2 | Nature 584: 437-442 |
| | VSVIYSGGSTYYADSVKGRFTISRD | | KAPKLMIYDVSNRPSGVSNRF | | | (2020) |
| | NSKNTLYLQMNSLRAEDTAVYYC | | SGSKSGNTASLTISGLQAEDEA | | | |
| | AREGEVEGYYDFWSGYSRDRYYF | | DYYCSSYTSSTTRVFGTGTRV | | | |
| | DYWGQGTLVTVSS | | TVL | | | |
| 210 | EVQLVESGGGLVKPGGSLRLSCAA | 211 | QSALTQPASVSGSPGQSITISCT | SARS- | | Robbiani, D., et al., |
| | SGLTFTAYRMNWVRQAPGKGLE | | GTSSDIGVYNYISWSQQHPGK | CoV2 | | Nature 584: 437-442 |
| | WLSSISNTNGDIYYADSVKGRFTIS | | APKVMIYDVTNRPSGVSNRFS | | | (2020) |
| | RDNAKNSLYLQMNSLRADDTAVY | | GSKSGNTASLTISGLQAEDEAD | | | |
| | YCARDVASNYAYFDLWGQGTLVT | | YYCSSYRGSSTPYVFGTGTKV | | | |
| | VSS | | TVL | | | |
| 212 | EVQLVQSGAEVKKPGESLKISCKG | 213 | QAVVTQEPSLTVSPGGTVTLT | SARS- | | Robbiani, D., et al., |
| | SGYRFTNYWIGWVRQMPGKGLE | | CGSSTGAVTSGHYPYWFQQKS | CoV2 | | Nature 584: 437-442 |
| | WMGIIYPGDSDTRYSPSFQGQVTIS | | GQAPRTLIYETSIKHSWTPARF | | | (2020) |
| | ADKSITTAYLQWSSLKASDTAMY | | SGSLLGGKAALTLSGAQPEDE | | | |
| | YCARLSDRWYSPFDPWGQGTLVT | | ADYYCLLSYSGARPVFGGGTK | | | |
| | VSS | | LTVL | | | |
| 214 | EVQLVESGGGLVQPGGSQRLSCAA | 215 | EIVMTQSPATLSVSPGERATLS | SARS- | | Robbiani, D., et al., |
| | SGFTVSSNYMSWIRQAPGKGLEW | | CRASQSVSSHLAWYQQKPGQ | CoV2 | | Nature 584: 437-442 |
| | VSVIYSGGSAYYVDSVKGRFTISR | | APRLLIYGASTRATGIPTRFSGS | | | (2020) |
| | DNSKNTLYLQMNSLRPEDTAVYY | | GSGTEFTLTISSLQSEDFAVYY | | | |
| | CARIANYMDVWGKGTTVTVSS | | CQQYNNWPPLTFGGGTKVEIK | | | |
| 216 | EVQLVESGGGLVQPGGSLRLSCVA | 217 | QSALTQPASVSGSPGQSITISCT | SARS- | | Robbiani, D., et al., |
| | SGFTFSSYWMHWVRQVPGKGPV | | GTSSDVGYYNFVSWYQQHPG | CoV2 | | Nature 584: 437-442 |
| | WVSHINSEGSSTNYADSVRGRFTIS | | KAPKLMIYEVSNRPSGVSNRFS | | | (2020) |
| | RDNAKDTLYLQMNNLRAEDTAVY | | GSKSGNTASLIISGLQAEDEAD | | | |
| | YCARPTAVAAAGNYFYYYGMDV | | YYCSSYRSSSTLVFGGGTKLT | | | |
| | WGQGTTVTVSS | | VL | | | |
| 218 | EVQLVESGGGLVKPGGSLRLSCAA | 219 | NFMLTQPHSVSESPGKTVTISC | SARS- | SARS- | Robbiani, D., et al., |
| | SGFTFSSYNMNWVRQAPGKGLEW | | TGSSGSIASNYVQWYQQRPGS | CoV2 | CoV2 | Nature 584: 437-442 |
| | VSCISSSSSYIYYADSVKGRFTISRD | | APTTVIYEDNQRPSGVPDRFSG | | (weak) | (2020) |
| | NAKNSLYLQMNSLRAEDTAVYYC | | SIDSSSNSASLTISGLKTEDEAD | | | |
| | ARERGYDGGKTPPFLGGQGTLVT | | YYCQSYDSSNYWVFGGGTKL | | | |
| | VSS | | TVL | | | |
| 220 | EVQLVESGGGLIQPGGSLRLSCAA | 221 | QSALTQPASVSGSPGQSITISCT | SARS- | SARS- | Nature 584: 437-442 |
| | SGFTVSSNYMSWVRQAPGKGLEW | | GTSSDVGSYNLVSWYQQHPG | CoV2 | CoV2 | (2020) |
| | VSVIYSGYSTYYVDSVKGRFTISRD | | KAPKLMIYEGSKRPSGVSNRFS | | (weak) | |
| | NSKNTLYLQMNSLRAEDTAVYYC | | GSKSGNTASLTISGLQAEDEAD | | | |
| | ARVGGAHSGYDGSFDYWGQGTL | | YYCCSYAGSSTWVFGGGTKLT | | | |
| | VTVSS | | VL | | | |
| 222 | QVQLVESGGGVVQPGRSLRLSCA | 223 | DIQMTQSPSSLSASVGDRVTIT | SARS- | SARS- | Robbiani, D., et al., |
| | ASGFTFSRYGMHWVRQAPGKGLE | | CQASQGISNYLNWYQQKPGK | CoV1, | CoV2 | Nature 584: 437-442 |
| | WVAVMSYDGSSKYYADSVKGRFT | | APKLLIYDASNLETGVPSRFSG | SARS- | (weak) | (2020) |
| | ISRDNSKNTLCLQMNSLRAEDTAV | | SGSGTDFTFTISSLQPEDIATYY | CoV2 | | |
| | YYCAKQAGPYCSGGSCYSAPFDY | | CQQYDNLPITFGQGTRLEIK | | | |
| | WGQGTLVTVSS | | | | | |
| 224 | EVQLVESGGGLIQPGGSLRLSCAA | 225 | EIVMTQSPATLSVSPGERATLS | SARS- | SARS- | Robbiani, D., et al., |
| | SGFIVSSNYMSWVRQAPGKGLEW | | CRASQSVSSNLAWYQQKPGQ | CoV2 | CoV2 | Nature 584: 437-442 |
| | VSVIYSGGSTFYADSVKGRFTISRD | | APRLLIYGASTRATAIPARFSG | | | (2020) |
| | NSKNTLYLQMNSLRAEDTAVYYC | | SGSGTEFTLTISSLQSEDFAVY | | | |
| | ARDFGEFYFDYWGQGTLVTVSS | | YCQQYNNWPRTFGQGTKVEI | | | |
| | | | K | | | |
| 226 | QVQLVESGGGVVQPGRSLRLSCA | 227 | SYELTQPPSVSVAPGQTARISC | SARS- | | Robbiani, D., et al., |
| | ASGFTFSNYGMHWVRQAPGKGLE | | GGNNIGSKNVHWYQQKPGQA | CoV2 | | Nature 584: 437-442 |
| | WVAVISYDGNNKYYADSVKGRFT | | PVLVVYDDSDRPSGIPERFSGS | | | (2020) |
| | ISRDNSKNTLYLQMNSLRAEDTAV | | NSGNTATLTISRVEAGDEADY | | | |
| | YYCAKDPFPLAVAGTGYFDYWGQ | | YCQVWDSSSDPWVFGGGTKL | | | |
| | GTLVTVSS | | TVL | | | |
| 228 | EVQLVESGGGLVQPGGSLRLSCAA | 229 | QSALTQPASVSGSPGQSITISCT | SARS- | SARS- | Robbiani, D., et al., |
| | SGFSVSTKYMTWVRQAPGKGLEW | | GTSNDVGSYTLVSWYQQYPG | CoV2 | CoV2 | Nature 584: 437-442 |
| | VSVLYSGGSDYYADSVKGRFTISR | | KAPKLLIFEVTKRSSGISNRFSG | | (weak) | (2020) |
| | DNSKNALYLQMNSLRVEDTGVYY | | SKSGNTASLTISGLQGEDEADY | | | |
| | CARDSSEVRDHPGHPGRSVGAFDI | | YCCSYAGASTFVFGGGTKLTV | | | |
| | WGQGTMVTVSS | | L | | | |
| 230 | QVQLVQSGAEVKKPGSSVKVSCK | 231 | EIVLTQSPGTLSLSPGERATLSC | SARS- | SARS- | Robbiani, D., et al., |
| | ASGGTFSSYAINWVRQAPGQGLE | | RASQSVSSTYLAWYQQKPGQ | CoV2 | CoV2 | Nature 584: 437-442 |
| | WMGRIIPIVGIANYAQKFQGRVTIT | | APRLLIYGASSRATGIPDRFSGS | | | (2020) |
| | ADKSSSTAYMELSSLRSEDTAVYY | | GSGTDFTLTISRLEPEDFAVYY | | | |
| | CARDLLDPQLDDAFDIWGQGTMV | | CQQYGSSPWTFGQGTKVEIK | | | |
| | TVSS | | | | | |
| 232 | EVQLVESGGGLVQPGRSLRLSCAA | 233 | IRMTQSPSSVSASVGDRVTITC | SARS- | | Robbiani, D., et al., |
| | SGFTFDDYAMHWVRQAPGKGLE | | RASQGISSWLAWYQQKPGKA | CoV2 | | Nature 584: 437-442 |
| | WVSGISWNSGSIGYADSVKGRFTIS | | PKLLIYVESSLQSGVPSRFSGS | | | (2020) |
| | RDNAKNSLYLQMNSLRAEDTALY | | GSGTDFTLTISSLQPEDFATYY | | | |
| | YCVKGVEYSSSSNFDYWGQGTLV | | CQQANSFPLTFGGGTKVEIK | | | |
| | TVSS | | | | | |
| 234 | EVQLVESGGGLVQPGGSLRLSCAA | 235 | DIQLTQSPSSLSASVGDRVTITC | SARS- | | Robbiani, D., et al., |
| | SGFTVSSNYMSWVRQAPGKGLEW | | QASQDISNYLNWYQQKPGKA | CoV2 | | Nature 584: 437-442 |
| | VSLIYSGGSTYYADSVKGRFTISRD | | PKLLIYDASNLETGVPSRFSGS | | | (2020) |
| | NSKNTLYLQMNSLRAEDTAVYYC | | GSGTDFTFTISSLQPEDIATYYC | | | |
| | ARDTLGRGGDYWGQGTLVTVSS | | QQYDNLPRSFGQGTKLEIK | | | |
| 236 | EVQLLESGGGLEQPGGSLRLSCAA | 237 | DIQLTQSPSSLSASVGDRVTITC | SARS- | | Robbiani, D., et al., |
| | SGFTFSTYAMSWVRQAPGKGLEW | | RASQSISSYLNWYQQKPGKAP | CoV1, | | Nature 584: 437-442 |
| | VSAISGSGAGTFYADSVKGRFTISR | | KLLIYAASSLQSGVPSRFSGSG | SARS- | | (2020) |
| | DNSKNTLYLQMNSLRAEDTAVYY | | SGTDFTLTISSLQPEDFATYYC | CoV2 | | |
| | CARESDCGSTSCYQVGWFDPWGQ | | QQSYSTPPWTFGQGTKVEIK | | | |
| | GTLVTVSS | | | | | |
| 238 | QVQLVQSGAEVKKPGASVKVSCK | 239 | EIVLTQSPGTLSLSPGERATLSC | SARS- | | Robbiani, D., et al., |
| | ASGHTFTSYYMHWVRQAPGQGLE | | RASQSVSSSYLAWYQQKPGQ | CoV2 | | Nature 584: 437-442 |
| | WMGIINPSGGSTSYAQKFQGRVTM | | APRLLIYGASSRATGIPDRFSGS | | | (2020) |
| | TRDTSTSTVYMELSSLRSEDTAVY | | GSGTDFTLTISRLEPEDFAVYY | | | |
| | YCARGPERGIVGATDYFDYWGQG | | CQQYVSSPWTFGQGTKVEIK | | | |
| | TLVTVSS | | | | | |
| 240 | EVQLLESGGGLVQPGGSLRLSCAA | 241 | EIVLTQSPATLSLSPGERATLSC | SARS- | SARS- | Robbiani, D., et al., |
| | SGFTFSSYAMSWVRQAPGKGLEW | | RASQSVSSYLAWYQQKPGQA | CoV2 | CoV2 | Nature 584: 437-442 |
| | VSAISGSGGSTYYADSVKGRFTISR | | PRLLIYDASNRATGIPARFSGS | | (weak) | (2020) |
| | DNSKNTLYLQMNSLRAEDTAVYY | | GSGTDFTLTISSLEPEDFAVYY | | | |
| | CAKEPIGQPLLWWDYWGQGTLVT | | CQQRSNWPRGFGQGTKVEIK | | | |
| | VSS | | | | | |
| 242 | EVQLVQSGAEVKKPGESLKISCKG | 243 | EIVLTQSPGTLSLSPGERATLSC | SARS- | | Robbiani, D., et al., |
| | SGYSFTSYWIGWVRQMPGKGLEW | | RASQSVSGSYLAWYQQRPGQ | CoV2 | | Nature 584: 437-442 |
| | MGIIYPGDSDTRYSPSFQGQVTISA | | APRLLIYGASSRATGIPDRFSGS | | | (2020) |
| | DKSISTAYLKWSSLKASDSAMYYC | | GSGTDFTLTISRLEPEDFAVYY | | | |
| | ARGPNLQNWFDPWGQGTLVTVSS | | CQQYGSSLTFGGGTKVEIK | | | |
| 244 | EVQLVESGGGLIQPGGSLRLSCAA | 245 | DIQLTQSPSFLSASVGDRVTITC | SARS- | SARS- | Robbiani, D., et al., |
| | SGFTVSSNYMSWVRQAPGKGLEW | | RASQGISSYLAWYQQKPGKAP | CoV2 | CoV2 | Nature 584: 437-442 |
| | VSVIYSGGSTFYADSVKGRFTFSRD | | KLLIYAASTLQSGVPSRFSGSG | | | (2020) |
| | NSKNTLYLQMNSLRAEDTAVYYC | | SGTEFTLTISSLQPEDFATYYC | | | |
| | ARDLMAYGMDVWGQGTTVTVSS | | QQLNSYPQGTFGGGTKVEIK | | | |
| 246 | EVQLVESGGGLVQPGGSLRLSCAA | 247 | EIVMTQSPATLSVSPGERATLS | SARS- | SARS- | Robbiani, D., et al., |
| | SEFTVSSNYMSWVRQAPGKGLEW | | CRASQSVSSNLAWYQQKPGQ | CoV2 | CoV2 | Nature 584: 437-442 |
| | VSVIYSGGSTFYADSVKGRFTISRD | | GPRLLIYGASTRATGIPARFSG | | | (2020) |
| | NSKNTLYLQMNSLRPEDTAVYYC | | SGSGTEFTLTISSLQSEDFAVY | | | |
| | ARDYGDFYFDFWGQGTLVTVSS | | YCQQYNNWPRTFGQGTKVEIK | | | |
| 248 | QVQLVQSGAEVKKPGASVKVSCK | 249 | LTQPASVSGSPGQSITISCTGTS | SARS- | | Robbiani, D., et al., |
| | ASGYTVTGYYIHWVRQAPGQGLE | | SDVGSYNLVSWYQQHPGKAP | CoV2 | | Nature 584: 437-442 |
| | WMGWISPNSGGTNYAQKFQGWV | | KLMIYEDSKRPSGVSNRFSGSK | | | (2020) |
| | TMTRDMSITTAYMELSRLRSDDTA | | SGNTASLTISGLQAEDEADYY | | | |
| | VYYCARERYFDLGGMDVWGQGT | | CCSYAGSSTRLFGGGTKLTVL | | | |
| | TVTVSS | | | | | |
| 250 | QVQLVESGGGVVQPGRSLRLSCA | 251 | DIQLTQSPSSLSASVGDRVTITC | SARS- | | Robbiani, D., et al., |
| | ASGFTFSSYGMHWVRQAPGKGLE | | RASQSISSYLTWYQQKPGKAP | CoV2 | | Nature 584: 437-442 |
| | WVAAIWYDGSNKHYADSVKGRFT | | KLLIYAASSLQSGVPSRFSGSG | | | (2020) |
| | ISRDNSKNTLYLQMNSLRAEDTAV | | SGTDFTLTISSLQPEDFATYYC | | | |
| | YYCARDVGRVTTWFDPWGQGTL | | QQSYSTPPWTFGQGTKVEIK | | | |
| | VTVSS | | | | | |
| 252 | EVQLLESGGGLVQPGGSLRLSCAA | 253 | DIQLTQSPSSLSASVGDRVTITC | SARS- | | Robbiani, D., et al., |
| | SGFTFSSYAMSWVRQAPGKGLEW | | RASQSISSYLNWYQQKPGKAP | CoV1, | | Nature 584: 437-442 |
| | VSAITDSGDGTFYADSVKGRFTISR | | KLLIYAASSLQSGVPSRFSGSG | SARS- | | (2020) |
| | DNSKNTLYLQMNSLRAEDTAVYY | | SGTDFTLTISSLQPEDFATYYC | CoV2 | | |
| | CASEEDYSNYVGWFDPWGQGTLV | | QQSYSTPPWTFGQGTKVEIK | | | |
| | TVSS | | | | | |
| 254 | EVQLVESGGGLVQPGGSLRLSCAA | 255 | DIQLTQSPSSLSASVGDRVTITC | SARS- | | Robbiani, D., et al., |
| | SGFTFSSYDMHWVRQATGKGLEW | | RASQSISSYLNWYQQKPGKAP | CoV2 | | Nature 584: 437-442 |
| | VSAIGTAGDTYYPDSVKGRFTISRE | | KLLIYVASSLQSGVPSRFSGSG | | | (2020) |
| | NAKNSLYLQMNSLRAGDTAVYYC | | SGTDFTLTISSLQPEDFATYYC | | | |
| | ARDRGSSGWYGWYFDLWGRGTL | | QQSYSTPPITFGQGTRLEIK | | | |
| | VTVSS | | | | | |
| 256 | EVQLVESGGGLVQPGGSLRLSCAA | 257 | DIVMTQSPSFLSASVGDRVTIT | SARS- | SARS- | Wu, Y., et al., |
| | SGFIVSSNYMSWVRQAPGKGLEW | | CRASQGISSYLAWYQQKPGKA | CoV2 | CoV2 | Science 368: 1274- |
| | VSVIYSGGSTYYADSVKGRFTISRH | | PKLLIYAASTLQSGVPSRFSGS | | | 1278 (2020) |
| | NSKNTLYLQMNSLRAEDTAVYYC | | GSGTEFTLTISSLQPEDFATYY | | | |
| | AREAYGMDVWGQGTTVTVSS | | CQQLNSYPPYTFGQGTKLEIK | | | |
| 258 | QVQLVQSGAEVKKPGASVKVSCK | 259 | DIQMTQSPLSLPVTPGEPASISC | SARS- | SARS- | Wu, Y., et al., |
| | ASGYTFTGYYMHWVRQAPGQGLE | | RSSQSLLDSDDGNTYLDWYLQ | CoV2 | CoV2 | Science 368: 1274- |
| | WMGRINPNSGGTNYAQKFQGRVT | | KPGQSPQLLIYTLSYRASGVPD | | | 1278 (2020) |
| | MTRDTSISTAYMELSRLRSDDTAV | | RFSGSGSGTDFTLKISRVEAED | | | |
| | YYCARVPYCSSTSCHRDWYFDLW | | VGVYYCMQRIEFPLTFGGGTK | | | |
| | GRGTLVTVSS | | VEIK | | | |
| 260 | QVQLVQSGAEVKKPGASVKVSCK | 261 | EIVLTQSPGTLSLSPGERATLSC | SARS- | SARS- | Pinto, D., et al., |
| | ASGYPFTSYGISWVRQAPGQGLEW | | RASQTVSSTSLAWYQQKPGQA | CoV1, | CoV2 | Nature 583: 290-295 |
| | MGWISTYNGNTNYAQKFQGRVT | | PRLLIYGASSRATGIPDRFSGSG | SARS- | and | (2020) |
| | MTTDTSTTTGYMELRRLRSDDTA | | SGTDFTLTISRLEPEDFAVYYC | CoV2 | SARS- | |
| | VYYCARDYTRGAWFGESLIGGFD | | QQHDTSLTFGGGTKVEIK | | CoV1 | |
| | NWGQGTLVTVSS | | | | | |
| 262 | EVQLVESGGGLVQPGGSLRLSCAA | 263 | DIQMTQSPSSLSASVGDRVTIT | SARS- | SARS- | Meulen, J., et al., |
| | SGFTFSDHYMDWVRQAPGKGLEW | | CRASQSISSYLNWYQQKPGKA | CoV1 | CoV1 | (2006), PLoS |
| | VGRTRNKANSYTTEYAASVKGRF | | PKLLIYAASSLQSGVPSRFSGS | | | Medicine, Vol. 3(7): |
| | TISRDDSKNSLYLQMNSLKTEDTA | | GSGTDFTLTISSLQPEDFATYY | | | e237, 1071-1079 |
| | VYYCARGISPFYFDYWGQGTLVT | | CQQSYSTPPTFGQGTKVEIK | | | |
| | VSS | | | | | |
| 264 | QVQLVESGGGLVKPGGSLRLSCAA | 265 | DIQMTQSPSSLSASVGDRVTIT | SARS- | SARS- | U.S. patent |
| | SGFTFSDYYMSWIRQAPGKGLEW | | CQASQDITNYLNWYQQKPGK | CoV2 | CoV2 | application |
| | VSYITYSGSTIYYADSVKGRFTISR | | APKLLIYAASNLETGVPSRFSG | | | Ser. No. |
| | DNAKSSLYLQMNSLRAEDTAVYY | | SGSGTDFTFTISGLQPEDIATYY | | | 10/787,501 |
| | CARDRGTTMVPFDYWGQGTLVTV | | CQQYDNLPLTFGGGTKVEIK | | | |
| | SS | | | | | |
| 266 | QVQLVESGGGVVQPGRSLRLSCA | 267 | QSALTQPASVSGSPGQSITISCT | SARS- | SARS- | U.S. patent |
| | ASGFTFSNYAMYWVRQAPGKGLE | | GTSSDVGGYNYVSWYQQHPG | CoV2 | CoV2 | application |
| | WVAVISYDGSNKYYADSVKGRFTI | | KAPKLMIYDVSKRPSGVSNRF | | | Ser. No. |
| | SRDNSKNTLYLQMNSLRTEDTAV | | SGSKSGNTASLTISGLQSEDEA | | | 10/787,501 |
| | YYCASGSDYGDYLLVYWGQGTLV | | DYYCNSLTSISTWVFGGGTKL | | | |
| | TVSS | | TVL | | | |
| 268 | QVQLVQSGAEVKKPGSSVKVSCK | 269 | DIQMTQSPSSLSASVGDRVTIT | SARS- | SARS- | INN Proposed List |
| | ASGGTFSNYAISWVRQAPGQGLE | | CRASQSISSYLSWYQQKPGKA | CoV2 | CoV2 | P124 |
| | WMGRIIPILGIANYAQKFQGRVTIT | | PKLLIYAASSLQSGVPSRFSGS | | | (who.int/medicines/ |
| | ADKSTSTAYMELSSLRSEDTAVYY | | GSGTDFTLTITSLQPEDFATYY | | | publications/ |
| | CARGYYEARHYYYYYAMDVWGQ | | CQQSYSTPRTFGQGTKVEIK | | | druginformation/ |
| | GTAVTVSS | | | | | innlists/PL124- |
| | | | | | | COVID.pdf) |
| 270 | EVQLLESGGGVVQPGGSLRLSCAA | 271 | DIVMTQSPLSLPVTPGEPASISC | SARS- | SARS- | Shuo Du et al., 2020 |
| | SGFAFTTYAMNWVRQAPGRGLEW | | RSSQSLLHSNGYNYLDWYLQ | CoV2 | CoV2 | (biorxiv.org/content/ |
| | VSAISDGGGSAYYADSVKGRFTIS | | KPGQSPQLLIYLGSNRASGVPD | | | 10.1101/ |
| | RDNSKNTLYLQMNSLRAEDTAVY | | RFSGSGSGTDFTLKISRVEAED | | | 2020.07.09.195263) |
| | YCAKTRGRGLYDYVWGSKDYWG | | VGVYYCMQALQTPGTFGQGT | | | |
| | QGTLVTVSS | | RLEIK | | | |
| 272 | QVQLVQSGAEVKKPGASVKVSCK | 273 | QSALTQPPSASGSPGQSVTISC | SARS- | SARS- | Lihong Liu et al., |
| | ASGYTFTGYYMHWVRQAPGQGLE | | TGTSSDVGGYNYVSWYQQHP | CoV2 | CoV2 | 2020 |
| | WMGWINPNSGGTNYTQMFQGRV | | GKAPKLMIYEVSKRPSGVPDR | | | (nature.com/articles/ |
| | TMTRDTSISTAYMEVSRLRSDDTA | | FSGSKSGNTASLTVSGLQAED | | | s41586-020-2571-7) |
| | VYYCARDRSWAVVYYYMDVWG | | EADYYCSSYAGSNNLVFGGGT | | | |
| | KGTTVTVSS | | KLTVL | | | |
| 274 | QITLKESGPTLVKPTQTLTLTCTFS | 275 | QSALAQPASVSGSPGQSITISCT | SARS- | SARS- | Lihong Liu et al., |
| | GFSLSTSGVGVGWIRQPPGKALEW | | GTSSDVGAYNYVSWYQQHPG | CoV2 | CoV2 | 2020 |
| | LALIYWDDDKRYSPSLKSRLTITK | | KAPKLMIYDVSKRPSGVSNRF | | | (nature.com/articles/ |
| | DTSKNQVVLTMTNMDPVDTATYY | | SGSKSGNTASLTISGLQAEDEA | | | s41586-020-2571-7) |
| | CAHHKIERIFDYWGQGTLVTVSS | | DYYCSSYTTSSTVFGGGTKLT | | | |
| | | | VL | | | |
| 276 | QVQLVQSGAEVKKPGASVRVSCK | 277 | QSALTQPASVSGSPGQSITISCT | SARS- | SARS- | Lihong Liu et al., |
| | ASGYTFTGYYMHWVRQAPGQGLE | | GTSSDVGGYNFVSWYQQHPG | CoV2 | CoV2 | 2020 |
| | WMGWINPISDGTNYAQKFQGWVT | | KAPKLMIYDVSKRPSGVSNRF | | | (nature.com/articles/ |
| | MTRDTSISTVYMELSRLRSDDTAV | | SGSKSGNTASLTISGLQAEDEA | | | s41586-020-2571-7) |
| | YYCARGGSRCSGGNCYGWAYDA | | DCYCSSYTSSSTFVFGTGTKVT | | | |
| | FDIWGQGTMITVSS | | VL | | | |
| 278 | QVQLVQSGAEVKKPGSSVKVSCK | 279 | EIVMTQSPATLSVSPGERATLS | SARS- | SARS- | Lihong Liu et al., |
| | ASGGTFSSYAISWVRQAPGQGLEW | | CRASQSVSSDLAWYQHKPGQ | CoV2 | CoV2 | 2020 |
| | MGGNIPIFGTANYAQKFQGRVTIT | | APRLLIYGASTRATGIPVRFSG | | | (nature.com/articles/ |
| | ADESTSTAYMELSSLRSEDTAVYY | | SGSGTEFTLTISSLQSEDFAVY | | | s41586-020-2571-7) |
| | CARGVGYRGVIPLNWFDPWGQGT | | YCQQYNNWPPFTFGGGTKVEI | | | |
| | VVTVSS | | K | | | |
| 280 | QVQLVQSGAEVKKPGASVKVSCK | 281 | QSALTQPASVSGSPGQSITISCT | SARS- | SARS- | Lihong Liu et al., |
| | ASGYTFTGYYMHWVRQAPGQGLE | | GTSSDVGGYNYVSWYQQHPG | CoV2 | CoV2 | 2020 |
| | WMGWINPNSGGTNYAQKFQGRV | | KAPKLMIYDVSKRPSGVSNRF | | | (nature.com/articles/ |
| | TMTRDTSITTAYMELRRLRSDDTA | | SGSKSGNTASLTISGLQAEDEG | | | s41586-020-2571-7) |
| | VYYCARGLGVGCSGGNCYLDYYY | | DYYCSSYTSSSTWVFGGGTKL | | | |
| | MDVWGKGTTVTVSS | | TVL | | | |
| 282 | QVQLVQSGAEVKKAGSSVKVSCK | 283 | SYELTQPPSVSVSPGQTASITCS | SARS- | SARS- | Lihong Liu et al., |
| | ASGGTFSSHTITWVRQAPGQGLEW | | GDKLGDKYACWYQQKPGQSP | CoV2 | CoV2 | 2020 |
| | MGRIIPILGIANYAQKFQGRVTITA | | VLVIYQDNKRPSGIPERFSGSN | | | (nature.com/articles/ |
| | DKSTSTAYMELSSLRSEDTAVYYC | | SGNTATLTISGTQAMDEADYY | | | s41586-020-2571-7) |
| | ASLQTVDTAIEKYYGMDVWGQGT | | CQAWDSSTAVFGGGTKLTVL | | | |
| | TVTVSS | | | | | |
| 284 | EVQLVESGGGLVQPGGSLRLSCAA | 285 | QSVLTQPPSVSGAPGQRVTISC | SARS- | SARS- | Rodda, L., 2021 (pub. |
| | SEITVSSNYMSWVRQAPGKGLEW | | TGSSSNIGAGYDVHWYQQLPG | CoV2 | CoV2 | 2020), Cell, doi: |
| | VSLIYSGGSTFYADSVKGRFIISRD | | TAPKLLIYGNSNRPSGVPDRFS | | | 10.1016/ |
| | NSKNTLYLQMNSLRAEDTAVYHC | | GSKSGTSASLAITGLQAEDEAD | | | j.ce11.2020.11.029 |
| | ARGGEEPLPFDPWGQGTLVTVSS | | YYCQSYDSSLSVSVVFGGGTK | | | |
| | | | LTVL | | | |
| 286 | QVQLVQSGAEVKKPGSSVKVSCK | 287 | QSVLTQPPSVSGAPGQRVIISC | SARS- | SARS- | Rodda, L., 2021 (pub. |
| | ASGGTFSSYPISWVRQAPGQGLEW | | TGSNSNIGAGYDVHWYQQLP | CoV2 | CoV2 | 2020), Cell, doi: |
| | MGRIIPILRVANFAQRFEGRVTITA | | GTAPKLLIYGNNNRPSGVPDR | | | 10.1016/ |
| | DKSTGTAYMELSSLRSEDTAMYY | | FSGSKSGTSASLAITGLQAEDG | | | j.cell.2020.11.029 |
| | CARDEAQTTVNTNWFDPWGQGTL | | ADYYCQSYDSSLSDVVFGGGT | | | |
| | VTVSS | | KLTVL | | | |
| 288 | EVQLVESGGGLVQPGGSLRLSCAV | 289 | DIQMTQSPSSLSASVGDRVTIT | SARS- | SARS- | Rodda, L., 2021 (pub. |
| | SGFTVSSNYMSWVRQAPGKGLEW | | CRASQSISNYLNWYHQKPGKA | CoV2 | CoV2 | 2020), Cell, doi: |
| | VSVIYTGGGTYYADSVKGRFTISR | | PKLLIYAASSLQSGVPSRFSGS | | | 10.1016/ |
| | DNSKNTLYLQMNTLRAEDTTVYY | | GSGTDFTLTISSLQPEDFATYY | | | j.cell.2020.11.029 |
| | CARGDGSYYRAFDYWGQGTLVTV | | CQQSYSPPPTFGPGTKVEIK | | | |
| | SS | | | | | |
| 290 | EVQLVESGGGLIQPGGSLRLSCAA | 291 | DIQMTQSPSSLSASVGDRVTIT | SARS- | SARS- | Rodda, L., 2021 (pub. |
| | SGFTVSRNYMNWVRQAPGKGLE | | CRASQSISSYLNWYQQKPGKA | CoV2 | CoV2 | 2020), Cell, doi: |
| | WVSVIYSGGSTFYADSVKGRFTISR | | PKLLIYASSSLQRGVPSRFSGS | | | 10.1016/ |
| | DNSKNTLYLQMNSLRAEDTAVYY | | GSGTDFTLTISSLQPEDFATYY | | | j.cell.2020.11.029 |
| | CARDASSYGIDWGQGTLVTVSS | | CQQSYSTPPITFGQGTRLEIK | | | |
| 292 | QVQLKQSGPGLVAPSQSLSITCTVS | 293 | QAVVTQESALTTSPGETVTLT | SARS- | SARS- | Alsoussi, W., 2020, |
| | GFSLINYAISWVRQPPGKGLEWLG | | CRSSTGAVTTSNYANWVQEKP | CoV2 | CoV2 | J Immunol, 205: 915- |
| | VIWTGGGTNYNSALKSRLSISKDN | | DHLFTGLIGGTNNRAPGVPAR | | | 922 |
| | SKSQVFLKMNSLQTDDTARYYCA | | FSGSLIGDKAALTITGAQTEDE | | | |
| | RKDYYGRYYGMDYWGQGTSVTV | | AIYFCALWYNNHWVFGGGTK | | | |
| | SS | | LTVL | | | |
| 294 | EVQLVQSGPEVKKPGTSVKVSCKA | 295 | EIVLTQSPGTLSLSPGERATLSC | SARS- | SARS- | Aaron Schmitz et al., |
| | SGFTFSSSAVQWVRQARGQRLEWI | | RASQSVSSSYLAWYQQKPGQ | CoV2 | CoV2 | 2021 |
| | GWIVVGSGNYAQKFQERVTITRD | | APRLLICATSSRATGIPDRFSGS | | | (biorxiv.org/content/ |
| | MSTNTAYMELSSLRSEDTAVYYC | | GSGTDFTLTIRRLEPEDFALYY | | | 10.1101/ |
| | AAAYCSGGSCSDGFDIWGQGTMV | | CQQYGSSPWTFGQGTKVEIK | | | 2021.03.24.436864v1) |
| | TVSS | | | | | |
| 296 | EVQLQQSGAELVKPGASVKLSCTT | 297 | DIVMTQSQKFMSTSVGDRVSV | SARS- | SARS- | PDB |
| | SGFNIIDTYMHWVKQRPEEGLEWI | | TCKASQNVGTHVAWYQQKPG | CoV2;SARS- | CoV2; | (rcsb.org/structure/ |
| | GGIDPVNGNSEYDPKFQDKATITA | | QSPKALIYSASYRYSGVPDRFT | CoV1 | SARS- | 7EAN) |
| | DTSSNTAYLHLSRLTSEDTAVYYC | | GSGVGTDFTLTITNVQSEDLAE | | CoV1 | |
| | ASAHYYGSSSSFPYWGQGTDLVT | | YFCQQYNSYFTFGSGTKLEIK | | | |
| | VSA | | | | | |
| 298 | QVQLQQWGAGLLKPSETLSLTCA | 299 | NFMLTQPHSVSASPGKTVTIPC | SARS- | SARS- | Asarnow et al., 2021, |
| | VYGGSFSGYYWSWIRQPPGKGLE | | TGSSGNIASNYVQWYQQRPGS | CoV2 | CoV2 | Cell 184, 3192-3204 |
| | WIGEINHSGSTNYNPSLKSRVTISV | | APTTVIYEDNQRPSGVPDRFSG | | (weak) | |
| | DTSKNQFSLKLSSVTAADTAVYYC | | SIDSSSNSASLTISGLKTEDEAD | | | |
| | ARRWWLRGAFDIWGQGTTVTVSS | | YYCQSYDNNIQVFGGGTKLTV | | | |
| | | | L | | | |
| 300 | QVQLQESGGGLVQPGGSLRLSCAA | 301 | DIQLTQSPSSLSASVGHRVTITC | SARS- | SARS- | Asarnow et al., 2021, |
| | SGFTFSSYEMNWVRQAPGKGLEW | | RASQSISSYLNWYQQKPGKAP | CoV2 | CoV2 | Cell 184, 3192-3204 |
| | VAVISYDGSNKYYADSVKGRFTIS | | KLLIYAASSLQSGVPSRFSGSG | | | |
| | RDNAKNSLYLQMNSLRAEDTAVY | | SGTDFTLTISSLQPEDFATYYC | | | |
| | YCARLITMVRGEDYWGQGTLVTV | | QQSYNLPRTFGGGTKLEVL | | | |
| | SS | | | | | |
| 302 | EVQLVESGGGLVQPGGSLRLSCAA | 303 | LTQPASVSGSPGQSITISCTGTS | SARS- | SARS- | Cao, Y., et al., Cell |
| | SGVTVSSNYMSWVRQAPGKGLEW | | SDVGSYNLVSWYQQRPGKAP | CoV2 | CoV2 | Research (2021) |
| | VSAVYSGGSTYYADSVKGRFTISR | | KLILYEVTKRPSGVSNRFSGSK | | | 31: 732-741 |
| | HNSKNTLYLQMKSLRPEDTAIYYC | | SGNTASLAISGLQAEDEADYY | | | |
| | ARLINHYYDSSGDGGAFDIWGQGT | | CCSYAGSSTWVFGGGTKLTVL | | | |
| | MVTVSS | | | | | |
| 304 | QVQLVQSGAEVKKPGSSVKVSCK | 305 | EIVLTQSPGTLSLSPGERATLSC | SARS- | SARS- | Scheid et al., Cell |
| | ASGGTFSSYAISWVRQAPGQGLEW | | RASQSVGSGYLAWYQQRPGQ | CoV2 | CoV2 | (2021) 184(12): 3205- |
| | MGRIISMFGIANNAQKFQGRLTITA | | APRLLIYGASSRATGIPDRFSGS | | | 3221.e24 |
| | DTSTSTAYMELSSLRSEDTAVYYC | | GSGTDFTLTISRLEPEDFAVYY | | | |
| | ARGPYYYDSGGYYLDYWGQGTL | | CQQYAGSPRTFGQGTRLEIK | | | |
| | VTVSS | | | | | |
| 306 | EVQLVESGGGLIQPGGSLRLSCAA | 307 | EIVLTQSPGTLSLSPGERATLSC | SARS- | SARS- | Scheid et al., 2021, |
| | SGITVVRNYMTWVRQAPGKGLEW | | RASQSVPSSYLAWYQQKPGQ | CoV2 | CoV2 | Cell 184, 3205-3221 |
| | VSVIYSGGTTYYADSVKGRFTISRD | | APRLLIYGASSRATGIPDRFSGS | | | |
| | NSKNTMYLQMNSLRAEDTAIYYC | | GSGTDFTLTISRLEPEDFAVYY | | | |
| | ARDLEVVGAMDVWGQGTTVTVS | | CQQYGSPMYTFGQGTKLEIK | | | |
| | S | | | | | |
| 308 | QVQLVQSGAEVKKPGASVKVSCK | 309 | EIVLTQSPGTLSLSPGERATLSC | SARS- | SARS- | Scheid et al., 2021, |
| | ASGYTFTSYAISWVRQAPGQGLE | | RASQSVSGTFLAWYQQKPGQ | CoV2 | CoV2 | Cell 184, 3205-3221 |
| | WMGWVSAYNGNTNYAQKLQGR | | APRLLISGASSRATGIPDRFSGS | | | |
| | VTMTTDTSTNTAYMELRSLRSDDT | | GSGTDFTLTISRLEPEDFAVYY | | | |
| | AIYYCAIPYSSVTFDCWGQGTLVT | | CQQYGSSRPTFGQGTKLEIK | | | |
| | VSS | | | | | |
| 310 | QVQLVQSGAEVKKAGASVKVSCK | 311 | QSVLTQPPSVSGAPGQRVTISC | SARS- | SARS- | Scheid et al., 2021, |
| | ASGYTFTSYDINWVRQASGQGLE | | TGSSSNIGAGYDVHWYHQLPG | CoV2 | CoV2 | Cell 184, 3205-3221 |
| | WMGWMNPISGNTGYAQKFQGRV | | TAPKFLIYGNSNRPSGVPDRFS | | | |
| | TMTRNTSITTAYMELSSLRSEDTA | | GSKSGTSASLAITRLQAEDEAD | | | |
| | VYFCARGGRYCSSTTCYSGVGMD | | YYCQSYDSSLSGWVFGGGTKL | | | |
| | VWGQGTTVTVPSA | | TVL | | | |
| 312 | EVQLVQSGAEVKKPGASVKVSCK | 313 | DIVMTQTPLSSPVTLGQPASIS | SARS- | SARS- | Noy-Porat et al., |
| | VSGYTLTELSMHWVRQAPGKGLE | | CRSSQSLVHSDGNTYLSWLQQ | CoV2 | CoV2 | 2021, iScience |
| | WMGGFDPEDAETIYAQKFQGRVT | | RPGQPPRLLIYKISNRFSGVPD | | | 24, 102479 |
| | MTEDTSTDTAYMELSSLRSEDTAV | | RFSGSGAGTDFTLKISRVEAED | | | |
| | YYCVTAPAITGSPEAYSYYYGMD | | VGVYYCMQATQFPYTFGQGT | | | |
| | VWGQGTTVTVSS | | KLEIK | | | |
| 314 | QVQLVQSGAEVKKPGASVKVSCK | 315 | SYELIQEPSVSVSPGQTARITCQ | SARS- | SARS- | Noy-Porat et al., |
| | VSGYTLPELSMHWVRQAPGKGLE | | GDSLRSYYASWYQQKPGQAP | CoV2 | CoV2 | 2021, iScience |
| | WMGGFDPEDGETIYAQKFQGRVT | | VLVIYNRNKRPSGIPDRFSGSS | | | 24, 102479 |
| | MTEDTSTDTAYMELSSLRSEDTAV | | SGNTASLTITGAQADDEADYY | | | |
| | YYCATGPAIAAAATGWFDPWGQG | | CNSRDNSGNHPFGGGTKVTVL | | | |
| | TLVTVSS | | | | | |
| 316 | QVQLQQSGAEVKKPGASVKVSCK | 317 | DIVMTQSPLSSPVTLGQPASISC | SARS- | SARS- | Noy-Porat et al., |
| | VSGYTLTELSMHWVRQAPGKGLE | | RSSQSLVHSDGNTYLSWLQQR | CoV2 | CoV2 | 2021, iScience |
| | WMGGFDPEDAETIYAQKFQGRVT | | PGQPPRLLIYKISNRFSGVPDRF | | | 24, 102479 |
| | MTEDTSTDTAYMELSSLRSEDTAV | | SGSGATDFTLKISRVEAEDVG | | | |
| | YYCVTAPVITGSPEAYSYYYGMD | | VYYCMQATQFPITFGGGTKVE | | | |
| | VWGQGTTVNVSS | | IK | | | |
| 318 | QMQLVQSWGRRGPAGRSLRLSCA | 319 | EIVLTQSPGTLSLSPGERATLSC | SARS- | SARS- | Noy-Porat et al., |
| | ASGSTFSSYGMHWVRQAPGKGLE | | RASQSVSSSYLAWYQQKPGQ | CoV2 | CoV2 | 2021, iScience |
| | WVAVISFDGSNKFYADSVKGRFTI | | APRLLIYGASSRATGIPDRFSGS | | | 24, 102479 |
| | SRDNSKNTLYLQMNSLRAEDTAV | | GSGTDFTLTISRLEPEDFAVYY | | | |
| | YYCAKDHDDGYYFYYYMDVWG | | CQQYGSSRTFGGGTRLEIK | | | |
| | KGTTVTVSS | | | | | |
| 320 | EVQLVQSGAEVKKPGASVKVSCK | 321 | QSVVTQPASVSGSPGQSITISCT | SARS- | SARS- | Noy-Porat et al., |
| | VSGYTLTELSMHWVRQAPGKGLE | | GSSSDIGGYNYVSWYQQHPGK | CoV2 | CoV2 | 2021, iScience |
| | WMGGFDPEDGETIYAQKFQGRVT | | APKLMIFEGSKRPSGVPDRFSG | | | 24, 102479 |
| | MTEDTSTDTAYMELSSLRSEDTAV | | SKSGNTASLTISGLQAEDEADY | | | |
| | YYCAASPAVRGSPSNFYYYHGMD | | YCSSYTSSSTLVFGGGTELTVL | | | |
| | VWGQGTMVTVSS | | | | | |
| 322 | QVQLQQSGAEVKKPGASVRVSCK | 323 | SQSALTQPGSLSGSPGQSITISC | SARS- | SARS- | Noy-Porat et al., |
| | VSGYTLTELSMHWVRQAPGKGLE | | TGTSSDVGSYNLVSWYQQHP | CoV2 | CoV2 | 2021, iScience |
| | WMGGFDPEDAETIYAQKFQGRVT | | GQTPKVIIYEVTNRASGVSNRC | | | 24, 102479 |
| | MTEDTSTDRAYMELSSLRSEDTAV | | SGSQSGNTATLTISRLLADDQA | | | |
| | YYCVAAPVITGSPEAYSYYYGMD | | DDYCSSYTRTSTLDVVFGGRT | | | |
| | VWGQGTTVTVSS | | ELTVL | | | |
| 324 | QLQLQESGPGLVKPSQTLSLTCTVS | 325 | NILTQPPSASGTPGQRVTISCSG | SARS- | SARS- | Noy-Porat et |
| | GGSISSGSYYWSWIRQPAGKGLEW | | SSSNIGSNTVN | CoV2 | CoV2 | al.,2021, iScience |
| | IGRIYTTGSTSYNPSLKSRVTISVDT | | WYQQLPGTAPKLLIYSKNQRP | | | 24,102479 |
| | SKNQFSLKLSSVTAADTAVYYCAR | | SGVPDRFSGSKSGTSASLAISG | | | |
| | MAYQVYYYDSSGYYDAFDIWGQ | | LRSEDEADYYCSAWDDSLRG | | | |
| | GTMVTVSS | | YVFGPGTKVTVL | | | |
| 326 | QVQLQESGPGLVKPSQTLSLTCTV | 327 | QSALTQPASASGSPGQSVTISC | SARS- | SARS- | Noy-Porat et al., |
| | SGGSISSGSYYWSWIRQPAGKGLE | | TGSSSDVGGYNFVSWYQQHP | CoV2 | CoV2 | 2021, iScience |
| | WIGRIYTTGSTNYNPSLKSRVTISV | | GKAPKLIIYEVSKRPSGVPNRF | | | 24, 102479 |
| | DTSKNQFSLKLSSVTAADTAVYYC | | SGSKSGNTASLTVSGLQADDE | | | |
| | ARMAYQVYYYDSSGYYDAFDIW | | ALYYCSSYAGSNNYVFGPGTK | | | |
| | GQGTMVTVSS | | VTVL | | | |
| 328 | EVQLVQSGAEVKKPGASVKVSCK | 329 | QSVLTQPASVSGSPGQSITISCT | SARS- | SARS- | Noy-Porat et al., |
| | VSGYTLPELSMHWVRQAPGKGLE | | GTSSDVGGYNYVSWYQQHPG | CoV2 | CoV2 | 2021, iScience |
| | WMGGFDPEDGGTIYAQKFQGRVT | | KAPKLMIYDVSNRPSGVPDRF | | | 24, 102479 |
| | MTEDTSTDTAYMELSSLRSEDTAV | | SGSKSGNTASLTISGLQAEDEA | | | |
| | YYCATSRVAGTPNWFHPWGQGTL | | DYYCSSYTSSSTLVFGTGTKVT | | | |
| | VTVSS | | VL | | | |
| 330 | EVQLLESGGGVVQPGRSLRLSCAA | 331 | NFMLTQPPSVSVSPGQTASITC | SARS- | SARS- | Noy-Porat et al., |
| | SGFTFSSYAMHWVRQAPGKGLQW | | SGDKLGDKYASWYQQKPGQS | CoV2 | CoV2 | 2021, iScience |
| | VALISYDGSNKYYADSVKGRFTIS | | PVLVIYQDSKRPSGIPERFSGSN | | | 24, 102479 |
| | RDNSKNTLYLQMNSLRAEDTAVY | | SGNTATLTISGTQAMDEADYY | | | |
| | YCARDLGSGWYPWGQGTLVTVSS | | CQAWDSSTVVFGGGTELTVL | | | |
| 332 | QVQLVQSGAGVKKPGASVKVSCK | 333 | QSALTQPVSVSGSPGQSITISCT | SARS- | SARS- | Noy-Porat et al., |
| | VSGYTLPELSMHWVRQAPGKGLE | | GTSSDVGRYNYVSWYQQHPG | CoV2 | CoV2 | 2021, iScience |
| | WMGGFDPEDGETIYAQKFQGRVT | | KAPKLITYDVKNRPSGVPDRFS | | | 24, 102479 |
| | MTEDTSTDTAYMELSSLRSEDTAV | | GSKSGNTASLTISGLQAEDEAD | | | |
| | YYCATSRVAGTPNWFHPWGQGTL | | YYCSSYTRSSTLDVVFGGGTE | | | |
| | VTVSS | | LTVL | | | |
| 334 | EVQLVESGGGLVQPGGSLRLSCAA | 335 | AIQMTQSPSFLSASVGDRVTIT | | | Andreano et al., |
| | SGLTVSSNYMSWVRQAPGKGLEW | | CRASQGISSYLAWYQQKPGKA | | | 2021, Cell, |
| | VSVIYSGGSTFYADSVKDRFTISRD | | PKLLIYAASTLQSGVPSRFSGS | | | 184(7): 1821-1835 |
| | NSKNTLYLQMNSLRAEDTAVYYC | | GSGTEFTLTISSLQPEDFATYY | | | |
| | VRDLYSYGMDVWGQGTTVTVSS | | CQQVNSYPTFGQGTRLEIK | | | |
| 336 | EVQLQQWGAGLLKPSETLSLTCAV | 337 | EIVLTQSPATLSLSPGERATLSC | SARS- | SARS- | Song et al., 2021, |
| | YGGSFSGYYWSWIRQPPGKGLEWI | | RASQSVSSYLAWYQQKPGQA | CoV2 | CoV2 | Communications |
| | GEVNHSGSTNYNPSLKSRVTISVD | | PRLLIYDASNRATGIPARFSGS | | | Biology volume 4, |
| | TSKNQLSLKLNSVTAADTAVYYC | | GSGTDFTLTISSLEPEDFAVYY | | | Article number: 500 |
| | ARGNTMVRGVIIPFEYWDKGTLVT | | CQQRSNWPLTFGGGTKVEIK | | | (2021) |
| | VSS | | | | | |
| 338 | EVQLLESGGGLVQPGGSLRLSCAA | 339 | SYELTQPPSVSVSPGQTARITC | SARS- | SARS- | Panpan Zhou et al., |
| | SGFTFSSYVMTWARQAPGKGLEW | | SGDALPKRYAYWYQQKSGQA | CoV2 | CoV2 | 2021 |
| | VSAISGTGYTYYADSVKGRFTVSR | | PILVIYEDKKRPSGIPERLSGSK | | | (biorxiv.org/content/ |
| | DNSKNTLFLQMSSLRAEDTAVYY | | SGTVATLTISGAQVEDEADYY | | | 10.1101/ |
| | CAITMAPVVWGQGTTVTVSS | | CYSTDSSGNHAVFGGGTQLTV | | | 2021.03.30.437769v1) |
| | | | L | | | |
| 340 | QVQLVESGGGLVKPGGSLRLSCAA | 341 | EIVLTQSPATLSLSPGERATLSC | SARS- | SARS- | Voss et al., 2021, |
| | SGFTFSDYYMSWIRQAPGKGLEW | | RASQSVSTYLAWYQQKPGQA | CoV2 | CoV2 | Science, 372(6546): |
| | VSSIDGSTSYTKYADSVKGRFTISR | | PRLLIFDASNRATGIPARFSGSG | | | 1108-1112 |
| | DNAKNSLYLQMNSLRAEDTAVYY | | SGTDFTLTISSLEPEDFAVYYC | | | |
| | CARVGPAVAGSPFDSWGQGTLVT | | QQRSNWLFSFGPGTKVDIK | | | |
| | VSS | | | | | |
| 342 | QVQLVQSGPEVKKPGASVKVSCQ | 343 | QSVLTQPASVSASPGQSITISCT | SARS- | SARS- | Voss et al., 2021, |
| | TSGYSFSDHYLHWVRQTPGQGFE | | GTSSDVGGYDYVSWYQHHPD | CoV2 | CoV2 | Science, |
| | WMGWIKPKSGATNSADNFQDRVT | | NAPKLLIFEVSNRPSGVSNRFS | | | 372(6546): 1108-1112 |
| | MTADASVNTAYMELSSLRPDDTA | | GSKSGNAASLTISGLLAEDEAD | | | |
| | VYYCARGHRIPSAISDKYDFWGQG | | YFCTSYSSGRTPYVFGTGTKV | | | |
| | TLVTVSS | | TVL | | | |
| 344 | EVQLVESGGGLIQPGGSLRLSCAA | 345 | DVVMTQSPGTLSLSPGERATL | SARS- | SARS- | Dejnirattisai et al., |
| | SGLTVSSNYMSWVRQAPGKGLEW | | SCRASQSVPSSYLAWYQQKPG | CoV2 | CoV2 | 2021, Cell, |
| | VSVIYSGGSTFYADSVKGRFTISRD | | QAPRLLIYGASTRATGIPDRFS | | | 184(11): 2939-2954 |
| | NSKNTLYLQMNSLGAEDTAVYYC | | GSGSGTDFTLTISRLEPEDFAV | | | |
| | ARGEGSPGNWFDPWGQGTLVTVS | | YYCQHYDTSPRFGGGTKVDIK | | | |
| | S | | | | | |
| 346 | EVQLVESGGGLVQPGGSLRLSCAA | 347 | AIQMTQSPSSLSASVGDRVTIT | SARS- | SARS- | Andreano et al., |
| | SGFTVSINYMSWVRQAPGKGLEW | | CRASQDIRNNLGWFQQKPGK | CoV2 | CoV2 | 2021, Cell, |
| | VSVIYSGGSTYYADSVKGRFTISRD | | APKRLIYAASTLQRGVPSRFSG | | | 184(7): 1821-1835 |
| | NSKNTLYLQMNSLRAEDTAVYYC | | SGSGTEFTLTISSLQPEDFATYY | | | |
| | AAPLLWADSYYMDVWGKGTTVT | | CLQHNSYLWTFGQGTKVEIK | | | |
| | VSS | | | | | |
| 348 | QVQLVQSGAEVKKPGPSVKVSCK | 349 | DIVMTQSPAALSVSPGERATLS | SARS- | SARS- | Andreano et al., |
| | ASGGTFSSYTINWVRQAPGQGLE | | CRASQSVSSNLAWYQQKPGQ | CoV2 | CoV2 | 2021, Cell, |
| | WMGRIIPMLGIAKYAQKFQGRVTI | | APRLLIYGASTRATGIPARFSG | | | 184(7): 1821-1835 |
| | TADKSTSTAYMELSSLRSEDTAVY | | SWSGTEFTLTISSLQSEDLAVY | | | |
| | YCARGIVGATPGYFDYWGQGTLV | | YCQQYNNWLTFGGGTKVEIK | | | |
| | TVSS | | | | | |
| 350 | QVQLVQSGAEVKKPGSSVKVSCK | 351 | EIVMTQSPATLSLSPGERATLS | SARS- | SARS- | Andreano et al., |
| | ASGGTFSSYAISWVRQAPGQGLEW | | CRASQSVSSYLAWYQQKPGQ | CoV2 | CoV2 | 2021, Cell, |
| | MGGITPIFHTANYAQKFQGRVTIT | | APRLLIYDASNRATGIPARFSG | | | 184(7): 1821-1835 |
| | ADESTSTVYMELSSLSSEDTAVYY | | SGSGTDFTLTISSLEPEDFAVY | | | |
| | CARETGDQGVTAPFDLWGRGTLV | | YCQLRGNWPPWTFGQGTKVEI | | | |
| | TVSS | | K | | | |
| 352 | QVQLVQSGPEVKKPGTSVKVSCK | 353 | EIVMTQSPGTLSLAPGERATLS | SARS- | SARS- | Andreano et al., |
| | ASGFTFTSSAMQWVRQARGQRLE | | CRASQSVSSSYLGWYQQKPGQ | CoV2 | CoV2 | 2021, Cell, |
| | WIGWIVVGSGNTDYVQKFQGRVTI | | APRLLIYGASSRATGIPDRFSGS | | | 184(7): 1821-1835 |
| | TRDMSTSTAYMELSSLRSEDTAVY | | GSGTDFTLTISRLEPEDFAVYY | | | |
| | YCAAPYCSSTTCHDGFDIWGQGT | | CQQYGRSPWTFGQGTKVEIK | | | |
| | MVTVSS | | | | | |
| 354 | QVQLVQSGAEVKKPGSSVKVSCK | 355 | EIVMTQSPATLSLSPGERATLS | SARS- | SARS- | Andreano et al., |
| | ASGGTFSSYTISWVRQAPGQGLEW | | CRASQSVSSYLAWYQQKPGQ | CoV2 | CoV2 | 2021, Cell, |
| | MGRIIPILDRVMYAQKFQGRVTITA | | APSLLIYDASNRATGIPARFSG | | | 184(7): 1821-1835 |
| | DKSTSTAYMELSSLRSEDTAVYYC | | SGSGTDFTLTISSLEPEDFAVY | | | |
| | ARRAIDSDTYVEQSHFDYWGQGT | | YCQQPLTFGGGTKVEIK | | | |
| | LVTVSS | | | | | |
| 356 | QVQLQESGAEVKKPGSSVKVSCK | 357 | EIVMTQSPATLSLSPGERATLS | SARS- | SARS- | Andreano et al., |
| | ASGGTFSSYAINWVRQAPGQGLE | | CRASQSVSSFLAWYQQKPGQA | CoV2 | CoV2 | 2021, Cell, |
| | WMGGLIPIFGTANYAQKFQGRVTI | | PRLLIYDASNRATGIPARFSGS | | | 184(7): 1821-1835 |
| | TADESTSTAYMELSSLRSEDTAVY | | GSGTDFTLTISSLEPEDFAVYY | | | |
| | YCANFIGDGYNYEEDYMDVWGK | | CQQRSNWPPFTFGPGTKVDIK | | | |
| | GTTVTVSS | | | | | |
| 358 | EVQLVESGGGLVKPGGSLRLFCAA | 359 | DIVMTQSPSSLSASVGDRVTIT | SARS- | SARS- | Andreano et al., |
| | SGFTFSDYYMSWFRQAPGKGLEW | | CQASQDISNYLNWYQQKPGK | CoV2 | CoV2 | 2021, Cell, |
| | VSYISSSGSNIYYADSVKGRFTVSR | | APKLLIYDASNLQTGVPSRFSG | | (weak) | 184(7): 1821-1835 |
| | DNAKNSLYLQMNSLRAEDTAVYF | | SGSGTDFTFTISSLQPEDIATYY | | | |
| | CARGRLWGWFDPWGQGTLVTVSS | | CQQYDHLLITFGQGTRLEIK | | | |
| 360 | QITLKESGPTLVKPTQTLTLTCTFS | 361 | QSALTQPASVSGSPGQSITISCT | SARS- | SARS- | Kathryn Westendorf |
| | GFSLSISGVGVGWLRQPPGKALEW | | ATSSDVGDYNYVSWYQQHPG | CoV | CoV2 | et al., 2021 |
| | LALIYWDDDKRYSPSLKSRLTISKD | | KAPKLMIFEVSDRPSGISNRFS | | | (biorxiv.org/content/ |
| | TSKNQVVLKMTNIDPVDTATYYC | | GSKSGNTASLTISGLQAEDEAD | | | 10.1101/ |
| | AHHSISTIFDHWGQGTLVTVSS | | YYCSSYTTSSAVFGGGTKLTVL | | | 2021.04.30.442182v3) |
| 362 | QVQVVQSGAEVKKPGASVKVSCK | 363 | QSALTQPASVSGSPGQSITISCT | SARS- | SARS- | Cao et al., 2021, |
| | VSGYTLIEISIHWVRQAPGKGLEW | | GTSSDVGSYNYVSWYQQHPG | CoV2 | CoV2 | Cell Research (2021) |
| | MGGFDPEAGETIYAQKFQGRVTM | | KAPKLMIYDVTKRPSGVPDRF | | | 0: 1-10; doi: |
| | TEDTSTDTAYMEVSSLRSEDTAVY | | SGSKSGNTASLTISGLQAEDEA | | | 10.1038/s41422-021- |
| | YCATGPAIAAAETNWFDLWGQGT | | DYYCSSYTSSSTWVFGGGTKL | | | 00514-9 |
| | LVTVSS | | TVL | | | |
| 364 | EVQLVESGGGLVKPGGSLRLSCAA | 365 | SYELTQPPSVSVSPGQTATITCS | SARS- | SARS- | Cao et al., 2021, Cell |
| | SEFTFSSYSMNWVRQAPGKGLEW | | GDKLGDKYACWYQQRPGQSP | CoV2 | CoV2 | Research (2021) 0: 1- |
| | VSSISSSGSQIYYADSVKGRFTISRD | | VLVIYQDSKRPSGIPERFSGSNS | | | 10; doi: |
| | NAKKSLYLQMNSLRVEDTAVYYC | | GNTATLTIGGTQAMDEAAYFC | | | 10.1038/s41422-021- |
| | ATNGGAHSSTWSFYGMDVWGQG | | QAWDSNTGVFGGGTKLTVL | | | 00514-9 |
| | TTVTVSS | | | | | |
| 366 | EVQLVESGGGLVKPGGSLRLSCAA | 367 | AIRMTQSPSSLSASVGDRVTIT | SARS- | SARS- | Rosa et al., 2021, |
| | SGFSFSSYSMNWVRQAPGKGLEW | | CQASQDISNYLNWYQQKPGK | CoV2 | CoV2 | Science Advances |
| | VSSISSNSNYIYYADSMKGRFTISR | | APKLLIYDASNLETGVPSRFSG | | | (2021): Vol. 7, no. |
| | DNAKNSLYLQMNSLRAEDTAVYY | | SGSGTDFTFTISSLQPEDIATYY | | | 22, eabg7607 |
| | CASNRSPYDSSNYYFDYWGQGTR | | CQHHDSLPLTFGGGTKVEIK | | | |
| | VTISS | | | | | |
| 368 | EVQLVESGGGVVQPGRSLRLSCAA | 369 | DIVMTQSPLSLPVTPGEPASISC | SARS- | SARS- | Fu et al., PLOS |
| | SGFTFSYYGMHWVRQAPGKGLEW | | RSSQSLLHSNGYNYLDWYLQ | CoV2 | CoV2 | Biology, (2021), doi: |
| | VAVIWYDGSNRFYADSVKGRFTIS | | KPGQSPQLLIYLGSNRASGVPD | | | 10.1371/journal.pbio. |
| | RDNSKSTLYLQMNSLRAEDTAVY | | RFSGSGSGTDFTLKISRVEAED | | | 3001209 |
| | YCATDPPGLRFRFDYWGQGTLVT | | VGVYYCMQGLQTPLTFGGGT | | | |
| | VSS | | KVEIK | | | |
| 370 | EVQLQQSGPELVKPGASVKISCKT | 371 | DIVMTQSQKFMSTSVGDRVSV | SARS- | SARS- | Fu et al., PLOS |
| | SGYTFTEYTMHWVKQSHGKSLEW | | TCKASQNVGTNVAWYQQKPG | CoV2 | CoV2 | Biology, (2021), doi: |
| | IGGINPNNGDNTYNQKLKGKATLT | | QSPKPLIYSASSRYSGVPDRFT | | | 10.1371/journal.pbio. |
| | VHKSSSTAYMELRSLTSEDSAVYY | | GSGSGTDFTLTISNVQSEDLAE | | | 3001209 |
| | CARDGYPYYYALDYWGQGTSVT | | YFCQQYNNYPWTFGGGTKLEI | | | |
| | VSS | | K | | | |
| 372 | QVQLVQSGAEVKKPGASVKLSCK | 373 | DIVLTQSPASLAVSPGQRATIT | SARS- | SARS- | Fu et al., PLOS |
| | ASGYSFTSYWVNWVRQAPGQGLE | | CRASESVDSYGNSFMHWYQQ | CoV2 | CoV2 | Biology, (2021), doi: |
| | WIGMIHPSDSETRLNQKFKDRVTIT | | KPGQPPKLLIYRASNLESGIPA | | | 10.1371/journal.pbio. |
| | VDKSTSTAYMELSSLRSEDTAVYY | | RFSGSGSGTDFTLTINPVEAND | | | 3001209 |
| | CARADGYEWYFDVWGRGTLVTV | | VANYYCQQSNEDPWTFGQGT | | | |
| | SS | | KVEIK | | | |
| 374 | QVQLQQSGAELVKPGASVKLSCK | 375 | DIVLTQSPASLAVSLGQRATIS | SARS- | SARS- | Guo et al., JBC |
| | ASGYTFTSYYIYWMKQRPGQGLE | | CRASESVEYYGTGLMQWYQQ | CoV2 | CoV2 | (2021), 296 |
| | WIGEINPNNGGTNFNEKFKSKATL | | KPGQPPKLLIYAASNVESGVPA | | | (100346): 1-9 |
| | TVDKSSSTAYMQLSSLTSEDSAVY | | RFSGSGSGTDFSLNIHPVEEDDI | | | |
| | YCTRGHSDYWGQGTTLTVSS | | AMYFCQQTRKVPYTFGGGTK | | | |
| | | | LEIK | | | |
| 376 | EVQLVESGGGLVNPGGSLRLSCAA | 377 | VTQPASVSGSPGQSITISCTGTS | SARS- | SARS- | rcsb.org/structure/ |
| | SGFTFSDYTIHWVRQAPGKGLEW | | SDVGGYNYVSWYQQHPGKAP | CoV2 | CoV2 | 7LXX |
| | VSSISSSSNYIYYADSVKGRFTISRD | | KLMIYDVSDRPSGVSNRFSGS | | | |
| | NAKNSLSLQMNSLRAEDTAVYYC | | KSGNTASLTISGLQAEDEADY | | | |
| | ARDGNAYKWLLAENVRFDYWGQ | | YCSSYTSSSTPNWVFGGGTKL | | | |
| | GTLVTVSS | | T | | | |
| 378 | VQLVESGGGVVQPGRSLRLSCAAS | 379 | YELTQPPSVSVSPGQTARITCS | SARS- | SARS- | rcsb.org/structure/ |
| | GFTFSSYGMHWVRQAPGKGLEWV | | GDALAKHYAYWYRQKPGQAP | CoV2 | CoV2 | 71y0 |
| | TVIWYDGSNRYYADSVKGRFTISR | | VLVIYKDSERPSGIPERFSGSSS | | | |
| | DNSKNTLYLQMDSLRAEDTAVYY | | GTTVTLTISGVQAEDEADYYC | | | |
| | CARAVAGEWYFDYWGQGTLVTV | | QSADSIGSSWVFGGGTKLTV | | | |
| | S | | | | | |
| 380 | VQLVESGGGVVQPGRSLRLSCAAS | 381 | YELTQPPSVSTARITCGGNNIE | SARS- | SARS- | rcsb.org/structure/ |
| | GFTFSNYGMHWVRQAPGKGLEW | | RKSVHWCQQKPGQAPALVVY | CoV2 | CoV2 | 7LXW |
| | VAVIWYDGSNKFYADSVKGRFTIS | | DDSDRPSGIPERFSGSNSGNTA | | | |
| | RDNSKNSLYLQMNSLRAEDTAVY | | TLTISRVEAGDEADYYCQVWD | | | |
| | FCARAFPDSSSWSGFTIDYWGQGT | | SGSDQVIFGGGTKLT | | | |
| | LVTV | | | | | |
| 382 | QVRLVQSGAEVKKSGESLKISCKG | 383 | QSVLTQPASVSGSPGQSITISCT | SARS- | SARS- | rcsb.org/structure/ |
| | SGYSFTSYWIGWVRQMPGKGLEW | | GISSDVGGYNSVSWYQQHPGK | CoV2 | CoV2 | 7M7W; Starr, TN, et |
| | MGIIYPGDSDTRYSPSFQGQVTISA | | APKLMIYDVTNRPSGVSNRFS | | | al., Nature, 2021 |
| | DKSISTVYLQWSSLKASDTAMYYC | | GSKSGNTASLTISGLQAEDEAD | | | Jul 14. doi: |
| | ARQWSHYTYDYYYWGQGTLVTIS | | YYCSSYTSSSTPPYVFGTGTKV | | | 10.1038/s41586- |
| | S | | SVL | | | 021-03807-6. |
| 384 | QVQLVQSGAEVKKPGSSVKVSCK | 385 | QTVLTQPPSVSGAPGQRVTISC | SARS- | SARS- | rcsb.org/structure/ |
| | ASGGIFNTYTISWVRQAPGQGLEW | | TGSNSNIGAGYDVHWYQQLP | CoV2, | CoV2, | 7MKL; Tortorici et |
| | MGRIILMSGMANYAQKIQGRVTIT | | GTAPKLLICGNSNRPSGVPDRF | SARS- | SARS- | al., Nature doi: |
| | ADKSTSTAYMELTSLRSDDTAVYY | | SGSKSGTSASLAITGLQAEDEA | CoV1 | CoV1 | 10.1038/s41586-021- |
| | CARGFNGNYYGWGDDDAFDIWG | | DYYCQSYDSSLSGPNWVFGG | | | 03817-4(2021) |
| | QGTLVTVYS | | GTKLTVL | | | |
| 386 | QVQLVQSGAEVKKPGASVKVSCK | 387 | DIVMTQTPLSSPVTLGQPASIS | SARS- | SARS- | Peter et al., 2021, |
| | VSGYTLTEVSVHWVRQAPEKGLE | | CRSSQSLVHSDGNTYLSWLQQ | CoV2 | CoV2 | BioRxiv, doi: |
| | WMGGFDPEDAETIYAQKFQGRVT | | RPGQPPRLIIYKVSNRFCGVPD | | | 10.1101/ |
| | MTEDTSTDTAYMELSSLRSEDTAV | | RFSGSGAGTDFTLKISRVEAED | | | 2021.04.16.440101 |
| | YFCATAPAVAGPFYYYYYGMDV | | VGVYYCTQATQFPHTFGQGTK | | | |
| | WGQGTTVTVSS | | LEIK | | | |
| 388 | QVQLVQSGAEVKKPGASVKVSCK | 389 | DIVMTQTPLSSPVTLGQPASIS | SARS- | SARS- | Peter et al., 2021, |
| | VSGYTLTEVSVHWVRQAPGKGLE | | CRSSQSLVHSDGNTYLSWLQQ | CoV2 | CoV2 | BioRxiv, doi: |
| | WMGGFDPEDAKTIYAQKFQGRVT | | RPGQPPRLLIYKVSNRFSGVPD | | | 10.1101/ |
| | MTEDTSTDTAYMELSSLSSEDTAV | | RFSGSGAGTDFTLKISRVEAED | | | 2021.04.16.440101 |
| | YFCATAPAVAGPFYYYYYGMDV | | VGVYYCTQATQFPHTFGQGTK | | | |
| | WGQGTTVTVSS | | LEIK | | | |
| 390 | QVQLVQSGAEVKKPGASVKVSCK | 391 | DIVMTQTPLSSPVTLGQPASIS | SARS- | SARS- | Peter et al., 2021, |
| | VSGYSLIEVSVHWVRQAPGKGLE | | CRSSQSLVHSDGNTYLSWLQQ | CoV2 | CoV2 | BioRxiv, doi: |
| | WMGGFDPENAETIYARGFRGRVT | | RPGQPPRLLIYKVSNRFSGVPD | | | 10.1101/ |
| | MTEDTSTDTAYMELSSLKYEDTA | | RFSGSGAGTEFTLKISRVEAED | | | 2021.04.16.440101 |
| | VYFCATAPAVAGPFYYYYYGMDV | | VGVYYCTQATQFPHTFGQGTK | | | |
| | WGQGTTVTVSS | | LEIK | | | |
| 392 | QVQVVESGGGVVQPGRSLRLSCA | 393 | NIQMTQSPSAMSASVGDSVTIT | SARS- | SARS- | Peter et al., 2021, |
| | ASGFTFSGYGMHWVRQAPGKGLE | | CRARQDINNYLAWFQQKPGK | CoV2 | CoV2 | BioRxiv, doi: |
| | WVAVIWYDGSNQYYADSVKGRFT | | VPKHLIYAASSLLSGVPSRFSG | | | 10.1101/ |
| | ISRDNSKNTLYLQMNSLRVEDTAV | | SGSGTEFTLTISSLQPEDFATYY | | | 2021.04.16.440101 |
| | YHCVRETVDGMDVWGQGTTVTV | | CLQHNSYPYTFGQGTKLEIK | | | |
| | SS | | | | | |
| 394 | QHQLVQSGAEVKKPGASVKVSCK | 395 | DIVMTQTPLSSPVTLGQPASIS | SARS- | SARS- | Peter et al., 2021, |
| | VSGYTLVEVSVHWVRQAPGKGFE | | CRSSQSLVHSDGNTYLSWLQQ | CoV2 | CoV2 | BioRxiv, doi: |
| | WMGGFDPENAATIYAQKFQGRVT | | RPGQPPRLLIYKVSNRFSGVPD | | | 10.1101/ |
| | MTEDTSTDTAYMELSSLRYEDTAV | | RFSGSGAGTEFTLKISRVEAED | | | 2021.04.16.440101 |
| | YFCATAPAVAGPLYYYYYGMDV | | VGVYYCTQATQFPHTFGQGTK | | | |
| | WGQGTTVTVSS | | LEIK | | | |
| 396 | QVQLVQSGAEVKKPGASVKVSCK | 397 | DIVMTQTPLSSPVTLGQPASIS | SARS- | SARS- | Peter et al., 2021, |
| | VSGYTLTEVSVHWVRQAPGKGLE | | CRSSQSLVHSDGNTYLSWLQQ | CoV2 | CoV2 | BioRxiv, doi: |
| | WMGGFDPEDAETIYAQKFQGRVT | | RPGQPPRLLIYKVSNRFSGVPD | | | 10.1101/ |
| | MTEDTSTDTAYMELSSLRYEDTAV | | RFSGSGAGTEFTLKISRVEAED | | | 2021.04.16.440101 |
| | YFCATAPAVAGPFYYYYYGMDV | | VGVYYCTQATQFPHTFGQGTK | | | |
| | WGQGTTVTVSS | | LEIK | | | |
| 398 | QVQVVESGGGVVQPGRSLRLSCA | 399 | NIQMTQSPSAMSASVGDSVTIT | SARS- | SARS- | Peter et al., 2021, |
| | ASGFTFSSYGMHWVRQAPGKGLE | | CRARQDINNYLAWFQQKPGK | CoV2 | CoV2 | BioRxiv, doi: |
| | WVAVIWYDGSNKYYADSVKGRFT | | VPKHLIYAASSLLSGVPSRFSG | | | 10.1101/ |
| | ISRDNSKNTLYLQMNSLRVEDTAV | | SGSGTEFTLTISSLQPEDFATYY | | | 2021.04.16.440101 |
| | YYCVRETVDGMDVWGQGTTVTV | | CLQHNSYPYTFGQGTKLEIK | | | |
| | SS | | | | | |
| 400 | QVQVVESGGGVVQPGRSLRLSCA | 401 | NIQMTQSPSAMSASVGDSVTIT | SARS- | SARS- | Peter et al., 2021, |
| | ASGFTFSSYGMHWVRQAPGKGLE | | CRARQDINNYLAWFQQKPGK | CoV2 | CoV2 | BioRxiv, doi: |
| | WVAVIWYDGSNKYYADSVKGRFT | | VPKHLIYAASSLLSGVPSRFSG | | | 10.1101/ |
| | ISRDNSKNTLYLQMNSLRVEDTAV | | SGSGTEFTLTISSLQPEDFATYY | | | 2021.04.16.440101 |
| | YYCVRETVDGMDVWGQGTTVTV | | CLQHNSYPYTFGQGTKLEIK | | | |
| | SS | | | | | |
| 402 | QVQLVQSGAEVKKPGASVKVSCK | 403 | DIVMTQTPLSSPVTLGQPASIS | SARS- | SARS- | Peter et al., 2021, |
| | VSGYTLTEVSVHWVRQAPGKGLE | | CRSSQSLVHSDGNTYLSWLQQ | CoV2 | CoV2 | BioRxiv, doi: |
| | WMGGFDPEDAETIYAQKFQGRVT | | RPGQPPRLLIYKVSNRFSGVPD | | | 10.1101/ |
| | MTEDTSTDTAYMELSSLRSEDTAV | | RFSGSGAGTDFTLKISRVEAED | | | 2021.04.16.440101 |
| | YFCATAPAVAGPFYNFYYGIDVW | | VGVYYCTQATQFPHTFGQGTK | | | |
| | GQGTTVTVSS | | LEIK | | | |
| 404 | QVQLVQSGAEVKKPGASVKVSCK | 405 | SYELTQPPSVSVSPGQTARITC | SARS- | SARS- | Wang et al., 2021, |
| | ASGYTFTSYGISWVRQAPGQGLE | | SGDALPKQYAYWYQQKPGQA | CoV2 | CoV2 | Science Translational |
| | WMGWISAYNGNTNYAQKLQGRV | | PVLVIYKDSERPSGIPERFSGSS | | (weak) | Medicine, |
| | TMTTDTSTSTAYMELRSLRSDDTA | | SGTTVTLTISGVQAEDEADYY | | | 13(577): eabf1555 |
| | VYYCARVPASYGDDDYYYYYGM | | CQSADSSGTLWVFGGGTKLTV | | | |
| | DVWGQGTTVTVSS | | L | | | |
| 406 | EVQLLESGGGLVQPGGSLRLSCAA | 407 | QTVVTQEPSLTVSPGGTVTLTC | SARS- | SARS- | Wang et al., 2021, |
| | SGFTFSNYAMSWVRQAPGKGLEW | | GSSTGAVTSGHYPYWFQQKSG | CoV2 | CoV2 | Science Translational |
| | VSGISNSGGSTYYEDSVKGRFTISR | | QAPRTLIYETNIKHSWTPARFS | | | Medicine, |
| | DNSKNTLYLQMNSLRAEDTAVYY | | GSLLGGKAALTLSGAQPDDEA | | | 13(577): eabf1555 |
| | CAKVGEYCGDDCYRGLDYWGQG | | DYYCLLSYSGARPVFGGGTKL | | | |
| | TLVTVSS | | TVL | | | |
| 408 | EVQLVESGGGLVQPGGSQRLSCAA | 409 | EIVMTQSPATLSVSPGERATLS | SARS- | SARS- | Wang et al., 2021, |
| | SGFTVSSNYMSWIRQAPGKGLEW | | CRASQSVSSHLAWYQQKPGQ | CoV2 | CoV2 | Science Translational |
| | VSVIYSGGSAYYVDSVKGRFTISR | | APRLLIYGASTRATGIPTRFSGS | | (weak) | Medicine, |
| | DNSKNTLYLQMNSLRPEDTAVYY | | GSGTEFTLTISSLQSEDFAVYY | | | 13(577): eabf1555 |
| | CARIANYMDVWGKGTTVTVSS | | CQQYNNWPPLTFGGGTKVEIK | | | |
| 410 | QVQLVESGGGLIQPGGSLRLSCAA | 411 | EIVLTQSPGTLSLSPGERATLSC | SARS- | SARS- | Wang et al., 2021, |
| | SGLTVSSNYMTWVRQAPGKGLEW | | RASQSVSSNYLAWYQQKPGQ | CoV2 | CoV2 | Science Translational |
| | VSVIYSGGSTFYADSVKGRFTISRD | | APRLLIYGASSRATGIPDRFSGS | | | Medicine, |
| | NSKNTLYLQMNSLRAEDTAVYYC | | GSGTDFTLTISRLEPEDFAVYY | | | 13(577): eabf1555 |
| | ARETYGMDVWGQGTTVTVSS | | CQQYGSSPWTFGQGTKVEIK | | | |
| 412 | QLQLQESGPGLVKPSETLSLTCTVS | 413 | QSALTQPASVSGSPGQSITISCT | SARS- | SARS- | Wang et al., 2021, |
| | GGSISRSDYYWGWIRQPPGKGLE | | GTSSDVGSYNLVSWYQQHPG | CoV2 | CoV2 | Science Translational |
| | WIGTIYYSGSTYYNPSLKSRVTISV | | KAPKLMIYEGSKRPSGVSSRFS | | | Medicine, |
| | DTSKNQFSLKLGSVTAADTAVYY | | GSKSGNTASLTISGLQGEDEAD | | | 13(577): eabf1555 |
| | CARQARTDASTYGHNFDSWGQGT | | YYCCSYAGSSTWVFAGGTKLT | | | |
| | LVT | | VL | | | |
| 414 | QLQLQESGPGLVKPSETLSLTCTVS | 415 | QSALTQPASVSGSPGQSITISCT | SARS- | SARS- | Wang et al., 2021, |
| | GGSISRSDFYWGWIRQPPGKGLEW | | GTSSDVGSYNLVSWYQQHPG | CoV2 | CoV2 | Science Translational |
| | IGTIYYSGSTYYNPSLKSRVTISVDT | | KAPKLMIYEGSKRPSGVSSRFS | | | Medicine, |
| | SKNQFSLKLDSVTAADTAVYYCA | | GSKFGNTASLTIYGLQGEDEA | | | 13(577): eabf1555 |
| | RQARTDGSTYGHNFDYWGQGTLV | | DYYCCSYAGSITWVFGGGTKL | | | |
| | TVSS | | TVL | | | |
| 416 | QVQLVQSGAEVKKPGASVKVSCK | 417 | QSVLTQPASVSGSPGQSITISCT | SARS- | SARS- | Wang et al., 2021, |
| | ASGYTFTGYYMHWVRQAPGQGLE | | GTSSDVGGYNYVSWYQQHPG | CoV2 | CoV2 | Science Translational |
| | WMGWINPNSGGTNYAQKFQGWV | | KAPKLMIYDVSNRPSGVSNRF | | | Medicine, |
| | TMTRDTSISTAYMELSRLRSDDTA | | SGSKSGNTASLTISGLQAEDEA | | | 13(577): eabf1555 |
| | VYYCARESHGGVWSAPGYYYGM | | DYYCSSYTNSSAVVFGGGTKL | | | |
| | DVWGQGT | | TVL | | | |
| 418 | EVQLVESGGGLVKPGGSLRLSCAA | 419 | EIVLTQSPGTLSLSPGERATLSC | SARS- | SARS- | Wang et al., 2021, |
| | SGFTFSSYSMNWVRQAPGKGLEW | | RASQSVSSSYLAWYQQKPGQ | CoV2 | CoV2 | Science Translational |
| | VSSISRDSKYIYYAESVKGRFTISR | | APRLLIYGASRRATGIPDRFSG | | | Medicine, |
| | DNAKNSLYLQMNSLIVEDTAVYY | | SGSGTDFTLTISRLEPEDFAVY | | | 13(577): eabf1555 |
| | CARDDGIAAASDYWGQGTLVTVSS | | YCQQYGSSPLLTFGGGTKVEIK | | | |
| 420 | QVQLQQWGAGLLKPSETLSRTCA | 421 | QSALTQPASVSGSPGQSITISCT | SARS- | SARS- | Wang et al., 2021, |
| | VYGGSFSGYYWSWIRQSPGKGLE | | GTSSDVGDYNYVSWYQQHPG | CoV2 | CoV2 | Science Translational |
| | WIGEINDSGSTNYNPSLKSRVTISV | | KAPKLMIYDVSNRPSGVSNRF | | | Medicine, |
| | DTSKNQFSLRLSSVTAADTAVYYC | | SGSKSGNTASLTISGLQAEDEA | | | 13(577): eabf1555 |
| | ARGHTQEKWELREGYYFDYWDQ | | DYYCSSYASGSTLYYVFGTGT | | | |
| | GTLVTVSS | | KVTVL | | | |
| 422 | EVQLLESGGGLVKPGGSLRLSCAA | 423 | NFMLTQPHSVSESPGKTVTISC | SARS- | SARS- | Wang et al., 2021, |
| | SGFTFSSYNMNWVRQAPGKGLEW | | TGSSGSIASNYVQWYQQRPGS | CoV2 | CoV2 | Science Translational |
| | VSSISSSSSHIYYADSVKGRFTISRD | | APTTVIYEDNQRPSGVPDRFSG | | (weak) | Medicine, |
| | NAKNSLYLQMNSLRAEDTAVYYC | | SIDSSSNSASLTISGLKTEDEAD | | | 13(577): eabf1555 |
| | ARERGYHGGKTSPFLGGQGTLVT | | YYCQSYDSSNYWVFGGGTKL | | | |
| | VSS | | TVL | | | |
| 424 | QVQLVESGAEVKKPGASVKVSCK | 425 | EIVLTQSPATLSLSPGERATLSC | SARS- | SARS- | Wang et al., 2021, |
| | ASGYTFTAYYMHWVRQAPGQGLE | | RASQSVSSYLAWYQQKPGQA | CoV2 | CoV2 | Science Translational |
| | WMGWINPNSGGTNYAQKFQGRV | | PRLLIYDASNRATGIPARFSGS | | | Medicine, |
| | TMTRDTSISTAYMELSRLRSDDTA | | GPGTDFTLTISSLEPEDFAVYY | | | 13(577): eabf1555 |
| | VYYCARPPRDYYDSSGYQIRDDHF | | CQQRSNCLFTFGPGTKVDIK | | | |
| | DYWGQGTLVTVSS | | | | | |
| 426 | EVQLVQSGAEVKKPGASVRVSCK | 427 | QSALTQPASVSGSPGQSITISCT | SARS- | SARS- | Wang et al., 2021, |
| | ASGYTFTSYDINWVRQATGQGLE | | GTSSDVGSYNLVSWYQQHPG | CoV2 | CoV2 | Science Translational |
| | WMGWMNPNSGNTGYAQKFQGRV | | KAPKLMIYEVSKRPSGVSNRFS | | | Medicine, |
| | TMTRNTSISTVYMELSSLRSEDTA | | GSKSGNTASLTISGLQAEDEAD | | | 13(577): eabf1555 |
| | VYYCARFPKVPAAIFPGDYYYGM | | YYCCSYAGSSTLVFGGGTKLT | | | |
| | DVWGQGTTVTVSS | | VL | | | |
| 428 | QVQLVESGGGVVQPGRSLRLSCA | 429 | DIQMTQSPSSLSASVGDRVTIS | SARS- | SARS- | Wang et al., 2021, |
| | ASGFTFSSYAMHWVRQAPGKGLE | | PKLLIYAASSLQSGVPSRFSGS | CoV2 | CoV2 | Science Translational |
| | CRASQSISSYLNWFQQKPGKA | | GSGTDFTLTISTLQPEDFATYY | | | Medicine, |
| | WVAVIWYDGSNKHYADSVKGRFT | | CQQSYGTPPWTFGQGTKVEIK | | | 13(577): eabf1555 |
| | ISRDNSKNTLYLQMNSLRAEDTAM | | | | | |
| | YYCARDEGSLTTTFDYWGQGTLV | | | | | |
| | TVSS | | | | | |
| 430 | EVQLVESGGGLVQPGGSLRLSCAA | 431 | QSALTQPPSVSGAPGQRVTISC | SARS- | SARS- | Wang et al., 2021, |
| | SGFTFSTYDMSWARQAPGKGLEW | | TGSSSNIGAGYDVHWYQQLPG | CoV2 | CoV2 | Science Translational |
| | VANIKQDGSEKYYVDSVKGRFTIS | | TAPKLLIYGNSNRPSGVPDRFS | | | Medicine, |
| | RDNAKNSLYLQMNSLRAEDTAVY | | GSKSGTSASLAITGLQAEDEAD | | | 13(577): eabf1555 |
| | YCARDTIPFWSGYYTSPDYYFDY | | YYCQSYDSSLSGVVFGGGTKL | | | |
| | WGQGTLVTVSS | | TVL | | | |
| 432 | QVQLQESGPGLVKPSQTLSLTCTV | 433 | QSALTQPPSASGSPGQSVTISC | SARS- | SARS- | Wang et al., 2021, |
| | SGGSISSGGYYWSWIRQHPGKGLE | | TGTSSDVGGYNYVSWYQQHP | CoV2 | CoV2 | Science Translational |
| | WIGYIYYSGTTYYNPSLKSRVTISV | | GKAPKLMIYEVSKRPSGVPDR | | | Medicine, |
| | DTSKNQFSLKLSSVTAADTAVYYC | | FSGSKSGNTASLTVSGLQAED | | | 13(577): eabf1555 |
| | ASSRYDFWSGSFDYWGQGTLVTV | | EADYYCSSYAGSNNWVFGGG | | | |
| | SS | | TKLTVL | | | |
| 434 | QVQLVQSGAEVKKPGASVKVSCK | 435 | QSALTQPASVSGSPGQSITISCT | SARS- | SARS- | Wang et al., 2021, |
| | TSGYIFTDYSMHWVRQAPGQGLE | | GTSSDVGGYNFVSWYQQHPG | CoV2 | CoV2 | Science Translational |
| | WMGWVNPNSGGTNYAQKFQGW | | KAPKLMIYEVSDRPSGVSNRFS | | | Medicine, |
| | VTMARDTSITTVYMELSRLRSDDT | | GSKSGNTASLTISGLQAEDEAD | | | 13(577): eabf1555 |
| | AVYYCARGPLPHRLVYDFWSGFH | | YYCSSYTSSSTWVFGGGTKLT | | | |
| | DAFDIWGQGTMVTVSS | | VL | | | |
| 436 | QVQLVESGGGVVQPGRSLRLSCA | 437 | DIQMTQSPSTLSASVGDRVTIT | SARS- | SARS- | Wang et al., 2021, |
| | ASGFTFSNYGMHWVRQAPGKGLE | | CRASQSISSWLAWYQQKPGKA | CoV2 | CoV2 | Science Translational |
| | WVAVIRYNGNNIYYADFVKGRFTI | | PKLLIYEASSLKSGVPSRFSGS | | (weak) | Medicine, |
| | SRDNSKNTLYLQMNSLRGEDTAV | | GSGTEFTLTISSLQPDDFATYY | | | 13(577): eabf1555 |
| | YYCAREGDGPDAFDIWGQGTMV | | CQQYNNYGITFGQGTRLEIK | | | |
| 438 | EVQLVESGGGLIQPGGSLRLSCAA | 439 | DIQLTQSPSFLSASVGDRVTITC | SARS- | SARS- | Wang et al., 2021, |
| | SGLTVSSNYMSWVRQAPGKGLEW | | RASQGVGSYLAWYQQKPGKA | CoV2 | CoV2 | Science Translational |
| | VSVIYSGGSTYYADSVRGRFTISRD | | PNLLIYAASTLQSGVPSRFSGS | | | Medicine, |
| | NSENTLYLQMNSLRAEDTAVYYC | | GSGTEFTLKITSLQPEDIATYY | | | 13(577): eabf1555 |
| | ARVGDWYFSWGQGTLVTVSS | | CQQLNSYPPLTFGGGTKVEIK | | | |
| 440 | QVQLQESGPGLVKPSETLSLTCTFS | 441 | EIVLTQSPGTLSLSPGERVTLSC | SARS- | SARS- | Wang et al., 2021, |
| | GGSISSYSWNWIRQPPGKGLEWIG | | RASQSITSTYLTWYQQKPGQA | CoV2 | CoV2 | Science Translational |
| | YIYTSGSTNYNPSLKSRVTMSVDT | | PRLLIYGASNRATGIPDRFSSSG | | (weak) | Medicine, |
| | SKNQFSLKLNSVTAADAAVYFCA | | SGTDFTLTISRLEPEDFAVYYC | | | 13(577): eabf1555 |
| | RGLFYNSGGYYSSNYYYYIDVWG | | QHYDRSPLCSFGQGTKLEIK | | | |
| | KGTTVTVSS | | | | | |
| 442 | EVQLVQSAAEVKKPGESLRISCKG | 443 | EIVLTQSPGILSLSPGERATLSC | SARS- | SARS- | Wang et al., 2021, |
| | SGYSFTNYWINWVRQMPGKGLEW | | RASQSVSSSYLAWYQQKPGQ | CoV2 | CoV2 | Science Translational |
| | MGRIDPSDSYTNYSPSFQGHVTISA | | APRLLIYGASSRAAGIPDRFSG | | | Medicine, |
| | DKSISTAYLQWSSLKASDTAMYYC | | SGSGTDFTLTISRLEPEDSAVY | | | 13(577): eabf1555 |
| | ARLNTDLRSRFGELYYYFDYWGQ | | YCQQYGSSLTWTFGQGTKVEI | | | |
| | GTLV | | K | | | |
| 444 | QVQLVQSGAEVKKPGASVKVSCK | 445 | DIQMTQSPSSLSASVGDRVTIT | SARS- | SARS- | Wang et al., 2021, |
| | ASGYTFTTYYLHWVRQAPGQGLE | | CQASQDITNHLNWYQQKPGK | CoV2 | CoV2 | Science Translational |
| | WMGIIDPSGGSTSYAQKFQGRVTM | | APKLLIYYASNLETGVPSRFSG | | | Medicine, |
| | TKDTSTSTVYMELSSLRSEDTAVY | | IGSGTDFTFTISSLQPEDIATYY | | | 13(577): eabf1555 |
| | YCARVGRGFSYGYFDYWGQGAL | | CQQYDNLPPLTFGGGTKVEIK | | | |
| | VTVSS | | | | | |
| 446 | QVQLVQSGAEVKKPGSSVKVSCK | 447 | DIVMTQSPLSLPVTPGEPASISC | SARS- | SARS- | Wang et al., 2021, |
| | ASGGTFNSYAISWVRQAPGQGLE | | RSSQSLLHSNGYNYLDWYLQ | CoV2 | CoV2 | Science Translational |
| | WMGGIIPIFGTANYAQKFQGRVTIT | | KPGQSPQLLIYLGSNRASGVPD | | (weak) | Medicine, |
| | ADESTSTAYMELSSLRSEDTAVYY | | RFSGSGSGTDFTLKISRVEAED | | | 13(577): eabf1555 |
| | CARVGAEWPRDHKYYYYGMDV | | VGVYYCMQALQTPLTFGGGT | | | |
| | WGQGTTVTVSS | | KVEIK | | | |
| 448 | QVQLVESGGGLVQPGGSLRLSCAA | 449 | DIQMTQSPSSLSASVGDRVTIT | SARS- | SARS- | Wang et al., 2021, |
| | SGFTFSNYDMHWVRQATGRGLEW | | CRASQSISSYLNWYQQKPGKA | CoV2 | CoV2 | Science Translational |
| | VSTIGTAGDTYYPGSVKGRFTISRE | | PKLLIYAASSLQSGVPSRFSGS | | (weak) | Medicine, |
| | NAKNSLYLQMNSLRAGDTAVYYC | | GSGTDFTLTISSLQPEDFATYY | | | 13(577): eabf1555 |
| | ARVKYGGYVGYFDYWGQGTLVT | | CQQSYSTPELTFGGGTKVEIK | | | |
| | VSS | | | | | |
| 450 | EVQLVESGGGLVQPGGSLRLSCAA | 451 | DIQMTQSPSSLSASVGDRVTIT | SARS- | SARS- | Wang et al., 2021, |
| | SGFTVSSNYMSWVRQAPGKGLEW | | CQASQDISNYLNWYQQKPGK | CoV2 | CoV2 | Science Translational |
| | VSLIYSGGTTYYADSVKGRFTISRD | | APKLLIYDASNLETGVPSRFSG | | | Medicine, |
| | NSKNTLYLQMNSLRAEDTAVYYC | | SGSGTDFTFTISSLQPEDIATYY | | | 13(577): eabf1555 |
| | ARETLGRGGDYWGQGTLVTVSS | | CQQYDNLPRSFGQGTKLEIK | | | |
| 452 | EVQLVESGGGLVQPGRSLRLSCAA | 453 | QSVLTQPPSASGTPGQRVTISC | SARS- | SARS- | Wang et al., 2021, |
| | SGFTFDDYAMHWVRQAPGKGLE | | SGSSSNIGSNTVNWYQQLPGT | CoV2 | CoV2 | Science Translational |
| | WVSGVSWNSGTIGYADSVKGRFTI | | APKLLIYSNNQRPSGVPDRFSG | | | Medicine, |
| | SRDNAKNSLYLQMNSLRAEDTAL | | SKSGTSASLAISGLQSEDEADY | | | 13(577): eabf1555 |
| | YYCAKIADIVRAYDFWSGQHFDAF | | YCAAWDDSLVVFGGGTKLTV | | | |
| | DIWGQGTMVT | | L | | | |
| 454 | EVQLVESGGGLVQPGRSLRLSCAA | 455 | DIQMTQSPSTLSASVGDRVTIT | SARS- | SARS- | Wang et al., 2021, |
| | SGFTFDDYAMHWVRQAPGKGLE | | CRASQSISSWLAWYQQKPGKA | CoV2 | CoV2 | Science Translational |
| | WVSIISWNSDNIGYADSVKGRFTIS | | PNLLIYTASNLESGVPSRYSGS | | | Medicine, |
| | RDNARNSLYLQMNSLRVEDTALY | | GSGTEFTLSISSLQPEDSATYFC | | | 13(577): eabf1555 |
| | YCAKDKGSGSGYGMDVWGRGTT | | QRYNSYPYSFGQGTKLEIK | | | |
| | VTVSS | | | | | |
| 456 | QLQLQESGPGLVKPSETLSLTCTVS | 457 | DIQMTQSPSSLSASVGDRVTIT | SARS- | SARS- | Wang et al., 2021, |
| | GGSISSSYYYWGWIRQPPGKGLEW | | CQASQDISNYLNWYQQKPGK | CoV2 | CoV2 | Science Translational |
| | IGSIYYSGSTYYNPSLKSRVTISVDT | | APKLLIYDASNLETGVPSRFSG | | (weak) | Medicine, |
| | SKNQFSLTLSSVTAADTAVYYCAT | | SGSGTDFTFTISSLQPEDIATYY | | | 13(577): eabf1555 |
| | GGRFWGWFDPWGQGTLVTVSS | | CQQYDNLPPKLTFGGGTKVEIK | | | |
| 458 | EVQLVQSGAEVKKPGESLKISCKG | 459 | QSVLTQPPSASGTPGQRVTISC | SARS- | SARS- | Wang et al., 2021, |
| | SGYSFISYWIGWVRQMPGKGLEW | | SGSSSNIGSNTVNWYQQLPGT | CoV2 | CoV2 | Science Translational |
| | MGIIYPGDSDTRYSPSFQGQVTISA | | APKLLIYSNNQRPSGVPDRFSG | | | Medicine, |
| | DKSINTAYLQWSSLKASDTAMYY | | SKSGTSASLAISGLQSEDEADY | | | 13(577): eabf1555 |
| | CARRVTIPNGWFDPWGQGTLVTV | | YCAAWDDSLNGVVFGGGTKL | | | |
| | SS | | TVL | | | |
| 460 | QVQLVQSGAEVKKPGSSVKVSCK | 461 | DIQMTQSPSSVSASVGDRVTIT | SARS- | SARS- | Wang et al., 2021, |
| | ASGGTFSSYAINWVRQAPGQGLE | | CRASQGISRWLAWYQQKPGK | CoV2 | CoV2 | Science Translational |
| | WMGGIIPIFGTANYAQKFQGRVTIT | | APKLLIYAAFSLQSGVPSRFSG | | | Medicine, |
| | ADESTSTAYMELSSLRSEDTALYY | | SGSGTDFTLTISSLQPEDFATY | | | 13(577): eabf1555 |
| | CARNRAVSEREDYYYGMDVWGQ | | YCQQANSFPLTFGGGTKVEIK | | | |
| | GTTVTVSS | | | | | |
| 462 | QVQLVQSGAEVKKPGASVKVSCK | 463 | QSVLTQPASVSGSPGQSITISCT | SARS- | SARS- | Wang et al., 2021, |
| | ASGYIFTDYSMITWVRQAPGQGLE | | GTSSDVGGYKFVSWYQQHPG | CoV2 | CoV2 | Science Translational |
| | WIGWVNPNSGGTNYAQKFQGWV | | KAPKLMIYEVSNRPSGVSNRFS | | | Medicine, |
| | TMARDTSITTVYMELSRLKSDDTA | | GSKSGNTASLTISGLQAEDEAD | | | 13(577): eabf1555 |
| | VYFCARGPLFHRLVYDFWSGYHD | | YYCNSYTSSSTWVFGGGTKLT | | | |
| | GFDMWGQGTMVTVSS | | VL | | | |
| 464 | EVQLVESGGGLIQPGGSLRLSCAA | 465 | EIVLTQSPGTLSLSPGERATLSC | SARS- | SARS- | Wang et al., 2021, |
| | SGLTVSRNYMNWVRQAPGKGLE | | RASQSFSSTYLAWYQQKPGQA | CoV2 | CoV2 | Science Translational |
| | WVSVMYSGGSTFYADSVKGRFTIS | | PRLLIYGASSRATGIPDRFSGSG | | | Medicine, |
| | RDNSKNTLYLQMNSLRAEDTAVY | | SGTDFTLTISRLEPEDFAVYYC | | | 13(577): eabf1555 |
| | YCARESYGMDVWGQGTTVTVSS | | QQYVTSPWTFGQGTKVEIK | | | |
| 466 | QVQLQQWGAGLLKPSETLSRTCA | 467 | QSALTQPASVSGSPGQSITISCT | SARS- | SARS- | Wang et al., 2021, |
| | VYGGSFSGYYWSWIRQSPGKGLE | | GTSSDVGDYNYVSWYQQHPG | CoV2 | CoV2 | Science Translational |
| | WIGEINDSGSTNYNPSLKSRVTISV | | KAPKLMIYDVSNRPSGVSNRF | | | Medicine, |
| | DTSKNQFSLRLSSVTAADTAVYYC | | SGSKSGNTASLTISGLQAEDEA | | | 13(577): eabf1555 |
| | ARGHTQEKWELREGYYFDYWDQ | | DYYCSSYASGSTLYYVFGTGT | | | |
| | GTLVTVSS | | KVTVL | | | |
| 468 | QVQLVESGGGVVQPGRSLRLSCA | 469 | DIQMTQSPSSLSASVGDRVTIT | SARS- | SARS- | Wang et al., 2021, |
| | ASGFPFSIYGMHWVRQAPGKGLE | | CRASQSISSYLNWYQQKPGKA | CoV2 | CoV2 | Science Translational |
| | WVAVISYDGGNKYYADSVKGRFT | | PKLLIYAASSLQSGVPSRFSGS | | | Medicine, |
| | ISRDNSKNTLYLQMNSLRAEDTAV | | VSGTDFTLTISSLLPEDFATYY | | | 13(577): eabf1555 |
| | YYCAKEGRPSDIVVVVAFDYWGQ | | CQQSYSTPRTFGQGTKVEIK | | | |
| | GTLVTVSS | | | | | |
| 470 | QVQLVQSGPEVKKPGTSVKVSCK | 471 | EIVLTQSPGTLSLSPGERATLSC | SARS- | SARS- | Wang et al., 2021, |
| | ASGFTFTSSAVQWVRQARGQRLE | | RASQSVSSSYLAWYQQKPGQ | CoV2 | CoV2 | Science Translational |
| | WIGWIVVGSGNTNYAQKFQERVTI | | APRLLIYGASSRATGIPDRFSGS | | | Medicine, |
| | TRDMSTSTAYMELSSLRSEDTAVY | | GSGTDFTLTISRLEPEDFAVYY | | | 13(577): eabf1555 |
| | YCAAPHCSGGSCYDAFDIWGQGT | | CQQYGSSPWTFGQGTKVEIK | | | |
| | MVTVSS | | | | | |
| 472 | QVQLVQSGPEVKKPGTSVKVSCK | 473 | EIVLTQSPGTLSLSPGERATLSC | SARS- | SARS- | Wang et al., 2021, |
| | ASGFTFTSSAVQWVRQARGQRLE | | RASQSVSSSYLAWYQQKPGQ | CoV2 | CoV2 | Science Translational |
| | WIGWIVVGSGNTNYAQKFQERVTI | | APRLLIYGASSRATGIPDRFSGS | | (weak) | Medicine, |
| | TRDMSTSTAYMELSSLRSEDTAVY | | GSGTDFTLTISRLEPEDFAVYY | | | 13(577): eabf1555 |
| | YCAAPHCSGGSCYDAFDIWGQGT | | CQQYGSSPWTFGQGTKVEIK | | | |
| | MVTVSS | | | | | |
| 474 | EVQLVESGGGLIQPGGSLRLSCAA | 475 | QSALTQPASVSGSPGQSITISCT | SARS- | SARS- | Wang et al., 2021, |
| | SGFTVSSNYMSWVRQAPGKGLEW | | GTSSDVGGYNYVSWYQQHPG | CoV2 | CoV2 | Science Translational |
| | VSVIYSGGSTYYADSVKGRFTISRD | | KAPKLMIYDVSNRPSGVSNRF | | | Medicine, |
| | NSKNTLYLQMNSLRAEDTAVYYC | | SGSKSGNTASLTISGLQAEDEA | | | 13(577): eabf1555 |
| | AREGDVEGYYDFWSGYSRDRYYF | | DYYCSSYTSSSTRVFGTGTKV | | | |
| | DYWGQGTLVTVSS | | TVL | | | |
| 476 | QVQLVQSGAEVKKPGASVKVSCK | 477 | QSALTQPPSASGSPGQSVTISC | SARS- | SARS- | Wang et al., 2021, |
| | ASGYTFTGYYMHWVRQAPGQGLE | | TGTSSDVGGYNYVSWYQQRP | CoV2 | CoV2 | Science Translational |
| | WMGWINPNSGGTNYAQKFQGRV | | GKAPKLMIDEVTKRPSGVPDR | | | Medicine, |
| | TMTRETSISTVYMELSRLRSDDTA | | FSGSKSGNTASLTVSGLQAED | | | 13(577): eabf1555 |
| | VYYCARDLGWSRLHGAFDIWGQG | | EADYYCSSYAGSNNWVFGGG | | | |
| | TMVTVSS | | TKLTVL | | | |
| 478 | QVQLQESGPGLVKPSGTLSLTCTV | 479 | QSVLTQPPSASGTPGQRVTISC | SARS- | SARS- | Wang et al., 2021, |
| | SSDSVSSYYWNWIRQTTGKGMEW | | SGISSNLGSNTVNWFQQLPGT | CoV2 | CoV2 | Science Translational |
| | IGRIDTGGRTNYNPSLSSRVAMSM | | APKLLIYNSNRRPSGVPDRFSG | | | Medicine, |
| | ATSKNQFSLKLSSVTAADTAVYFC | | SKSGTSASLAISGLQSEDEGDY | | | 13(577): eabf1555 |
| | ARGLSYYPLYGSHINYIDYWGQGT | | YCAEWDDSLSTWVFGGGTHL | | | |
| | LVTVSS | | TVL | | | |
| 480 | QVQLQESGPGLVKPSGTLSLTCTV | 481 | QSVLTQPPSASGTPGQRVTISC | SARS- | SARS- | Wang et al., 2021, |
| | SSDSVSSYYWNWIRQTTGKGMEW | | SGISSNLGSNTVNWFQQLPGT | CoV2 | CoV2 | Science Translational |
| | IGRIDTGGRTNYNPSLSSRVAMSM | | APKLLIYNSNRRPSGVPDRFSG | | | Medicine, |
| | ATSKNQFSLKLSSVTAADTAVYFC | | SKSGTSASLAISGLQSEDEGDY | | | 13(577): eabf1555 |
| | ARGLSYYPLYGSHINYIDYWGQGT | | YCAEWDDSLSTWVFGGGTHL | | | |
| | LVTVSS | | TVL | | | |
| 482 | QVQLQESGPGLVKPSGTLSLTCAV | 483 | EIVLTQSPATLSLSPGERATLSC | SARS- | SARS- | Wang et al., 2021, |
| | SGGSISSSNWWSWVRQPPGKGLE | | RASQSVSSYLAWYQQKPGQA | CoV2 | CoV2 | Science Translational |
| | WIGEIYHSGSTNYNPSLKSRVTISV | | PRLLIYDASNRATGIPARFSGS | | (weak) | Medicine, |
| | DKSKNQFSLKLSSVTAADTAVYYC | | GSGTDFSLTISSLEPEDFAVYY | | | 13(577): eabf1555 |
| | ATHLRLGELRGVDYWGQGTLVTV | | CQQRSNWPPWTFGQGTKVEIK | | | |
| | SS | | | | | |
| 484 | EVQLLESGGGLVKPGGSLRLSCAA | 485 | NFMLTQPHSVSESPGKTVTISC | SARS- | SARS- | Wang et al., 2021, |
| | SGFTFSSYSMNWVRQAPGKGLEW | | TGSSGSIASNYVQWYQQRPGS | CoV2 | CoV2 | Science Translational |
| | VSSISSSSSYIYYADSVKGRFTISRD | | APTTVIYEDNQRPSGVPDRFSG | | | Medicine, |
| | NAKNSLYLQMNSLRAEDTAVYYC | | SIDSSSNSASLTISGLKTEDEAD | | | 13(577): eabf1555 |
| | ARERGYYGGKTPPFLGGQGTLVT | | YYCQSYDSSNYWVFGGGTKL | | | |
| | VSS | | TVL | | | |
| 486 | QVQLVQSGAEVKKPGSSVKVSCK | 487 | EIVLTQSPATLSLSPGERATLSC | SARS- | SARS- | Wang et al., 2021, |
| | ASGGTFSSYAISWVRQAPGQGLEW | | RASQSVSSYLAWYQQKPGQA | CoV2 | CoV2 | Science Translational |
| | MGGIIPIFGTANYAQKFQGRVTITA | | PRLLIYDASNRATGIPARFSGS | | | Medicine, |
| | DESTSTAYMELSSLRSEDTAVYYC | | GSGTDFTLTISSLEPEDFAVYY | | | 13(577): eabf1555 |
| | ARGNRLLYCSSTSCYLDAVRQGY | | CQQRSNWPLTFGGGTKVEIK | | | |
| | YYYYYMDVWGKGTTVTVSS | | | | | |
| 488 | QVQLVQSGAEVKKPGASVKVSCK | 489 | QSALTQPASVSGSPGQSITISCT | SARS- | SARS- | Wang et al., 2021, |
| | ASGYTFTGYYMHWVRQAPGQGLE | | GTSSDVGGYNYVSWYQQHPG | CoV2 | CoV2 | Science Translational |
| | WMGWINPNSGGTNYAQKFQGRV | | KAPKLMIYDVSNRPSGVSNRF | | | Medicine, |
| | TMTRDTSIITAYMELSRLRSDDTA | | SGSKSGNTASLTISGLQAEDEA | | | 13(577): eabf1555 |
| | VYYCARAPPFYDFWSGIDYWGQG | | DYYCSSYTSSSTLVFGGGTKLT | | | |
| | TLVTVSS | | V | | | |
| 490 | QVQLVQSGAEVKKPGSSVKVSCK | 491 | DIQMTQSPSSLSASVGDRVTIT | SARS- | SARS- | Wang et al., 2021, |
| | ASGGTFSTYAISWVRQAPGQGLE | | CRASQSISSYLNWYQQKPGKA | CoV2 | CoV2 | Science Translational |
| | WMGGIIPIFGTANYAQKFQGRVTIT | | PKLLIYVASSLQSGVPSRFSGS | | (weak) | Medicine, |
| | ADESTSTAYMELSSLRSEDTAVYY | | GSGTDFTLTISSLQPEDFATYY | | | 13(577): eabf1555 |
| | CARDLRNCSSTSCYYWFDPWGQG | | CQQSYSTRTFGQGTKVEIK | | | |
| | TLVTVSS | | | | | |
| 492 | QVQLVQSGAEVKKPGSSVKVSCK | 493 | EIVLTQSPATLSLSPGERATLSC | SARS- | SARS- | Wang et al., 2021, |
| | ASGGTFSSYAISWVRQAPGQGLEW | | RASQSVSSYLAWYQQKPGQA | CoV2 | CoV2 | Science Translational |
| | MGGIIPIFGTANYAQKFQGRVTITA | | PRLLIYDASNRATGIPARFSGS | | | Medicine, |
| | DESTSTAYMELSSLRSEDTAVYYC | | GSGTDFTLTISSLEPEDFAVYY | | | 13(577): eabf1555 |
| | ARGNRLLYCSSTSCYLDAVRQGY | | CQQRSNWPLTFGGGTKVEIK | | | |
| | YYYYYMDVWGKGTTVTVSS | | | | | |
| 494 | QVQLVQSGAEVKKPGASVKVSCK | 495 | DIQMTQSPSSLSASVGDRVTIT | SARS- | SARS- | Wang et al., 2021, |
| | ASGYTFTGYYMHWVRQAPGQGLE | | CQASQDISNYLNWYQQKPGK | CoV2 | CoV2 | Science Translational |
| | WMGWINPISGGTNYAQKFQGRVT | | APKLLIYDASNLETGVPSRFSG | | | Medicine, |
| | MTRDTSISTAYMELSRLRSDDTAV | | SGSGTDFTFTISSLQPEDIATYY | | | 13(577): eabf1555 |
| | YYCASPASRGYSGYDHGYYYYMD | | CQQYDNLPITFGQGTRLEIK | | | |
| | VWGKGTTVTVSS | | | | | |
| 496 | QVQLVQSGAEVKKPGASVKVSCK | 497 | QSALTQPASVSGSPGQSITISCT | SARS- | SARS- | Wang et al., 2021, |
| | ASGYTFTGYFMHWVRQAPGQGLE | | GTSSDVGGYNYVSWYQQHPG | CoV2 | CoV2 | Science Translational |
| | WMGWINPNSGGTNYAQKFQGRV | | KAPKVMIYDVSNRPSGVSNRF | | (weak) | Medicine, |
| | TMTRDTSITTAYMELSRLRSDDTA | | SGSKSGNTASLTISGLQAEDEA | | | 13(577): eabf1555 |
| | VYYCARDDTLLRYSDWLPTTSFG | | DYYCSSYTSSSTNVFGTGTKV | | | |
| | GMDVWGQGTTVTVSS | | TVL | | | |
| 498 | QVQLVESGGGVVQPGRSLRLSCA | 499 | QSALTQPRSVSGSPGQSVTISC | SARS- | SARS- | Wang et al., 2021, |
| | ASGFTFSSYGMHWVRQAPGKGLE | | TGTSSDVGGYNYVSWYQQHP | CoV2 | CoV2 | Science Translational |
| | WVAVIRYDGSNKYYADSVKGRFT | | GKAPKLMIYDVSKRPSGVPDR | | | Medicine, |
| | ISRDNSKNKLYLQMNSLRAEDTAV | | FSGSKSGNTASLTISGLQAEDE | | | 13(577): eabf1555 |
| | YYCAREDYYDSSGSLDYWGQGTL | | ADYYCCSYAGSPWVFGGGTK | | | |
| | VTVSS1 | | LTVL | | | |
| 500 | QVQLVQSGPEVKKPGTSVKVSCK | 501 | EIVLTQSPGTLSLSPGERATLSC | SARS- | SARS- | Wang et al., 2021, |
| | ASGFTFTSSAVQWVRQARGQRLE | | RASQSVSSSYLAWYQQKPGQ | CoV2 | CoV2 | Science Translational |
| | WIGWIVVGSGNTNYAQKFQERVTI | | APRLLIYGASSRATGIPDRFSGS | | | Medicine, |
| | TRDMSTSTAYMELSSLRSEDTAVY | | GSGTDFTLTISRLEPEDFAVYY | | | 13(577): eabf1555 |
| | YCAAPHCSGGSCYDAFDIWGQGT | | CQQYGSSPYTFGQGTKLEIK | | | |
| | MVTVSS | | | | | |
| 502 | QVQLVQSGAEVKKPGASVKVSCK | 503 | QSALTQPASVSGSPGQSITISCT | SARS- | SARS- | Wang et al., 2021, |
| | ASGYTFTGYFLHWVRQAPGQGLE | | GTSSDVGYYNFVSWYQQHPG | CoV2 | CoV2 | Science Translational |
| | WVGWISPISGGTNYALKFQGRVT | | KAPKLMIFDVSNRPSGVSNRFS | | | Medicine, |
| | MTRDTSSTTAYMDLSRLRSDDTA | | GSKSGNTASLTISGLQAEDEAD | | | 13(577): eabf1555 |
| | VYYCAREALGYGDFPHDGFDLWG | | YYCSSYTSSSARVFGGGTKVT | | | |
| | KGTMVTVSS | | VL | | | |
| 504 | QVQLVESGGGVVQPGRSLRLSCA | 505 | DIQMTQSPSSLSASVGDRVTIT | SARS- | SARS- | Wang et al., 2021, |
| | ASGFTFSSYTMHWVRQAPGKGLE | | CRASQGISNYLAWYQQKPGK | CoV2 | CoV2 | Science Translational |
| | WVAVISYDGSNKYYADSVKGRFTI | | VPKLLIYAASTLQSGVPSRFSG | | | Medicine, |
| | SRDNSKNTLYLQMNSLRAEDTAV | | SGSGTDFTLTISSLQPEDVATY | | | 13(577): eabf1555 |
| | YYCARGPRGYYDFWSGYENEYYF | | YCQKYNSAPLTFGQGTRLEIK | | | |
| | DYWGQGTLVTVSS | | | | | |
| 506 | QVQLVESGGGVVQPGRSLRLSCA | 507 | EIVLTQSPATLSLSPGERATLSC | SARS- | SARS- | Wang et al., 2021, |
| | ASGFTFSSYAMHWVRQAPGKGLE | | RASQSVSSYLAWYQQKPGQA | CoV2 | CoV2 | Science Translational |
| | WVAVISYDGSNKYYADSVKGRFTI | | PRLLIYDASNRATGIPARFSGS | | | Medicine, |
| | SRDNSKNTLYLQMNSLRAEDTAV | | GSGTDFTLTISSLEPEDFAVYY | | | 13(577): eabf1555 |
| | YYCAREGAGPYYYDSSGYYTLFD | | CQQRSNWPTFGQGTRLEIK | | | |
| | DWGQGTLVTVSS | | | | | |
| 508 | QVQLVQSGAEVKKPGSSVRVSCK | 509 | QLVLTQPASVSGSPGQSIIVSCT | SARS- | SARS- | Wang et al., 2021, |
| | ASGGTFSTYPISWVRQAPGQGLEW | | GTSSDVGGYKFVSWYQQHPG | CoV2 | CoV2 | Science Translational |
| | MGGIIPIFGTAKSAQKFQGRVTITA | | KAPRVMIYDVSNRPSGVSNRF | | | Medicine, |
| | DEFTSTAYMEMSSLRSEDTAMYY | | SGSKSGNTASLTISGLQADDEA | | | 13(577): eabf1555 |
| | CAREGRRYGSGWYISTGYFDYWG | | DYYCSSYTNSSTVVFGGGTKV | | | |
| | QGTLVTVSS | | TVL | | | |
| 510 | QVQLVQSGGGVVQPGRSLRLSCV | 511 | SYELTQPPSVSVSPGQTATITCS | MERS- | MERS- | Tang, X. et al., 2014, |
| | ASEFTFNTYGMHWVRQAPGKGLE | | GDELGDKFAFWYQQKPGQSP | CoV | CoV | PNAS, |
| | WVAAISYDGTKKFYADSLKGRFTI | | VLVIYQDSKRPSGIPERFSGSNS | | | 111(19): E2018-26 |
| | SRDNSKNTLYLQMNSLRSEDTAV | | GNTATLTISGTQALDEADYYC | | | |
| | YYCARSGDSDAFDIWGQGTMVTV | | QAWDSNSYVFGTGTKVTVL | | | |
| | SS | | | | | |
| 512 | EVQLVQSGAEVKEPGSSVKVSCKA | 513 | QPVLTQPPSASGTPGQRVTISC | MERS- | MERS- | Tang, X. et al., 2014, |
| | SGGTFGSYAINWVRQAPGQRLEW | | SGSSSNIGSNYVFWYQQLPGM | CoV | CoV | PNAS, |
| | MGWIDAANGNTKYSQKFQGRVTI | | APKLLISRNNQRPSGVPDRFSG | | | 111(19): E2018-26 |
| | TGDTSASTAYMELSSLRSEDTAVY | | SKSGTSASLAISGPQSEDEADY | | | |
| | YCARDRWMTTRAFDIWGQGTMV | | YCAAWDDSLRGPVFGGGTRV | | | |
| | TVSS | | TVL | | | |
| 514 | QVQLVQSGAEVKKPGSSVKVSCK | 515 | ETTLTQSPATLSVSPGERATLS | MERS- | MERS- | Tang, X. et al., 2014, |
| | ASGGTFSSYAVSWVRQAPGQLEW | | CRASESVGSNLAWYQQKPGQ | CoV | CoV | PNAS, |
| | VGRIIPIFGKANYAQKFQGRVTITA | | APSLLIYGASTRATGIPDRFSGS | | | 111(19): E2018-26 |
| | DKSTSTAYMELSSLRPEDTAVYYC | | GSGTDFTLTISSLQSEDFAAYY | | | |
| | ARDQGISANFKDAFDIWGQGTTVT | | CQQYNNWPLTFGPGTKVEIK | | | |
| | VSS | | | | | |
| 516 | QVQLVQSGAEVKKPGSSVKVSCK | 517 | ETTLTQSPGTLSLSPGERATLS | MERS- | MERS- | Tang, X. et al., 2014, |
| | ASGGTFSSYAISWVRQAPGQGLEW | | CRASQSVSSSIAWYQQKPGQA | CoV | CoV | PNAS, |
| | MGGIIPIFGTANYAQKFQGRVTITA | | PRLLMFDSSTRATGIPDRFSGS | | | 111(19): E2018-26 |
| | DKSTSTAYMELSSLRSEDTAVYYC | | GSGTDFTLNISSLEPEDFAVYY | | | |
| | ARVGYCSSTSCHIGAFDIWGQGTT | | CQQYSSSPYTFGQGTKLEIK | | | |
| | VTVSS | | | | | |
| 518 | QVQLVQSGAEVKKPGSSVKVSCK | 519 | DIQMTQSPDSLAVSLGERATIN | MERS- | MERS- | Tang, X. et al., 2014, |
| | ASGGTFSSYAISWVRQAPGQGLEW | | CKSSQSVLYSSNNKNYLAWY | CoV | CoV | PNAS, |
| | MGGIIPIFGTANYAQKFQGRVTITA | | QQKPGQPPKLLIYWASARESG | | | 111(19): E2018-26 |
| | DKSTSTAYMELSSLRSEDTAVYYC | | VPDRFSGSGSGTDFTLTISSLQP | | | |
| | ARASYCSTTSCASGAFDIWGQGTL | | EDVAIYYCQQYYSVPFTFGPG | | | |
| | VTVSS | | TKVEIK | | | |
| 520 | EVQLVQSGAEVKKPGASVKVSCK | 521 | QPGLTQPPSVSKGLRQTATLTC | MERS- | MERS- | Tang, X. et al., 2014, |
| | ASGYTFNVYAINWVRQAPGQGLE | | TGNSNNVGNQGAAWLQQHQ | CoV | CoV | PNAS, |
| | WMGRIIPILGIANYAQKFQGRVTIT | | GHPPKLLSYTNNNRPSGISERL | | | 111(19): E2018-26 |
| | ADKSTSTAYMELSSLRSEDTAVYY | | SASRSGNTASLAITGLQPEDEA | | | |
| | CARDYYGSGARGFDYWGQGTLVT | | DYYCASWDSSLSVWVIGGGT | | | |
| | VSS | | KLTVL | | | |
| 522 | DVKLVESGGGLVKPGGSLKLSCA | 523 | DIQMTQTTSSLSASLGDRVTIS | MERS- | MERS- | Li, Y. et al., 2015, |
| | ASGFTFSSYTMSWVRQTPEKRLEW | | CRASQDISNYLNWYQQKPDGT | CoV | CoV | Cell Research, |
| | VATISSGGSYTYYPDSVKGRFTISR | | VKLLIYYTSRLHSGVPSRFSGS | | | 25: 1237-1249 |
| | DNAKNTLYLQMSSLKSEDTAMYY | | GSGTDYSLTISNLEQEDIATYF | | | |
| | CTRDGNDYDYWGQGTTLTVSS | | CQQGNTLPRTFGGGTKLEIK | | | |
| 524 | QVQLVQSGAEVKKPGSSVKVSCK | 525 | QSALTQPPSASGTPGQRVTISC | MERS- | MERS- | Choi et al., |
| | ASGGTFRSHAISWVRQAPGQGLE | | SGSSSNIGSNTVNWYQQLPGT | CoV | CoV | WO2019/039891 |
| | WMGGIIPFASANYAQKFQGRVTIT | | APKLLIYSNNQRPSGVPDRFSG | | | |
| | ADESTSTAYMDLSSLRSDDTAVYY | | SKSGTSASLAISGLQSEDEADY | | | |
| | CAKNVSPKSYSGRYSISYFYGVDV | | YCAAWDDSLSGHYVFGTGTK | | | |
| | WGQGTTVTVSS | | VTVL | | | |
| 526 | EVQLLESGGGLVQPGGSLRLSCAD | 527 | QSALTQPPSVSAAPGQKVTISC | MERS- | MERS- | Choi et al., |
| | SGLTFSSYAMSWVRQAPGKGLEW | | SGSSSNIGNNYVSWYQHLPGT | CoV | CoV | WO2019/039891 |
| | VSAISVSGGSTYYSDSVKGRFTISR | | APKLLIYDNMRPSGIPDRFSGS | | | |
| | DNSKNTLSLQMNSLRAEDTAVYY | | KSGTSATLGITGLQTGDEADY | | | |
| | CVKARSIVGPFDYWGQGTLVTVSS | | YCGTWDTSLSAVVFGGGTKLT | | | |
| | | | VL | | | |
| 528 | EVQLVESGAEVVKPGASVKMSCK | 529 | DIVMTQSPASLTVSLGQRATIS | MERS- | MERS- | Zhou et al., 2019, |
| | ASGYPFTSYNIHWIKQTPGQGLEW | | CRASKSVSASGYNYLHWYQQ | CoV | CoV | Nature |
| | IGAIYPGNGDTSYTQKFKVKATLT | | RPGQPPKLLIYLAFNLESGVPA | | | Communications, |
| | SDKSSSTAYMQLSSLTSEDSAVYF | | RFNGSGSGTDFTLNIHPVEEED | | | 10: 3068 |
| | CARYGNYPSYAMDYWGQGTSVT | | AATYYCQHSRDLPFTFGSGTK | | | |
| | VSS | | LEIK | | | |
| 530 | QVQLVQSGAEVKKPGSSVKVSCK | 531 | QSVLTQPPSVSGAPGQRVTISC | MERS- | MERS- | Choi et al., |
| | ASGGTFSSYTINWVRQAPGQGLE | | TGSSSNIGAGYDVHWYQQLPG | CoV | CoV | WO2019/039891 |
| | WMGGIIPIFGTANYAQKFQGRVTIT | | TAPKVLIYGNSNRPSGVPDRFS | | | |
| | ADASTSTAYMELSSLRSEDTAVYY | | GSKSDTSASLAITGLQAEDEAD | | | |
| | CARVLLRSSSWFSSNWFDPWGQG | | YYCQSYDSSLSVVFGGGTKLT | | | |
| | TLVTVSS | | VL | | | |
| 532 | QVQLVQSGAEVKRPGSSVKVSCK | 533 | EIVLTQSPATLSLSPGERATLSC | MERS- | MERS- | Choi et al., |
| | TSGGTFNNNAINWVRQAPGQGLE | | GASQSVSSSYLAWYQQKPGLA | CoV | CoV | WO2019/039891 |
| | WMGGIIPFFGIAKYAQKFQGRVTIT | | PRLLIYDASSRATGIPDRFSGSG | | | |
| | ADESTSTAYMELSSLRSEDTAVYY | | SGTDFTLTISRLEPEDFAVYYC | | | |
| | CARDLPRESSYGSGSYYTHYYAM | | QQYGSSPLTFGGGTKVEIK | | | |
| | DVWGQGTTVTVSS | | | | | |
| 534 | QVQLVQSGAEVKKPGASVKVSCK | 535 | DIQMTQSPSTLSASVGDRVTIT | MERS- | MERS- | Choi et al., |
| | ASGYTFTTYYMHWVRQAPGQGLE | | CRASQTISTWLAWYQQKPGK | CoV | CoV | WO2019/039891 |
| | WMGIINPSGGSTSYAQKFQGRVTM | | APKLLIYKASSLESGVPSRFSG | | | |
| | TRDTSTSTVYMELSSLRSEDTAVY | | SGSGTEFTLTISSLQPDDFATY | | | |
| | YCARGAVVVILDYWGQGTLVTVS | | YCQQYNSYSYTFGQGTKLEIK | | | |
| | S | | | | | |
| 536 | EVQLLESGAEVKKPGSSVKVSCRA | 537 | QSVLTQPPSVSGAPGQRVTPSC | MERS- | MERS- | Niu et al., 2018, J. |
| | SGGTFSSYAISWVRQAPGQGLEW | | TGSSSNIGAGYDVHWYQQLPG | CoV | CoV | Infectious Diseases, |
| | MGRIIPILGIANYAQKFQGRVTITA | | TAPKLLIYGNSNRPSGVPDRFS | | | 218: 1249-1260 |
| | DKATSTAYMELSSLRSEDTAVYYC | | GSKSGTSASLAITGLQAEDEAD | | | |
| | ATRSGDYYGSGSYSAFDIWGQGT | | YYCQSYDSSLSGLMFGGGTKL | | | |
| | MVTVSS | | TVL | | | |
| 538 | QVQLVQSGAEVEKAGASVKVSCK | 539 | QSVLTQPPSVSAAPGQKVTISC | MERS- | MERS- | Niu et al., 2018, J. |
| | ASGGTFSSYAISWVRQAPGQGLEW | | SGSRSNIGNNYVSWYQQLPGT | CoV | CoV | Infectious Diseases, |
| | MGRIIPILGIANYAQKFQGRVTITA | | APKLLIYDNNKRPSGIPDRSSG | | | 218: 1249-1260 |
| | DKSTSTAYMELSSLRSEDTAVYYC | | SKSGTSAALGITGLQTGDEAD | | | |
| | ARRSGDYYGSGSYSAFDIWGQGT | | YYCGTWDNSLSAVVFGGGTK | | | |
| | MVTVSS | | LTVL | | | |
| 540 | QVQLVQSGAEVKKPGSSVKVSCK | 541 | QSVLTQPPSVSGAPGQRVTPSC | MERS- | MERS- | Niu et al., 2018, J. |
| | ASGGTFSSYAISWVRQAPGQGLEW | | TGSSSNIGAGYDVHWYQQLPG | CoV | CoV | Infectious Diseases, |
| | MGRIIPILGIANYAQKFQGRVTITA | | TAPKLLIYGNSNRPSGVPDRFS | | | 218: 1249-1260 |
| | DKSTSTAYMELSSLRSEDTAVYYC | | GSKSGTSASLAITGLQAEDEAD | | | |
| | ARRSGDYYGSGSYSAFDIWGQGT | | YYCQSYDSSLSGLMFGGGTKL | | | |
| | MVTVSS | | TVL | | | |
| 542 | QVQLVQSGAEVKKPGSSVKVSCK | 543 | QSVLTQPPSVSGAPGQRVTISC | MERS- | MERS- | Niu et al., 2018, J. |
| | ASGGTFSSYAISWVRQAPGQGLEW | | TGSSSNIGAGYDVHWYQQLPG | CoV | CoV | Infectious Diseases, |
| | MGRIIPILGIANYAQKFQGRVTITA | | TAPKLLIYGNSNRPSGVPDRFS | | | 218: 1249-1260 |
| | DKSTSTAYMELSSLRSEDTAVYYC | | GSKSGTSASLAITGLQAEDEAD | | | |
| | ARRSGDYYGSGSYSAFDIWGQGT | | YYCGTWDSSLSAYVVFGGGT | | | |
| | MVTVSS | | KLTVL | | | |
| 544 | QVQLVQSGAEVKKPGSSVKVSCK | 545 | SYELTQPASVSGSPGQSITISCT | MERS- | MERS- | Niu et al., 2018, J. |
| | ASGGTFSSYAISWVRQAPGQGLEW | | GSSTDVGSSIYVSWYQQHPGK | CoV | CoV | Infectious Diseases, |
| | MGRIIPILGIANYAQKFQGRVTITA | | APQLILYDVTNRPSGVSTRFSG | | | 218: 1249-1260 |
| | DKSTSTAYMELSSLRSEDTAVYYC | | SKSGNTASLTISGLRAEDEADY | | | |
| | ARATRSDYYGSGSYSAFDIWGQGT | | YCNSYTTSNTLVFGGGTKLTV | | | |
| | MVTVSS | | L | | | |
| 546 | QMQLVQSGAEVKKPGASVKVSCK | 547 | NFMLTQPPSVSGAPGQRVTISC | MERS- | MERS- | Niu et al., 2018, J. |
| | ASGGTFSSYAISWVRQAPGQGLEW | | TGSSSNIGAGYDVHWYQQLPG | CoV | CoV | Infectious Diseases, |
| | MGRIIPILGIANYAQKFQGRVTITA | | TAPKLLIYGNSNRPSGVPDRFS | | | 218: 1249-1260 |
| | DKSTSTAYMELSSLRSEDTAVYYC | | GSKSGTSASLAISGLRSEDEAD | | | |
| | ARRSGDYYGSGSYSAFDIWGQGT | | YYCAAWDDSLSGYVFGTGTK | | | |
| | MVTVSS | | VTVL | | | |
| 548 | QMQLVQSGAEVKKPGASVKVSCK | 549 | QSVLTQPPSVSGAPGQRVTISC | MERS- | MERS- | Niu et al., 2018, J. |
| | ASGGTFSSYAISWVRQAPGQGLEW | | TGSSSNIGAGYDVHWYQQLPG | CoV | CoV | Infectious Diseases, |
| | MGRIIPILGIANYAQKFQGRVTITA | | TAPKLLIYGNSNRPSGVPDRFS | | | 218: 1249-1260 |
| | DKSTSTAYMELSSLRSEDTAVYYC | | GSKSGTSASLAITGLQAEDEAD | | | |
| | ARRSGDYYGSGSYSAFDIWGQGT | | YYCGTWDSSLSAYVVFGGGT | | | |
| | MVTVSS | | KLTVL | | | |
| 550 | QVQLVQSGAEVKKPGSSVKVSCK | 551 | DVVMTQSPLSLPVTPGEPASIS | MERS- | MERS- | Niu et al., 2018, J. |
| | ASGGTFSSYAISWVRQAPGQGLEW | | CRSSQSLLHSNGYNYLDWYLQ | CoV | CoV | Infectious Diseases, |
| | MGRIIPILGIANYAQKFQGRVTITA | | KPGQSPQLLIYLGSNRASGVPD | | | 218: 1249-1260 |
| | DKSTSTAYMELSSLRSEDTAVYYC | | RFSGSGSGTDFTLKISRVEAED | | | |
| | ARDSYGDYTGSGYYYGMDVWGQ | | VGVYYCMQALQTPLTFGGGT | | | |
| | GTTVTVSS | | KVEIK | | | |
| 552 | EVQLVQSGAEVKKPGSSVKVSCK | 553 | DIVMTQSPLSLPVTPGEPASISC | MERS- | MERS- | Niu et al., 2018, J. |
| | ASGGTFSSYAISWVRQAPGQGLEW | | RSSQSLLHSNGYNYLDWYLQ | CoV | CoV | Infectious Diseases, |
| | MGRIIPILGIANYAQKFQGRVTITA | | KPGQSPQLLIYLGSNRASGVPD | | | 218: 1249-1260 |
| | DKSTSTAYMELSSLRSEDTAVYYC | | RFSGSGSGTDFTLKISRVEAED | | | |
| | ARDSYGDYTGSGYYYGMDVWGQ | | VGVYYCMQALQTPLTFGGGT | | | |
| | GTTVTVSS | | KVEIK | | | |
| 554 | EVRLVQSGAEVEKAGASVKVSCK | 555 | QSVLTQPPSVSAAPGQKVTISC | MERS- | MERS- | Niu et al., 2018, J. |
| | ASGGTFSSYAISWVRQAPGQGLEW | | SGSRSNIGNNYVSWYQRPPGT | CoV | CoV | Infectious Diseases, |
| | MGRIIPILGIANYAQKFQGRVTITA | | APKLLIYDNNKRPSGIPDRFSG | | | 218: 1249-1260 |
| | DKSTSTAYMELSSLRSEDTAVYYC | | SKSGTSATLGITGLQTGDEAD | | | |
| | ARRSGDYYGSGSYSAFDIWGQGT | | YYCGTWDNSLSAVVNGGGTK | | | |
| | MVTVSS | | NTVV | | | |
| 556 | QVQLVQSGAEVKKPGASVKVSCK | 557 | QSVLTQPPSVSGAPGQRVTISC | MERS- | MERS- | Niu et al., 2018, J. |
| | ASGGTFSSYAISWVRQAPGQGLEW | | TGSSSNIGAGYDVHWYQQLPG | CoV | CoV | Infectious Diseases, |
| | MGRIIPILGIANYAQKFQGRVTITA | | TAPKLLIYGNSNRPSGVPDRFS | | | 218: 1249-1260 |
| | DKSTSTAYMELSSLRSEDTAVYYC | | GSKSGTSASLAITGLQAEDEAD | | | |
| | ARRSGDYYGSGSYSAFDIWGQGT | | YYCGTWDSSLSAYVVFGGGT | | | |
| | MVTVSS | | KLTVL | | | |
| 558 | QVQLVQSGAEVKKPGSSVKVSCK | 559 | QYALTQPASVSGSPGQSITISCT | MERS- | MERS- | Niu et al., 2018, J. |
| | ASGGTFSSYAISWVRQAPGQGLEW | | GSSTDVGSSIYVSWYQQHPGK | CoV | CoV | Infectious Diseases, |
| | MGRIIPILGIANYAQKFQGRVTITA | | APQLILYDVTNRPSGVSTRFSG | | | 218: 1249-1260 |
| | DKSTSTAYMELSSLRSEDTAVYYC | | SKSGNTASLTISGLRAEDEADY | | | |
| | ARRSGDYYGSGSYSAFDIWGQGT | | YCNSYTTSNTLVFGGGTKLTV | | | |
| | MVTVSS | | L | | | |
| 560 | QVQLVQSGAEVKKPGSSVKVSCK | 561 | QSALTQPRSVSGSPGQSVTISC | MERS- | MERS- | Niu et al., 2018, J. |
| | ASGGTFSTYALSWVRQAPGQGLE | | TGTSSDVGGYNYVSWYQQHP | CoV | CoV | Infectious Diseases, |
| | WMGGIIPIFGTANYAQKFQGRVTIT | | GKAPKLMIYDVSKRPSGVPDR | | | 218: 1249-1260 |
| | ADESTSTAYMELNSLRSEDTAVYY | | FSGSKSGNTASLTISGLQAEDE | | | |
| | CARGSRSSSSAEYFQHWGQGTLVT | | ADYYCCSYAGSYTLEWFGGG | | | |
| | VSS | | TKLTVL | | | |
| 562 | QVQLVESGGGLVKPGGSLRLSCAA | 563 | QSALTQPPSVSGAPGQRVTISC | MERS- | MERS- | Niu et al., 2018, J. |
| | SGFTFSDYYMSWIRQAPGKGLEW | | TGSSSNIGASYDVHWYQHLPG | CoV | CoV | Infectious Diseases, |
| | VSYISSSGSTIYYADSVKGRFTISRD | | TAPKLLIYGNTNRPSGVPDRFS | | | 218: 1249-1260 |
| | NAKNSLYLQMNSLRAEDTAVYYC | | GSKSGTSASLAITGLQAEDEAD | | | |
| | ARVGLGSGWYDWFDPWGQGTLV | | YYCQSYDSSLSGWFSGGTKLT | | | |
| | TVSS | | VL | | | |
| 564 | QVQLVQSGAEVKKPGSSVKVSCK | 565 | DVVMTQSPLSLPVTPGEPASIS | MERS- | MERS- | Niu et al., 2018, J. |
| | ASGGTFSIYAISWVRQAPGQGLEW | | CRSSQSLLHSNGYNYLDWYLQ | CoV | CoV | Infectious Diseases, |
| | MGGIIPIFGTANYAQKFQGRVTITA | | KPGQSPQLLIYLGSNRASGVPD | | | 218: 1249-1260 |
| | DKSTSTAYMELSSLRSEDTAVYYC | | RFSGSGSGTDFTLKISRVEAED | | | |
| | AREGGHQGYCSGGSCYDFDYWG | | VGVYYCMQALQTPAFGGGTK | | | |
| | QGTLVTVSS | | LEIK | | | |
| 566 | QLQLQESGPGLVKPSETLSLTCTVS | 567 | QSALTQPASVSGSPGQSITISCT | MERS- | MERS- | Niu et al., 2018, J. |
| | GGSISSSSYYWGWIRQPPGKGLEW | | GTSSDVGGYNYVSWCQQHPG | CoV | CoV | Infectious Diseases, |
| | IGSIYYSGSTYYNPSLKSRVTISVDT | | KAPKLMIYEVSNRPSGVSNRFS | | | 218: 1249-1260 |
| | SKNQFSLKLSSVTAADTAVYYCAS | | GSKSGNTASLTISGLQAEDEAD | | | |
| | LLRPLIYCSGGSCTDYWGQGTLVT | | YYCSSYTSNITLVFGTGTKVTV | | | |
| | VSS | | L | | | |
| 568 | EVKLVESGGGLVKPGGSLKLSCAA | 569 | DIQMTQTTSSLSASLGDRVTIIC | MERS- | MERS- | Wang et al., 2015, |
| | SGFTFSSYAMSWVRQTPEKRLEW | | RASQDINNYLNWYQQKPDGT | CoV | CoV | Nature |
| | VATISSGGTYTYYPDSVKGRFTISR | | VKLLIYYTSRLHSGVPSRFSGS | | | Communications, |
| | DNAENTLYLQMSSLRSEDTAMYY | | GSGSDYSLTISNLEQEDIATYF | | | 6: 7712 |
| | CVRDGNSMDYWGQGTSVTVSS | | CQQANTLPPTFGAGTKLELR | | | |
| 570 | EVKLEESGGGLVKPGGSLKLSCAA | 571 | DVIMTQIPLSLPVSLGDQASISC | MERS- | | Wang et al., 2015, |
| | SGFTFSRYAMSWWQTPEKRLEWV | | RSSQSIVHSNGNTYLEWYLQK | CoV | | Nature |
| | ATINNGGSYSYYPDSVKGRLTISRD | | PGQSPKPLIYKVSNRISGVPDR | | | Communications, |
| | NAKNTLYLQMSSLRSEDTALYYC | | FSGSGSGTDFTLKISRVEAEDL | | | 6: 7712 |
| | ARHYDYDGYYYTMDFWGQGTSV | | GVYYCFQGSHVPYTFGGGTNL | | | |
| | TVSS | | EIK | | | |
| 572 | QVQLQESGPGLVKPSETLSLTCAV | 573 | DIQMSQTPSSLSASVGDRVTIT | MERS- | | Wang et al. |
| | SGGSISSNYWYWIRQSPVKGLEWI | | CRASQGINDYLNWYQQKPGK | CoV | | WO2016138160 |
| | GYIYGGSGGTEYNPSLKSRVTISTD | | APKLLIYYGNSLASGVPSRFSG | | | |
| | TSKNQFFLKLSSVTAADTAVYYCA | | SGSGTDFSLTISSLQPEDFATY | | | |
| | RSFYSWNGESWGQGVWTVSS | | YCQQGDSFPLTFGGGTKVDIK | | | |
| 574 | EVQLVQSGAEVKKPGASVKVSCK | 575 | QSALTQPPSLSASPGASARLPC | MERS-CoV | MERS-CoV | Wang et al. |
| | ASGHIFTSYVINWLQAEPGQGFEW | | TLSSDLSVGSKNMYWYQQKP | | | WO2016138160 |
| | MGGIHPGNGGRDYAQKFQGRVTI | | GSAPRLFLYYYSDSDKQLGPG | | | |
| | TADMSTSTVYMELRSLRSEDMAV | | VPNRVSGSKETSSNTAFLLISG | | | |
| | YYCAASSGSYGVSSLDVWGRGVL | | LQPEDEADYYCQVYDSSANW | | | |
| | VTVSS | | VFGGGTRLTVL | | | |
| 576 | QVQLQQSGGELVKPGASVKLSCK | 577 | QLVLTQSPASLAVSLGQRATIS | MERS- | MERS- | Wang et al., 2019, |
| | TSGFTFSSSYISWLKQKPGQSLEWI | | CRASESVDNYGISFMNWF'QQK | CoV | CoV | Cell Reports, |
| | AWIYAGTGGTEYNQKFTGKAQVT | | PGQPPKLLIHTASNQGSGVPAR | | | 28(13): 3395-3405 |
| | VDTSSSTAYMQFSSLTTEDSAIYYC | | FSGSGSGTDFSLNIHPVEDDDT | | | |
| | ARGGSSFAMDYWGQGTSVTVSS | | AMYFCQQSEEVPLTFGAGTKL | | | |
| | | | EIK | | | |
| 578 | QVQLQQSGPELVRPGVSVKISCKG | 579 | DIVLTQSPASLAVSLGQRATIS | MERS- | MERS- | Pallesen et al., |
| | SGYTFTDYAIHWVKQSHAKSLEWI | | CRASESVDNYGISFMNWFQQK | CoV | CoV | PNAS, 2017, |
| | GVFSTYYGNTNYNQKFKGRATMT | | PGQPPKLLISATSNQGSGVPAR | | | 114(35): E7348-E7357 |
| | VDKSSSTAYMELARLTSEDSAIYY | | FIGSGSGTDFSLNIHPVEEDDT | | | |
| | CARKSYYVDYVDAMDYWGQGTS | | AMYFCQQSKEVPRTFGGGTKL | | | |
| | VTVSS | | EIK | | | |
| 580 | EVQLVESGGGLVQPGGSLRLSCSA | 581 | DIQMTQSPSTLSASVGDRVTIT | MERS- | MERS- | WO2015179535 |
| | SGFYFSSYDMSWVRQAPGKGLEW | | CRASQTISSWLAWYQQKPGK | CoV | CoV | |
| | VSAIRGSGHTTYYADSVKGRFTISR | | APKLLIYKASSLESGVPSRFSG | | | |
| | DNSKNTLYLEMNSLRAEDTAVYY | | SGSGTEFTLTISSLQPDDFATY | | | |
| | CVKDGSIVGFDPWGQGTLVTVSS | | YCQQYNSYSYTFGQGTKLEIK | | | |
| 582 | QVQLVQSGAEVKKPGSSVKVSCK | 583 | DIVMTQSPLSLPVTPGEPASISC | MERS- | MERS- | WO2015179535 |
| | ASGGSFSVYAISWVRQAPGQGLE | | RSSQSLLHGNGYNYLDWYLQ | CoV | CoV | |
| | WMGGIIPIFGTANYAQKFQDRFTIT | | KPGQSPQLLIYLVSHRASGVPD | | | |
| | TDESTSTAYMELSSLRSEDTAMYY | | RFSGSGSGTDFTLKISRVEAED | | | |
| | CAREGDIVVLPAGKGGMDVWGQ | | VGVYYCMQALQSPWTFGQGT | | | |
| | GTTVTVSS | | KVEIK | | | |
| 584 | EVQLVESGGGLVQPGGSLRLSCVV | 585 | DIQMTQSPSALSASVGDRVTIT | MERS- | MERS- | WO2015179535 |
| | SGFTFSNYDMSWVRQAPGRGLEW | | CRASQSISSWLAWYQQKPGKA | CoV | CoV | |
| | VSAIRGSGFNTYYADSVKGRFTISR | | PKLLIYKASSLESGVPSRFSGS | | | |
| | DNSKNTLYLQMNSLRAEDTAVYY | | GSGTEFTLTISSLQPDDFATYY | | | |
| | CAKDGSIVSMDYWGQGTLVTVSS | | CQQYNSYSWTFGQGTKVEIK | | | |
| 586 | QVQLQESGPGLVKPSETLSLTCTVS | 587 | EIVMTQSPATLSLSPGERATLS | MERS- | MERS- | WO2015179535 |
| | GGSISSYYWSWIRQPPGKGLEWIG | | CRASQSVSSNLAWYQQKPGQ | CoV | CoV | |
| | YIYYSGSPNYNPSLKSRVTISVDTS | | APRLLIYGASTRATGIPARFSG | | | |
| | KNQFSLKLTSVTAADTAVYYCARS | | SGSGTEFTLTISSLQSEDFAVY | | | |
| | LNWGPPFDYWGQGTLVTVSS | | YCQQFNNWPYTFGQGTKLEIK | | | |
| 588 | EVQLLESGGGLVQPGGSLRLSCAA | 589 | DIQMTQSPSSLSASVGDRVTIT | MERS- | MERS- | WO2015179535 |
| | SGFTFSSYAMSWVRQAPGKGLEW | | CQASQDISNYLNWYQQKPGK | CoV | CoV | |
| | VSAISGRGGNTYYADSVKGRFTIS | | APKFLIYDASNLETGVPSRFSG | | | |
| | RDNSKNTLFLQMNTLRAEDTAVY | | SGSGTDFTFTISSLQPEDIATYY | | | |
| | YCAKDRGFGFFDIWGRGTLATVSS | | CQQYDNLPFTFGPGTKINIK | | | |
| 590 | EVQLLESGPGVVRPSETLSLSCAVS | 591 | EIVMTQSPATLSLSPGERATLS | MERS- | MERS- | WO2016138160 |
| | GGSISDSYRWSWIRQPPGKGLEWV | | CRASQSVSSNLAWYQQKPGQ | CoV | CoV | |
| | GYIFATGTTTNYNPSLKSRVTISKD | | APRLLIHSASSRATGIPDRFSGS | | | |
| | TSKNQFSLKLSSVTAADTAVYYCA | | GSGTEFSLTISSLEAEDVGVYH | | | |
| | REPFKYCSGGVCYAHKDNSLDVW | | CYQHSSGYTFGPGTKLDIK | | | |
| | GQGVLVTVSS | | | | | |
| 592 | QVQLQESGPGLVKPSETLSLTCAV | 593 | DIVMTQTPFTLPVTPGEAASIS | MERS- | MERS- | WO2016138160 |
| | SGGSISSNYWNWIRQSPGKGLEWI | | CRSSQSLFDSDYGNTYLDWYL | CoV | CoV | |
| | GYIYGGSGSTTYNPSLKSRVAISTD | | QKPGQSPQLLIYMLSNRASGV | | | |
| | TSKDQFSLKLSSVTAADTAVYYCA | | PDRFSGSGSGTDFTLKISRVEA | | | |
| | RLLPLGGGYCFDYWGQGVLVTVS | | EDVGLYYCMQSVEYPFTFGPG | | | |
| | S | | TKLDIK | | | |
| 594 | QVQLQESGPGLVKPSETLSLTCAV | 595 | DIQMTQSPSSLSASVGDRVTIT | MERS- | MERS- | WO2016138160 |
| | SGDSISSNYWSWIRQPPGKGLEWI | | CRASQDINNYLSWYQQKPGK | CoV | CoV | |
| | GRFSGSGGSTDFNPSLKSRVTISTD | | APKPLIYYASSLETGVPSRFSG | | | |
| | TSKNQFSLNLRSVTAADTAVYYCA | | SRSGTDYTLTISSLQLEDFATY | | | |
| | KTYSGTFDYWGQGVLVTVSS | | YCQQYNNSPYSFGQGTKVEIK | | | |
| 596 | EVQLVESGGGLVKPGGSLRLSCAA | 597 | ELVLTQPPSASGTPGQRVNISC | MERS- | MERS- | KR101828794 |
| | SGFAFSSYSMIWVRQAPGKGLEW | | SGSRSNVGSNAVTWYQQVPG | CoV | CoV | |
| | VSSISTSSGYIYYADSVKGRFTISRD | | TAPKLLIYNNSKRPSGVPDRFS | | | |
| | NAKNSLYLQMNSLRAEDTAVYYC | | GSKSGTSASLAISGLQSEEEAD | | | |
| | ARAPIDAVAFDIWGQGTMVTVSS | | YYCAAWDDSLNGPVFGGGTK | | | |
| | | | VTVL | | | |
| 598 | EVQLLESGGGLVKPGGSLRLSCEA | 599 | QSALTQPASVSGSPGQSITISCT | MERS- | MERS- | Corti et al., PNAS, |
| | SGLTFSNVWMSWVRQAPGKGLE | | GTSSDVGTYDLVSWYQQHPG | CoV | CoV | 2015, 112(33): 10473- |
| | WVGRIKRKSEGAFIDYGAPVKGR | | KSPKLMIYADIKRPSGVSHRFS | | | 10478 |
| | FTLSRDDSKNTVYLQMNSLKIDDT | | GSKSGNTASLTISGLQSADEAD | | | |
| | AVYYCSTLTRGGDVWSSSYYFDY | | YYCCLYAGSSTSVIFGGGTKVT | | | |
| | WGQGALVTVSS | | | | | |
| 600 | QVQLKGSGPGLVAPSQSLSITCTVS | 601 | DIQMTQSPASLSASVGETVTIT | MERS- | MERS- | Li et al., Cell Res., |
| | GFSLTGYGVNWVRQPPGKGLEWL | | CRASENIYSYLAWYQQKQGKS | CoV | CoV | 2015, |
| | GMIWGDGSTDYNSALKSRLSISKD | | PQLLVYNAKTLAEGVPSRFSG | | | |
| | NSKSQVFLKMNSLQTDDTARYYC | | SGSGTQFSLKINSLQPEDFGSY | | | |
| | ARVGDYGDYFDYWGQGTTLTVSS | | YCQHHYGTPWFGGGTKLEIK | | | |
| 602 | EVQLVQSGAEVKKPGSSVKVSCK | 603 | ETTLTQSPATLSVSPGERAILSC | MERS- | MERS- | Tang et al., PNAS, |
| | ASGGTFSSYAISWVRQAPGQGLEW | | RASQSISNDLAWYQQKPGQAP | CoV | CoV | 2014, E2018-E2026. |
| | MGGIIPIFGIANYAQKFQGRVTITA | | RLLIYGASSRATGIPDRFSGSGS | | | |
| | DKSTSTAYMELSSLRSEDTAVYYC | | GTDFTFTISRLESEDFAVYYCQ | | | |
| | ASSNYYGSGSYYPRSAFDIWGQGT | | QYGVSPLTFGGGTKVEIK | | | |
| | TVTVSS | | | | | |
| 604 | QVQLVQSGAEVKKPGSSVKVSCK | 605 | DIQLTQSPSSLSASVGDRVTITC | MERS- | MERS- | Ying et al, J. of |
| | ASGGTFSSYAISWVRQAPGQGLEW | | RASQGIRNDLGWYQQKPGKA | CoV | CoV | Virology, 2014, |
| | MGGIIPIFGTASYAQKFQGRVTITA | | PKLLIYAASSLQSGVPSRFSGS | | | 88(14)7796-7805 |
| | DKSTSTAYMELSSLRSEDTAVYYC | | GSGTDFTLTISSLQPEDFATYY | | | |
| | ARVGYCSSTSCNRGAFDIWGQGT | | CQQLNSYPLTFGGGTKVEIK | | | |
| | MVTVSS | | | | | |
| 606 | QVQLQQSGAEVKKPGSSVKVSCK | 607 | EIVLTQSPLSLPVTPGEPASISC | MERS- | MERS- | Ying et al, J. of |
| | ASGGTFSSYTISWVRQAPGQGLEW | | RSSQSLLHSNGYNYLDWYLQ | CoV | CoV | Virology, 2014, |
| | MGRIIPIFGTANYAQKFQGRVTITA | | KPGKSPQLLIYLGSNRASGVPD | | | 88(14): 7796-7805 |
| | DKSTSTAYMELSSLRSEDTAVYYC | | RFSGSGSGTDFTLKISRVEAED | | | |
| | ARDLGPGGDSSGYYYGPGAFDIW | | VGVYYCMQALQTPLTFGGGT | | | |
| | GQGTMVTVSS | | KVEIK | | | |
| 608 | QVQLQQSGAEVKKRGSSVKVSCK | 609 | EIVMTQSPVTLSLSPGERATLS | MERS- | MERS- | Ying et al, J. of |
| | ASGGTFSSYTISWVRQAPGQGLEW | | CRASQSVSSYLAWYQQKPGQ | CoV | CoV | Virology, 2014, |
| | MGRIIPILGIANYAQKFQGRVTITA | | APRLLIYDASNRATGIPARFSG | | | 88(14): 7796-7805 |
| | DKSTSTAYMELSSLRSEDTAVYYC | | SGSGTDFTLTISSLEPEDFAVY | | | |
| | ARDLYDSSGYYRNTDAFDIWGQG | | YCQQYGSSPWTFGQGTKVEIK | | | |
| | TMVTVSS | | | | | |
| 610 | ATRLEESGAEVKKPGSSVKVSCKA | 611 | DVELTQSPGTLSLSPGERATLS | MERS- | MERS- | Chen et al., J. of |
| | SGGTFSSYAISWVRQAPGQGLEW | | CRASQSVSSSYLAWYQQKPGQ | CoV | CoV | Infectious Diseases, |
| | MGRIIPILGIANYAQKFQGRVTITA | | APRLLIYGASSRATGIPDRFSGS | | | 2017, 215(12): 1807- |
| | DKSTSTAYMELSSLRSEDTAVYYC | | GSGTDFTLTISRLEPEDFAVYY | | | 1815 |
| | ASKQGDYYDRTSYAFDIWGQGTM | | CQQYGSSPITFGQGTRLEIK | | | |
| | VTVSS | | | | | |
| 612 | VQLLETGGGLVKPGGSLRLSCAAS | 613 | DIRLTQSPSFLSASVGDRVTITC | MERS- | MERS- | Jiang et al., Science |
| | GFSLSDYYMNWIRQAPGKGLEWV | | RASQDINSFLAWYQQRPGKAP | CoV | CoV | Translational |
| | AYISSSSGYTNYGDSVKGRFTISRD | | KLLIYGASNLETGVPSRFSGGG | | | Medicine, 2014, |
| | HAKNSLYLQMNSLRVEDTAVYYC | | SGTDFTLTISSLQPEDIATYYCQ | | | 6(234): 234ra59 |
| | VRDRDDFWSGYYKHWGLGTLVT | | QYDKLPTFGQGTRLEIK | | | |
| | VSS | | | | | |
| 614 | VQLVESGGGLVQPGRSLRLSCAAS | 615 | QPVLTQSPSASGTPGQRVTISC | MERS- | MERS- | Zhang et al., 2018 |
| | GFTFSNYAMYWVRQAPGKGLEW | | SGSSSNIGNNYVYWYQQLPGT | CoV | CoV | Cell Reports, |
| | VALISYDISTDYYADSVKGRFTISR | | APKLLIYWNDQRPSGVPDRFS | | | 24(2): 441-452 |
| | DNSKNTIYLQMNNLRTEDTALYY | | GSKSGTSASLAISGLRSEDEAD | | | |
| | CAGNDYWGQGTLVTVSS | | YYCAAWDDSLSGAVFGGGTQ | | | |
| | | | LTVL | | | |
| 616 | EVQLVESGGGLVQPGRSLRLSCAA | 617 | GSQPVLTQSPSASGTPGQRVTI | MERS- | MERS- | Zhang et al., 2018 |
| | SGFTFSNYAMYWVRQAPGKGLEW | | SCSGSSSNIGNNYVYWYQQLP | CoV | CoV | Cell Reports, |
| | VALISYDISTDYYADSVKGRFTISR | | GTAPKLLIYWNDQRPSGVPDR | | | 24(2): 441-452 |
| | DNSKNTIYLQMNNLRTEDTALYY | | FSGSKSGTSASLAISGLRSEDE | | | |
| | CTNTYYWGQGTLVTVS | | ADYYCAAWDDSLSGAVFGGG | | | |
| | | | TQLTVL | | | |
| 618 | EVQLLESGGGLVQPGGSLRLSCAA | 619 | DIQMTQSPSSLSASVGDRVTIT | MERS- | MERS- | Pascal et al, PNAS, |
| | SGFTFSSYAMSWVRQAPGKGLEW | | CQASQDISNYLNWYQQKPGK | CoV | CoV | 2015, 112(28): 8738- |
| | VSAISGRGGNTYYADSVKGRFTIS | | APKFLIYDASNLETGVPSRFSG | | | 8743 |
| | RDNSKNTLFLQMNTLRAEDTAVY | | SGSGTDFTFTISSLQPEDIATYY | | | |
| | YCAKDRGFGFFDIWGRGTLATVSS | | CQQYDNLPFTFGPGTKINIK | | | |
| 620 | EVQLVESGGGLVQPGGSLRLSCVV | 621 | DIQMTQSPSALSASVGDRVTIT | MERS- | MERS- | Pascal et al, PNAS, |
| | SGFTFSNYDMSWVRQAPGRGLEW | | CRASQSISSWLAWYQQKPGKA | CoV | CoV | 2015, 112(28): 8738- |
| | VSAIRGSGFNTYYADSVKGRFTISR | | PKLLIYKASSLESGVPSRFSGS | | | 8743 |
| | DNSKNTLYLQMNSLRAEDTAVYY | | GSGTEFTLTISSLQPDDFATYY | | | |
| | CAKDGSIVSMDYWGQGTLVTVSS | | CQQYNSYSWTFGQGTKVEIK | | | |
| 622 | EVQLVESGGGLVQPGGSLRLSCAA | 623 | DIQMTQSPSSLSASVGDRVTIT | MERS- | MERS- | KR101828794 |
| | SGFTFSSYAMHWVRQAPGKGLEW | | CRASQSISNYLNWYQQKPGKA | CoV | CoV | |
| | VSSIYSSGGYIYYADSVKGRFTISR | | PKLLIYDASRLQSGVPSRFSGS | | | |
| | DNSKNTLYLQMNSLRAEDTAVYY | | GSGTDFTLTISSLQPEDFATYY | | | |
| | CAKDQYVSTDFDIWGQGTLVTVSS | | CQQSYSYPWTFGQGTKVEIK | | | |
| 624 | EVQLVESGGGLVQPGGSLRLSCAA | 625 | DIQMTQSPSSLSASVGDRVTIT | MERS- | MERS- | KR101828794 |
| | SGFTFSSYGMSWVRQAPGKGLEW | | CRASQSIGSYLNWYQQKPGKA | CoV | CoV | |
| | VSAISQSGGYIYYADSVKGRFTISR | | PKLLIYAASNLQSGVPSRFSGS | | | |
| | DNSKNTLYLQMNSLRAEDTAVYY | | GSGTDFTLTISSLQPEDFATYY | | | |
| | CAKHLYGSWAFDIWGQGTLVTVSS | | CQQSYSFPFTFGQGTKVEIK | | | |
| 626 | EVQLVESGGGLVQPGGSLRLSCAA | 627 | DIQMTQSPSSLSASVGDRVTIT | MERS- | MERS- | KR101828794 |
| | SGFTFSDYAMSWVRQAPGKGLEW | | CRASQSISSYLNWYQQKPGKA | CoV | CoV | |
| | VSAISQSGSYTNYADSVKGRFTISR | | PKLLIYGASSLQSGVPSRFSGS | | | |
| | DNSKNTLYLQMNSLRAEDTAVYY | | GSGTDFTLTISSLQPEDFATYY | | | |
| | CAKVSSQTLRFDYWGQGTLVTVSS | | CQQSYSFPFTFGQGTKVEIK | | | |
| 628 | QVQLQESGPGLVKPSETLSLTCSVS | 629 | EIVMTQSPATLSVSPGERATLS | SARS- | SARS- | Wang et al., Nature |
| | GGSISSHYWSWIRQPPGKGLEWIG | | CRASQSVSSSLAWYQQKPGQA | CoV1, | CoV2 and | Communications, |
| | YIYYSGSTNHNPSLKSRVTISVDTS | | PRLLIYGASTRAPGIPARFSGSG | SARS- | SARS- | (2020) 11: 2251 |
| | KNQFSLKLSSVTAADTAVYYCAR | | SGTEFTLTISSLQSEDFAVYYC | CoV2 | CoV1 | |
| | GVLLWFGEPIFEIWGQGTMVTVSS | | QQYNNWPLTFGGGTKVEI | | | |
| 630 | EVQLQQSGPVLVKPGASVRMSCK | 631 | NIMMTQSPSSLAVSAGEKVTM | SARS- | MERS- | Sauer et al., Nature |
| | ASGYTITDYYLNWVKQSHGKSLE | | SCKSSQSVLHSSDQKNYLAWY | CoV2, | CoV, | Structural & |
| | WLGVLNPYSGGSLYSQTFKGKAT | | QQKPGQSPKLLIYWASTRESG | MERS- | HKU4, | Molecular Biology |
| | LTVDRSSSTAYLELNSLTSEDSAVY | | VPDRFTGSGSGTDFTLTISSVQ | CoV, | OC43 | 28: 478-486(2021) |
| | YCARQLGRGNGLDYWGQGTSVT | | AEDLAVYFCHQYLSSYTFGGG | HKU4, | | |
| | VSS | | TKLEIK | OC43 | | |
| 632 | EVQLVQSGAEVKKPGESLKISCKG | 633 | QSVLTQPPSASGTPGQRVTISC | SARS- | SARS- | Scheid et al., 2021 |
| | SGYSFTSYWIGWVRQMPGKGLEW | | SGSSSNIGSDHVYWYQQLPGT | CoV2; | CoV2; | (sciencedirect.com/ |
| | MGVIYPGDSDTRYSPSFQGQVTISA | | APKLFIYRNNQRPSGVPDRFSG | SARS- | SARS- | science/article/pii/ |
| | DKSISTAYLQWSSLKASDTAMYYC | | SKSGTSASLAISGLRSEDEADY | CoV1 | CoV1 | S0092867421005353) |
| | ARTQWGYNYGSHFFYMDVWGKG | | YCAAWDASLSGYVFGTGTKV | | | |
| | TTVTVSS | | TVL | | | |
| 634 | QVQLVQSGAEVKKPGSSVKVSCK | 635 | EIVLTQSPGTLSLSPGERATLSC | SARS- | SARS- | Rogers et al., 2020 |
| | ASGGTFSSSAISWVRQAPGQGLEW | | RASQSVSSSYLAWYQQKPGQ | CoV1, | CoV2 | (science.sciencemag. |
| | MGGIIPILDITNYAQKFQGRVTITA | | APRLLIYGASSRATGIPDRFSGS | SARS- | and | org/content/early/ |
| | DKSTSTAFMELSSLRSEDTAVYYC | | GSGTDFTLTISRLEPEDFAVYY | CoV2 | SARS- | 2020/06/15/ |
| | ALRNQWDLLVYWGQGTLVTVSS | | CQHYGSSLWTFGQGTKLEIK | | CoV1 | science.abc7520) |
| 636 | VQLQQESGPGLVKPSETLSLTCTVS | 637 | DIVMTQSPSSLSASVGDRVTIT | SARS- | SARS- | Brouwer et al., 2020 |
| | GGSISSSSYYWGWIRQPPGKGLEW | | CRASQSISNYLNWYQQKPGKA | CoV1, | CoV1, | (science.sciencemag. |
| | IGSIYYSGSTYYNPSLKSRVTISVDT | | PKLLLYAASDLQSGVPSRFSGS | SARS- | SARS- | org/content/early/ |
| | SKNQFSLKLSSVTAADTAVYYCAR | | GSGTDFTLTISSLQPEDFATYY | CoV2 | CoV2 | 2020/06/15/science. |
| | RSTSRWGYYYMDVWGKGTRVTV | | CQQSYSTHMSTFGQGTKVDIK | | (weak) | abc5902) |
| | SS | | | | | |
| 638 | QVQLQESGPGLVKPSETLSLTCTVS | 639 | SYVLTQPPSVSVAPGQTARITC | SARS- | SARS- | Jennewein et al., |
| | GGSISSYYWSWIRQPPGKGLEWIG | | GGNNIGSKTVHWYQQKPGQA | CoV2; | CoV2; | 2021(biorxiv.org/ |
| | YVYYSGSTNYNPSLKSRVTISVDTS | | PVLVVYDDSDRPSGIPERFSGS | SARS- | SARS- | content/10.1101/2021. |
| | KNEFSLKLSSVTAADTAVYYCASS | | NSGNTATLTISRVEAGDEADY | CoV1 | CoV1 | 03.23.436684v1) |
| | QRPDGNLYYFDYWGQGTLVTVSS | | YCQVWDSSSDHYVFGTGTKV | | | |
| | | | TVL | | | |
| 640 | EVQLVQSGAEVKKPGATVKISCKV | 641 | IVLTQSPFQSVSPKEKVTITCRA | SARS- | SARS- | Zhe Lv et al., 2020 |
| | SGYSFSNYYIHWVKQAPGKSLEWI | | SQSISSNLHWYQQKPDQSPKL | CoV1, | CoV1, | (Science, doi: |
| | GYIDPFNGGTSDNLKFKGAATLTA | | LIKYASQSISGIPSRFSGSGSGT | SARS- | SARS- | 10.1126/science. |
| | DTSTDTAYMELSSLRSEDTAVYYC | | DFTLTINSLEAEDFGIYFCQQT | CoV2 | CoV2 | abc5881) |
| | ARSEYDPYYVMDYWGQGTTVTVSS | | NFWPYIFGQGTKLEIL | | | |
| 642 | QVQLQQSGAEVKKPGSSVKVSCK | 643 | SYELTQPPSVSVAPGKTARITC | SARS- | SARS- | Rouet et al., 2021 |
| | ASGGTFSTYSISWVRQAPGQGLEW | | GGNNIGSKSVHWYQQKPGQA | CoV1, | CoV1, | (MAbs doi: |
| | MGGIAPSHGFANYAQKFQGRVTIT | | PVLVVYDDSDRPSGIPERFSGS | SARS- | SARS- | 10.1080/19420862. |
| | TDESTSTAYMELSSLRSEDTAVYY | | NSGNTATLTISRVEAGDEADY | CoV2 | CoV2 | 2021.1922134) |
| | CARDTATGGMDVWGQGTTVTVSS | | YCQVWDTYSDYVFGTGTKVT | | | |
| | | | VL | | | |
| 644 | VQLVESGGGLVQPGGSLRLSCAAS | 645 | DIEMTQSPSSLSAAVGDRVTIT | SARS- | SARS- | Pinto et al., Nature |
| | GFTFSSYDMHWVRQTTGKGLEWV | | CRASQSIGSYLNWYQQKPGKA | CoV1, | CoV1, | 583: 290-295 (2020) |
| | STIGTAGDTYYPDSVKGRFTISRED | | PKLLIYAASSLQSGVPSRFSGS | SARS- | SARS- | |
| | AKNSLYLQMNSLRAGDTAVYYCA | | GSGTDFTLTISSLQPEDFAIYYC | CoV2 | CoV2 | |
| | RGDSSGYYYYFDYWGQGTLLTVSS | | QQSYVSPTYTFGPGTKVDIK | | (weak) | |
| 646 | QVQLVQSGPEVKKPGTSVRVSCK | 647 | DIVLTQTPGTLSLSPGERATLS | SARS- | SARS- | Starr, TN, et al., |
| | ASGFTFTSSAVQWVRQARGQRLE | | CRASQSVSSSYLAWYQQKPGQ | CoV2, | CoV2, | Nature, 2021 Jul 14. |
| | WVGWIVVGSGNTNYAQKFHERVT | | APRLLIYGASSRATGIPDRFSGS | SARS- | SARS- | doi: 10.1038/s41586- |
| | ITRDMSTSTAYMELSSLRSEDTAV | | GSGTDFTLTISRLEPEDFAVYY | CoV1 | CoV1 | 021-03807-6. |
| | YYCASPYCSGGSCSDGFDIWGQGT | | CQQYVGLTGWTFGQGTKVEIK | | | |
| | MVTVSS | | | | | |
| 648 | EVQLVESGGGLVNPGGSLRLSCAA | 649 | VTQPASVSGSPGQSITISCTGTS | SARS- | SARS- | PDB: 7LXX; |
| | SGFTFSDYTIHWVRQAPGKGLEW | | SDVGGYNYVSWYQQHPGKAP | CoV2 | CoV2 | McCallum et al., |
| | VSSISSSSNYIYYADSVKGRFTISRD | | KLMIYDVSDRPSGVSNRFSGS | | | (2021) Cell 184: |
| | NAKNSLSLQMNSLRAEDTAVYYC | | KSGNTASLTISGLQAEDEADY | | | 2332 |
| | ARDGNAYKWLLAENVRFDYWGQ | | YCSSYTSSSTPNWVFGGGTKLT | | | |
| | GTLVTVSS | | | | | |
| 650 | VQLVESGGGVVQPGRSLRLSCAAS | 651 | YELTQPPSVSVSPGQTARITCS | SARS- | SARS- | PDB: 7LY2; |
| | GFTFSSYGMHWVRQAPGKGLEWV | | GDALAKHYAYWYRQKPGQAP | CoV2 | CoV2 | McCallum et al., |
| | TVIWYDGSNRYYADSVKGRFTISR | | VLVIYKDSERPSGIPERFSGSSS | | | (2021) Cell 184: |
| | DNSKNTLYLQMDSLRAEDTAVYY | | GTTVTLTISGVQAEDEADYYC | | | 2332 |
| | CARAVAGEWYFDYWGQGTLVTVS | | QSADSIGSSWVFGGGTKLTV | | | |
| 652 | VQLVESGGGVVQPGRSLRLSCAAS | 653 | YELTQPPSVSTARITCGGNNIE | SARS- | SARS- | PDB: 7LXW; |
| | GFTFSNYGMHWVRQAPGKGLEW | | RKSVHWCQQKPGQAPALVVY | CoV2 | CoV2 | McCallum et al., |
| | VAVIWYDGSNKFYADSVKGRFTIS | | DDSDRPSGIPERFSGSNSGNTA | | | (2021) Cell 184: |
| | RDNSKNSLYLQMNSLRAEDTAVY | | TLTISRVEAGDEADYYCQVWD | | | 2332 |
| | FCARAFPDSSSWSGFTIDYWGQGT | | SGSDQVIFGGGTKLT | | | |
| | LVTV | | | | | |
| 654 | EVQLVESGGGLVKPGGSLRLSCAA | 655 | QPVLTQPPSVSGAPGQRITISCT | SARS- | SARS- | Rappazzo, et al |
| | SGFTFSSYYMNWVRQAPGKGLEW | | GSSSNIGAGYDVHWYQQLPGT | CoV2 | CoV2 | Science 371: 823-829 |
| | VSSISSDGYNTYYPDSLKGRFTISR | | APKLLIYGSSSRPSGVPDRFSG | | | DOI: 10.1126/ |
| | DSAKNSLYLQMNSLRADDTAVYY | | SKSGTSASLAITGLQAEDEADY | | | science.abf4830 |
| | CARDFSGHTAVAGTGFEYWGQGT | | YCQSYDSSLSVLYVFGTGTKV | | | |
| | LVTVSS | | TVL | | | |

*Sequence, binding, and neutralization data derived from the CoV-AbDab database: opig.stats.ox.ac.uk/webapps/covabdab/(last visited July 22, 2021)(Raybould, MIJ, et al., Bioinformatics doi = 10.1093/bioinformatics/btaa739(2020)).

What is claimed is:

1. A multimeric binding molecule comprising two to six bivalent binding units or variants or fragments thereof, wherein each binding unit comprises two IgM or IgA heavy chain constant regions or multimerizing fragments or variants thereof, each associated with a binding domain, wherein three to twelve of the binding domains are identical and specifically bind to a human coronavirus, and wherein the binding molecule is more potent than a bivalent reference IgG antibody comprising two of the binding domains that specifically bind to the human coronavirus.

2. The multimeric binding molecule of claim 1, wherein the human coronavirus is SARS-CoV, MERS-CoV, SARS-CoV-2, variants thereof, derivatives thereof, or any combination thereof.

3. The multimeric binding molecule of claim 1, wherein the binding molecule can neutralize infectivity of the human coronavirus at a lower 50% effective concentration (EC₅₀) than the bivalent reference IgG antibody, and wherein the EC₅₀ is at least two-fold, at least five-fold, at least ten-fold, at least fifty-fold, at least 100-fold, at least 500-fold, or at least 1000-fold lower than the EC₅₀ of the bivalent reference IgG antibody.

4. The multimeric binding molecule of claim 1, wherein the binding molecule can inhibit binding of the human coronavirus to its receptor at a lower 50% inhibitory concentration (IC₅₀) than the bivalent reference IgG antibody.

5. The multimeric binding molecule of claim 4, wherein the human coronavirus is SARS-CoV or SARS-CoV-2 and the receptor is human angiotensin-converting enzyme 2 (ACE2), or wherein the human coronavirus is MERS-CoV and the receptor is human dipeptidyl-peptidase 4 (DPP4).

6. The multimeric binding molecule of claim 1, wherein the three to twelve binding domains that specifically bind to a human coronavirus bind a human coronavirus structural protein or fragment thereof, wherein the human coronavirus structural protein comprises a nucleocapsid (N) protein, a membrane (M) protein, an envelope (E) protein, a spike (S) protein, any fragment thereof, any subunit thereof, or any combination thereof.

7. The multimeric binding molecule of claim 6, wherein the three to twelve binding domains specifically bind to the human coronavirus S protein, and wherein the binding domains bind the S protein subunit 1 (S1), the S protein receptor binding domain (RBD), the S protein subunit 2 (S2), the S protein furin cleavage site, or any combination thereof.

8. The multimeric binding molecule of claim 1, wherein the three to twelve identical binding domains are immunoglobulin antigen binding domains comprising a heavy chain variable region (VH) and a light chain variable region (VL), and wherein each binding unit comprises two heavy chains each comprising a VH and two light chains each comprising a VL.

9. The multimeric binding molecule of claim 8, wherein the human coronavirus is SARS-CoV-2, wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL of SEQ ID NO: 88 and SEQ ID NO: 89, SEQ ID NO: 260 and SEQ ID NO: 261, SEQ ID NO: 264 and SEQ ID NO: 265, SEQ ID NO: 266 and SEQ ID NO: 267, SEQ ID NO: 274 and SEQ ID NO: 275, SEQ ID NO: 278, and SEQ ID NO: 279, SEQ ID NO: 280 and SEQ ID NO: 281, SEQ ID NO: 282 and SEQ ID NO: 283, SEQ ID NO: 292 and SEQ ID NO: 293, SEQ ID NO:384 and SEQ ID NO: 385, or SEQ ID NO: 646 and SEQ ID NO: 647, respectively or wherein the VH and VL comprise amino acid sequences at least 80%, at least 85%, at least 90%, at least 95% or 100% identical to the recited VH and VL amino acid sequences, respectively, and wherein the bivalent reference IgG antibody comprising two of the binding domains can neutralize SARS-CoV-2.

10. The multimeric binding molecule of claim 8, wherein the human coronavirus is SARS-CoV, wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL of SEQ ID NO: 84 and SEQ ID NO: 85, SEQ ID NO: 260 and SEQ ID NO: 261, SEQ ID NO: 262 and SEQ ID NO: 263, SEQ ID NO: 296 and SEQ ID NO: 297, SEQ ID NO: 628 and SEQ ID NO: 629, SEQ ID NO: 632 and SEQ ID NO: 633, SEQ ID NO: 634 and SEQ ID NO: 635, SEQ ID NO: 636 and SEQ ID NO: 637, SEQ ID NO: 638 and SEQ ID NO: 639, SEQ ID NO: 640 and SEQ ID NO: 641, SEQ ID NO: 642 and SEQ ID NO: 643, or SEQ ID NO: 644 and SEQ ID NO: 645, respectively or wherein the VH and VL comprise amino acid sequences at least 80%, at least 85%, at least 90%, at least 95% or 100% identical to the recited VH and VL amino acid sequences, respectively, and wherein the bivalent reference IgG antibody comprising two of the binding domains can neutralize SARS-CoV.

11. The multimeric binding molecule of claim 8, wherein the human coronavirus is MERS-CoV, wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL of SEQ ID NO: 522 and SEQ ID NO: 523, SEQ ID NO: 576 and SEQ ID NO: 577, SEQ ID NO: 610 and SEQ ID NO: 611, SEQ ID NO: 612 and SEQ ID NO: 613, SEQ ID NO: 614 and SEQ ID NO: 615, or SEQ ID NO: 630 and SEQ ID NO: 631, respectively or wherein the VH and VL comprise amino acid sequences at least 80%, at least 85%, at least 90%, at least 95% or 100% identical to the recited VH and VL amino acid sequences, respectively, and wherein the bivalent reference IgG antibody comprising two of the binding domains can neutralize MERS-CoV.

12. The multimeric binding molecule of claim 1, which can neutralize escape mutants of the bivalent reference IgG antibody comprising two of the binding domains.

13. The multimeric binding molecule of claim 1, comprising two or four bivalent IgA or IgA-like binding units and a J chain or functional fragment or variant thereof wherein each binding unit comprises two IgA heavy chains each comprising a VH situated amino terminal to the IgA constant region or multimerizing fragment or variant thereof, and two immunoglobulin light chains each comprising a VL situated amino terminal to an immunoglobulin light chain constant region.

14. The multimeric binding molecule of claim 13, wherein the IgA heavy chain constant regions or multimerizing fragments or variants thereof are human IgA constant regions.

15. The multimeric binding molecule of claim 13, wherein the J-chain or functional fragment or variant thereof further comprises a heterologous polypeptide, wherein the heterologous polypeptide is directly or indirectly fused to the J-chain or functional fragment or variant thereof.

16. The multimeric binding molecule of claim 1, comprising five or six bivalent IgM or IgM-like binding units, wherein each binding unit comprises two IgM heavy chain constant regions or multimerizing fragments or variants thereof, wherein each binding unit comprises two IgM heavy chains each comprising a VH situated amino terminal to an IgM constant region or multimerizing fragment or variant thereof, and two immunoglobulin light chains each comprising a VL situated amino terminal to an immunoglobulin light chain constant region.

17. The multimeric binding molecule of claim 16, wherein the IgM heavy chain constant regions or multimerizing fragments or variants thereof are human IgM constant regions.

18. The multimeric binding molecule of claim 16 which is pentameric, and further comprises a J-chain or functional fragment or variant thereof.

19. The multimeric binding molecule of claim 18, wherein the J-chain or functional fragment or variant thereof further comprises a heterologous polypeptide, wherein the heterologous polypeptide is directly or indirectly fused to the J-chain or functional fragment or variant thereof.

20. A composition comprising the multimeric binding molecule of claim 1; or comprising two or more nonidentical multimeric binding molecules according to claim 1, wherein the two or more multimeric binding molecules bind to different epitopes of a single human coronavirus.

21. A polynucleotide comprising a nucleic acid sequence that encodes a polypeptide subunit of the binding molecule of claim 1.

22. A vector comprising the polynucleotide of claim 21.

23. A host cell comprising the polynucleotide of claim 21, wherein the host cell can express a multimeric binding molecule comprising two to six bivalent binding units or variants or fragments thereof, wherein each binding unit comprises two IgM or IgA heavy chain constant regions or multimerizing fragments or variants thereof, each associated with a binding domain, wherein three to twelve of the binding domains are identical and specifically bind to a human coronavirus, and wherein the binding molecule is more potent than a bivalent reference IgG antibody comprising two of the binding domains that specifically bind to the human coronavirus.

24. A method for treating or preventing a human coronavirus disease in a subject in need of treatment comprising administering to the subject an effective amount of the multimeric binding molecule of claim 1, wherein the multimeric binding molecule exhibits greater potency than an equivalent amount of a bivalent reference IgG antibody comprising two of the binding domains that specifically bind to the human coronavirus.

25. The method of claim 24, wherein the human coronavirus disease is severe acute respiratory syndrome (SARS), coronavirus disease 2019 (COVID-19), or Middle East Respiratory Syndrome (MERS).

26. The method of claim 24, wherein the subject is human.

27. The method of claim 24, wherein administering comprises intravenous, subcutaneous, intramuscular, intranasal, and/or inhalation administration.
